(19) United States
(12) Patent Application Publication
(10) Pub. No.: US 2024/0217947 A1
(43) Pub. Date: Jul. 4, 2024

(54) Morpholinylquinazoline Compound, Pharmaceutical compositions thereof and Use Thereof

(71) Applicant: SHANGHAI YINGLI PHARMACEUTICAL CO., LTD, Shanghai (CN)

(72) Inventors: Zusheng Xu, Shanghai (CN); Yangtong Lou, Shanghai (CN); Tiegang Xie, Shanghai (CN); Linlin XU, Shanghai (CN)

(73) Assignee: SHANGHAI YINGLI PHARMACEUTICAL CO., LTD, Shanghai (CN)

(21) Appl. No.: 18/056,877

(22) Filed: Nov. 18, 2022

(51) Int. Cl. C07D 401/04; C07D 401/14; C07D 405/14; C07D 413/14

(52) U.S. Cl. CPC C07D 401/04; C07D 401/14; C07D 405/14; C07D 413/14

(57) ABSTRACT

Disclosured are a morpholinylquinazoline compound, pharmaceutical compositions thereof and use thereof. The present disclosure provides a morpholinylquinazoline compound represented by formula I, a pharmaceutically acceptable salt thereof, a solvate thereof, a solvate of the pharmaceutically acceptable salt thereof, a polycrystalline form thereof or a precursor thereof. It is expected that the morpholinylquinazolines compound have dual inhibitory activities towards PI3Kδ and HDAC, which are expected to achieve better efficacy and wider application.

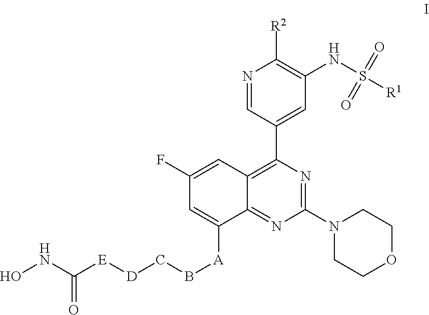

### TECHNICAL FIELD

The present disclosure relates to a morpholinylquinazoline compound, pharmaceutical compositions thereof and use thereof.

### BACKGROUND

Phosphoinositide 3-kinase(PI3K) is an intracellular phosphatidylinositol kinase which can catalyze the phosphorylation of hydroxyl group at 3-position of the phosphatidylinositol. The PI3K is classified into Class I, Class II and Class III kinase and the most extensively studied one is the Class I PI3K which can be activated by cell surface receptors. The Class I PI3K in mammalian cells is further divided into Class 1a and Class Ib based on the structure and receptor, which transducts signals from tyrosine kinase-coupled receptors and G protein-coupled receptors, respectively. The Class Ia PI3K includes PI3Kα, PI3Kβ and PI3Kδ subtypes, and the Class Ib PI3K includes PI3Kγ subtype (Trends Biochem.Sci., 1997, 22, 267-272). The Class Ia PI3K is a dimeric protein consisting of a p110 catlytic subunit and a p85 regulatory subunit and having dual activities of a lipid kinase and a protein kinase (Nat. Rev. Cancer 2002, 2, 489-501), and is considered to be correlated with cell proliferation and cancer development, immune diseases and inflammation related diseases.

Several PI3K δ inhibitors have been marketed successfully worldwide, such as Idelalisib, Copanlisib, and Duvelisb, all for the treatment of circulatory cancers. Linperlisib, chemical name known as N-(5-(6-fluoro-8-((4-(2-hydroxypropyl-2-yl)piperidin-1-yl)methyl)-2-morpholine-quinazolin-4-yl)-2-methoxypyridin-3-yl)methanesulfonamide, is a novel class of PI3Kδ inhibitor. Clinical studies have shown that Linperlisib shows a favorable safety profile and clinical benefit in patients with relapsed and/or refractory B-cell malignant hematologic neoplasms when administered orally at 80 mg once daily for 4 weeks.

Histone deacetylases (HDACs) are an important class of epigenetic enzymes that regulate gene expression by removing acetyl groups from N-terminal lysine residues on histones. Dysregulation of HDACs is closely related with tumorigenesis and proliferation, and inhibition of HDACs can inhibit tumor cell proliferation, induce cell differentiation and (or) apoptosis, by increasing intracellular histone acetylation, increasing the expression level of genes such as p21. Currently, several histone HDAC inhibitors have been approved for marketing, such as Vorinostat and Panobinostat.

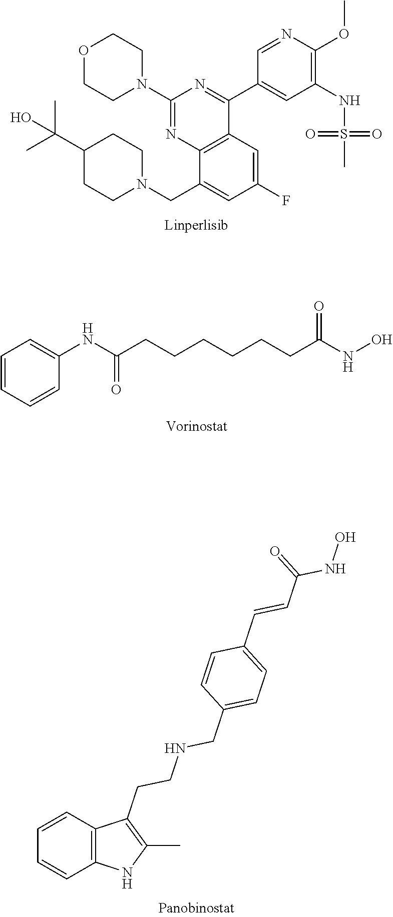 [SMILES: COc1ncc(-c2nc(N3CCOCC3)nc3c(CN4CCC(C(C)(C)O)CC4)cc(F)cc23)cc1NS(C)(=O)=O ; O=C(CCCCCCC(=O)Nc1ccccc1)NO ; Cc1[nH]c2ccccc2c1CCNCc1ccc(/C=C/C(=O)NO)cc1]

Although both PI3K δ inhibitors and HDAC inhibitors have been approved for marketing, however, the use of single drug often suffers from a limited range of indications and is prone to drug resistance. The development of therapeutic agents that incorporate two bioactive groups into a single molecule with dual inhibitory effects of PI3K δ and HDAC, it is expected to achieve better efficacy and wider application.

### Content of the Present Disclosure

The technical problem to be solved by the present disclosure is to provide a morpholinylquinazoline compound, pharmaceutical compositions thereof and use thereof for the lack of drugs with dual PI3K δ and HDAC inhibitory effects. The morpholinylquinazolines of the present disclosure have dual inhibitory activities towards PI3Kδ and HDAC, which are expected to achieve better efficacy and wider application.

The present disclosure solves the above-mentioned technical problem through the following technical solutions.

The present disclosure provides a morpholinylquinazoline compound represented by formula I, a pharmaceutically acceptable salt thereof, a solvate thereof, a solvate of the pharmaceutically acceptable salt thereof, a polycrystalline form thereof or a precursor thereof, wherein:

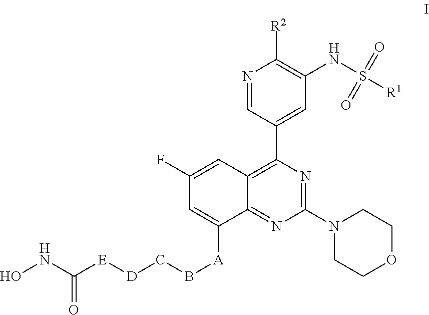

R¹ is C₁₋₄ alkyl, C₁₋₄ alkyl substituted with one or more halogen, or C₃₋₈ cycloalkyl;

R² is hydrogen or —OC₁₋₄ alkyl;

A, C and E are independently a bond, or —(U₁)ₙ₁—(R^{L-1})₂—(U₂)ₙ₃—(R^{L-2})ₙ₄—(U₃)ₙ₅—(R^{L-3})ₙ₆—;

U₁, U₂ and U₃ are independently —O—, —S—, —NH— or —NR³—, R³ is C₁₋₆ alkyl;
R^{L}-1, R^{L-2} and R^{L-3} are independently C₁₋₆alkylene, C₂₋₆ alkenyl or C₂₋₆ alkynyl;

n1, n3 and n5 are independently 0 or 1;

n2, n4 and n6 are independently 0, 1, 2, 3 or 4;

B and D are independently a bond, C₃₋₁₀ cycloalkyl, C₃₋₁₀ cycloalkyl substituted with one or more R¹⁻¹, “4-10 membered heterocycloalkyl containing 1-3 heteroatoms selected from O and N”, “4-10 membered heterocycloalkyl containing 1-3 heteroatoms selected from O and N” substituted with one or more R¹⁻², C₆₋₂₀ aryl, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, “8-12 membered benzoheterocycloalkenyl containing 1-3 heteroatoms selected from O, S and N”, “8-12 membered benzoheterocycloalkenyl containing 1-3 heteroatoms selected from O, S and N” substituted with one or more R¹⁻⁵-5, 5-7 membered cycloalkenyl, or 5-7 membered cycloalkenyl substituted with one or more R¹⁻⁶; when multiple substituents are present, the substituents are the same or different;

R¹⁻¹, R¹⁻², R¹⁻³, R¹⁻⁴, R¹⁻⁵ and R¹⁻⁶ are independently halogen or C₁₋₆ alkyl.

In a certain embodiment, with regard to a morpholinylquinazoline compound represented by formula I, a pharmaceutically acceptable salt thereof, a solvate thereof, a solvate of the pharmaceutically acceptable salt thereof, a polycrystalline form thereof or a precursor thereof, some groups are defined as follows, and the unmentioned group definitions are as described in any one of the embodiments of the present disclosure (this content is hereinafter referred to simply as “in a certain embodiment”), with regard to a morpholinylquinazoline compound represented by formula I.

In a certain embodiment, U₁ is O, n1 is 1, n2, n3, n4, n5 and n6 are 0, at this point, A is —O—.

In a certain embodiment, A is —O—, C₁₋₆ alkylene, —OC₁₋₆ alkylene, or C₁₋₆ alkylene—NH—.

In a certain embodiment, B is a bond, C₃₋₁₀ cycloalkyl, “4-10 membered heterocycloalkyl containing 1-3 heteroatoms selected from O and N”, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or, “8-12 membered benzoheterocycloalkenyl containing 1-3 heteroatoms selected from O, S and N” substituted with one or more R¹⁻⁵.

In a certain embodiment, C is a bond, C₁₋₆ alkylene, C₂₋₆ alkenyl, —OC₁₋₆ alkylene, —OC₁₋₆ alkylene-OC₁₋₆ alkylene, C₁₋₆ alkylene-OC₁₋₆ alkylene or C₁₋₆ alkylene-NH—;

In a certain embodiment, D is a bond, C₆₋₂₀ aryl, C₆₋₂₀ aryl substituted with one or more R¹⁻³, C₃₋₁₀ cycloalkyl, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or, “4-10 membered heterocycloalkyl containing 1-3 heteroatoms selected from O and N”.

In a certain embodiment, regardless of whether E is a bond or an arbitrary group, the described morpholinylquinazolines as shown in Formula I have dual inhibitory activities towards PI3K δ and HDAC.

In a certain embodiment, E is a bond.

In a certain embodiment, in the morpholinylquinazoline compound represented by formula I,

R¹ is C₁₋₄ alkyl, C₁₋₄ alkyl substituted with one or more halogen, or C₃₋₈ cycloalkyl;

R² is hydrogen or —OC₁₋₄ alkyl;

A is —O—, C₁₋₆ alkylene, —OC₁₋₆ alkylene or C₁₋₆ alkylene-NH—;

B is a bond, C₃₋₁₀ cycloalkyl, “4-10 membered heterocycloalkyl containing 1-3 heteroatoms selected from O and N”, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or, “8-12 membered benzoheterocycloalkenyl containing 1-3 heteroatoms selected from O, S and N” substituted with one or more R¹⁻⁵;

C is a bond, C₁₋₆ alkylene, C₂₋₆ alkenyl, —OC₁₋₆ alkylene, —OC₁₋₆ alkylene-OC₁₋₆ alkylene, C₁₋₆ alkylene-OC₁₋₆ alkylene or C₁₋₆ alkylene-NH—;

D is a bond, C₆₋₂₀ aryl, C₆₋₂₀ aryl substituted with one or more R¹⁻³, “5-12 membered heteroaryl containing 1-4 heteroatoms selected from O, S and N”, C₃₋₁₀ cycloalkyl, “5-12 membered heteroaryl containing 1-4 heteroatoms selected from O, S and N” substituted with one or more R¹⁻⁴; or, “4-10 membered heterocycloalkyl containing 1-3 heteroatoms selected from O and N”;

E is a bond.

In a certain embodiment, in the morpholinylquinazoline compound represented by formula I,

R¹ is C₁₋₄ alkyl, C₁₋₄ alkyl substituted with one or more halogen, or C₃₋₈ cycloalkyl;

R² is hydrogen or —OC₁₋₄ alkyl;

A is —O—, C₁₋₆ alkylene, —OC₁₋₆ alkylene or C₁₋₆ alkylene-NH—;

B is a bond, C₃₋₁₀ cycloalkyl, “4-10 membered heterocycloalkyl containing 1-3 heteroatoms selected from O and N”, C₆₋₂₀ aryl substituted with one or more R¹⁻³, or, “5-12 membered heteroaryl containing 1-4 heteroatoms selected from O, S and N” substituted with one or more R¹⁻⁴;

C is a bond or C₁₋₆ alkylene;

D is a bond, C₆₋₂₀ aryl, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴;

E is a bond.

In a certain embodiment, when R¹ is C₁₋₄ alkyl or C₁₋₄ alkyl substituted with one or more halogens, the C₁₋₄ alkyl and the C₁₋₄ alkyl in the C₁₋₄ alkyl substituted with one or more halogens are methyl, ethyl, n-propyl, isopropyl, n-butyl, sec-butyl, isobutyl or tert-butyl, for example, methyl.

In a certain embodiment, when R¹ is C₃₋₈ cycloalkyl, the C₃₋₈ cycloalkyl is cyclohexyl, cyclopentyl, cyclobutyl or cyclopropyl, for example, cyclopropyl.

In a certain embodiment, when R² is —OC₁₋₄ alkyl, the C₁₋₄ alkyl in the —OC₁₋₄ alkyl is -methyl,-ethyl, n-propyl, isopropyl, n-butyl, sec-butyl, isobutyl or tert-butyl, for example, methyl.

In a certain embodiment, when R¹⁻¹, R¹⁻², R¹⁻³, R¹⁻⁴, R¹⁻⁵ and R¹⁻⁶ are independently halogen, the halogen is fluorine, chlorine, bromine or iodine.

In a certain embodiment, when R³, R¹⁻¹, R¹⁻², R¹⁻³, R¹⁻⁴, R¹⁻⁵ and R¹⁻⁶ are independently C₁₋₆ alkyl, the C₁₋₆ alkyl is C₁₋₄ alkyl, preferably methyl, ethyl, n-propyl, isopropyl, n-butyl, sec-butyl, isobutyl or tert-butyl.

In a certain embodiment, when A is C₁₋₆ alkylene, —OC₁₋₆ alkylene or C₁₋₆ alkylene-NH—, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—, for example, —CH₂—.

In a certain embodiment, when A is —OC₁₋₆ alkylene, the C₁₋₆ alkylene end is connected with B.

In a certain embodiment, when B is C₃₋₁₀ cycloalkyl, the C₃₋₁₀ cycloalkyl is cyclohexyl, cyclopentyl, cyclobutyl or cyclopropyl, for example, cyclohexyl (for example,

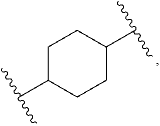 [SMILES: *C1CCC(*)CC1]

preferably,

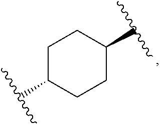 [SMILES: *[C@H]1CC[C@H](*)CC1]

more preferably, it is linked with two sides of group by way of

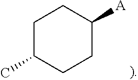

In a certain embodiment, when B is “4- to 10-membered heterocycloalkyl containing 1-3 heteroatoms selected from O, S and N”, the “4- to 10-membered heterocycloalkyl containing 1-3 heteroatoms selected from O, S and N” is “3- to 6-membered heterocycloalkyl containing 1-2 heteroatoms selected from N”, for example,

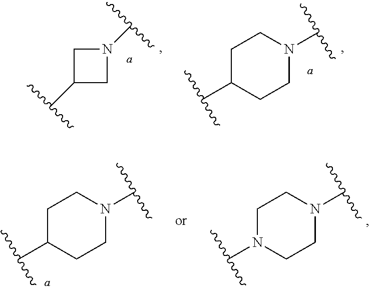 [SMILES: *C1CN(*)C1 ; *C1CCN(*)CC1 ; *C1CCN(*)CC1 ; *N1CCN(*)CC1]

preferably, the position a is connected with A.

In a certain embodiment, when B is C₆₋₂₀ aryl substituted with one or more R¹⁻³, the C₆₋₂₀ aryl is phenyl, for example,

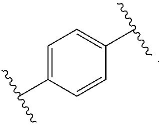 [SMILES: *c1ccc(*)cc1]

In a certain embodiment, when B is “5- to 12-membered heteroaryl containing 1-4 heteroatoms selected from O, S and N”, the “5- to 12-membered heteroaryl containing 1-4 heteroatoms selected from O, S and N” is “5- to 6-membered heteroaryl containing 1-2 heteroatoms selected from N”, for example,

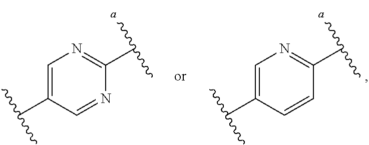 [SMILES: *c1cnc(*)nc1 ; *c1ccc(*)nc1]

preferably, the position a is connected with A.

In a certain embodiment, when B is “5- to 12-membered heteroaryl containing 1-4 heteroatoms selected from O, S and N” substituted with one or more R¹⁻⁴, the B is

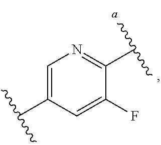 [SMILES: *c1cnc(*)c(F)c1]

preferably, the position a is connected with A.

In a certain embodiment, when B is “8- to 12-membered benzoheterocycloalkenyl containing 1-3 heteroatoms selected from O, S and N” substituted with one or more R¹⁻⁵, the B is “9- to 10-membered benzoheterocycloalkenyl containing 1 heteroatoms of N”, for example,

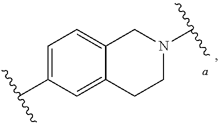 [SMILES: *c1ccc2c(c1)CCN(*)C2]

preferably, the position a is connected with A.

In a certain embodiment, when B is “8- to 12-membered benzocyclcloalkenyl containing 1-3 heteroatoms selected from O, S and N” substituted with one or more R¹⁻⁵, the B is

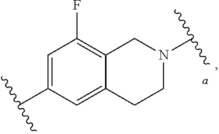 [SMILES: *c1cc(F)c2c(c1)CCN(*)C2]

preferably, the position a is connected with A.

In a certain embodiment, when C is C₁₋₆ alkylene, —OC₁₋₆ alkylene-OC₁₋₆ alkylene, C₁₋₆ alkylene-OC₁₋₆ alkylene or C₁₋₆ alkylene-NH—, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—.

In a certain embodiment, when C is —OC₁₋₆ alkylene or —OC₁₋₆ alkylene-OC₁₋₆ alkylene, the —O end is connected with B.

In a certain embodiment, when C is C₁₋₆ alkylene-NH—, the C₁₋₆ alkylene end is connected with B.

In a certain embodiment, when C is C₂₋₆ alkenyl, the C is vinyl, propenyl or allyl, preferably, when C is vinyl, the vinyl is

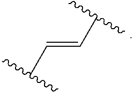 [SMILES: */C=C/*]

In a certain embodiment, when D is C₆₋₂₀ aryl or C₆₋₂₀ aryl substituted with one or more R¹⁻³, the C₆₋₂₀ aryl is phenyl or naphthyl, for example,

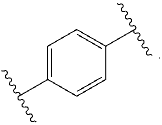 [SMILES: *c1ccc(*)cc1]

In a certain embodiment, when D is C₆₋₂₀ aryl substituted with one or more R¹⁻³, the D is

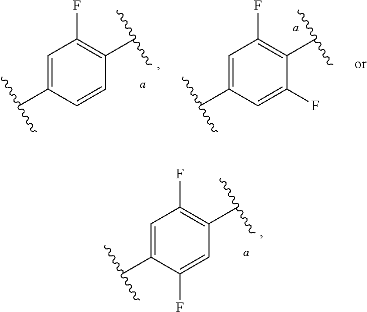 [SMILES: *c1ccc(*)c(F)c1 ; *c1cc(F)c(*)c(F)c1 ; *c1cc(F)c(*)cc1F]

preferably, the position a is connected with A.

In a certain embodiment, when D is “5- to 12-membered heteroaryl containing 1-4 heteroatoms selected from O, S and N” or “5- to 12-membered heteroaryl containing 1-4 heteroatoms selected from O, S and N” substituted with one or more R¹⁻⁴, the “5- to 12-membered heteroaryl containing 1-4 heteroatoms selected from O, S and N” is “5- to 6-membered heteroaryl containing 1-2 heteroatoms selected from O and N”, for example,

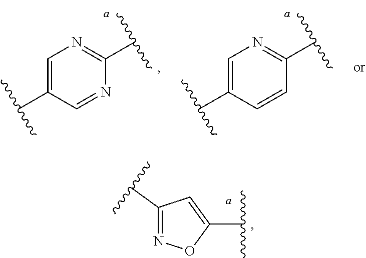 [SMILES: *c1cnc(*)nc1 ; *c1ccc(*)nc1 ; *c1cc(*)on1]

preferably, the position a is connected with A.

In a certain embodiment, when D is “5- to 12-membered heteroaryl containing 1-4 heteroatoms selected from O, S and N” substituted with one or more R¹⁻⁴, the D is

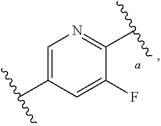 [SMILES: *c1cnc(*)c(F)c1]

preferably, the position a is connected with C.

In a certain embodiment, when D is “4- to 10-membered heterocycloalkyl containing 1-3 heteroatoms selected from O, S and N”, the D is “5- to 6-membered heterocycloalkyl containing 1-2 heteroatoms selected from O and N”, for example,

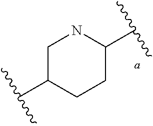 [SMILES: *C1CCC(*)NC1]

preferably, the position a is connected with C.

In a certain embodiment, A is —O—, —CH₂—,

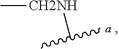

—OCH₂—^{a}, —OCH₂CH₂—^{a},

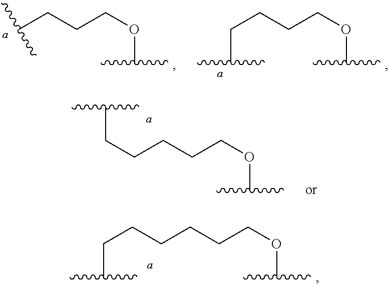

preferably, the position a is connected with B.

In a certain embodiment, B is a bond

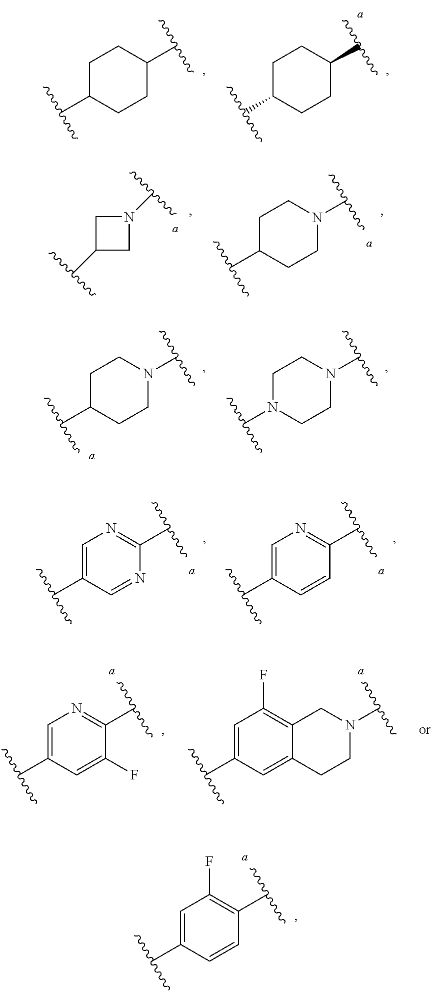

preferably, the position a is connected with A.

In a certain embodiment, C is —CH₂—, ^{a}—OCH₂CH₂—,

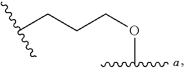 [SMILES: *CCCO*]

—CH₂CH₂—, —CH₂CH₂CH₂—,

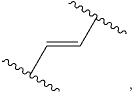 [SMILES: */C=C/*]

—CH₂CH₂CH₂CH₂—,^{a}—OCH₂CH₂OCH₂— or ^{a}—C(CH₃)₂OCH₂—, preferably, the position a is connected with B.

In a certain embodiment, D is a bond,

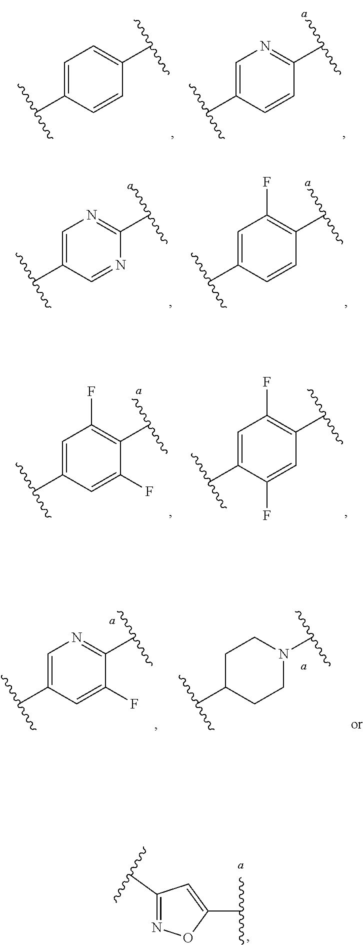

preferably, the position a is connected with C.

In a certain embodiment, -D-C-B-A- is

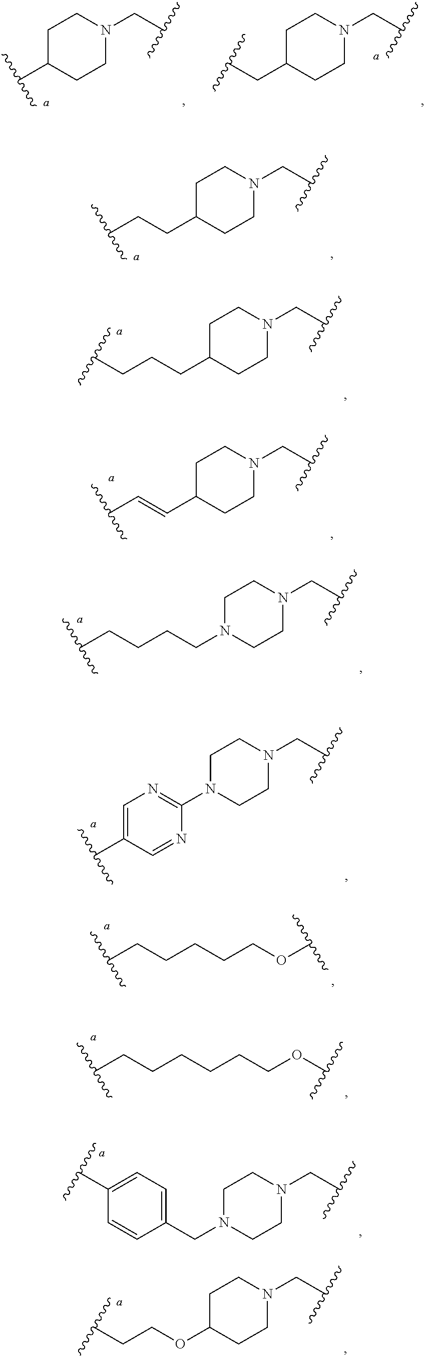

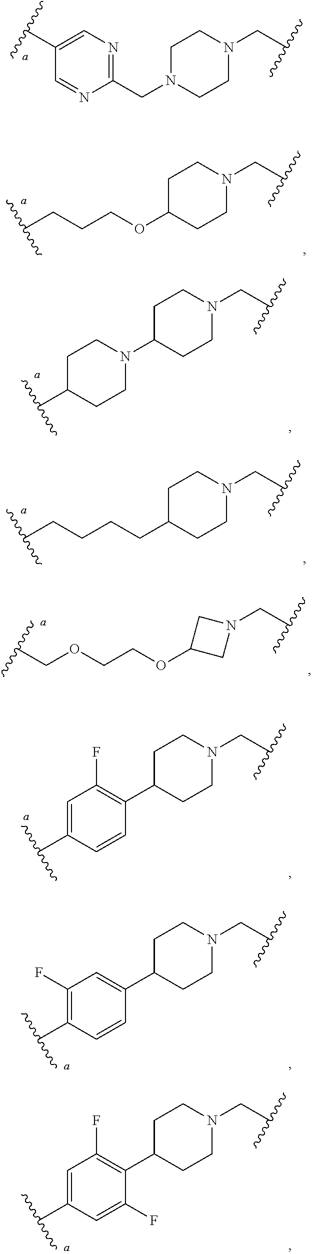

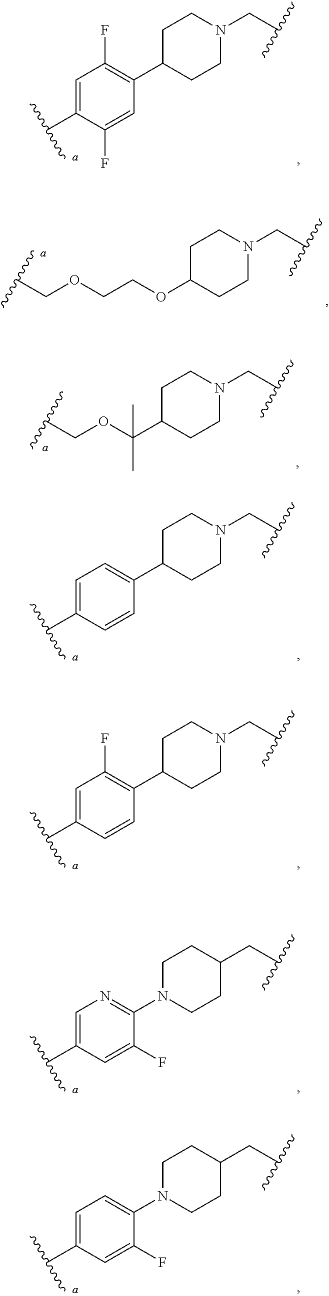

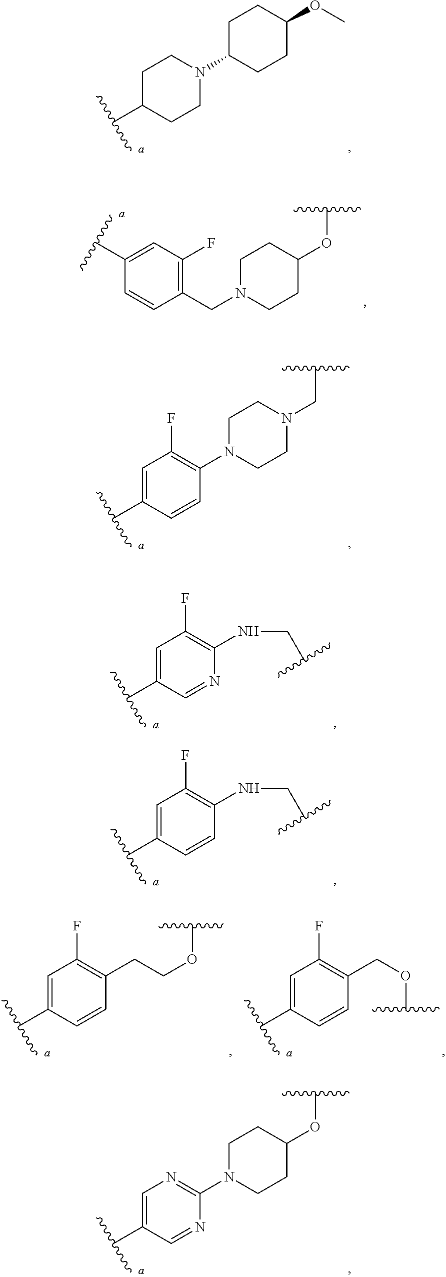

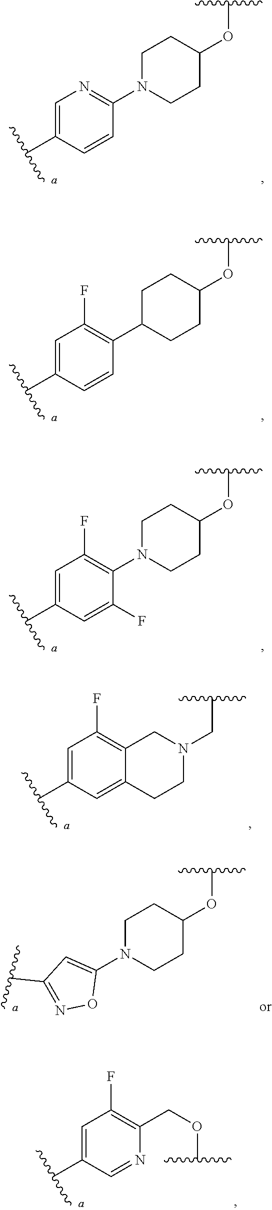 [SMILES: *OC1CCN(c2ccc(*)cn2)CC1 ; *OC1CCC(c2ccc(*)cc2F)CC1 ; *OC1CCN(c2c(F)cc(*)cc2F)CC1 ; *CN1CCc2cc(*)cc(F)c2C1 ; *OC1CCN(c2cc(*)no2)CC1 ; *OCc1ncc(*)cc1F]

preferably, the position a is connected with E.

In a certain embodiment, -E-D-C-B-A- is

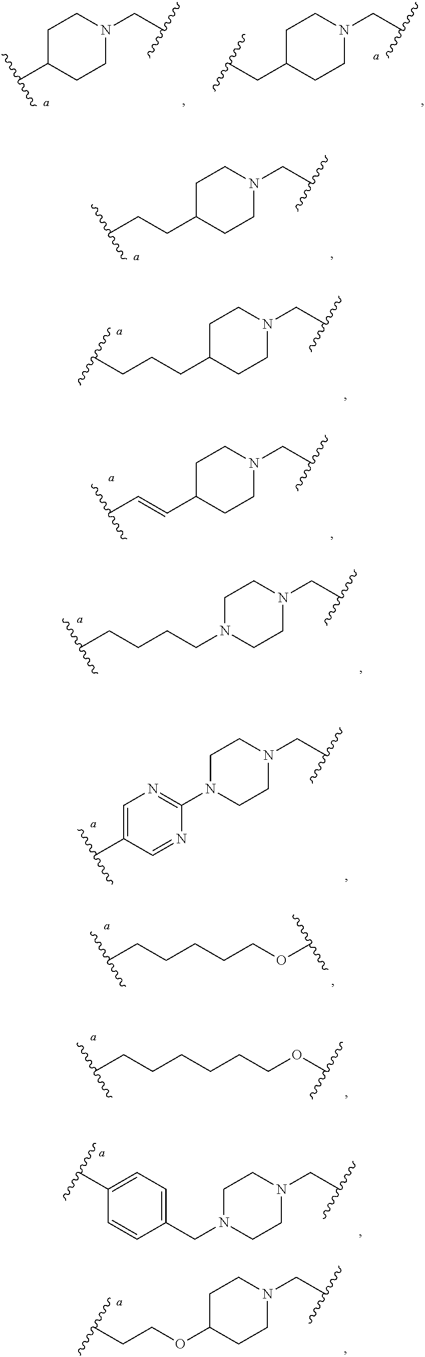

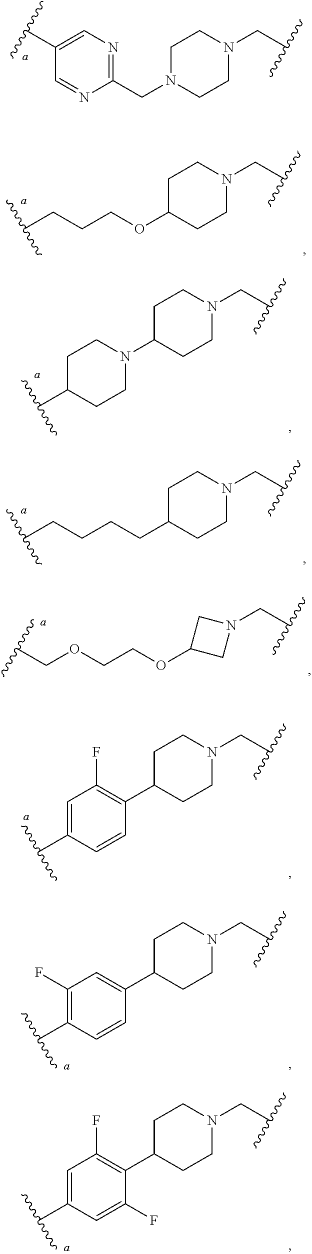

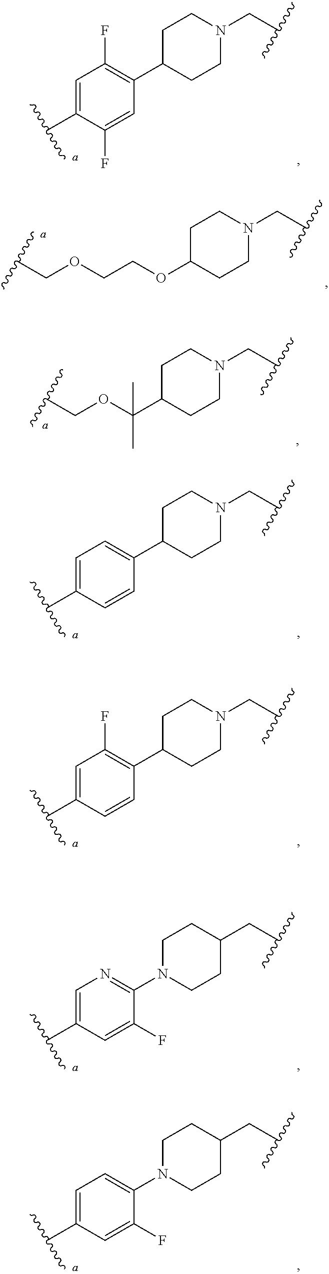

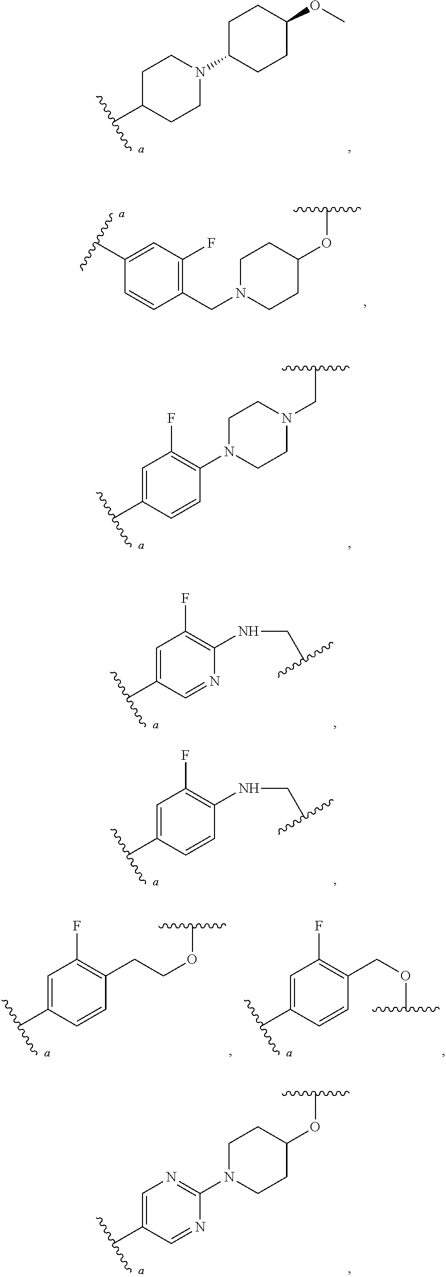

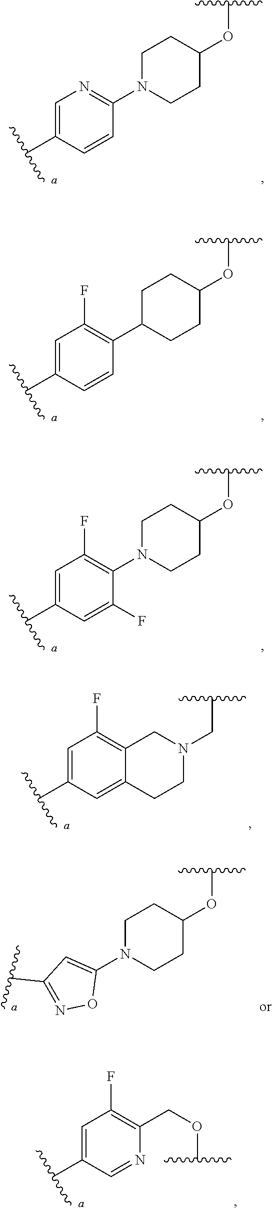 [SMILES: *OC1CCN(c2ccc(*)cn2)CC1 ; *OC1CCC(c2ccc(*)cc2F)CC1 ; *OC1CCN(c2c(F)cc(*)cc2F)CC1 ; *CN1CCc2cc(*)cc(F)c2C1 ; *OC1CCN(c2cc(*)no2)CC1 ; *OCc1ncc(*)cc1F]

preferably, the position a is connected with

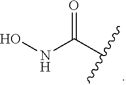 [SMILES: *C(=O)NO]

In a certain embodiment, the morpholinylquinazoline compound represented by formula I has any one of the following structures:

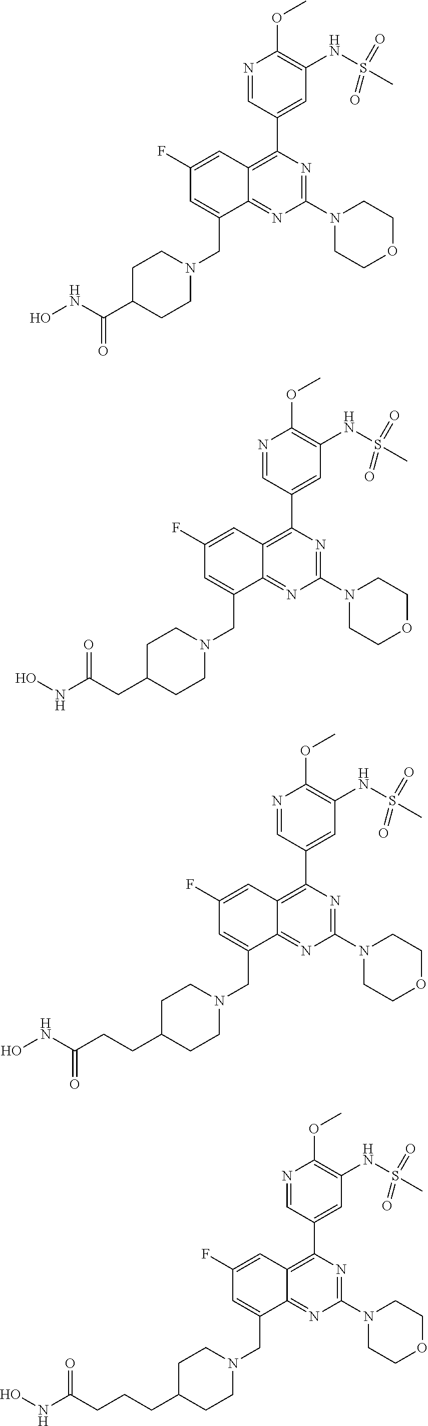 [SMILES: COc1ncc(-c2nc(N3CCOCC3)nc3c(CN4CCC(C(=O)NO)CC4)cc(F)cc23)cc1NS(C)(=O)=O ; COc1ncc(-c2nc(N3CCOCC3)nc3c(CN4CCC(CC(=O)NO)CC4)cc(F)cc23)cc1NS(C)(=O)=O ; COc1ncc(-c2nc(N3CCOCC3)nc3c(CN4CCC(CCC(=O)NO)CC4)cc(F)cc23)cc1NS(C)(=O)=O ; COc1ncc(-c2nc(N3CCOCC3)nc3c(CN4CCC(CCCC(=O)NO)CC4)cc(F)cc23)cc1NS(C)(=O)=O]

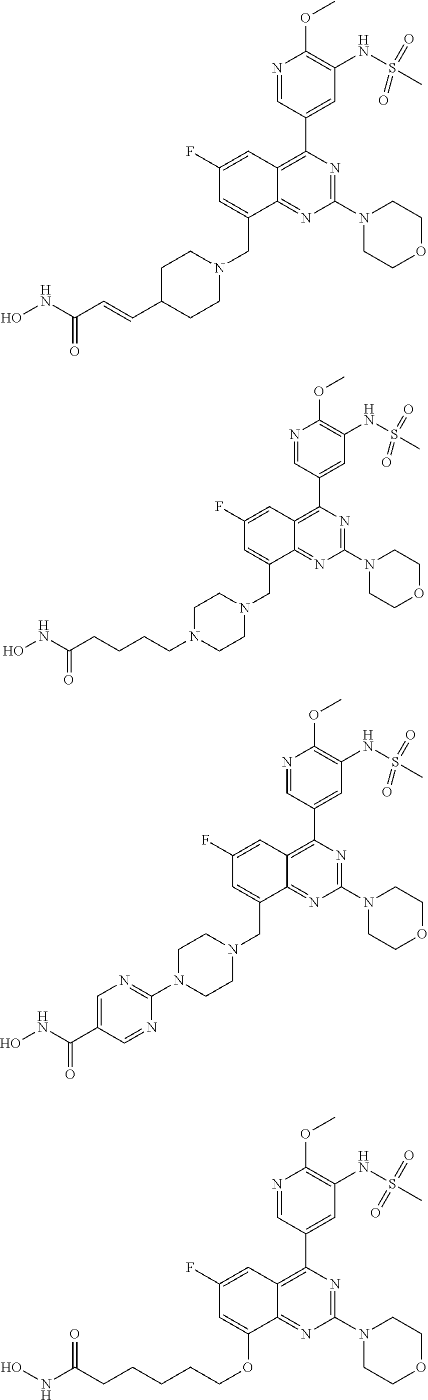 [SMILES: COc1ncc(-c2nc(N3CCOCC3)nc3c(CN4CCC(/C=C/C(=O)NO)CC4)cc(F)cc23)cc1NS(C)(=O)=O ; COc1ncc(-c2nc(N3CCOCC3)nc3c(CN4CCN(CCCCC(=O)NO)CC4)cc(F)cc23)cc1NS(C)(=O)=O ; COc1ncc(-c2nc(N3CCOCC3)nc3c(CN4CCN(c5ncc(C(=O)NO)cn5)CC4)cc(F)cc23)cc1NS(C)(=O)=O ; COc1ncc(-c2nc(N3CCOCC3)nc3c(OCCCCCC(=O)NO)cc(F)cc23)cc1NS(C)(=O)=O]

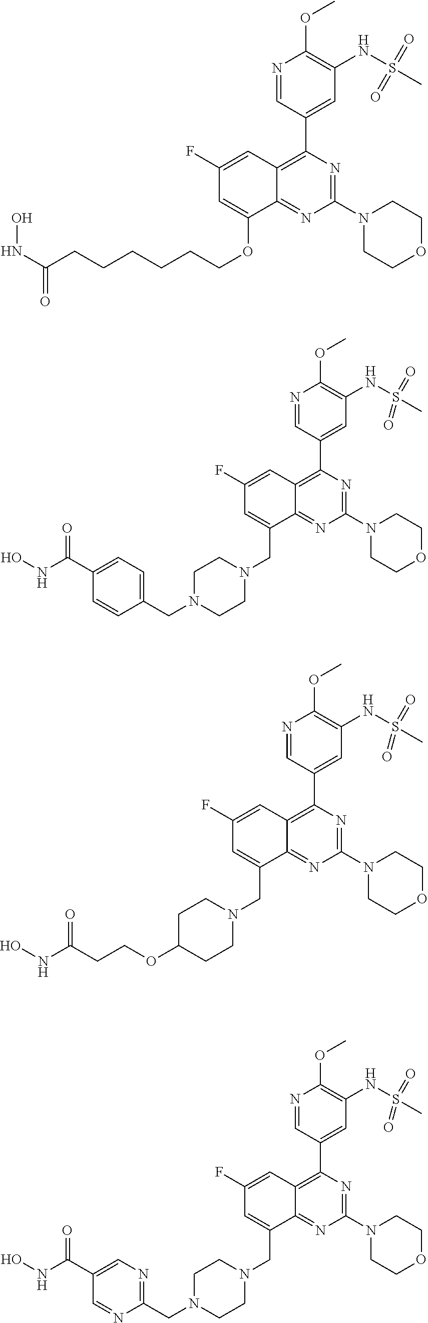 [SMILES: COc1ncc(-c2nc(N3CCOCC3)nc3c(OCCCCCCC(=O)NO)cc(F)cc23)cc1NS(C)(=O)=O ; COc1ncc(-c2nc(N3CCOCC3)nc3c(CN4CCN(Cc5ccc(C(=O)NO)cc5)CC4)cc(F)cc23)cc1NS(C)(=O)=O ; COc1ncc(-c2nc(N3CCOCC3)nc3c(CN4CCC(OCCC(=O)NO)CC4)cc(F)cc23)cc1NS(C)(=O)=O ; COc1ncc(-c2nc(N3CCOCC3)nc3c(CN4CCN(Cc5ncc(C(=O)NO)cn5)CC4)cc(F)cc23)cc1NS(C)(=O)=O]

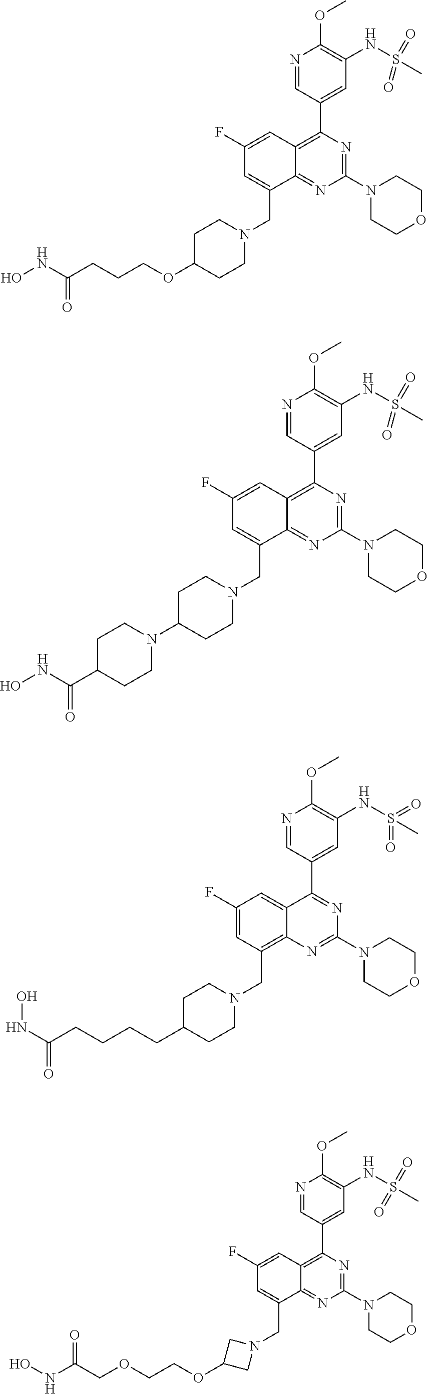 [SMILES: COc1ncc(-c2nc(N3CCOCC3)nc3c(CN4CCC(OCCCC(=O)NO)CC4)cc(F)cc23)cc1NS(C)(=O)=O ; COc1ncc(-c2nc(N3CCOCC3)nc3c(CN4CCC(N5CCC(C(=O)NO)CC5)CC4)cc(F)cc23)cc1NS(C)(=O)=O ; COc1ncc(-c2nc(N3CCOCC3)nc3c(CN4CCC(CCCCC(=O)NO)CC4)cc(F)cc23)cc1NS(C)(=O)=O ; COc1ncc(-c2nc(N3CCOCC3)nc3c(CN4CC(OCCOCC(=O)NO)C4)cc(F)cc23)cc1NS(C)(=O)=O]

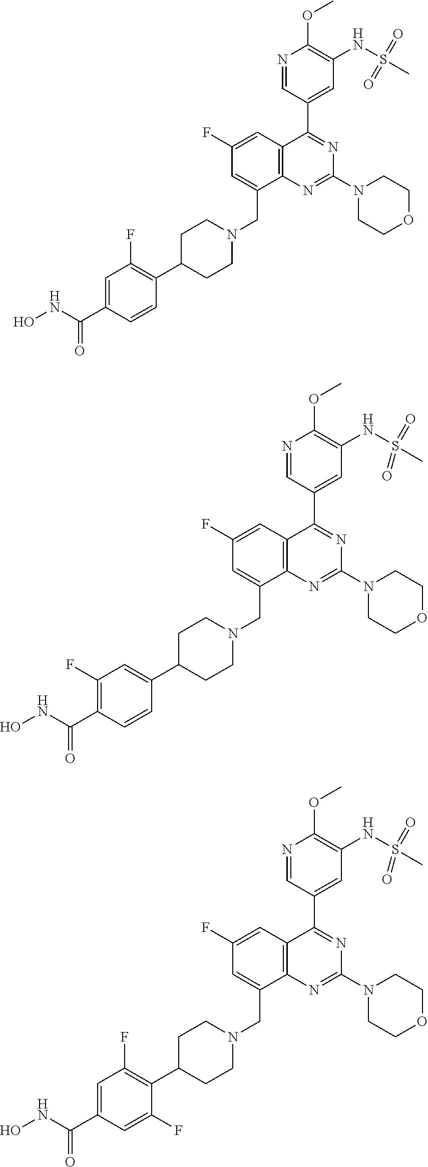 [SMILES: COc1ncc(-c2nc(N3CCOCC3)nc3c(CN4CCC(c5ccc(C(=O)NO)cc5F)CC4)cc(F)cc23)cc1NS(C)(=O)=O ; COc1ncc(-c2nc(N3CCOCC3)nc3c(CN4CCC(c5ccc(C(=O)NO)c(F)c5)CC4)cc(F)cc23)cc1NS(C)(=O)=O ; COc1ncc(-c2nc(N3CCOCC3)nc3c(CN4CCC(c5c(F)cc(C(=O)NO)cc5F)CC4)cc(F)cc23)cc1NS(C)(=O)=O]

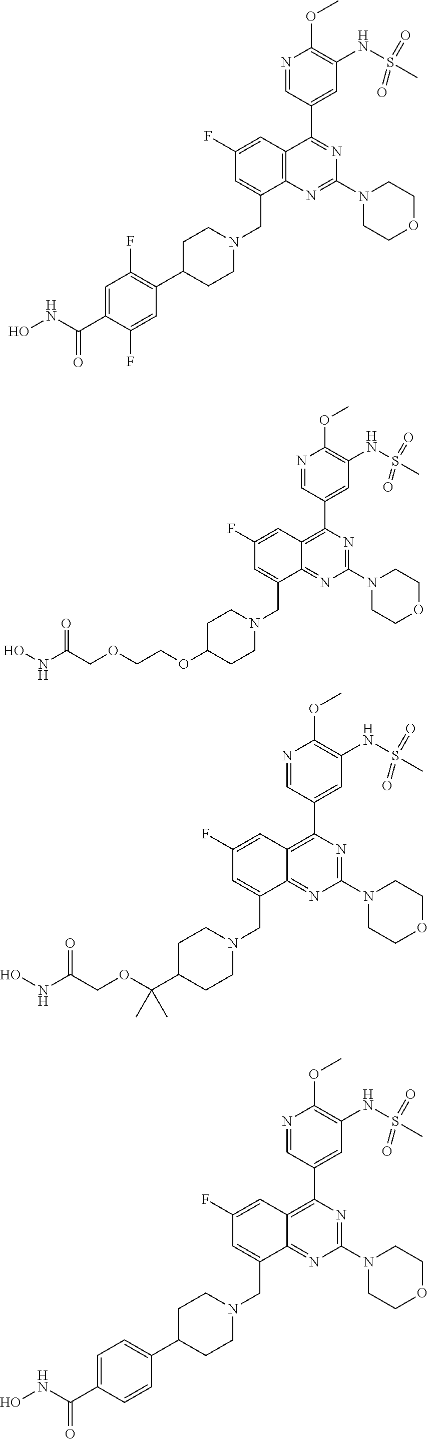 [SMILES: COc1ncc(-c2nc(N3CCOCC3)nc3c(CN4CCC(c5cc(F)c(C(=O)NO)cc5F)CC4)cc(F)cc23)cc1NS(C)(=O)=O ; COc1ncc(-c2nc(N3CCOCC3)nc3c(CN4CCC(OCCOCC(=O)NO)CC4)cc(F)cc23)cc1NS(C)(=O)=O ; COc1ncc(-c2nc(N3CCOCC3)nc3c(CN4CCC(C(C)(C)OCC(=O)NO)CC4)cc(F)cc23)cc1NS(C)(=O)=O ; COc1ncc(-c2nc(N3CCOCC3)nc3c(CN4CCC(c5ccc(C(=O)NO)cc5)CC4)cc(F)cc23)cc1NS(C)(=O)=O]

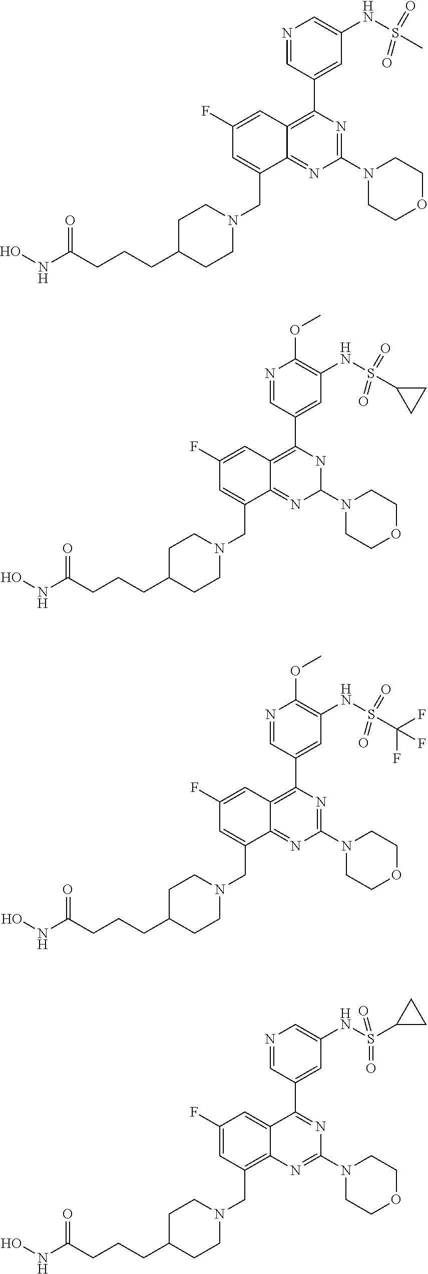

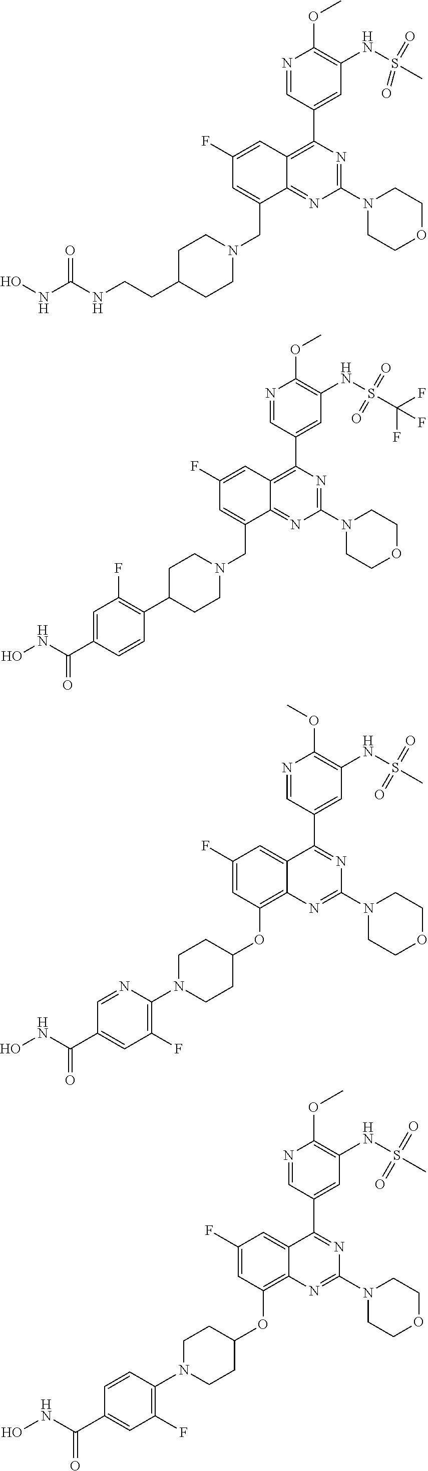 [SMILES: COc1ncc(-c2nc(N3CCOCC3)nc3c(CN4CCC(CCNC(=O)NO)CC4)cc(F)cc23)cc1NS(C)(=O)=O ; COc1ncc(-c2nc(N3CCOCC3)nc3c(CN4CCC(c5ccc(C(=O)NO)cc5F)CC4)cc(F)cc23)cc1NS(=O)(=O)C(F)(F)F ; COc1ncc(-c2nc(N3CCOCC3)nc3c(OC4CCN(c5ncc(C(=O)NO)cc5F)CC4)cc(F)cc23)cc1NS(C)(=O)=O ; COc1ncc(-c2nc(N3CCOCC3)nc3c(OC4CCN(c5ccc(C(=O)NO)cc5F)CC4)cc(F)cc23)cc1NS(C)(=O)=O]

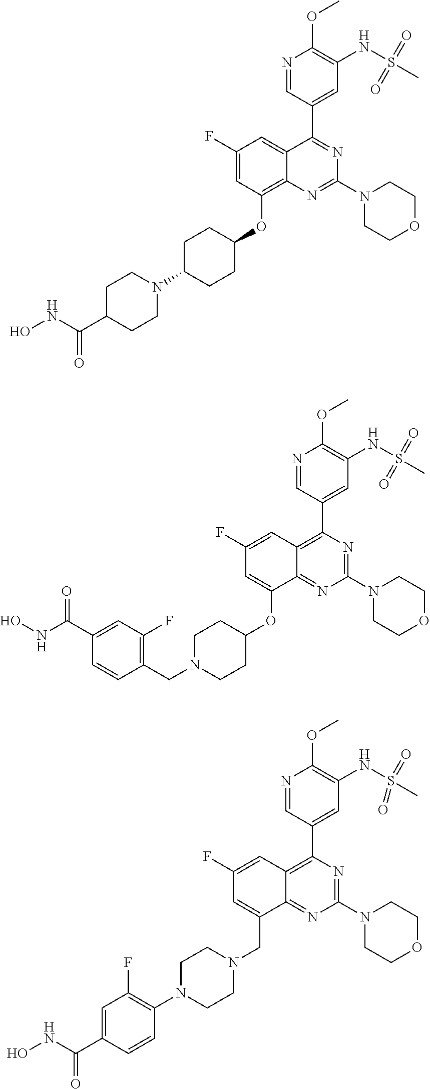 [SMILES: COc1ncc(-c2nc(N3CCOCC3)nc3c(O[C@H]4CC[C@H](N5CCC(C(=O)NO)CC5)CC4)cc(F)cc23)cc1NS(C)(=O)=O ; COc1ncc(-c2nc(N3CCOCC3)nc3c(OC4CCN(Cc5ccc(C(=O)NO)cc5F)CC4)cc(F)cc23)cc1NS(C)(=O)=O ; COc1ncc(-c2nc(N3CCOCC3)nc3c(CN4CCN(c5ccc(C(=O)NO)cc5F)CC4)cc(F)cc23)cc1NS(C)(=O)=O]

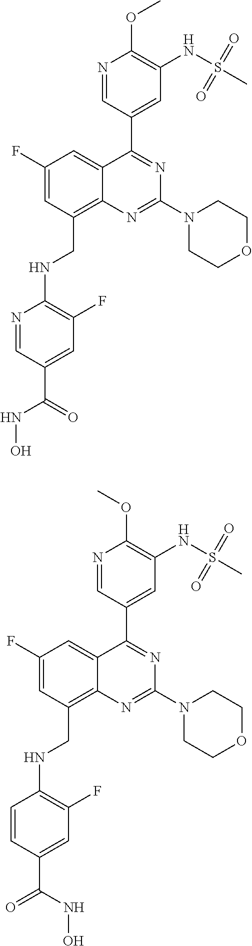 [SMILES: COc1ncc(-c2nc(N3CCOCC3)nc3c(CNc4ncc(C(=O)NO)cc4F)cc(F)cc23)cc1NS(C)(=O)=O ; COc1ncc(-c2nc(N3CCOCC3)nc3c(CNc4ccc(C(=O)NO)cc4F)cc(F)cc23)cc1NS(C)(=O)=O]

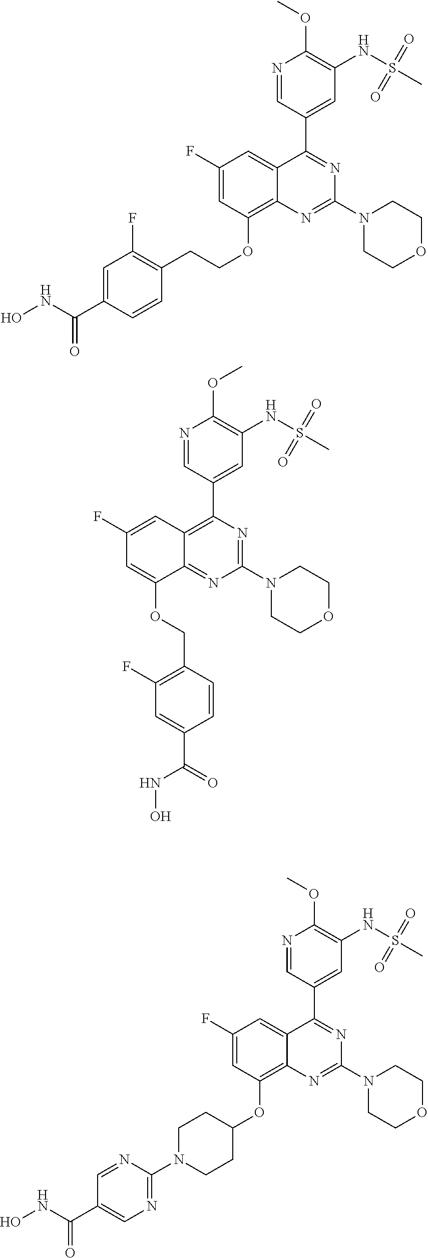 [SMILES: COc1ncc(-c2nc(N3CCOCC3)nc3c(OCCc4ccc(C(=O)NO)cc4F)cc(F)cc23)cc1NS(C)(=O)=O ; COc1ncc(-c2nc(N3CCOCC3)nc3c(OCc4ccc(C(=O)NO)cc4F)cc(F)cc23)cc1NS(C)(=O)=O ; COc1ncc(-c2nc(N3CCOCC3)nc3c(OC4CCN(c5ncc(C(=O)NO)cn5)CC4)cc(F)cc23)cc1NS(C)(=O)=O]

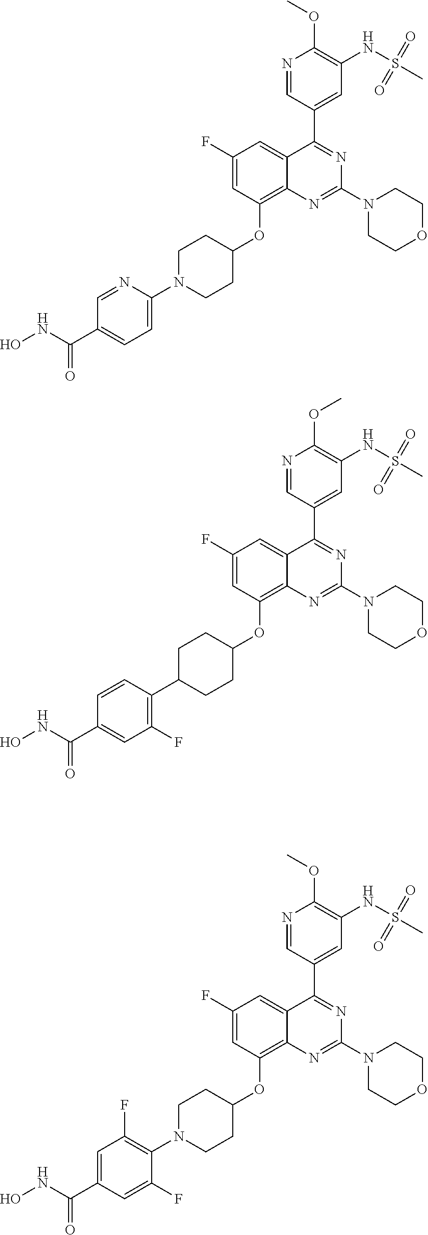 [SMILES: COc1ncc(-c2nc(N3CCOCC3)nc3c(OC4CCN(c5ccc(C(=O)NO)cn5)CC4)cc(F)cc23)cc1NS(C)(=O)=O ; COc1ncc(-c2nc(N3CCOCC3)nc3c(OC4CCC(c5ccc(C(=O)NO)cc5F)CC4)cc(F)cc23)cc1NS(C)(=O)=O ; COc1ncc(-c2nc(N3CCOCC3)nc3c(OC4CCN(c5c(F)cc(C(=O)NO)cc5F)CC4)cc(F)cc23)cc1NS(C)(=O)=O]

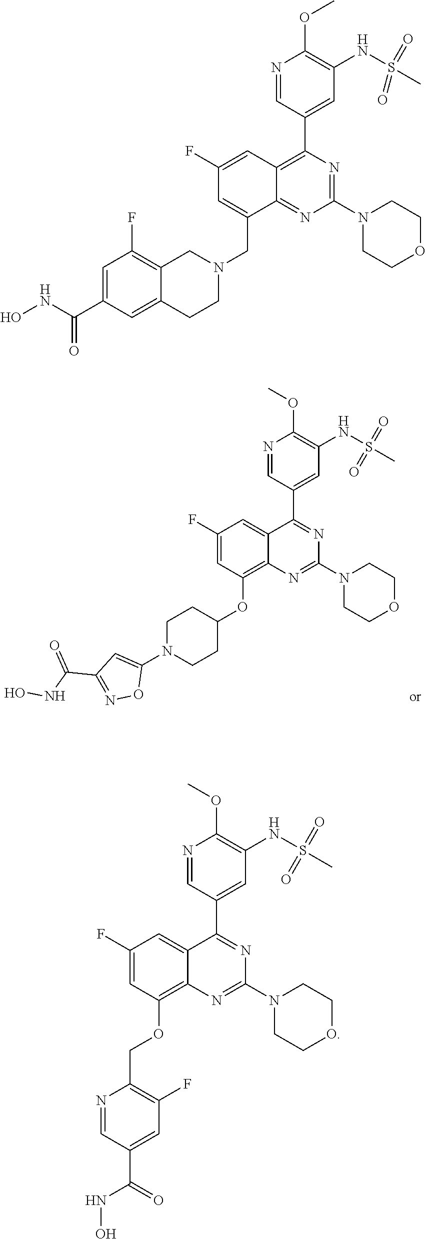 [SMILES: COc1ncc(-c2nc(N3CCOCC3)nc3c(CN4CCc5cc(C(=O)NO)cc(F)c5C4)cc(F)cc23)cc1NS(C)(=O)=O ; COc1ncc(-c2nc(N3CCOCC3)nc3c(OC4CCN(c5cc(C(=O)NO)no5)CC4)cc(F)cc23)cc1NS(C)(=O)=O ; COc1ncc(-c2nc(N3CCOCC3)nc3c(OCc4ncc(C(=O)NO)cc4F)cc(F)cc23)cc1NS(C)(=O)=O]

The present disclosure also provides a method for preparing the above-mentioned morpholinylquinazoline compound represented by formula I, the method comprising route one, route two, route three, route four or route five:

route one:

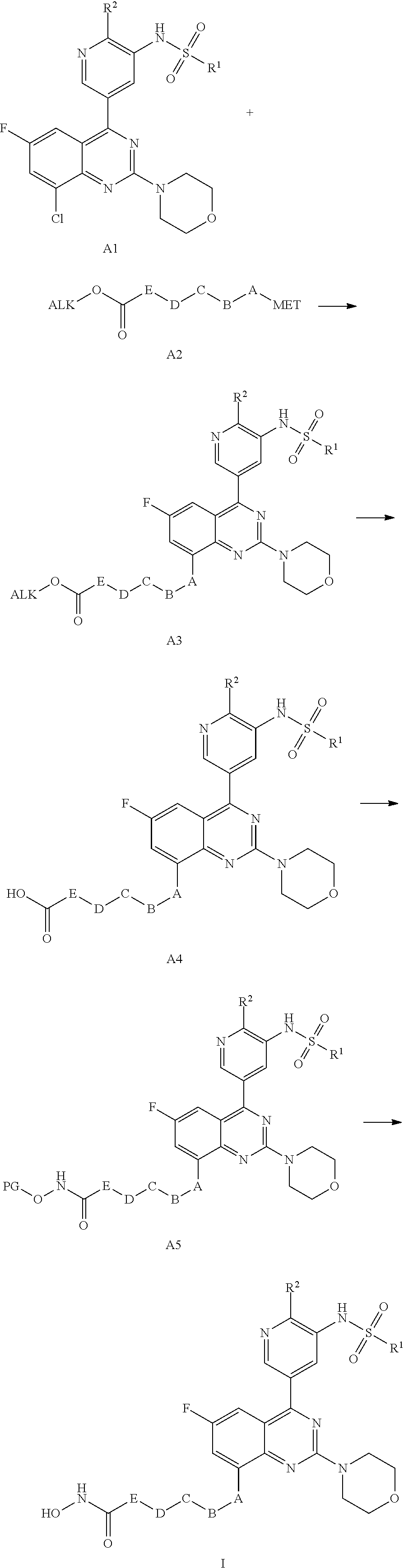

wherein R¹, R², A, B, C, D or E are all as defined above; MET is independently a metal group, for example, BF₃K or B(OH)₂; PG is a protecting group, for example, THIP; ALK is C₁₋₆ alkyl, for example, methyl or ethyl; The steps for route one is described as follows: compound A1 reacted with compound A2 to form compound A3 by means of coupling reaction, A3 is hydrolyzed to give compound A4, A4 is condensed and converted to compound A5, and A5 is de-protected to give compound I.

Route two:

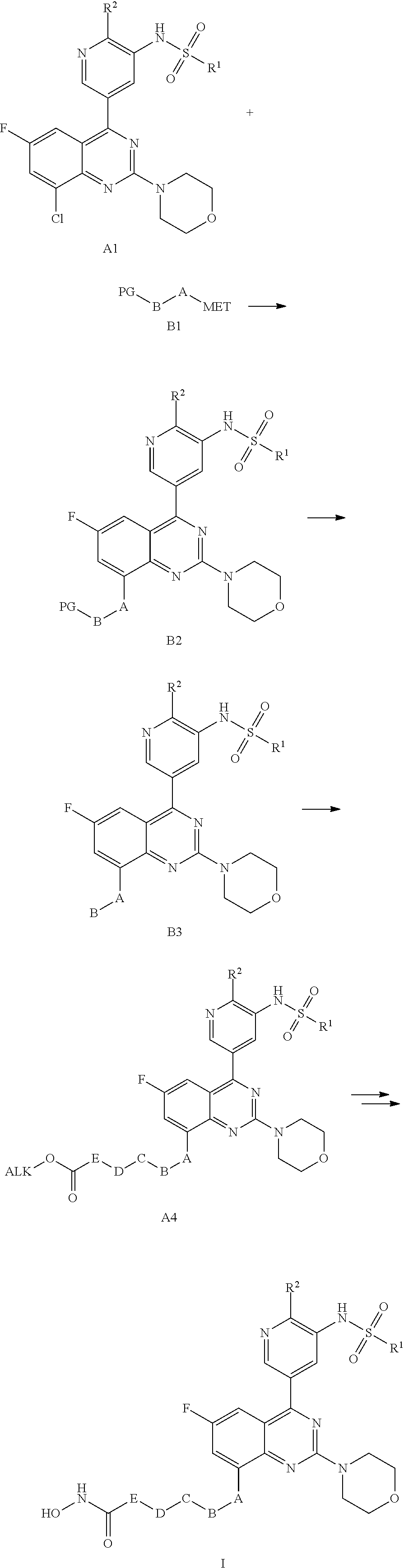

wherein R¹, R², A, B, C, D or E are all as defined above; MET is independently a metal group, for example, BF₃K or B(OH)₂; PG is a protecting group, for example, Boc; ALK is C₁₋₆ alkyl, for example, methyl or ethyl; The steps for route two is described as follows: compound A1 reacted with compound B1 to form compound B2 by means of coupling reaction, B2 is deprotected and converted to compound B3, B3 is converted to compound A4 by means of coupling reaction, A4 is further transformed to obtain compound I.

Route three:

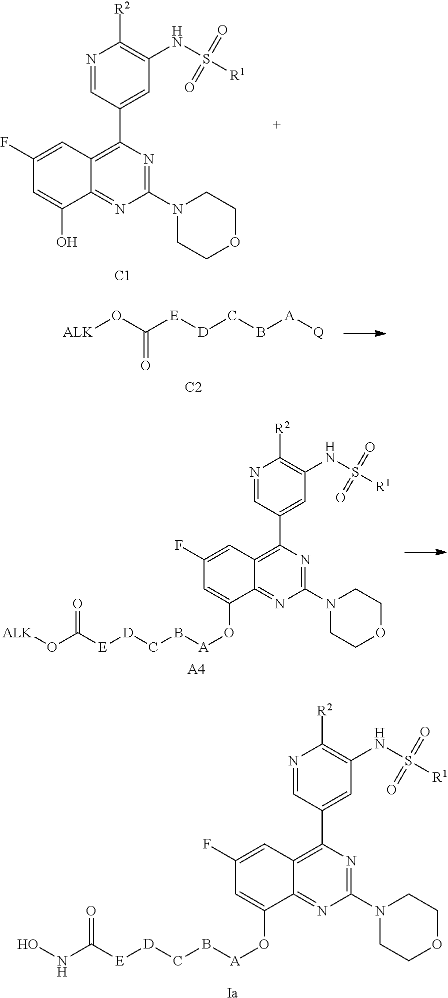

wherein R¹, R², A, B, C, D or E are all as defined above; ALK is C₁₋₆ alkyl, for example, methyl or ethyl; Q is independently leaving group, for example, OTs or Br; The steps for route three is described as follows: compound C1 is coupled with compound C2 to obtain compound A4, A4 is further transformed to obtain compound Ia.

Route four:

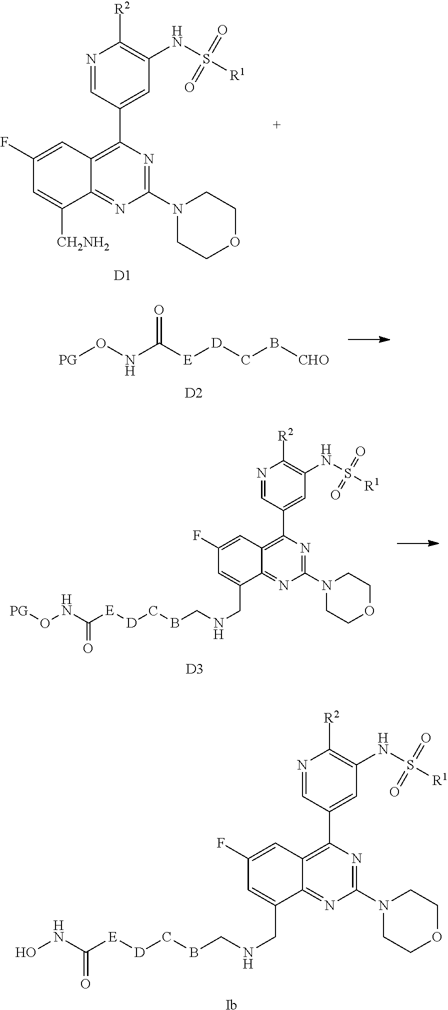

wherein R¹, R², A, B, C, D or E are all as defined above; PG is a protecting group, for example, THP; The steps for route four is described as follows: compound D1 reacted with compound D2 to obtain compound D3 by reductive-amination reaction, D3 is converted to compound Ib by deprotection.

Route five:

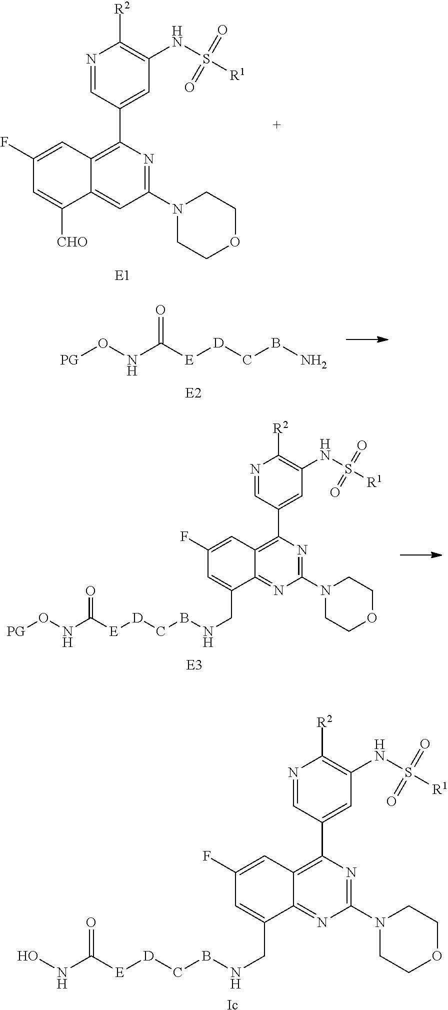

wherein R¹, R², A, B, C, D or E are all as defined above; PG is a protecting group, for example, THP; The steps for route four is described as follows: compound E1 reacted with compound E2 to obtain compound E3 by reductive-amination reaction, E3 is converted to compound Ic by deprotection reaction.

The present disclosure also provides a compound:

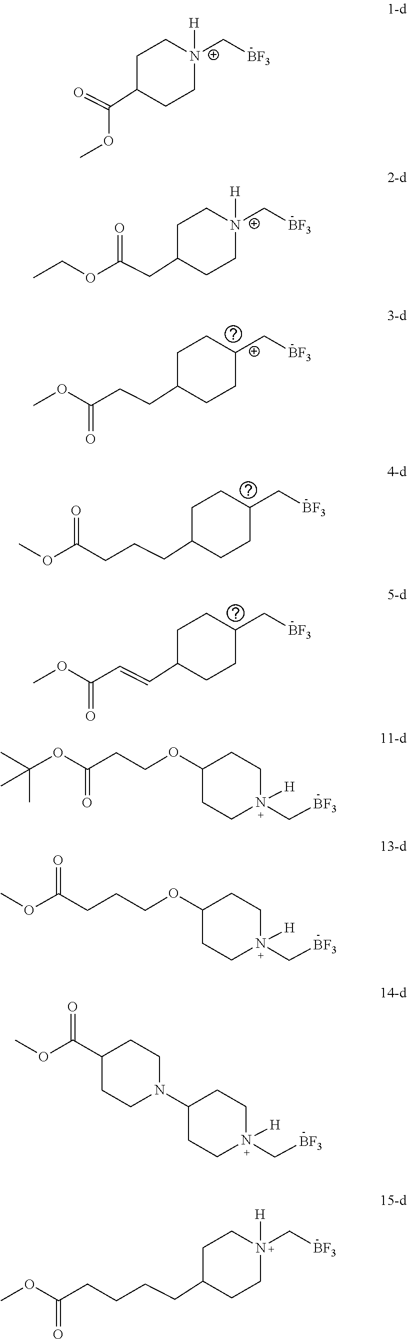

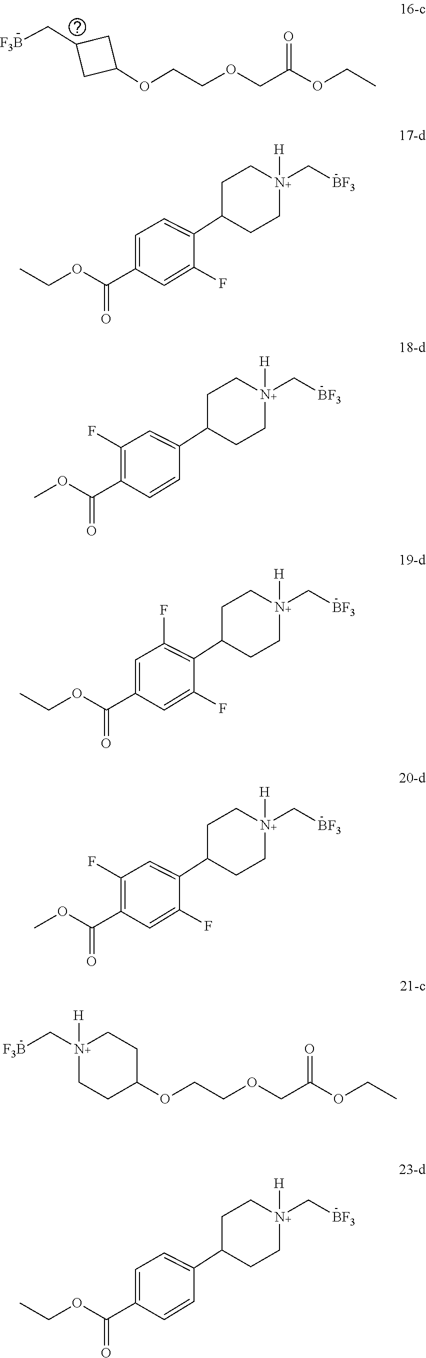

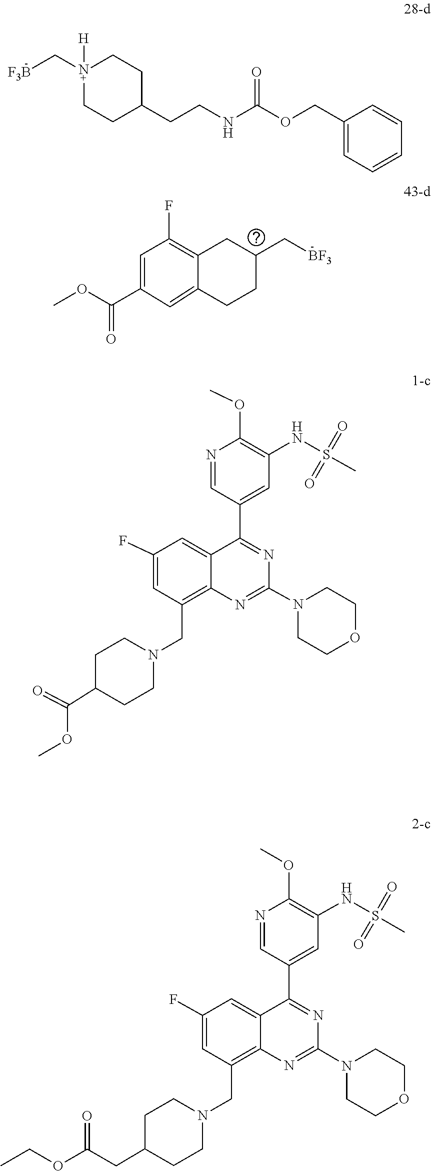

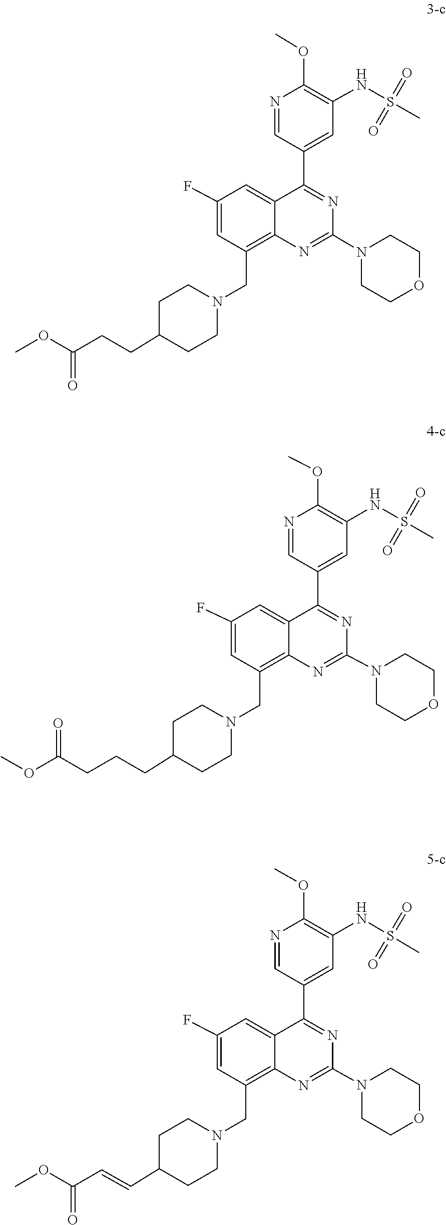

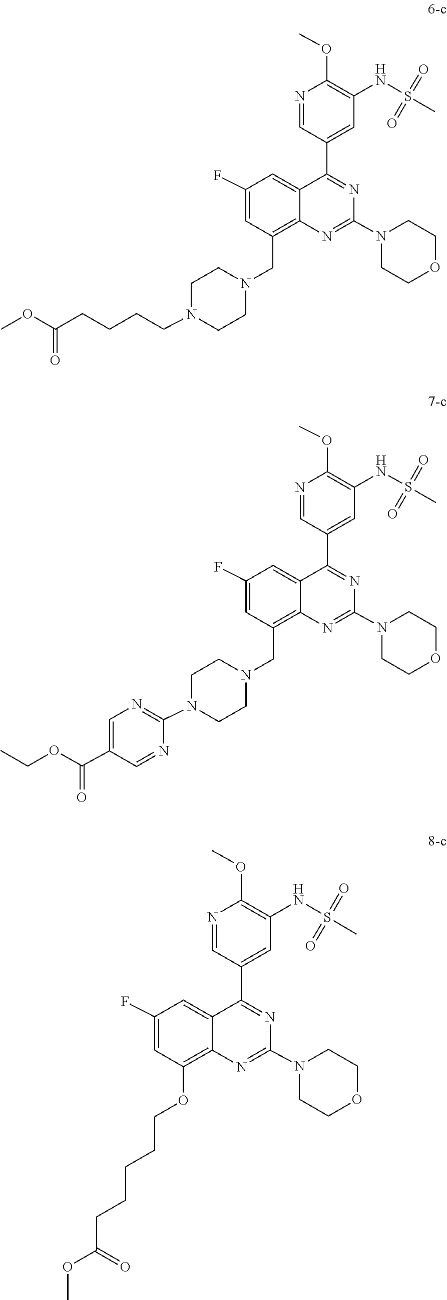 [SMILES: COC(=O)CCCCN1CCN(Cc2cc(F)cc3c(-c4cnc(OC)c(NS(C)(=O)=O)c4)nc(N4CCOCC4)nc23)CC1 ; CCOC(=O)c1cnc(N2CCN(Cc3cc(F)cc4c(-c5cnc(OC)c(NS(C)(=O)=O)c5)nc(N5CCOCC5)nc34)CC2)nc1 ; COC(=O)CCCCCOc1cc(F)cc2c(-c3cnc(OC)c(NS(C)(=O)=O)c3)nc(N3CCOCC3)nc12]

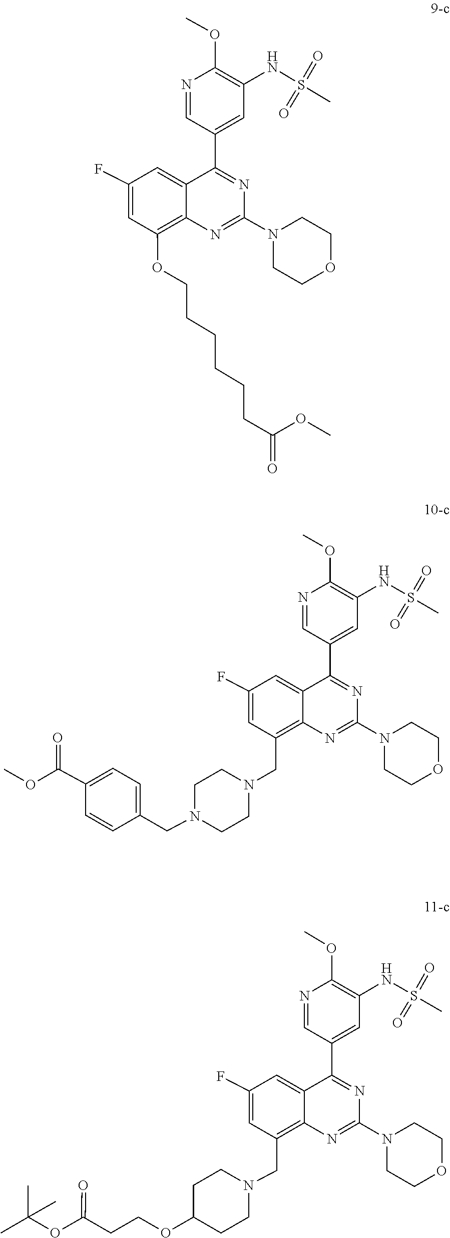 [SMILES: COC(=O)CCCCCCOc1cc(F)cc2c(-c3cnc(OC)c(NS(C)(=O)=O)c3)nc(N3CCOCC3)nc12 ; COC(=O)c1ccc(CN2CCN(Cc3cc(F)cc4c(-c5cnc(OC)c(NS(C)(=O)=O)c5)nc(N5CCOCC5)nc34)CC2)cc1 ; COc1ncc(-c2nc(N3CCOCC3)nc3c(CN4CCC(OCCC(=O)OC(C)(C)C)CC4)cc(F)cc23)cc1NS(C)(=O)=O]

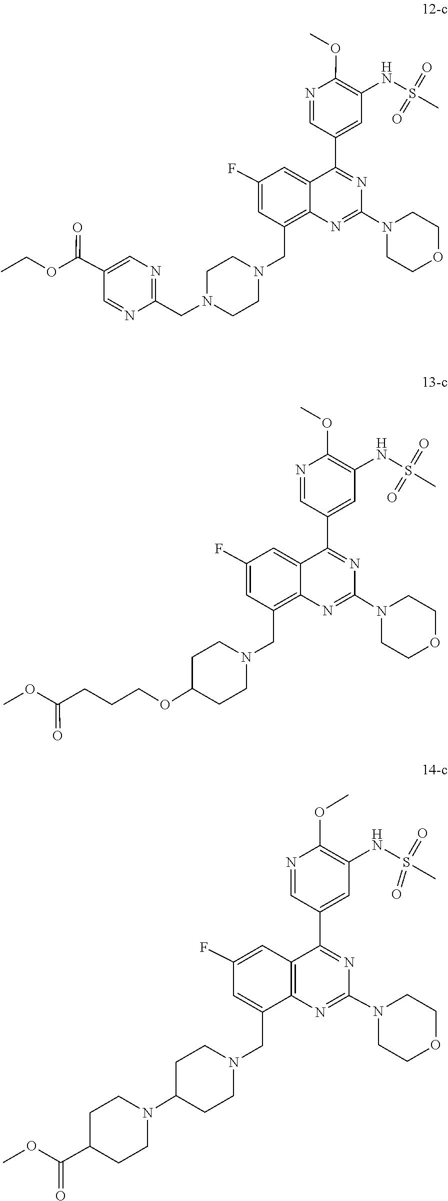

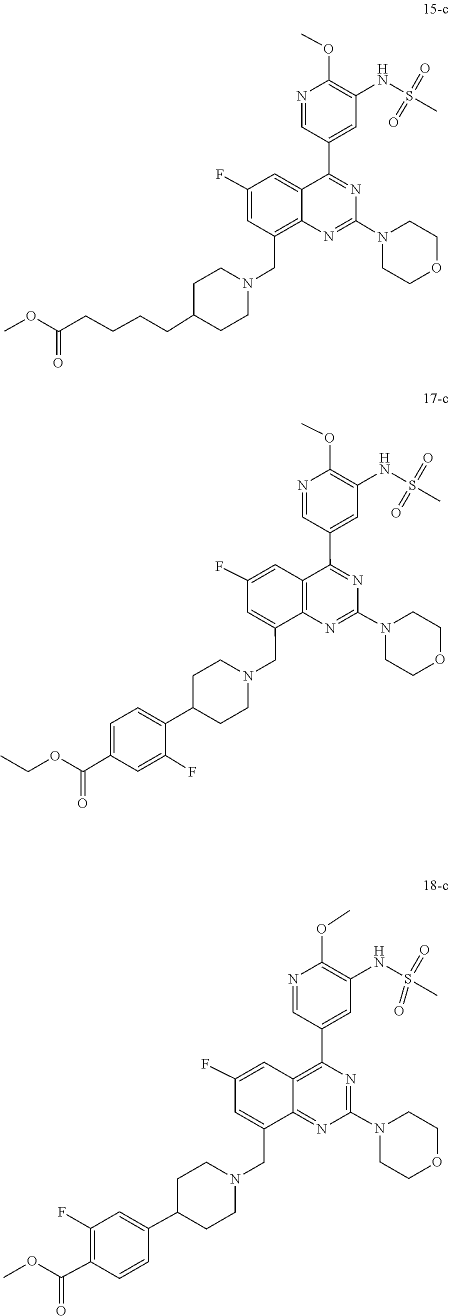

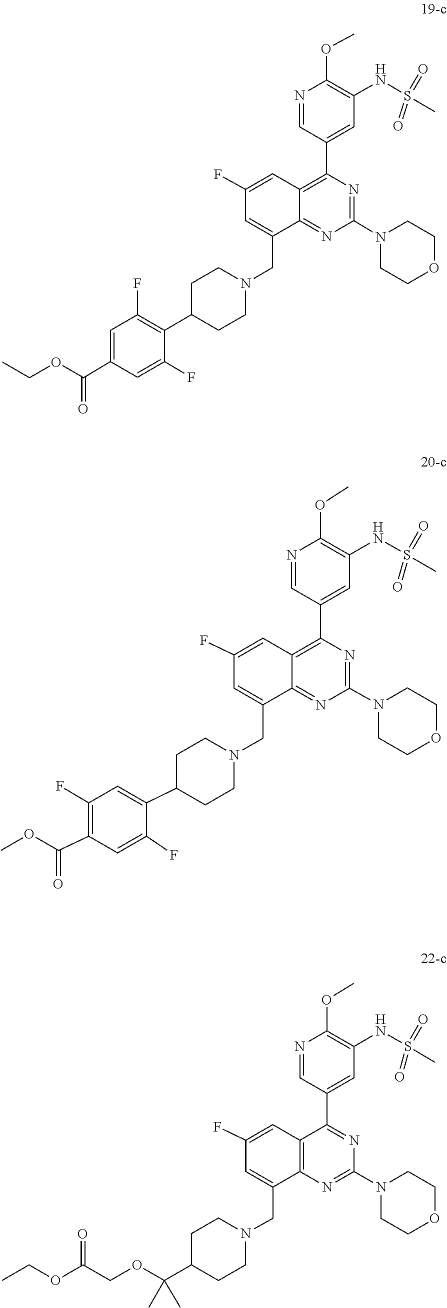

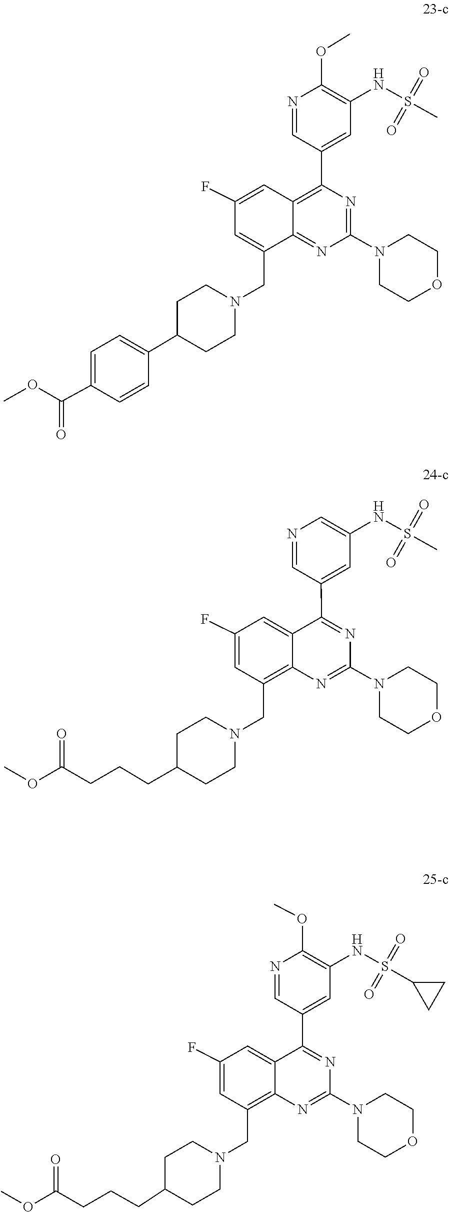

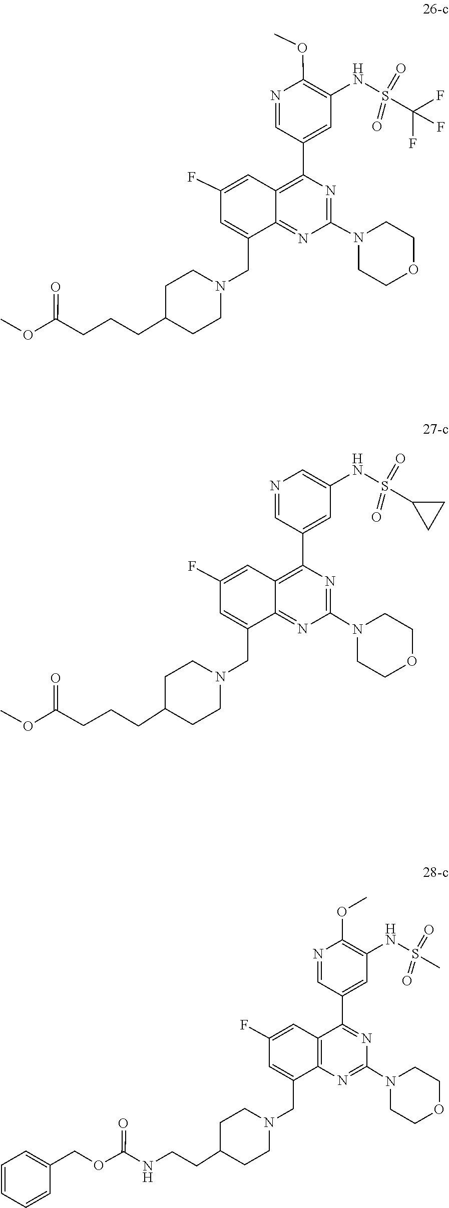 [SMILES: COC(=O)CCCC1CCN(Cc2cc(F)cc3c(-c4cnc(OC)c(NS(=O)(=O)C(F)(F)F)c4)nc(N4CCOCC4)nc23)CC1 ; COC(=O)CCCC1CCN(Cc2cc(F)cc3c(-c4cncc(NS(=O)(=O)C5CC5)c4)nc(N4CCOCC4)nc23)CC1 ; COc1ncc(-c2nc(N3CCOCC3)nc3c(CN4CCC(CCNC(=O)OCc5ccccc5)CC4)cc(F)cc23)cc1NS(C)(=O)=O]

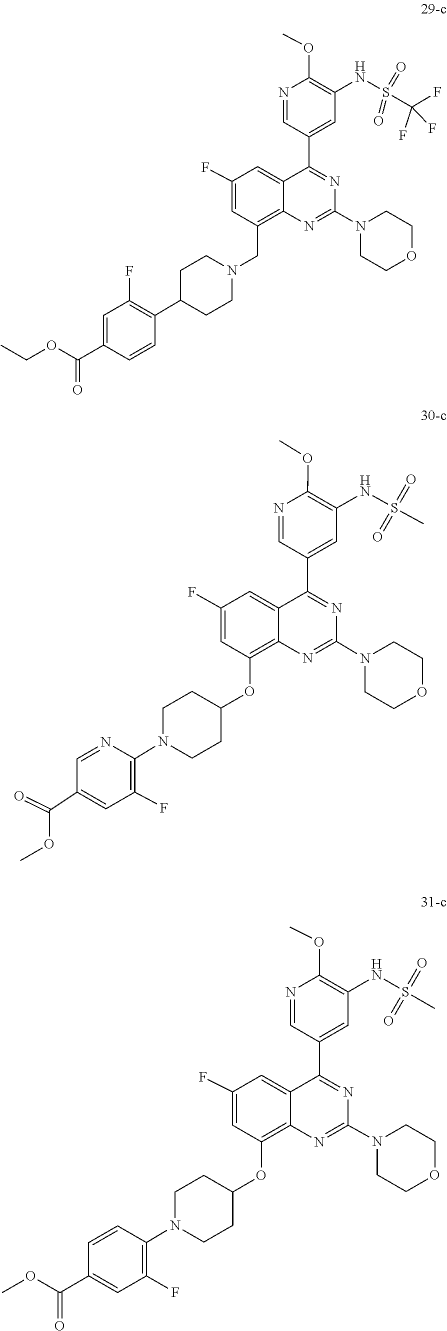

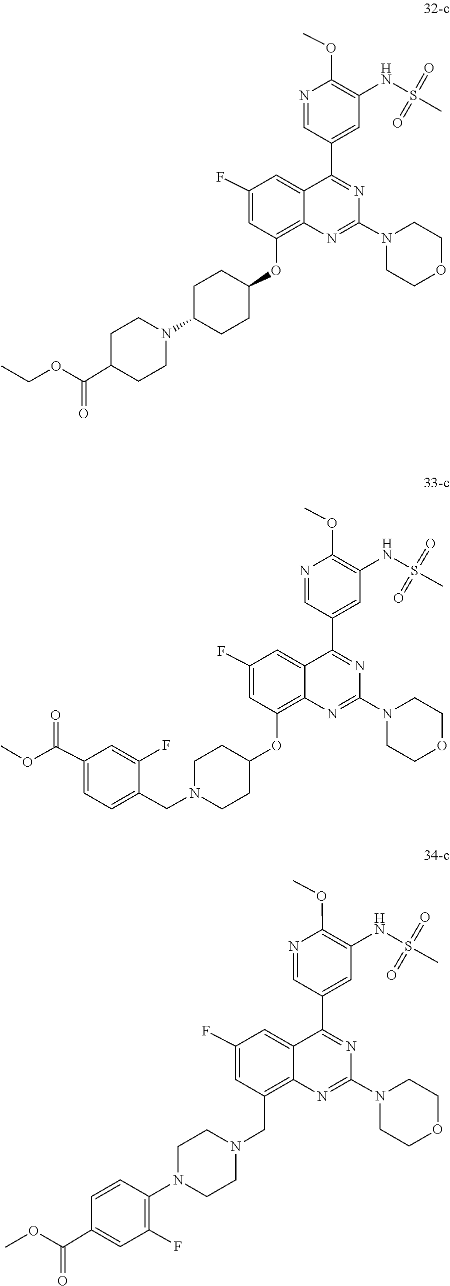

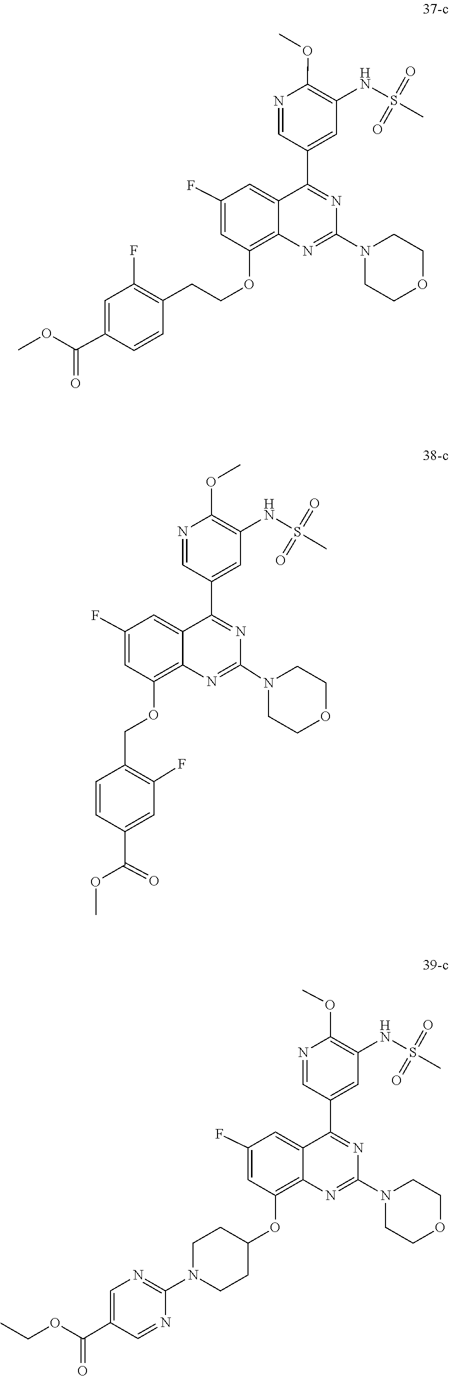

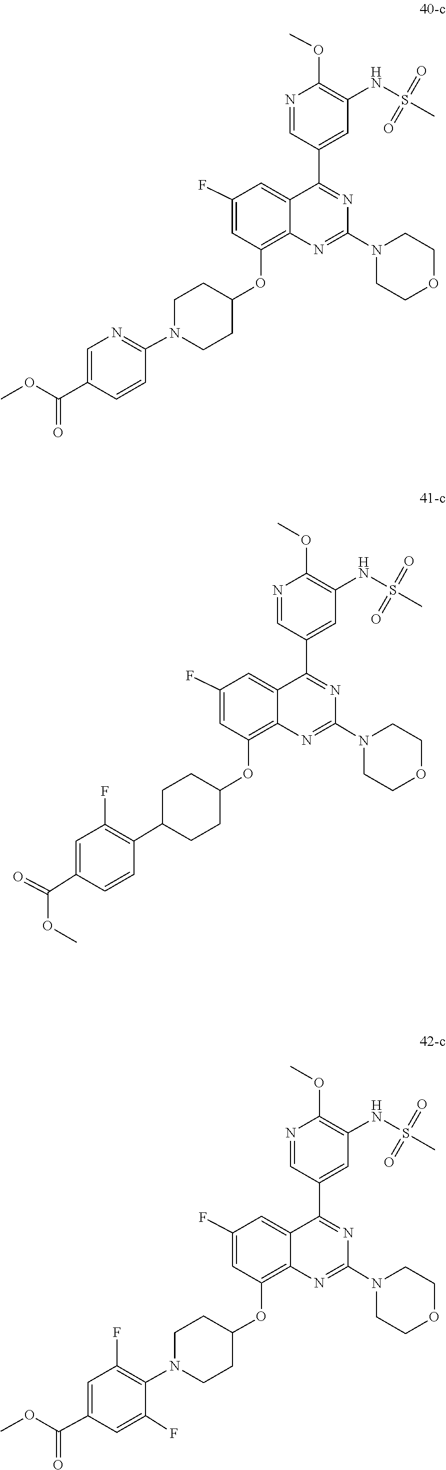

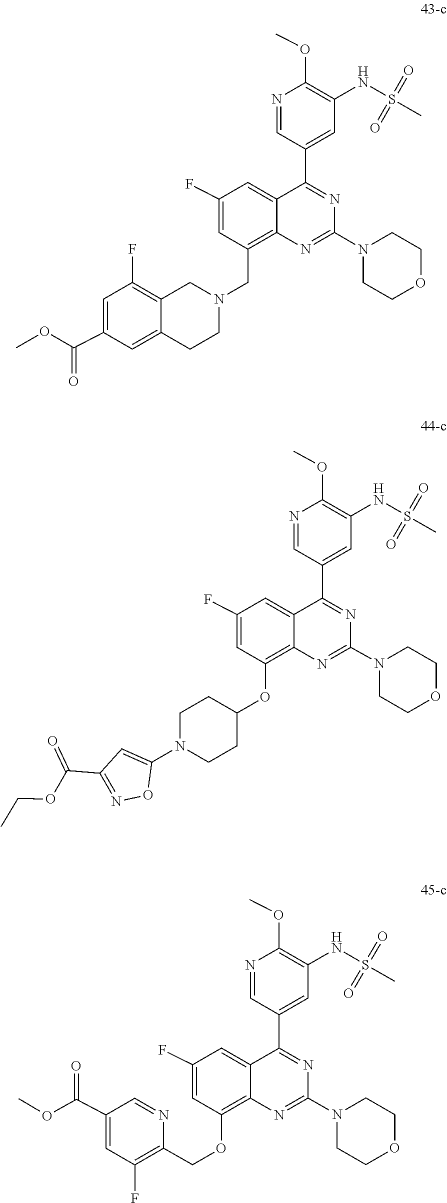

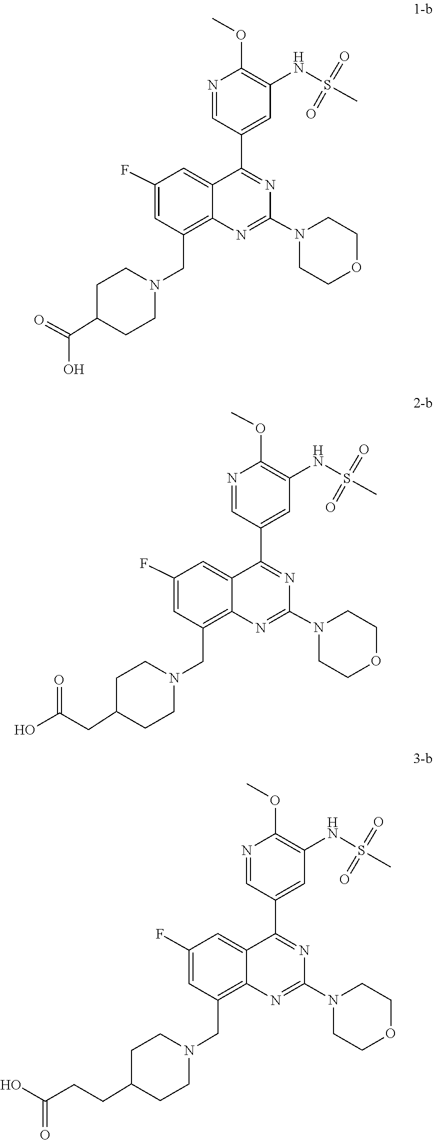 [SMILES: COc1ncc(-c2nc(N3CCOCC3)nc3c(CN4CCC(C(=O)O)CC4)cc(F)cc23)cc1NS(C)(=O)=O ; COc1ncc(-c2nc(N3CCOCC3)nc3c(CN4CCC(CC(=O)O)CC4)cc(F)cc23)cc1NS(C)(=O)=O ; COc1ncc(-c2nc(N3CCOCC3)nc3c(CN4CCC(CCC(=O)O)CC4)cc(F)cc23)cc1NS(C)(=O)=O]

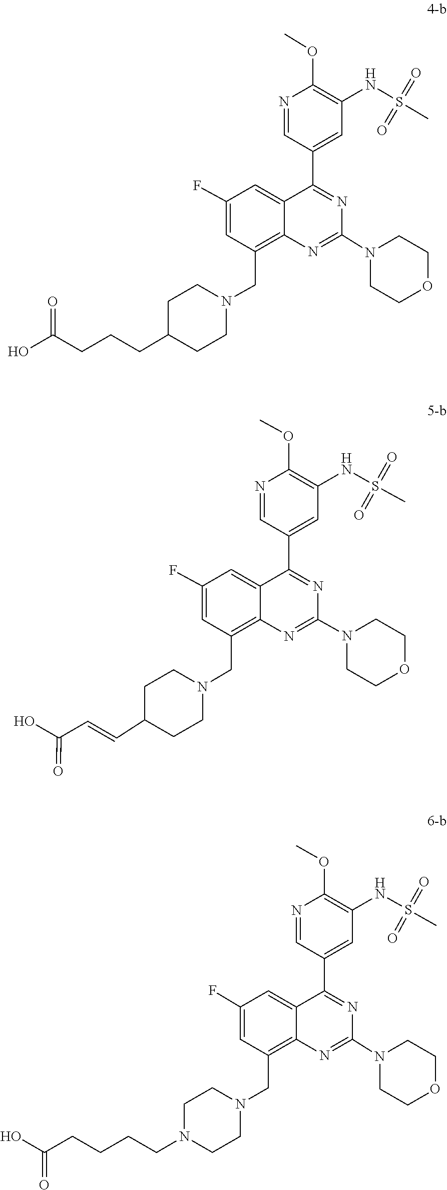 [SMILES: COc1ncc(-c2nc(N3CCOCC3)nc3c(CN4CCC(CCCC(=O)O)CC4)cc(F)cc23)cc1NS(C)(=O)=O ; COc1ncc(-c2nc(N3CCOCC3)nc3c(CN4CCC(/C=C/C(=O)O)CC4)cc(F)cc23)cc1NS(C)(=O)=O ; COc1ncc(-c2nc(N3CCOCC3)nc3c(CN4CCN(CCCCC(=O)O)CC4)cc(F)cc23)cc1NS(C)(=O)=O]

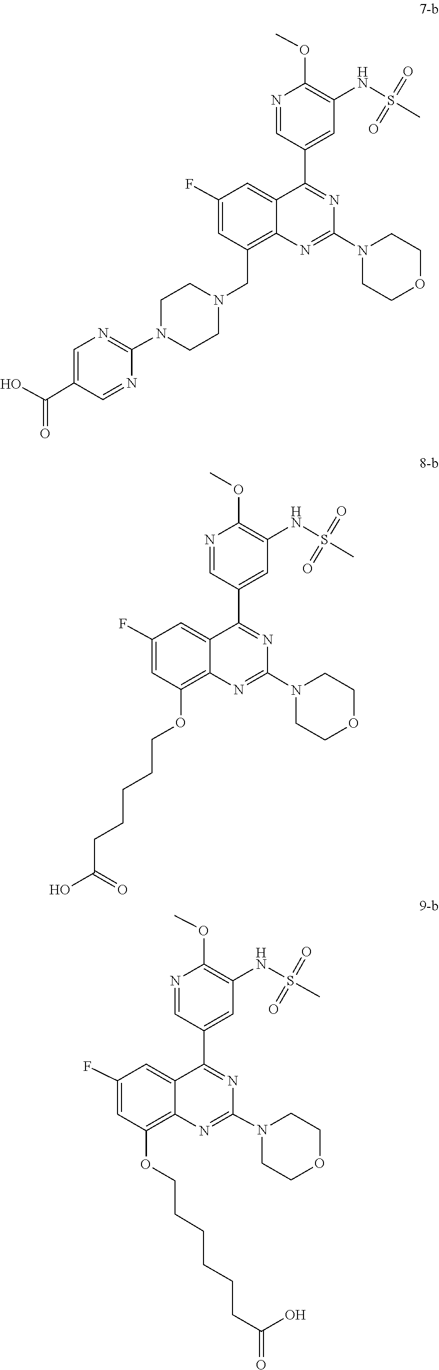 [SMILES: COc1ncc(-c2nc(N3CCOCC3)nc3c(CN4CCN(c5ncc(C(=O)O)cn5)CC4)cc(F)cc23)cc1NS(C)(=O)=O ; COc1ncc(-c2nc(N3CCOCC3)nc3c(OCCCCCC(=O)O)cc(F)cc23)cc1NS(C)(=O)=O ; COc1ncc(-c2nc(N3CCOCC3)nc3c(OCCCCCCC(=O)O)cc(F)cc23)cc1NS(C)(=O)=O]

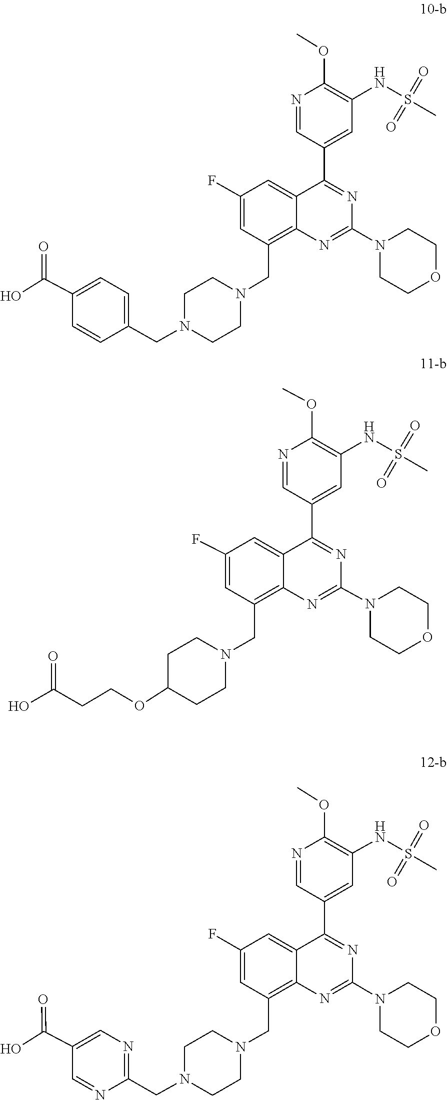 [SMILES: COc1ncc(-c2nc(N3CCOCC3)nc3c(CN4CCN(Cc5ccc(C(=O)O)cc5)CC4)cc(F)cc23)cc1NS(C)(=O)=O ; COc1ncc(-c2nc(N3CCOCC3)nc3c(CN4CCC(OCCC(=O)O)CC4)cc(F)cc23)cc1NS(C)(=O)=O ; COc1ncc(-c2nc(N3CCOCC3)nc3c(CN4CCN(Cc5ncc(C(=O)O)cn5)CC4)cc(F)cc23)cc1NS(C)(=O)=O]

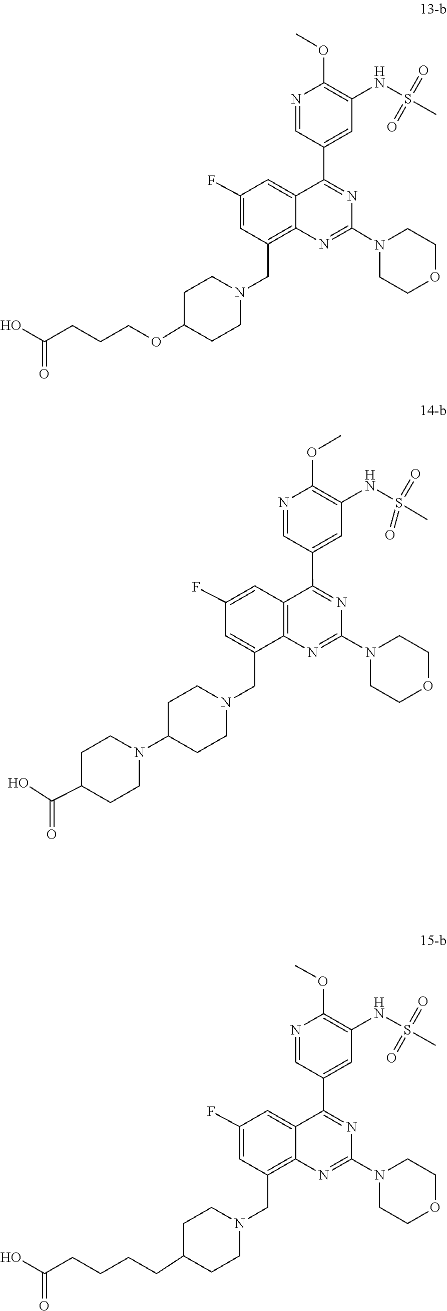 [SMILES: COc1ncc(-c2nc(N3CCOCC3)nc3c(CN4CCC(OCCCC(=O)O)CC4)cc(F)cc23)cc1NS(C)(=O)=O ; COc1ncc(-c2nc(N3CCOCC3)nc3c(CN4CCC(N5CCC(C(=O)O)CC5)CC4)cc(F)cc23)cc1NS(C)(=O)=O ; COc1ncc(-c2nc(N3CCOCC3)nc3c(CN4CCC(CCCCC(=O)O)CC4)cc(F)cc23)cc1NS(C)(=O)=O]

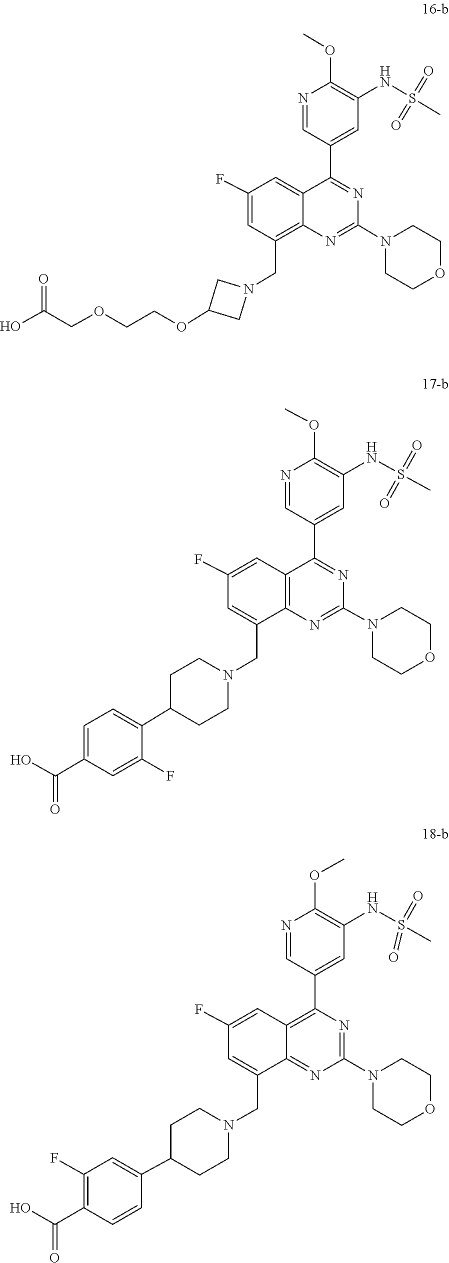 [SMILES: COc1ncc(-c2nc(N3CCOCC3)nc3c(CN4CC(OCCOCC(=O)O)C4)cc(F)cc23)cc1NS(C)(=O)=O ; COc1ncc(-c2nc(N3CCOCC3)nc3c(CN4CCC(c5ccc(C(=O)O)cc5F)CC4)cc(F)cc23)cc1NS(C)(=O)=O ; COc1ncc(-c2nc(N3CCOCC3)nc3c(CN4CCC(c5ccc(C(=O)O)c(F)c5)CC4)cc(F)cc23)cc1NS(C)(=O)=O]

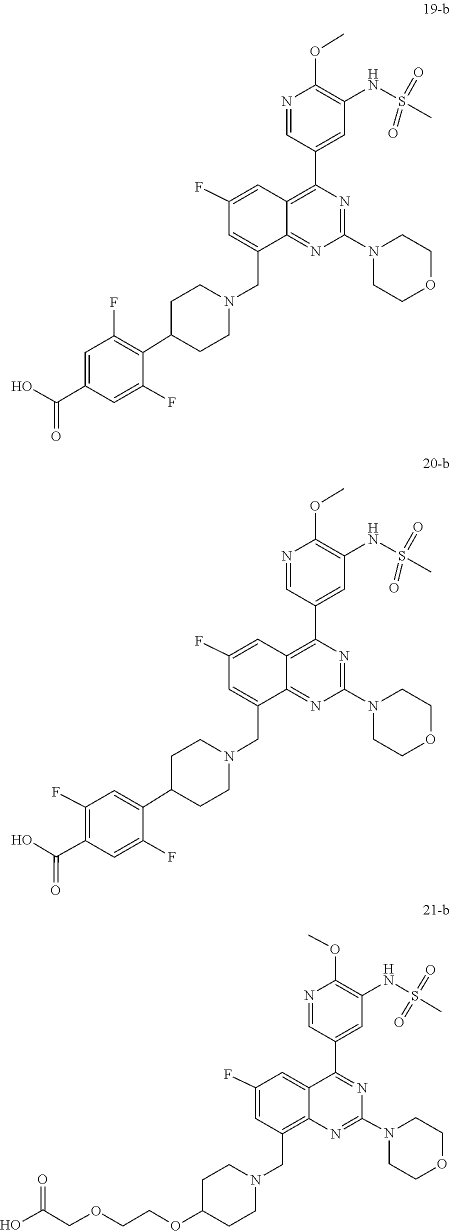 [SMILES: COc1ncc(-c2nc(N3CCOCC3)nc3c(CN4CCC(c5c(F)cc(C(=O)O)cc5F)CC4)cc(F)cc23)cc1NS(C)(=O)=O ; COc1ncc(-c2nc(N3CCOCC3)nc3c(CN4CCC(c5cc(F)c(C(=O)O)cc5F)CC4)cc(F)cc23)cc1NS(C)(=O)=O ; COc1ncc(-c2nc(N3CCOCC3)nc3c(CN4CCC(OCCOCC(=O)O)CC4)cc(F)cc23)cc1NS(C)(=O)=O]

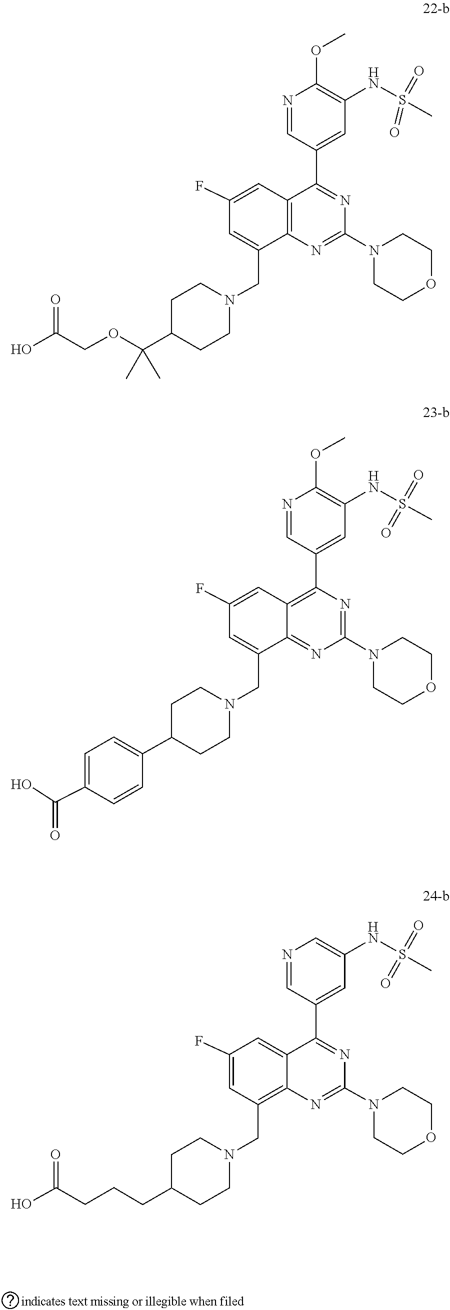 [SMILES: COc1ncc(-c2nc(N3CCOCC3)nc3c(CN4CCC(C(C)(C)OCC(=O)O)CC4)cc(F)cc23)cc1NS(C)(=O)=O ; COc1ncc(-c2nc(N3CCOCC3)nc3c(CN4CCC(c5ccc(C(=O)O)cc5)CC4)cc(F)cc23)cc1NS(C)(=O)=O ; CS(=O)(=O)Nc1cncc(-c2nc(N3CCOCC3)nc3c(CN4CCC(CCCC(=O)O)CC4)cc(F)cc23)c1]

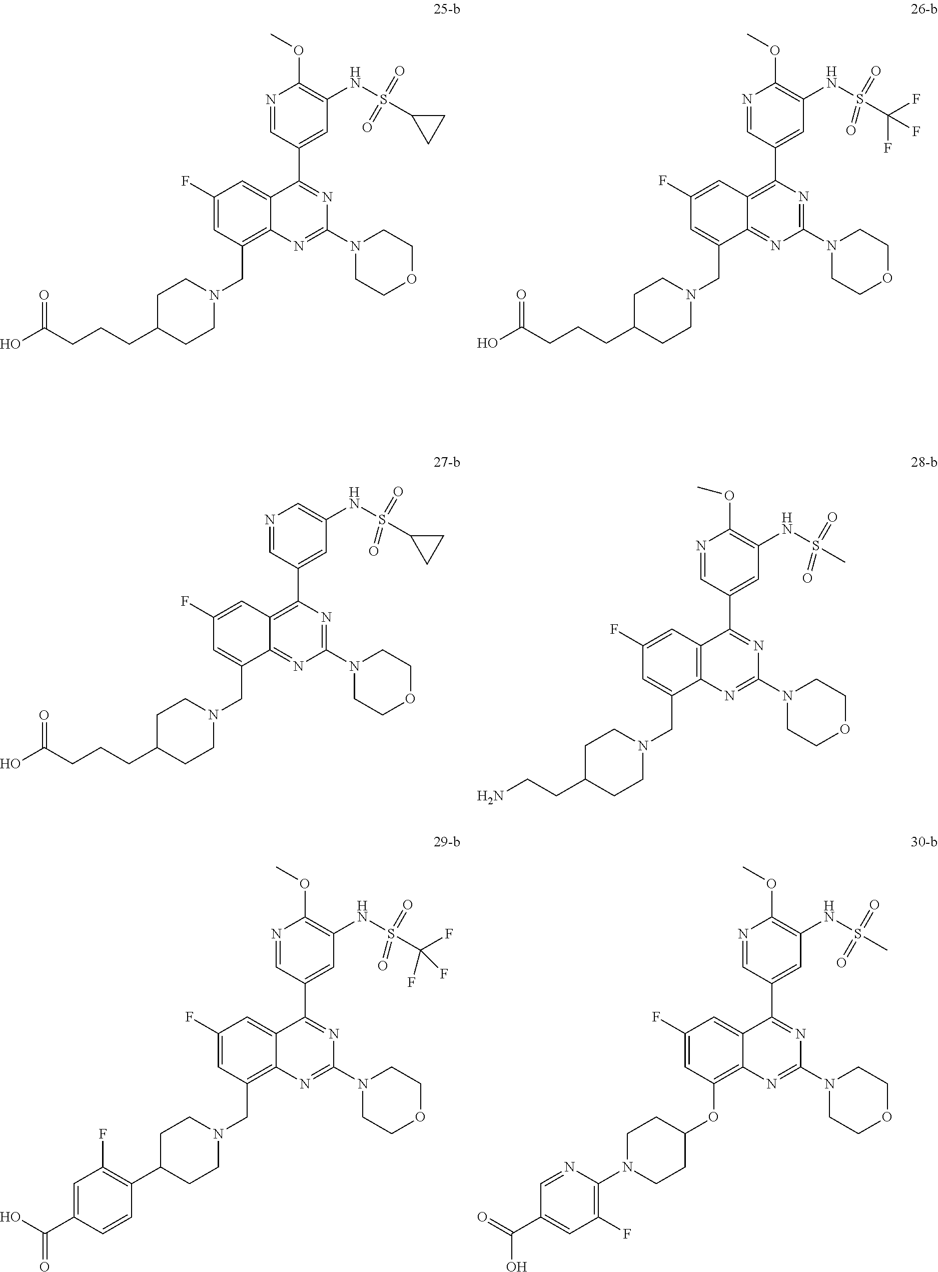 [SMILES: COc1ncc(-c2nc(N3CCOCC3)nc3c(CN4CCC(CCCC(=O)O)CC4)cc(F)cc23)cc1NS(=O)(=O)C1CC1 ; COc1ncc(-c2nc(N3CCOCC3)nc3c(CN4CCC(CCCC(=O)O)CC4)cc(F)cc23)cc1NS(=O)(=O)C(F)(F)F ; O=C(O)CCCC1CCN(Cc2cc(F)cc3c(-c4cncc(NS(=O)(=O)C5CC5)c4)nc(N4CCOCC4)nc23)CC1 ; COc1ncc(-c2nc(N3CCOCC3)nc3c(CN4CCC(CCN)CC4)cc(F)cc23)cc1NS(C)(=O)=O ; COc1ncc(-c2nc(N3CCOCC3)nc3c(CN4CCC(c5ccc(C(=O)O)cc5F)CC4)cc(F)cc23)cc1NS(=O)(=O)C(F)(F)F ; COc1ncc(-c2nc(N3CCOCC3)nc3c(OC4CCN(c5ncc(C(=O)O)cc5F)CC4)cc(F)cc23)cc1NS(C)(=O)=O]

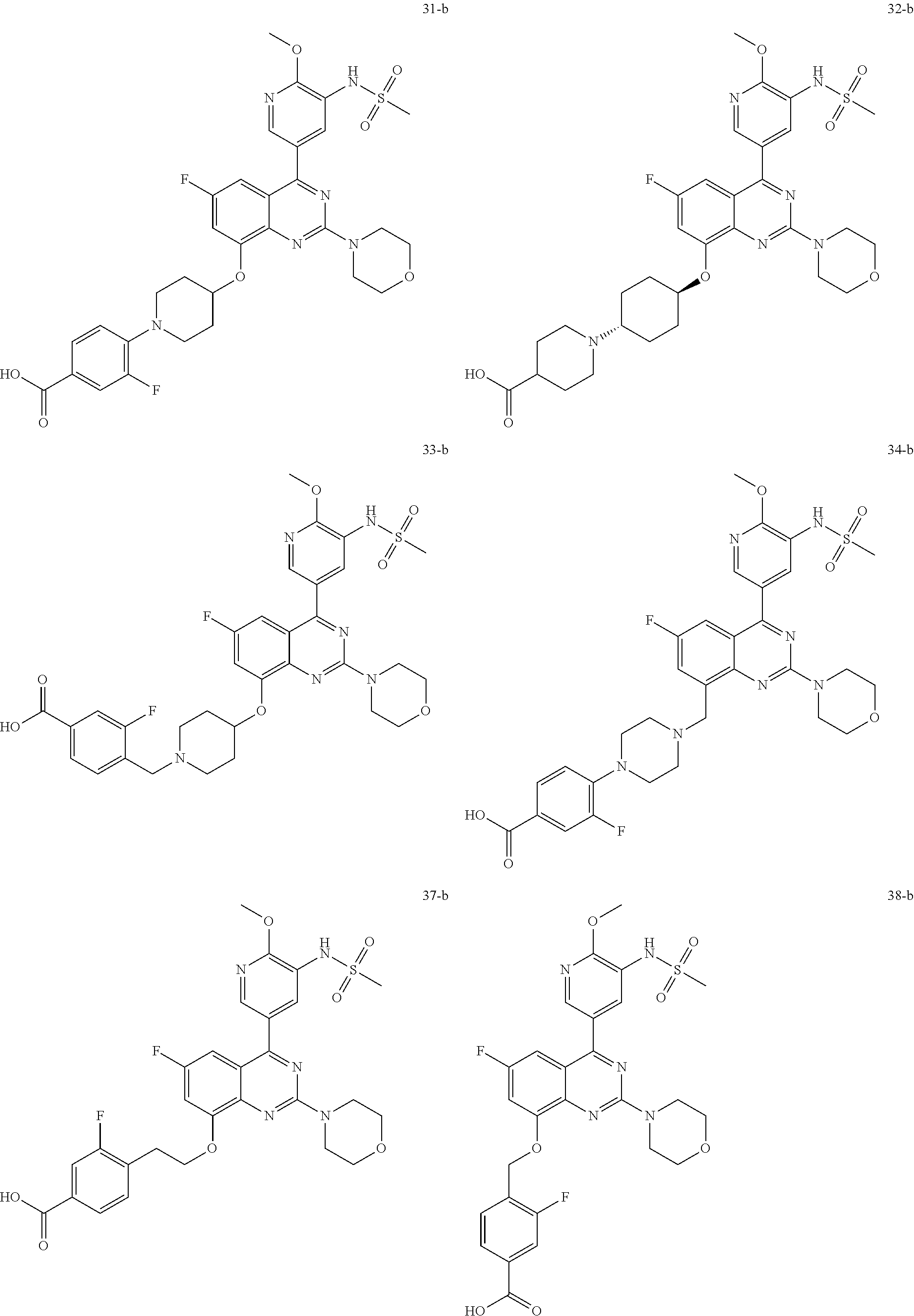 [SMILES: COc1ncc(-c2nc(N3CCOCC3)nc3c(OC4CCN(c5ccc(C(=O)O)cc5F)CC4)cc(F)cc23)cc1NS(C)(=O)=O ; COc1ncc(-c2nc(N3CCOCC3)nc3c(O[C@H]4CC[C@H](N5CCC(C(=O)O)CC5)CC4)cc(F)cc23)cc1NS(C)(=O)=O ; COc1ncc(-c2nc(N3CCOCC3)nc3c(OC4CCN(Cc5ccc(C(=O)O)cc5F)CC4)cc(F)cc23)cc1NS(C)(=O)=O ; COc1ncc(-c2nc(N3CCOCC3)nc3c(CN4CCN(c5ccc(C(=O)O)cc5F)CC4)cc(F)cc23)cc1NS(C)(=O)=O ; COc1ncc(-c2nc(N3CCOCC3)nc3c(OCCc4ccc(C(=O)O)cc4F)cc(F)cc23)cc1NS(C)(=O)=O ; COc1ncc(-c2nc(N3CCOCC3)nc3c(OCc4ccc(C(=O)O)cc4F)cc(F)cc23)cc1NS(C)(=O)=O]

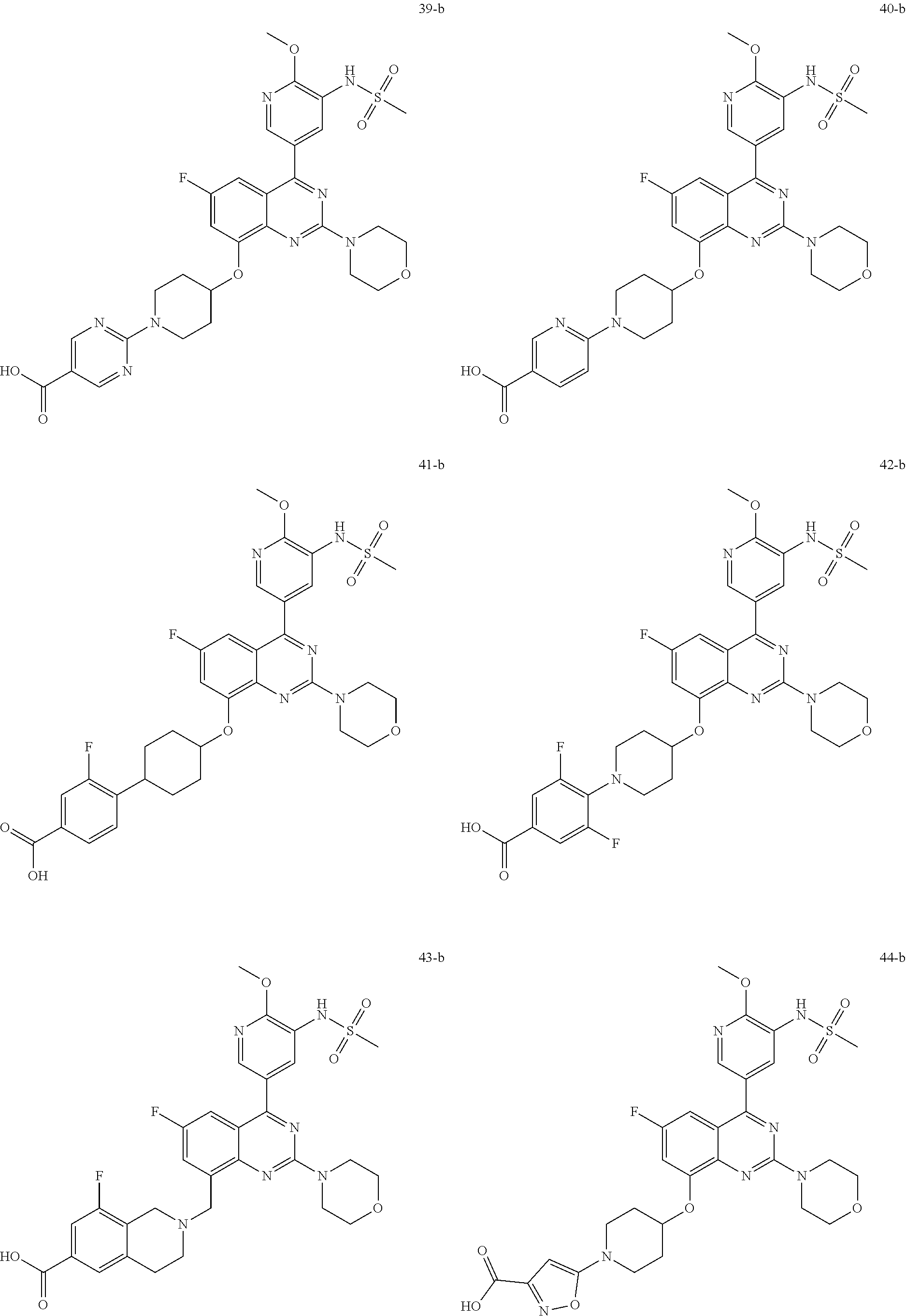 [SMILES: COc1ncc(-c2nc(N3CCOCC3)nc3c(OC4CCN(c5ncc(C(=O)O)cn5)CC4)cc(F)cc23)cc1NS(C)(=O)=O ; COc1ncc(-c2nc(N3CCOCC3)nc3c(OC4CCN(c5ccc(C(=O)O)cn5)CC4)cc(F)cc23)cc1NS(C)(=O)=O ; COc1ncc(-c2nc(N3CCOCC3)nc3c(OC4CCC(c5ccc(C(=O)O)cc5F)CC4)cc(F)cc23)cc1NS(C)(=O)=O ; COc1ncc(-c2nc(N3CCOCC3)nc3c(OC4CCN(c5c(F)cc(C(=O)O)cc5F)CC4)cc(F)cc23)cc1NS(C)(=O)=O ; COc1ncc(-c2nc(N3CCOCC3)nc3c(CN4CCc5cc(C(=O)O)cc(F)c5C4)cc(F)cc23)cc1NS(C)(=O)=O ; COc1ncc(-c2nc(N3CCOCC3)nc3c(OC4CCN(c5cc(C(=O)O)no5)CC4)cc(F)cc23)cc1NS(C)(=O)=O]

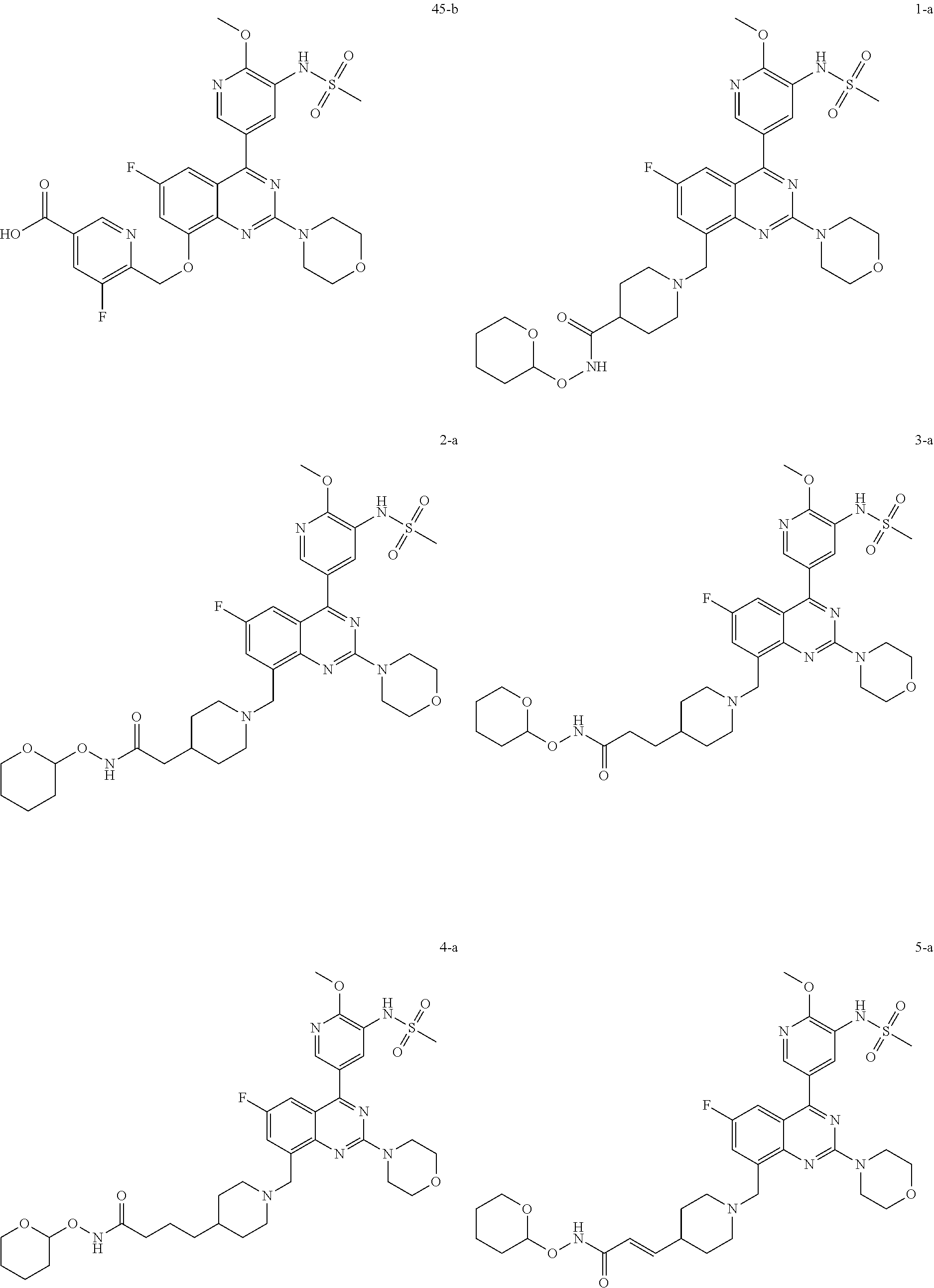

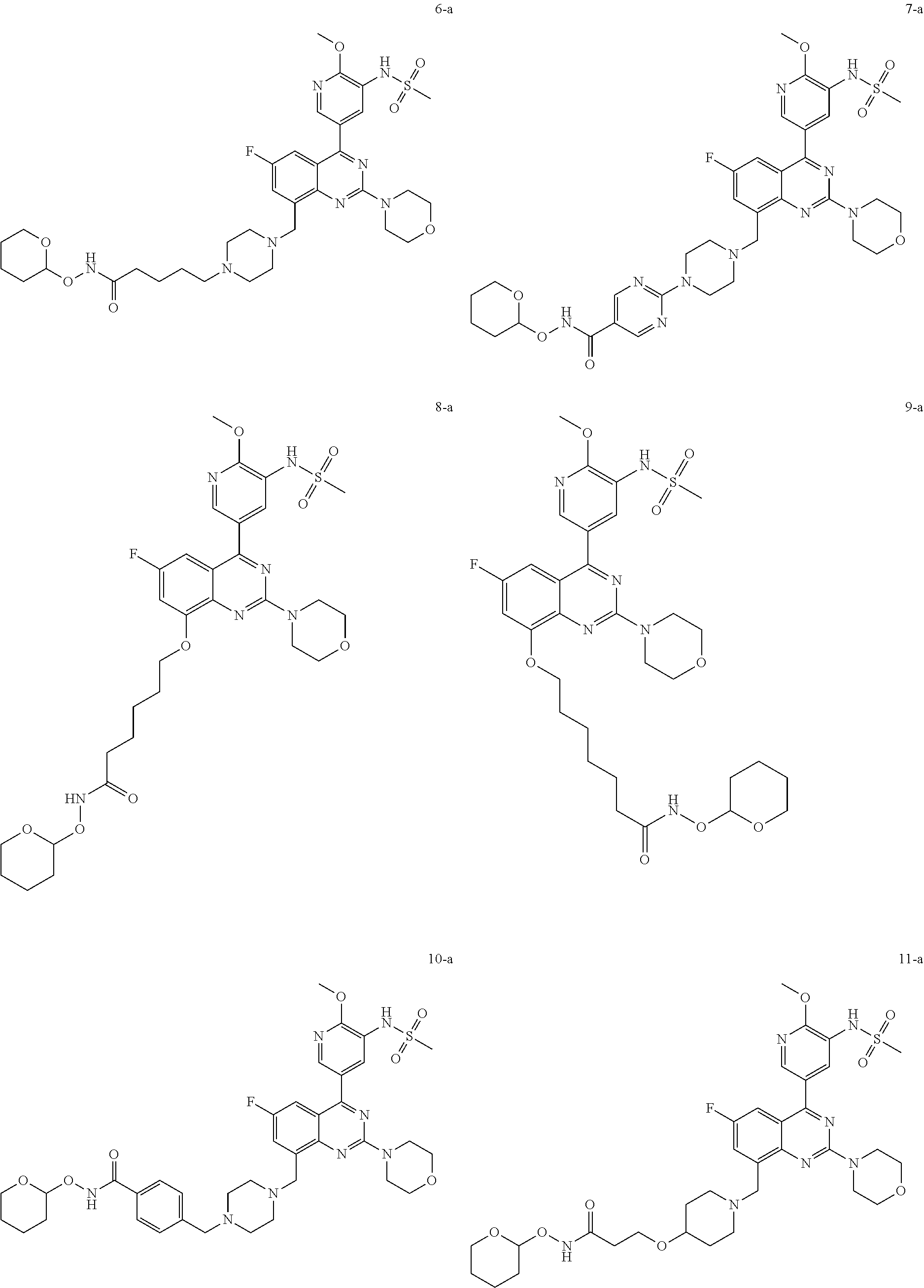

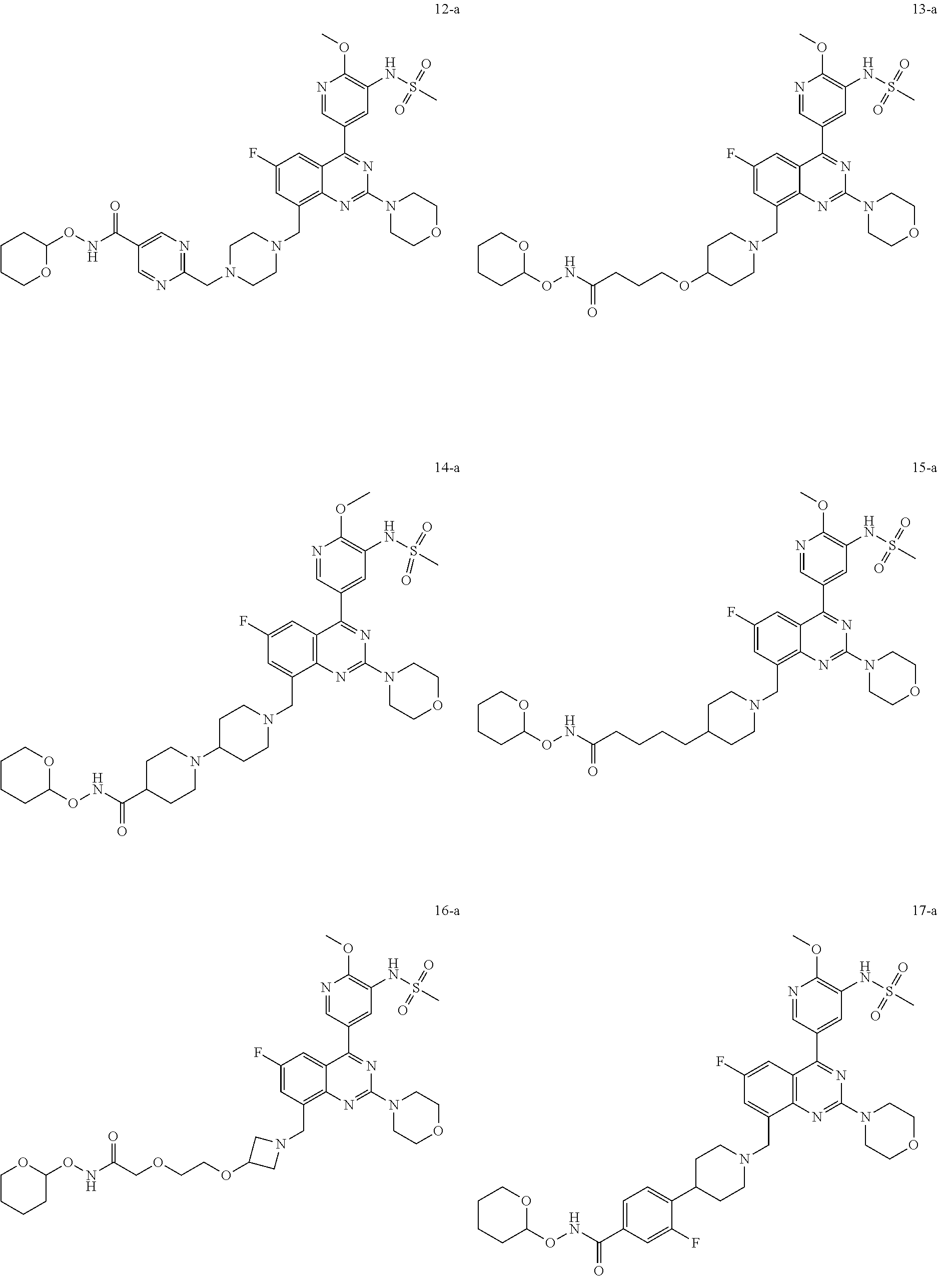

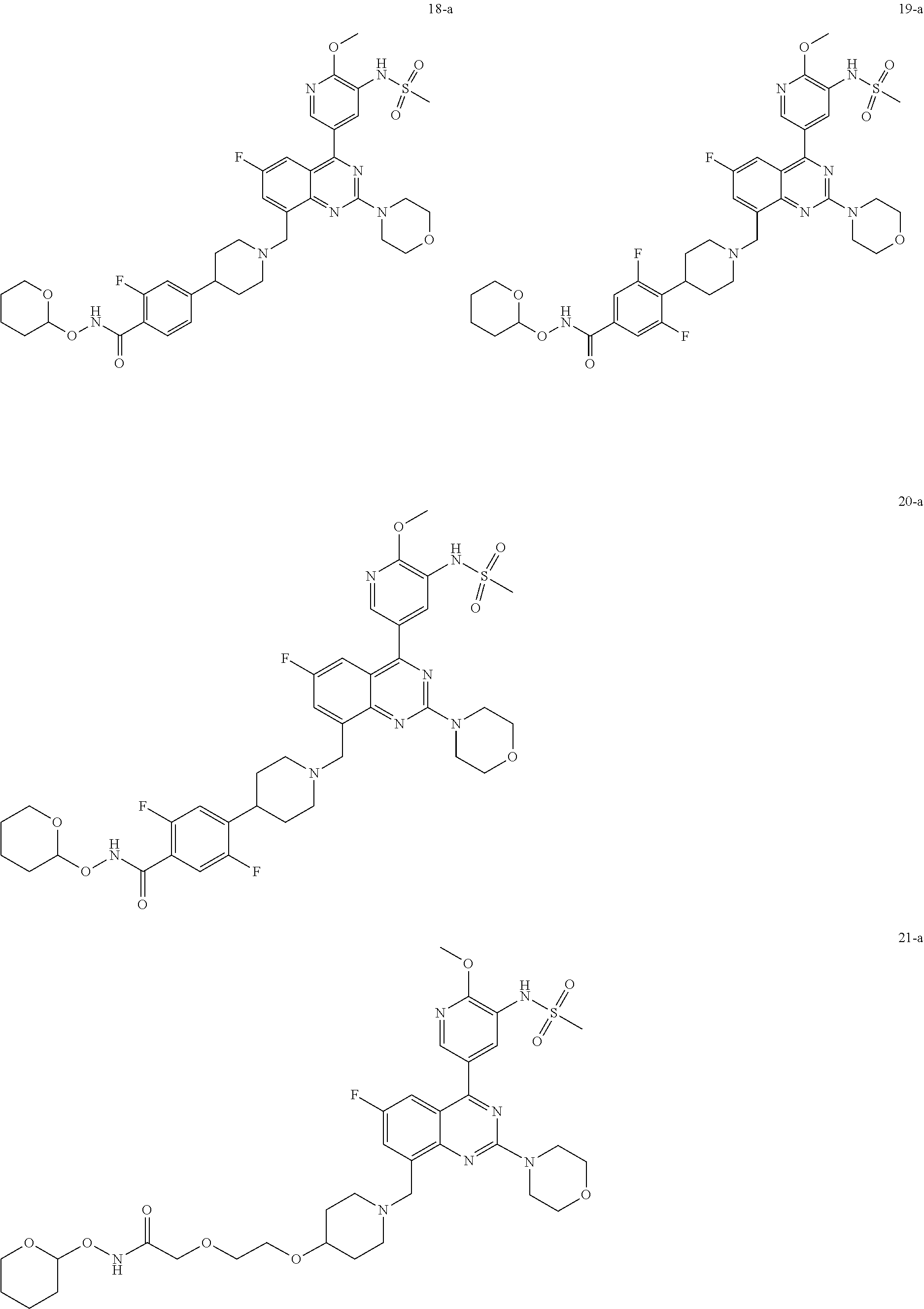 [SMILES: COc1ncc(-c2nc(N3CCOCC3)nc3c(CN4CCC(c5ccc(C(=O)NOC6CCCCO6)c(F)c5)CC4)cc(F)cc23)cc1NS(C)(=O)=O ; COc1ncc(-c2nc(N3CCOCC3)nc3c(CN4CCC(c5c(F)cc(C(=O)NOC6CCCCO6)cc5F)CC4)cc(F)cc23)cc1NS(C)(=O)=O ; COc1ncc(-c2nc(N3CCOCC3)nc3c(CN4CCC(c5cc(F)c(C(=O)NOC6CCCCO6)cc5F)CC4)cc(F)cc23)cc1NS(C)(=O)=O ; COc1ncc(-c2nc(N3CCOCC3)nc3c(CN4CCC(OCCOCC(=O)NOC5CCCCO5)CC4)cc(F)cc23)cc1NS(C)(=O)=O]

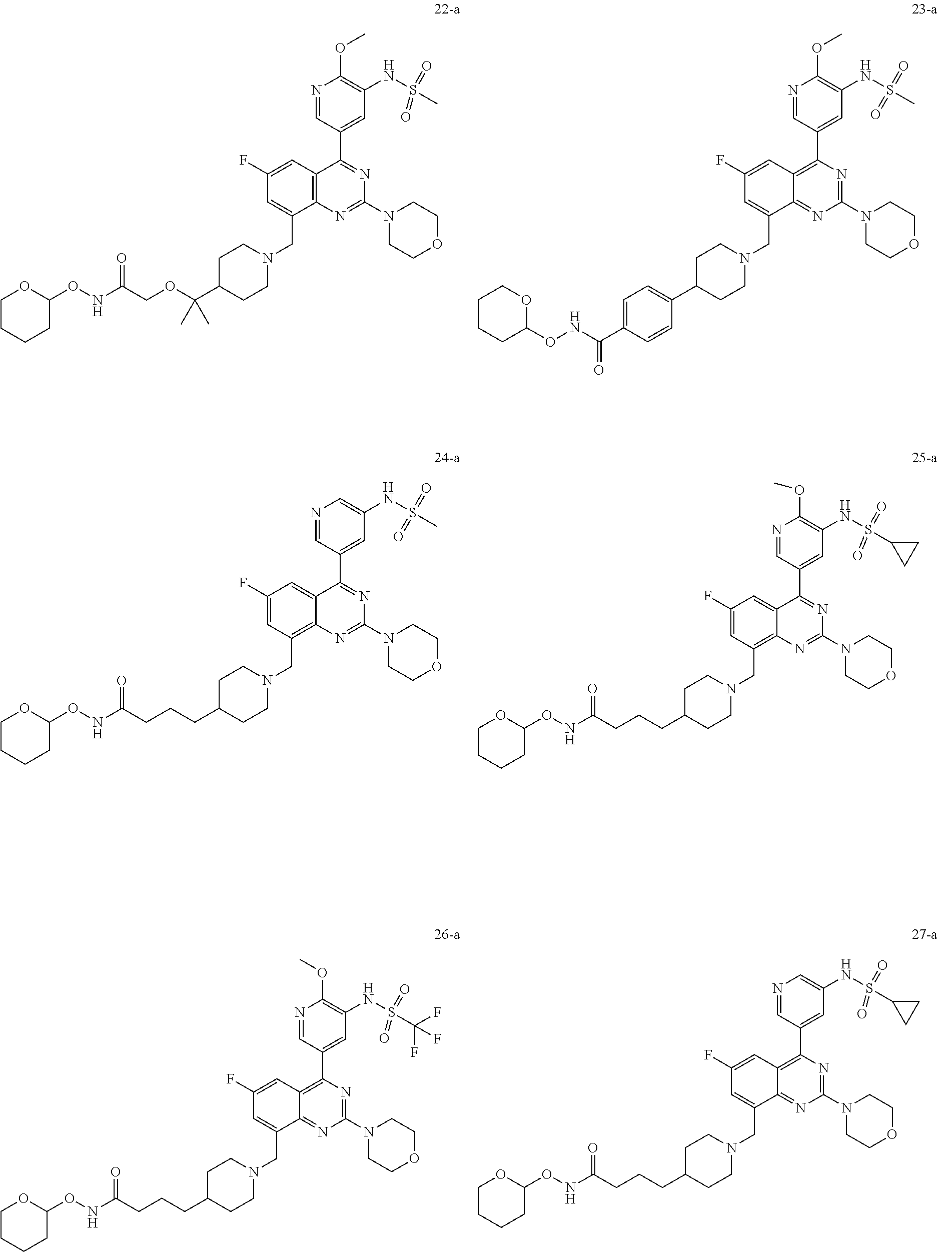

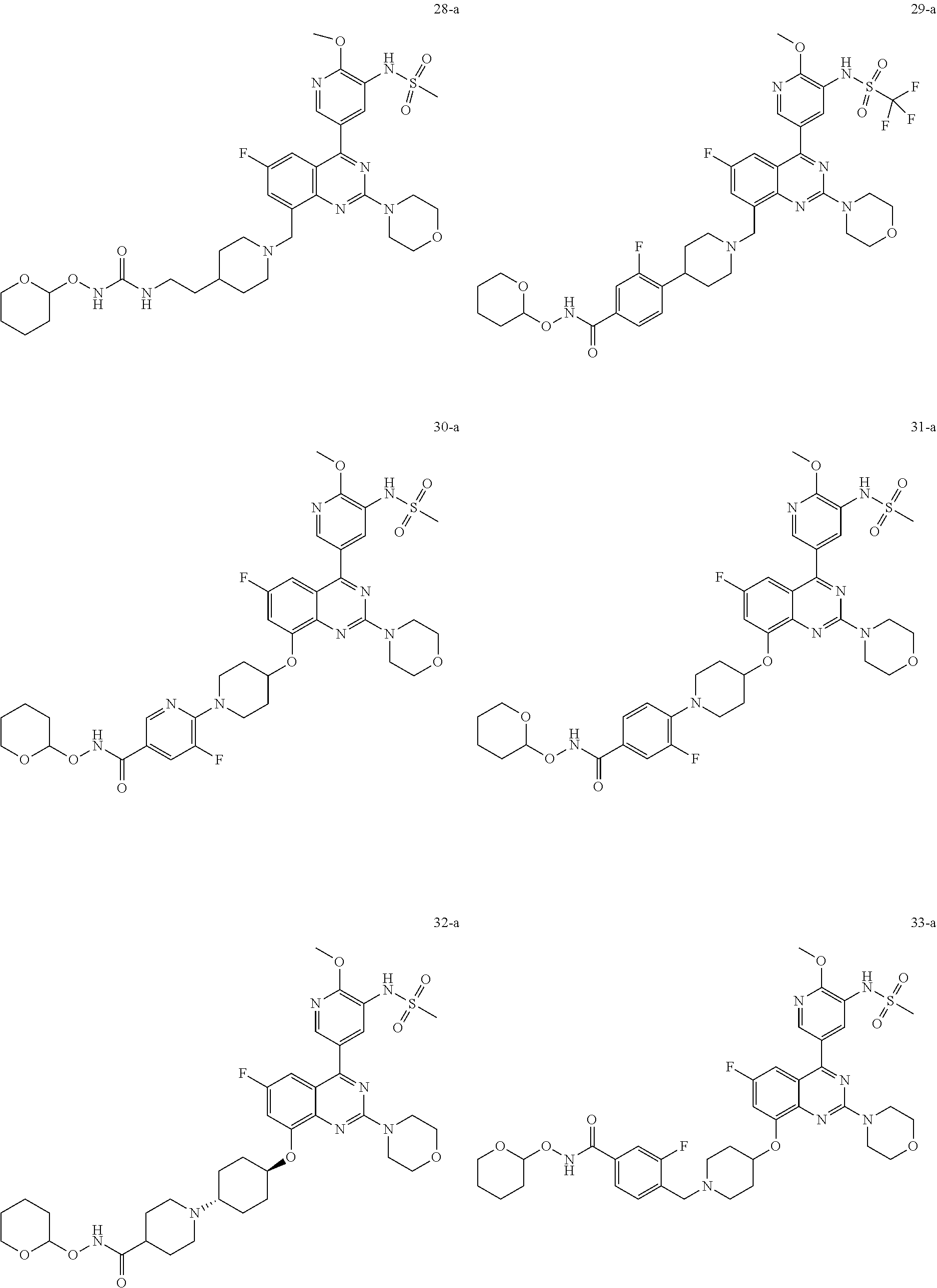

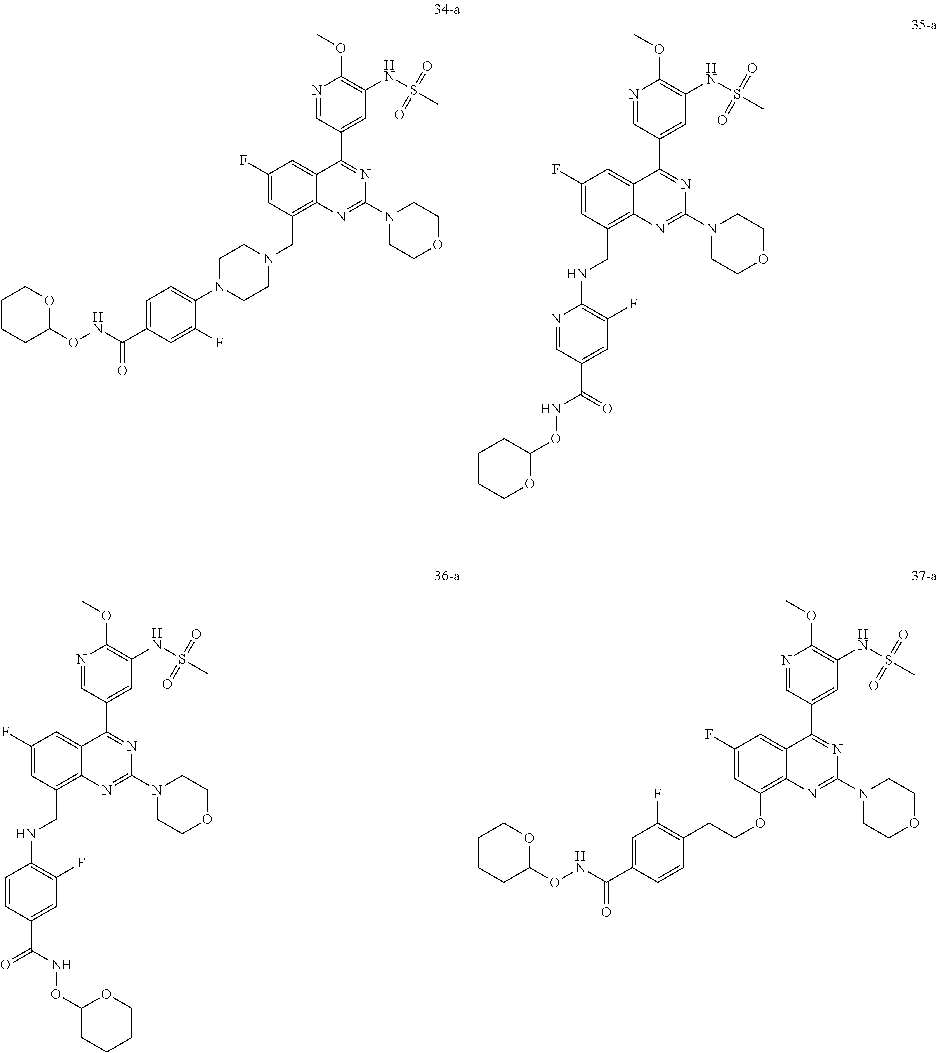 [SMILES: COc1ncc(-c2nc(N3CCOCC3)nc3c(CN4CCN(c5ccc(C(=O)NOC6CCCCO6)cc5F)CC4)cc(F)cc23)cc1NS(C)(=O)=O ; COc1ncc(-c2nc(N3CCOCC3)nc3c(CNc4ncc(C(=O)NOC5CCCCO5)cc4F)cc(F)cc23)cc1NS(C)(=O)=O ; COc1ncc(-c2nc(N3CCOCC3)nc3c(CNc4ccc(C(=O)NOC5CCCCO5)cc4F)cc(F)cc23)cc1NS(C)(=O)=O ; COc1ncc(-c2nc(N3CCOCC3)nc3c(OCCc4ccc(C(=O)NOC5CCCCO5)cc4F)cc(F)cc23)cc1NS(C)(=O)=O]

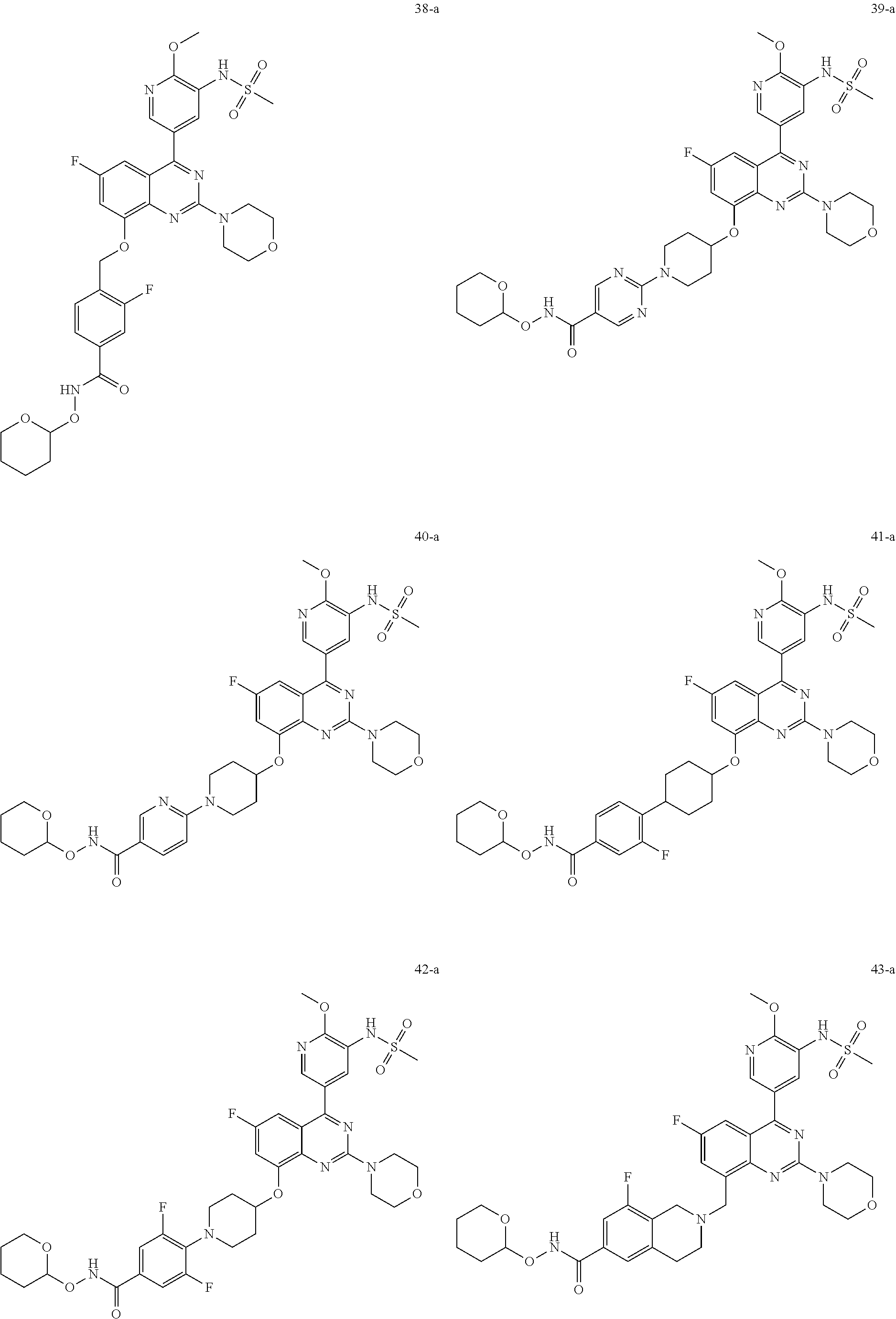

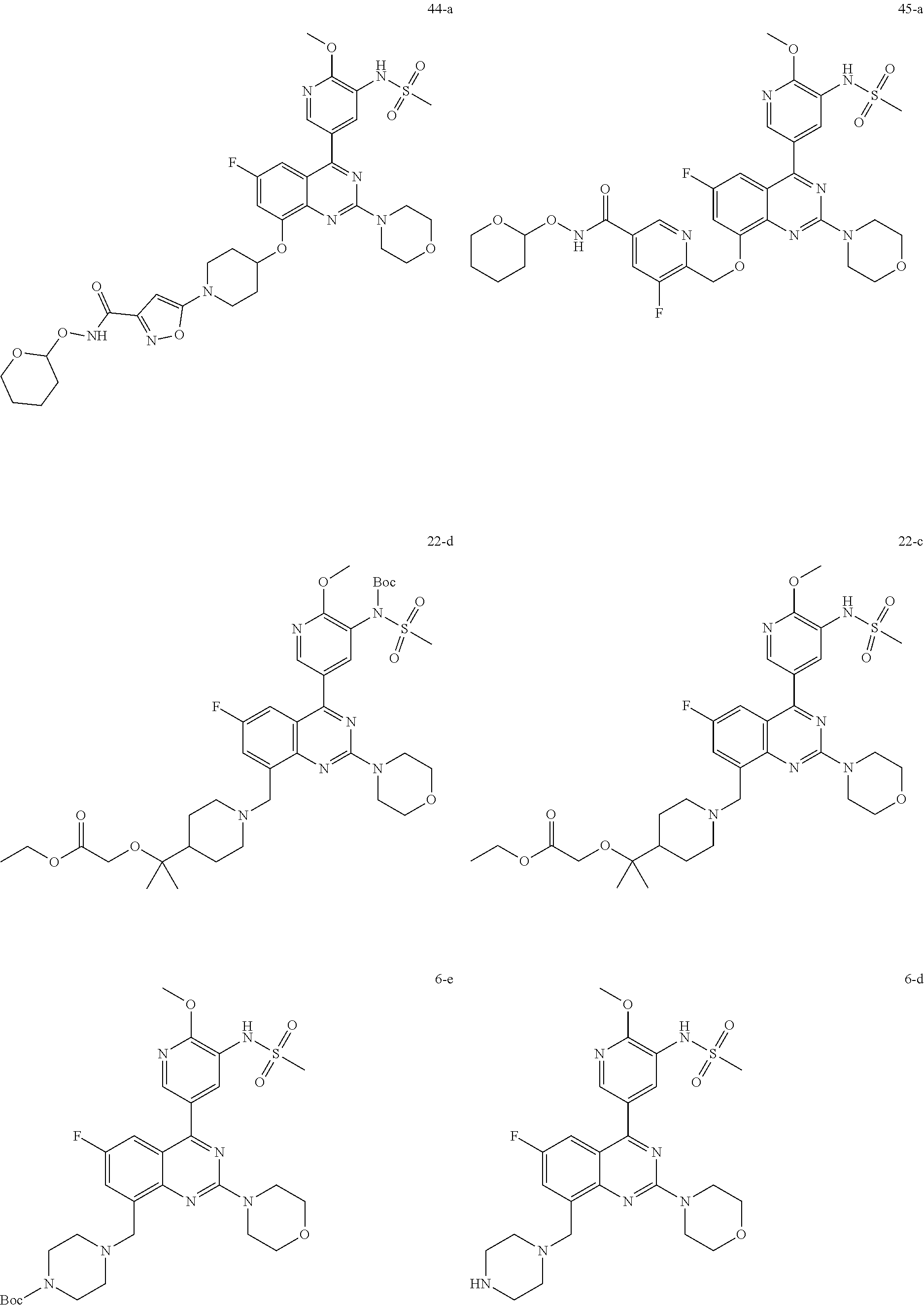

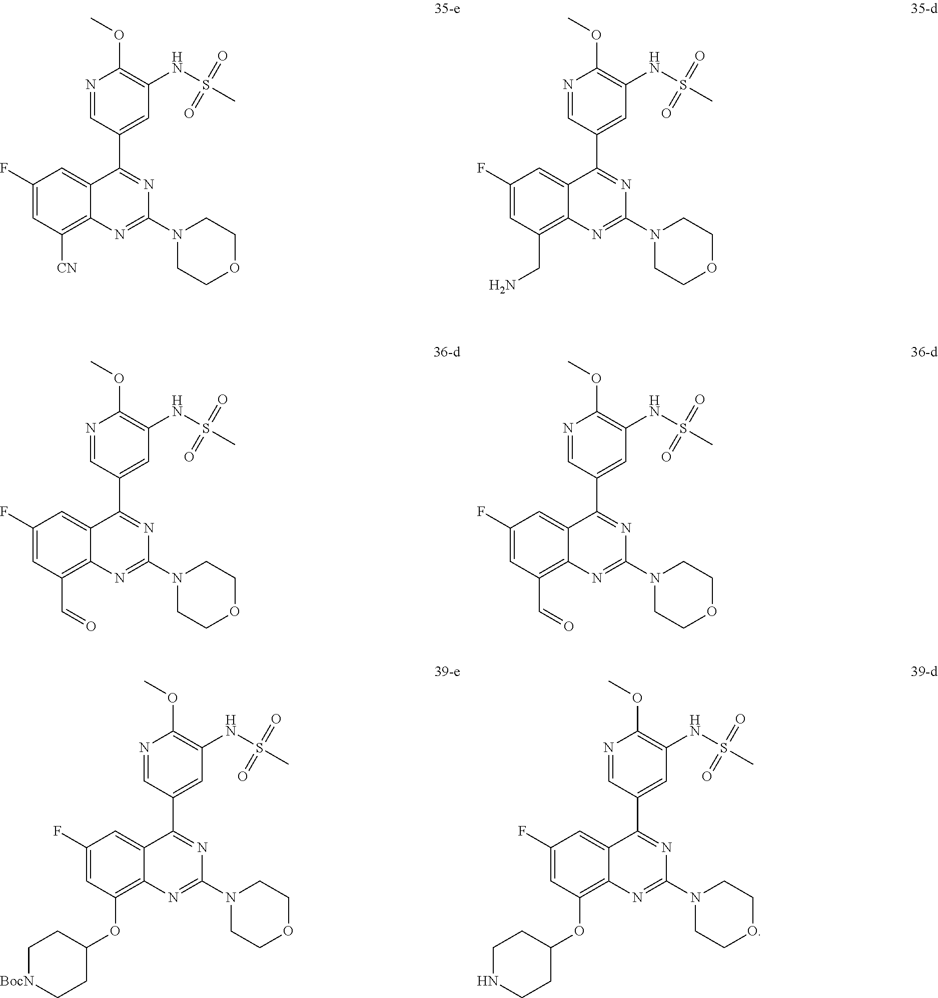 [SMILES: COc1ncc(-c2nc(N3CCOCC3)nc3c(C#N)cc(F)cc23)cc1NS(C)(=O)=O ; COc1ncc(-c2nc(N3CCOCC3)nc3c(CN)cc(F)cc23)cc1NS(C)(=O)=O ; COc1ncc(-c2nc(N3CCOCC3)nc3c(C=O)cc(F)cc23)cc1NS(C)(=O)=O ; COc1ncc(-c2nc(N3CCOCC3)nc3c(C=O)cc(F)cc23)cc1NS(C)(=O)=O ; COc1ncc(-c2nc(N3CCOCC3)nc3c(OC4CCN(C(=O)OC(C)(C)C)CC4)cc(F)cc23)cc1NS(C)(=O)=O ; COc1ncc(-c2nc(N3CCOCC3)nc3c(OC4CCNCC4)cc(F)cc23)cc1NS(C)(=O)=O]

The present disclosure also provides a pharmaceutical composition comprising a substance A and a pharmaceutically acceptable excipient, wherein the substance A is a morpholinylquinazoline compound represented by formula I, a pharmaceutically acceptable salt thereof, a solvate thereof, a solvate of the pharmaceutically acceptable salt thereof, a polycrystalline form thereof or a precursor thereof as described above.

The present disclosure also provides a method for preventing or treating disease involving abnormal cell proliferation, differentiation or survival, comprising: administering an effective amount of a substance A to a subject, wherein the substance A is the morpholinylquinazoline compound of formula I, the pharmaceutically acceptable salt thereof, the solvate thereof, the solvate of the pharmaceutically acceptable salt thereof, the polycrystalline form thereof or the precursor thereof as described above.

In a certain embodiment, the disease is cancer.

In a certain embodiment, the cancer is preferably caused by the proliferation of malignant neoplastic cells, such as tumors, flab, sarcoma, leukemia or lymphoma.

The present disclosure also provides a method for reducing the number of lymphocytes circulating in a subject, comprising: administering an effective amount of a substance A to the subject, wherein the substance A is the morpholinylquinazoline compound of formula I, the pharmaceutically acceptable salt thereof, the solvate thereof, the solvate of the pharmaceutically acceptable salt thereof, the polycrystalline form thereof or the precursor thereof as described above.

In a certain embodiment, the subject preferably is subject with hematologic underlying conditions, subject with autoimmune diseases, and subject requiring modulation of the immune response.

The term “more” refers to 2, 3, 4 or 5.

The term“pharmaceutically acceptable salt” refers to a salt of the compound disclosed herein which is prepared using relatively safe and pharmaceutically acceptable acids or bases. When the compound disclosed herein contains a relatively acidic functional group, a base addition salt can be obtained by contacting a neutral form of the compound with a sufficient amount of a pharmaceutically acceptable base in a pure solution or a suitable inert solvent. Pharmaceutically acceptable base addition salts include, but are not limited to: lithium salt, sodium salt, potassium salt, calcium salt, aluminum salt, magnesium salt, zinc salt, bismuth salt, ammonium salt, and diethanolamine salt. When the compound disclosed herein contains a relatively basic functional group, an acid addition salt can be obtained by contacting a neutral form of the compound with a sufficient amount of a pharmaceutically acceptable acid in a pure solution or a suitable inert solvent. The pharmaceutically acceptable acid includes inorganic acids, including, but not limited to: hydrochloric acid, hydrobromic acid, hydroiodic acid, nitric acid, carbonic acid, phosphoric acid, phosphorous acid, and sulfuric acid. The pharmaceutically acceptable acids include organic acids, including, but not limited to: acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, bitartrate, ascorbic acid, gentisic acid, fumaric acid, gluconic acid, saccharic acid, formic acid, ethanesulfonic acid, pamoic acid (i.e., 4,4′-methylene-bis(3-hydroxy-2-naphthoic acid)), and amino acids (e.g., glutamic acid and arginine). When a compound disclosed herein contains both relatively acidic functional group and relatively basic functional group, it can be converted to either base addition salt or acid addition salt. For details, see Berge et al., “Pharmaceutical Salts”, Journal of Pharmaceutical Science 66:1-19 (1977), or Handbook of Pharmaceutical Salts: Properties, Selection, and Use (P. Heinrich Stahl and Camille G. Wermuth, ed., Wiley-VCH, 2002).

The term “halogen” refers to fluorine, chlorine, bromine or iodine.

The term “alkyl” refers to a linear or branched alkyl group having a specified number of carbon atoms. Examples of alkyl include methyl, ethyl, n-propyl, isopropyl, n-butyl, tert-butyl, isobutyl, sec-butyl, n-pentyl, n-hexyl, n-heptyl, n-octyl, and the like.

The term “alkylene” refers to a linking group between two other species, which may be linear or branched. Examples include, but are not limited to, —CH₂—, —CH₂CH₂—, —CH₂CH₂CH₂CH(CH₃)— and —CH₂CH(CH₂CH₃)CH₂—.

The terms “cycloalkyl” and “carbocyclic ring” refer to a saturated cyclic group consisting only of carbon atoms having a specified number of carbon atoms (e.g., C₃-C₆), which is a monocyclic, bridged or spiro ring. The cycloalkyl includes, but is not limited to, cyclopropyl, cyclobutyl, cyclopentyl, and cyclohexyl.

The term “aryl” refers to an aromatic group consisting of carbon atoms, each ring having aromaticity. For example, phenyl or naphthyl.

The term “benzoheterocycloalkenyl” refers to a cyclic group having a specified number of of heteroatoms (e.g., 1, 2, or 3) and specified heteroatom species (one or more of N, O and S) connected with two adjacent carobon of benzene.

The term “heteroaryl” refers to a cyclic group having a specified number of ring atoms (e.g., 5-12 members), a specified number of heteroatoms (e.g., 1, 2, or 3) and specified heteroatom species (one or more of N, O and S), which is monocyclic or polycyclic, and has at least one aromatic ring (according to the Hickel's rule). Heteroaryls are linked to other fragments of the molecule through aromatic or non-aromatic rings. Heteroaryls include, but are not limited to, furyl, pyrrolyl, thienyl, pyrazolyl, imidazolyl, oxazolyl, thiazolyl, pyridinyl, pyrimidinyl, and indolyl.

The term “heterocycloalkyl” refer to a cyclic group having a specified number of ring atoms (e.g., 3-8 members), a specified number of heteroatoms (e.g., 1, 2, or 3) and specified heteroatom species (one or more of N, O and S), which is monocyclic, bridged, or spiro, and where each ring is saturated. Heterocycloalkyls include, but are not limited to, azetidinyl, tetrahydropyrrolyl, tetrahydrofuryl, morpholinyl, piperidinyl, and the like.

The above preferred conditions may be combined arbitrarily to obtain preferred embodiments of the present disclosure without departing from the general knowledge in the art.

The reagents and starting materials used in the present disclosure are commercially available.

The positive/progressive effects of the present disclosure are as follows: the present disclosure provides a morpholinylquinazoline compound, pharmaceutical compositions thereof and use thereof, wherein the morpholinylquinazolines of the present disclosure have dual inhibitory activities towards PI3Kδ and HDAC, which are expected to achieve better efficacy and wider application.

### DETAILED DESCRIPTION OF THE EMBODIMENTS

The present disclosure is further illustrated by the following examples, which are not intended to limit the present disclosure. Experimental procedures without specified conditions in the following examples were performed in accordance with conventional procedures and conditions, or in accordance with instructions.

In the present disclosure, room temperature refers to ambient temperature, or 10-35° C.

Overnight refers to 8-15 hours. Reflux refers to the reflux temperature of a solvent at atmospheric pressure.

### THE FOLLOWING IS A LIST OF ABBREVIATIONS USED IN THE EXAMPLES

PE Petroleum ether

EA Ethyl acetate

DCM Dichloromethane

THE Tetrahydrofuran

DMF N,N-dimethylformamide

DIPEA diisopropylethylamine

DABCO diazabicyclooctane

TFA trifluoroacetic acid

Tf trifluoromethanesulfonyl

Tf2O trifluoromethanesulfonic anhydride

TsCl p-Toluenesulfonyl chloride

Cbz-Cl benzyl chloroformate

Boc₂O di-tert-butyl dicarbonate

Pd(PPh₃)₄ tetrakis(triphenylphosphine)palladium

Pd₂(dba)₃ tris(dibenzylideneacetone)dipalladium

Pd(dppf)Cl₂ [1,1′-bis(diphenylphosphino)ferrocene]dichloropalladium

X-Phos 2-dicyclohexylphosphino-2′,4′,6′-triisopropylbiphenyl

HATU 2-(7-azobenzotriazol)-tetramethylurea hexafluorophosphate

PCC pyridinium chlorochromate

DMAP p-dimethylaminopyridine

CDI N,N-carbonyldiimidazole

t-Butyl-x-Phos 2-di-tert-butylphosphino-2′,4′,6′-triisopropylbiphenyl

### Synthetic route of compound 1

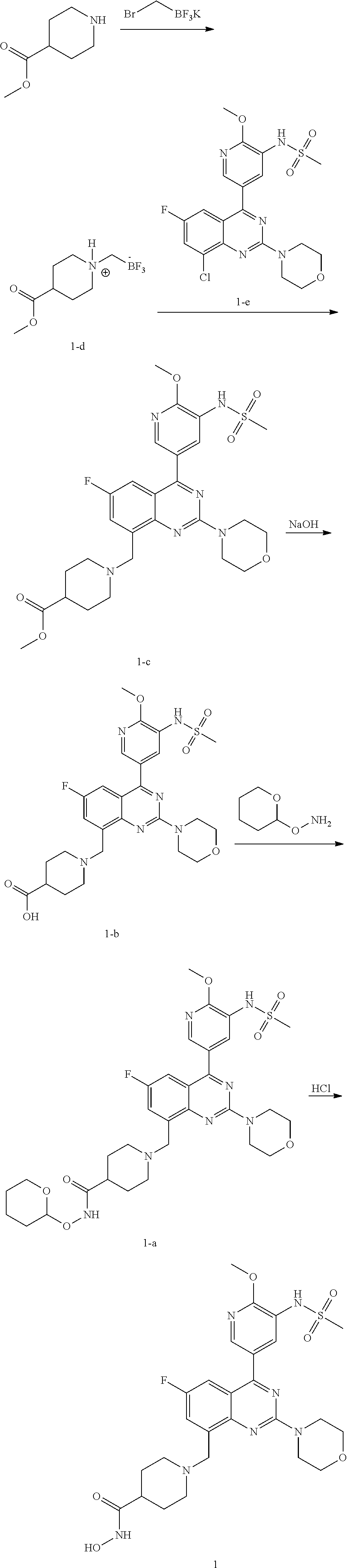

### Synthesis of compound 1-e

Compound 1-e was synthesized with reference to the method described in Patent CN104557872A.

### Synthesis of compound 1-d

The compound methyl piperidine-4-carboxylate (3.15 g, 22.00 mmol) was dissolved in acetonitrile (50 mL), followed by the serial addition of potassium iodide (199 mg, 1.20 mmol), and potassium bromomethylfluoroborate (4 g, 19.92 mmol) at room temperature and the resulting mixture was stirred at 80° C. under nitrogen atmosphere for 16 hours. Cooled to room temperature, the mixture was filtered, and the filter cake was washed with acetonitrile (10 mL), then the filtrate was concentrated at reduced pressure to obtain the crude product. The crude product was dissolved in acetonitrile (5 mL) and methyl tert-butyl ether (100 mL) was added dropwise with stirring. Then the mixture was filtered, and the filter cake was washed with methyl tert-butyl ether (20 mL). The solid was collected and dried under vacuum to give compound 1-d (4.5 g, 100%). LC-MS (ESI): m/z=243.1 [M+18]⁺.

### Synthesis of compound 1-c

Compound 1-e (600 mg, 1.28 mmol) was dissolved in 50 mL THF at room temperature, followed by serial addition of water (5 mL), 1-d (1.0 g, 4.48 mmol), palladium acetate (29 mg, 0.13 mmol), X-Phos (122 mg, 0.26 mmol), and cesium carbonate (1.25 g, 3.84 mmol). The resulting mixture was stirred at 80° C. under nitrogen atmosphere for 36 hours. 50 mL water was added to the mixture, and the organic phase was extracted with EA (100 mL×2). The organic phase was washed with brine (100 mL×2), dried over anhydrous sodium sulfate, filtered and concentrated at reduced pressure to obtain the crude product. The crude product was purified by flash chromatography (PE/EA=1:1) to give compound 1-c (60 mg, 8%). LC-MS (ESI): m/z=589.2 [M+1]⁺.

### Synthesis of compound 1-b

THF (5 mL), water (2.5 mL), and sodium hydroxide (41 mg, 1.02 mmol) were added sequentially to a solution of Compound 1-c (60 mg, 0.10 mmol) in methanol (5 mL) at room temperature. The mixture was stirred at room temperature for 2 hours, concentrated at reduced pressure. The residue was dissolved in water (10 mL), and pH was adjusted to 3 with 1M HCl aqueous solution. Then the mixture was extracted with mixed solvent (DCM/MeOH=10:1, 100 mL×2), and the organic phase was dried over anhydrous sodium sulfate, filtered, concentrated at reduced pressure to give the crude compound 1-b (58 mg, 100%). LC-MS (ESI): m/z=575.2 [M+1]⁺.

### Synthesis of compound 1-a

HATU (70 mg, 0.18 mmol) and DIPEA (101 μL, 0.61 mmol) were added sequentially into a solution of compound 1-b (59 mg, 0.10 mmol) in DMF (5 mL) in an ice bath, and the resulting mixture was stirred at 0° C. for 10 min, followed by the addition of O-(tetrahydro-2H-pyran)-2-hydroxylamine (29 mg. 0.24 mmol). The mixture was brought to room temperature and stirred for 16 hours. The reaction was quenched with water (20 mL), and the mixture was extracted with ethyl acetate (80 mL×2). The organic phase was washed with brine (100 mL×3), dried over anhydrous sodium sulfate, filtered and concentrated at reduced pressure to give the crude product. The crude product was purified by flash chromatography (DCM/MeOH=10:1) to give compound 1-a (40 mg, 58%). LC-MS (ESI): m/z=674.2 [M+1]⁺.

### Synthesis of compound 1

A solution of hydrogen chloride in 1,4-dioxane (4 M, 0.5 mL) was added dropwise slowly to a solution of compound 1-a (40 mg, 0.059 mmol) in DCM (10 mL) at 0° C. After addition, the mixture was stirred at 0° C. for 10 min, and the reaction was quenched with saturated sodium bicarbonate solution (10 mL). The mixture was extracted with mixed solvent (DCM/MeOH=10:1, 100 mL×2), and the organic phase was dried over anhydrous sodium sulfate, filtered and concentrated at reduced pressure to give the crude product, which was purified by PREP-TLC (DCM/MeOH=6:1) to give compound 1(6 mg, 17%). LC-MS (ESI): m/z=590.2 [M+1]⁺; ¹H NMR (400 MHz, CDCl₃): δ 8.25(1H, d, J=2.0 Hz), 8.09 (1H, s), 8.05-8.00 (2H, m), 7.61-7.57 (1H, m), 7.32-7.28 (1H, m), 4.67-4.58 (1H, m), 4.12-4.01 (1H, m), 4.04 (3H, s), 4.00-3.93 (2H, m), 3.91-3.81 (4H, m), 3.80-3.70 (4H, m), 3.37 (1H, s), 3.05-2.91 (1H, m), 2.99 (3H, s), 2.31-2.04 (5H, m), 1.58-1.49 (2H, m).

### Synthetic route of compound 2

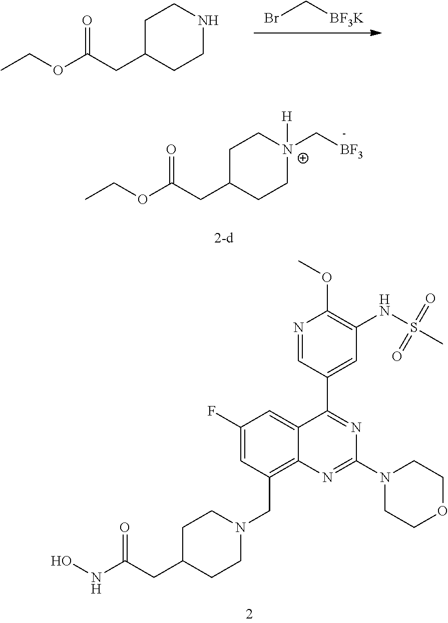

### Synthesis of compound 2-d

Ethyl piperidine-4-acetate (2.50 g, 14.60 mmol) was dissolved in acetonitrile (50 mL), followed by serial addition of potassium iodide (132 mg, 0.80 mmol), and potassium bromomethylfluoroborate (2.67 g, 13.28 mmol) at room temperature and the resulting mixture was stirred for 16 hours at 80° C. under nitrogen atmosphere. Cooled to room temperature, the mixture was filtered, and the filter cake was washed with acetonitrile (10 mL). The filtrate was concentrated at reduced pressure to obtain the crude product. The crude product was dissolved in acetonitrile (3 mL) and methyl tert-butyl ether (30 mL) was added dropwise with stirring. The mixture was filtered and the filter cake was washed with methyl tert-butyl ether (10 mL), the solid was collected and dried under vacuum to give compound 2-d (3.3 g, 100%). LC-MS (ESI): m/z=271.2 [M+18]⁺.

### Synthesis of compound 2

Synthesized according to synthesis of compound 1, compound 2-d was used instead of compound 1-d to obtain compound 2 (82 mg). LC-MS (ESI): m/z=604.2 [M+1]⁺; ¹H NMR (400 MHz, DMSO-d₆): δ10.42 (1H, s), 9.59 (1H, brs), 8.72 (1H, s), 8.42 (1H, d, J=1.2 Hz), 8.01 (1H, d, J=1.2 Hz), 7.59 (1H, brs), 4.67-4.63 (1H, m), 4.03 (3H, s), 3.96-3.81 (4H, m), 3.79-3.64 (4H, m), 3.56 (4H, s), 3.11 (3H, s), 1.96-1.83 (2H, m), 1.79-1.64 (2H, m), 1.63-1.53 (1H, m), 1.52-1.28 (3H, m).

### Synthetic route of compound 3

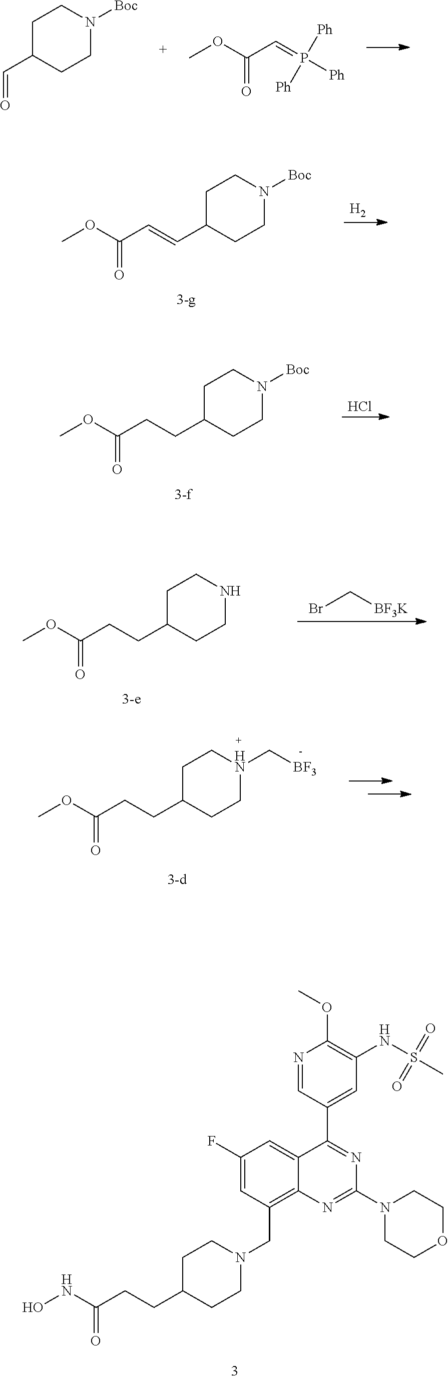

### Synthesis of compound 3-g

Methoxycarbonylmethyltriphenylphosphine (5.5 g, 16.45 mmol) was added o a solution of compound 1-Boc-4-formylpiperidine (3.5 g, 16.40 mmol) in chloroform (50 mL), and the resulting mixture was refluxed for 16 hours. The mixture was cooled to room temperature, concentrated at reduced pressure. The residue was suspended in ethyl ether (50 mL), filtered. The filter cake was washed with ethyl ether (10 mL), and the filtrate was concentrated at reduced pressure to give the crude product, which was purified by flash chromatography (PE/EA=5:1) to give compound 3-g (4.4 g, 100%). LC-MS (ESI): m/z=292.1 [M+Na]⁺.

### Synthesis of compound 3-f

Compound 3-g (3 g, 11.14 mmol) was dissolved in methanol (100 mL), and then Pd/C (50% wet, 10%, 0.6 g) was added to it. The reaction system was degassed and purged with nitrogen three times and then hydrogen three times, and then the mixture was stirred at room temperature under hydrogen atmosphere for 16 hours. The mixture was filtered and the filter cake was washed with methanol (20 mL), and the filtrate was concentrated at reduced pressure to give compound 3-f (2.56 g, 85%). LC-MS (ESI): m/z=294.1 [M+Na]⁺.

### Synthesis of compound 3-e

Compound 3-f (2.56 g, 9.43 mmol) was dissolved in methanol (10 mL), then a solution of hydrogen chloride in methanol (4 M, 30 mL) was added to it at room temperature, and the mixture was stirred at room temperature for 2 days. The mixture was concentrated at reduced pressure, and the crude product was suspended in saturated sodium bicarbonate solution (30 mL), concentrated at reduced pressure and dried under vacuum for 1 hour. The crude product was suspended in THF (50 mL), filtered, and the filter cake was washed with THF (10 mL), then the filtrate was concentrated at reduced pressure to give compound 3-e (520 mg, 32%). LC-MS (ESI): m/z=172.2 [M+H]⁺.

### Synthesis of compound 3-d

Compound 3-e (520 mg, 3.04 mmol) was dissolved in acetonitrile (20 mL), followed by serial addition of potassium iodide (27 mg, 0.17 mmol) and potassium bromomethylfluoroborate (554 mg, 2.76 mmol) at room temperature. The resulting mixture was stirred at 80° C. under nitrogen atmosphere for 16 hours. The mixture was cooled to room temperature, filtered, and the filter cake was washed with acetonitrile (5 mL), then the filtrate was concentrated at reduced pressure to obtain the crude product. The crude product was dissolved in acetonitrile (2 mL) and methyl tert-butyl ether (50 mL) was added dropwise with stirring. The mixture was filtered, and the filter cake was washed with methyl tert-butyl ether (10 mL), then the solid was collected and dried under vacuum to give compound 3-d (635 mg, 91%). LC-MS (ESI): m/z=271.2 [M+18]⁺.

### Synthesis of compound 3

Synthesized according to synthesis of compound 1, compound 3-d was used instead of compound 1-d to obtain compound 3 (120 mg). LC-MS (ESI): m/z=618.3 [M+1]⁺; ¹H NMR (400 MHz, CDCl₃): δ 8.32 (1H, d, J=1.2 Hz), 8.15 (1H, s), 7.68 (1H, brs), 7.42-7.31 (1H, m), 5.02-4.87 (1H, m), 4.11 (2H, s), 4.11-4.05 (2H, m), 3.93 (4H, s), 3.82 (4H, s), 3.70 (3H, s), 3.07 (3H, s), 3.11-2.95 (2H, m), 2.36-2.08 (4H, m), 1.90-1.46 (7H, m).

### Synthetic route of compound 4

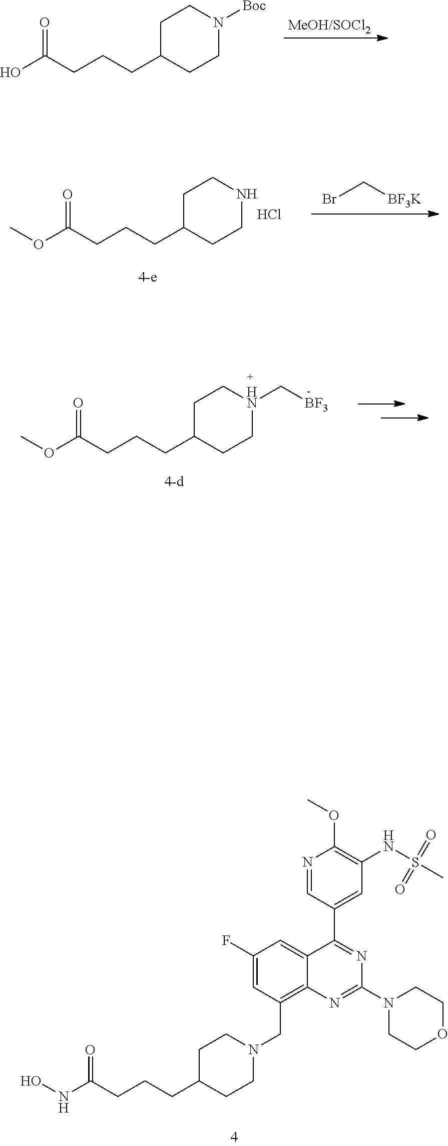

### Synthesis of compound 4-e

Sulfoxide chloride (1 mL) was added dropwise into a solution of compound 1-Boc-4-piperidine-butyric acid (1 g, 3.68 mmol) in methanol (10 mL) at 0° C. and the mixture was brought to room temperature and stirred at this temperature for 5 hours. The mixture was concentrated at reduced pressure to give compound 4-e (816 mg, 100%). LC-MS (ESI): m/z=186.2 [M+H]⁺.

### Synthesis of compound 4-d

Compound 4-e (816 mg, 3.68 mmol) was dissolved in acetonitrile (20 mL), and then potassium iodide (33 mg, 0.20 mmol), potassium carbonate (693 mg, 5.02 mmol), and potassium bromomethylfluoroborate (673 mg, 3.35 mmol) were added sequentially at room temperature. The resulting mixture was stirred at 80° C. under nitrogen atmosphere for 16 hours. The mixture was cooled to room temperature, filtered, and the filter cake was washed with acetonitrile (10 mL), then the filtrate was concentrated at reduced pressure to obtain the crude product. The crude product was dissolved in acetonitrile (2 mL)and methyl tert-butyl ether (80 mL) was added dropwise with stirring. The mixture was filtered and the filter cake was washed with methyl tert-butyl ether (10 mL), then the solid was collected and dried under vacuum to give compound 4-d (838 mg, 94%). LC-MS (ESI): m/z=285.2 [M+18]⁺.

### Synthesis of compound 4

Synthesized according to synthesis of compound 1, compound 4-d was used instead of compound 1-d to obtain compound 4 (50 mg). LC-MS (ESI): m/z=632.2 [M+1]⁺; ¹H NMR (400 MHz, CD₃OD): δ 8.29 (1H, d, J=2.0 Hz), 8.04 (1H, d, J=2.4 Hz), 7.78 (1H, d, J=6.4 Hz), 7.65 (1H, dd, J₁=8.8 Hz, J₂=2.4 Hz), 4.68 (2H, s), 4.02 (3H, s), 3.88 (4H, t, J=4 Hz), 3.72 (4H, t, J=5.2 Hz), 3.59-3.45 (2H, m), 3.09 (2H, t, J=12.4 Hz), 2.95 (3H, s), 1.98 (1H, t, J=7.6 Hz), 1.94-1.64 (3H, m), 1.60-1.46 (3H, m), 1.44-1.20 (4H, m).

### Synthetic route of compound 5

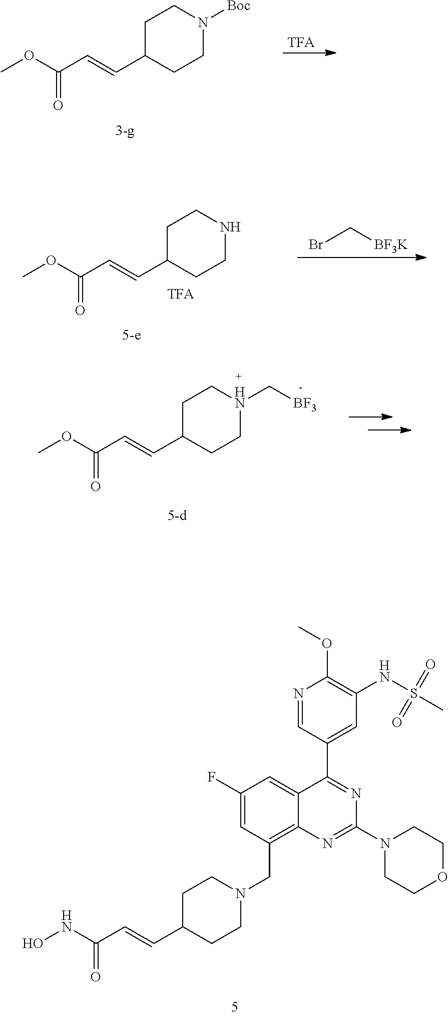

### Synthesis of compound 5-e

To a solution of compound 3-g (1.7 g, 6.31 mmol) in DCM (20 mL) cooled in an ice bath was added TFA (4 mL), and then the mixture was stirred for 5 hours at room temperature. The mixture was concentrated at reduced pressure to give compound 5-e (1.78 g, 100%). LC-MS (ESI): m/z=170.2 [M+H]⁺.

### Synthesis of compound 5-d

Compound 5-e (1.78 g, 6.28 mmol) was dissolved in acetonitrile (40 mL), then potassium iodide(56.9 mg, 0.34 mmol), potassium carbonate (1.18 g, 8.56 mmol), and potassium bromomethylfluoroborate (1.15 g, 5.71 mmol) were added sequentially to it at room temperature. The resulting mixture was stirred at 80° C. under nitrogen atmosphere for 16 hours. The mixture was cooled to room temperature, filtered, and the filter cake was washed with acetonitrile (10 mL), then the filtrate was concentrated at reduced pressure to obtain the crude product. The crude product was dissolved in acetonitrile (5 mL) and methyl tert-butyl ether (100 mL) was added dropwise with stirring. The mixture was filtered, and the filter cake was washed with methyl tert-butyl ether (10 mL), then the solid was collected and dried under vacuum to give the crude compound 5-d (1.58 g, 100%). LC-MS (ESI): m/z=269.2 [M+18]⁺.

### Synthesis of compound 5

Synthesized according to synthesis of compound 1, compound 5-d was used instead of compound 1-d to give compound 5 (50 mg, 88%). LC-MS (ESI): m/z=616.2 [M+1]⁺; ¹H NMR (400 MHz, CD₃OD): δ 8.38 (1H, d, J=2.4 Hz), 8.15 (1H, d, J=2.0 Hz), 7.77 (1H, d, J=7.6 Hz), 7.62 (1H, d, J=10.0 Hz), 6.77 (1H, dd, J₁=15.6 Hz, J₂=6.8 Hz), 5.84 (1H, d, J=15.2 Hz), 4.52-4.28 (2H, m), 4.13 (3H, s), 4.04-3.89 (4H, m), 3.89-3.77 (4H, m), 3.41-3.35 (1H, m), 3.07 (3H, s), 2.99-2.86 (1H, m), 2.86-2.32 (3H, m), 1.92 (2H, d, J=13.2 Hz), 1.75-1.53 (2H, m).

### Synthetic route of compound 6

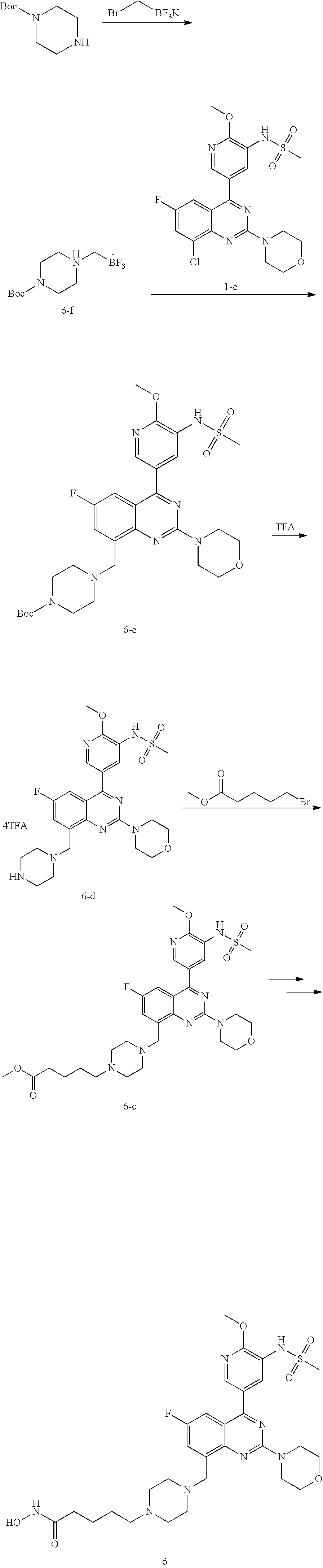

### Synthesis of compound 6-f

Compound 1-Boc-piperazine (2 g, 10.74 mmol) was dissolved in acetonitrile (50 mL), and then potassium iodide (97 mg, 0.58 mmol) and potassium bromomethylfluoroborate (1.95 g, 9.71 mmol) were added sequentially to the above solution at room temperature. The resulting mixture was stirred for at 80° C. under nitrogen atmosphere 16 hours. The mixture was cooled to room temperature, filtered, and the filter cake was washed with acetonitrile (10 mL), then the filtrate was concentrated at reduced pressure to obtain the crude product. The crude product was dissolved in acetonitrile (3 mL) and methyl tert-butyl ether (100 mL) was added dropwise with stirring. The mixture was filtered and the filter cake was washed with methyl tert-butyl ether (10 mL), then the solid was collected and dried under vacuum to give compound 6-f (1.9 g, 73%). LC-MS (ESI): m/z =286.2 [M+18]⁺.

### Synthesis of compound 6-e

Compound 1-e (600 mg, 1.28 mmol) was suspended in THF (100 mL) at room temperature, and then water (10 mL), 6-f (1.03 g, 3.84 mmol), palladium acetate (29 mg, 0.13 mmol), x-Phos (122 mg, 0.26 mmol) and cesium carbonate (1.25 g, 3.85 mmol) were added sequentially to the above mixture. The resulting mixture was stirred at 80° C. under nitrogen atmosphere for 36 hours.

The mixture was added water (100 mL) and was extracted with ethyl acetate (100 mL×2). The organic phase was washed with brine (100 mL×2), dried over anhydrous sodium sulfate, filtered, and concentrated at reduced pressure to obtain the crude product. The crude product was purified by flash chromatography (PE/EA=1:1˜EA) to obtain compound 6-e (810 mg, 100%). LC-MS (ESI): m/z=632.2 [M+1]⁺.

### Synthesis of compound 6-d

TFA (9 mL) was added to a solution of compound 6-e (810 mg, 1.28 mmol) in DCM(36 mL) at 0° C. The resulting mixture was brought to room temperature, and stirred at this temperature for 3 hours. The mixture was concentrated at reduced pressure, dried under vacuum for 1 hour to give crude compound 6-d (1.3 g, 100%). LC-MS (ESI): m/z=532.2 [M+1]⁺.

### Synthesis of compound 6-c

Compound 6-d (400 mg, 0.40 mmol) was dissolved in DMF (20 mL), and then potassium carbonate (386 mg, 2.80 mmol) and methyl 5-bromo-pentanoate (63 μL, 0.44 mmol) were added sequentially to it. The resulting mixture was stirred at room temperature under nitrogen atmosphere for 2 days. Water (30 mL) was added to the above mixture, and the mixture was extracted with EA (100 mL×3). The organic phase was washed with brine (100 mL×3), dried over anhydrous sodium sulfate, filtered and concentrated at reduced pressure to give the crude product, which was purified by flash chromatography (DCM/MeOH=10:1) to give compound 6-c (240 mg, 93%). LC-MS (ESI): m/z=646.2 [M+1]⁺.

### Synthesis of compound 6

Synthesized according to synthesis of compound 1, compound 6-c was used instead of compound 1-c to give compound 6 (46 mg). LC-MS (ESI): m/z=647.1 [M+1]⁺; ¹H NMR (400 MHz, DMSO-d₆): δ 10.40 (1H, s), 8.71 (1H, s), 8.39 (1H, d, J=2 Hz), 7.98 (1H, d, J=2.0 Hz), 7.71 (1H, d, J=8.4 Hz), 7.47 (1H, d, J=8.8 Hz), 4.04 (2H, s), 4.02 (3H, s), 3.91-3.79 (4H, m), 3.78-3.67 (4H, m), 3.09 (3H, s), 3.02-2.55 (7H, m), 2.04-1.88 (2H, m), 1.64-1.37 (4H, m), 1.37-1.07 (4H, m).

### Synthetic route of compound 7

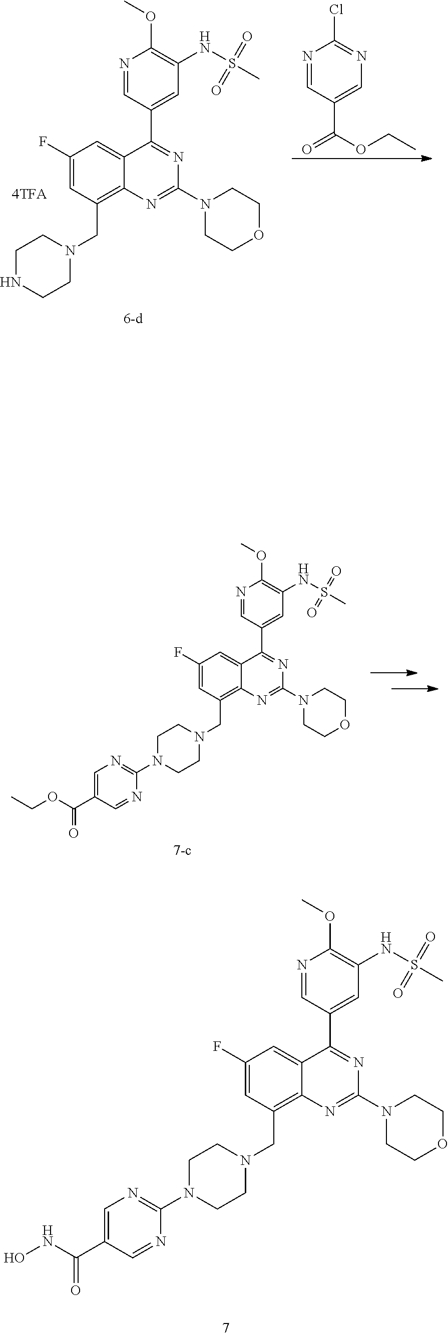 [SMILES: COc1ncc(-c2nc(N3CCOCC3)nc3c(CN4CCNCC4)cc(F)cc23)cc1NS(C)(=O)=O ; CCOC(=O)c1cnc(Cl)nc1 ; CCOC(=O)c1cnc(N2CCN(Cc3cc(F)cc4c(-c5cnc(OC)c(NS(C)(=O)=O)c5)nc(N5CCOCC5)nc34)CC2)nc1 ; COc1ncc(-c2nc(N3CCOCC3)nc3c(CN4CCN(c5ncc(C(=O)NO)cn5)CC4)cc(F)cc23)cc1NS(C)(=O)=O]

### Synthesis of compound 7-c

DIPEA (660 μL, 4 mmol) and ethyl 2-chloro-pyrimidine-4-carboxylate (82 mg, 0.44 mmol) were added sequentially to a solution of Compound 6-d (400 mg, about 0.40 mmol) was dissolved in DMF (20 mL). The mixture was stirred at room temperature under nitrogen atmosphere for 16 hours, then was added water (30 mL), and extracted with ethyl acetate (100 mL×2). The organic phase was washed with brine (100 mL×3), dried over anhydrous sodium sulfate, filtered, and concentrate at reduced pressure to obtain the crude product, which was purified by flash chromatography (DCM/MeOH=10:1) to give compound 7-c (200 mg, 73%). LC-MS (ESI): m/z=682.2 [M+1]⁺.

### Synthesis of compound 7

Synthesized according to synthesis of compound 1, compound 7-c was used instead of compound 1-c to obtain compound 7 (130 mg). LC-MS (ESI): m/z=669.2 [M+1]⁺; ¹H NMR (400 MHz, DMSO-d₆): δ 11.07 (1H, s), 9.51 (1H, s), 9.00 (1H, s), 8.67 (2H, d, J=5.2 Hz), 8.41 (1H, d, J=2.4 Hz), 8.01 (1H, d, J=2.0 Hz), 7.77 (1H, dd, J=2.8 Hz, J₂=8.8 Hz), 7.47 (1H, dd, J₁=3.2 Hz, J₂=9.6 Hz), 4.04 (2H, s), 4.03 (3H, s), 3.93-3.78 (8H, m), 3.77-3.63 (4H, m), 3.10 (3H, s), 2.62-2.54 (4H, m).

### Synthetic route of compound 8

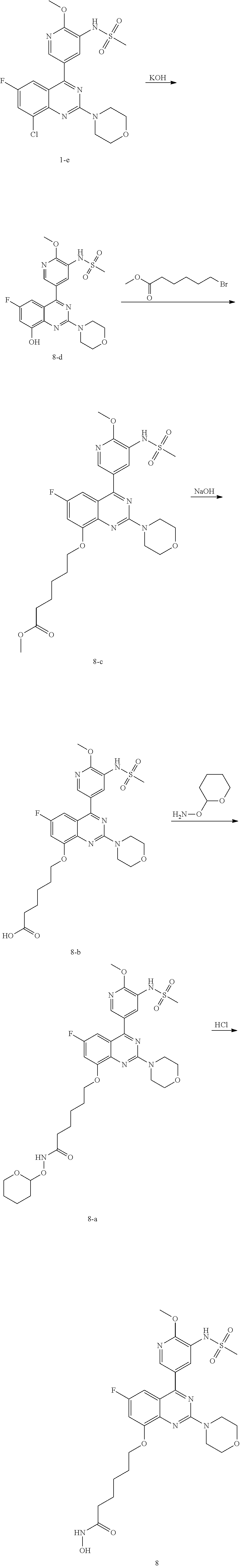

### Synthesis of compound 8-d

Compound 1-e (600 mg, 1.28 mmol) was dissolved in 1,4-dioxane (25 ML), followed by serial addition of water (15 mL), Pd₂(dba)₃ (117 mg, 0.13 mmol), t-Butyl-x-Phos (217 mg, 0.51 mmol) and KOH (3.1 g, 55.04 mmol). The mixture was stirred at 100° C. under nitrogen atmosphere for 16 hours. The mixture was cooled to 0° C., and 1M hydrochloric acid (about 55 mL, pH=4˜5) was added dropwise slowly to it. The aqueous phase was extracted with DCM (200 mL×2), and the organic phase was combined and dried over anhydrous sodium sulfate, filtered, then the filtrate was concentrated at reduced pressure to obtain the crude product. The crude product was purified by flash chromatography (dichloromethane:methanol=10:1) to give compound 8-d (575 mg, 100%). LC-MS (ESI): m/z=450.1 [M+1]⁺.

### Synthesis of compound 8-c

The compound 6-bromo-hexanoic acid methyl ester (92 mg, 0.44 mmol) was dissolved in DMF (10 mL) at room temperature, followed by addition of cesium carbonate (217 mg, 0.67 mmol). After the mixture was stirred at 80° C. under nitrogen atmosphere for 10 min, compound 8-d (200 mg, 0.44 mmol) was added to the above reaction system and the resulting mixture was stirred at 80° C. under nitrogen atmosphere for 16 hours. The mixture was cooled to room temperature, and water (50 mL) was added. The mixture was extracted with ethyl acetate (100 mL×2), and the organic phase was dried over anhydrous sodium sulfate, filtered, concentrated under pressure to obtain the crude product, which was purified by flash chromatography (petroleum ether: ethyl acetate=1:1) to give compound 8-c (157 mg, 62%). LC-MS (ESI): m/z=578.2 [M+1]⁺.

### Synthesis of compound 8-b

Compound 8-c (157 mg, 0.27 mmol) was suspended in methanol(10 mL), then THE (10 mL), water (5 mL) and sodium hydroxide (54 mg, 1.36 mmol) were added sequentially to the above mixture at room temperature. The mixture was stirred at room temperature for 2 hour, concentrated at reduced pressure. The residue was dissolved in water (20 mL), and the mixture was adjusted the pH to 4˜5 with 1M HCl aqueous solution, extract with DCM (100 mL×2). The organic phase was dried over anhydrous sodium sulfate, filtered, and concentrate at reduced pressure to give compound 8-b (153 mg, 100%). LC-MS (ESI): m/z=564.2 [M+1]⁺.

### Synthesis of compound 8-a

To a solution of compound 8-b (153 mg, 0.27 mmol) in DMF(10 mL) cooled in an ice bath was added HATU (154 mg, 0.41 mmol) and DIPEA (224 uL, 1.36 mmol) sequentially, and the mixture was stirred at 0° C. for 10 min, followed by the addition of O-(tetrahydro-2H-pyran)-2-hydroxylamine (64 mg, 0.54 mmol). The resulting mixture was brought to room temperature and stirred for 16 hours. The reaction was quenched with water(30 mL), and the mixture was extracted with ethyl acetate (100 mL×2). The organic phase was washed with brine (100 mL×3), dried over anhydrous sodium sulfate, filtered and concentrated at reduced pressure to give the crude product. The crude product was purified by pre-HPLC to give compound 8-a (125 mg, 70%). LC-MS (ESI): m/z=663.2[M+1]⁺.

### Synthesis of compound 8

A solution of hydrogen chloride in 1,4-dioxane (4 M, 1 mL) was added dropwise slowly into a mixture of compound 8-a (125 mg, 0.19 mmol) in DCM (20 mL) at 0° C. After the addition, the mixture was stirred at 0° C. for 10 min, and the reaction was quenched with saturated sodium bicarbonate solution (30 mL).The mixture was extracted with mixed solvent (DCM/MeOH=10:1, 100 mL *5), and the organic phase was dried over anhydrous sodium sulfate, filtered and concentrated at reduced pressure to give compound 8 (85 mg, 78%). LC-MS (ESI): m/z=579.2 [M+1]⁺; ¹H NMR (400 MHz, DMSO-d₆): δ 10.36 (1H, s), 9.51 (1H, s), 8.39 (1H, dd, J=2.4 Hz), 7.99 (1H, dd, J=1.6 Hz), 7.22 (1H, dd, J₁=2.4 Hz, J₂=10.8 Hz), 7.07 (1H, dd, J₁=2.4 Hz, J₂=9.6 Hz), 4.14 (2H, t, J=6.4 Hz), 4.03 (3H, s), 3.89-3.76 (4H, m), 3.76-3.62 (4H, m), 3.62-3.53 (1H, m), 3.10 (3H, s), 1.99 (2H, t, J=7.2 Hz), 1.90-1.75 (2H, m), 1.68-1.56 (2H, m), 1.54-1.39 (2H, m).

### Synthetic route of compound 9

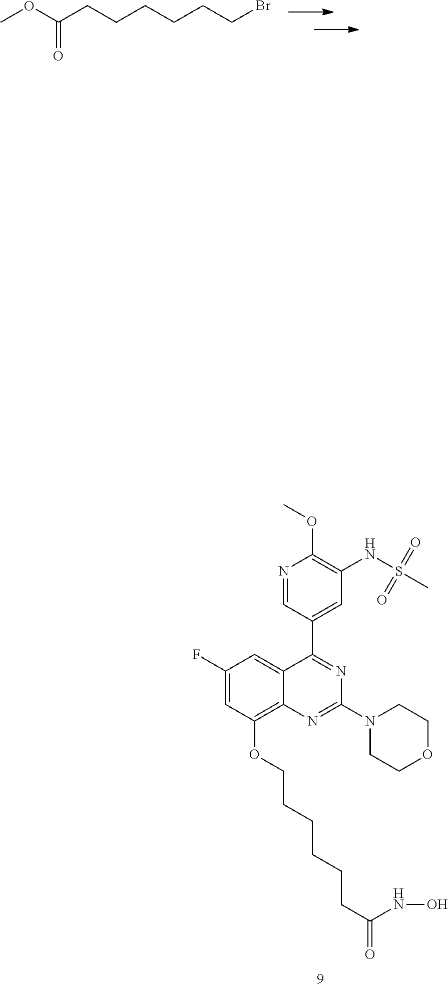 [SMILES: COC(=O)CCCCCCBr ; COc1ncc(-c2nc(N3CCOCC3)nc3c(OCCCCCCC(=O)NO)cc(F)cc23)cc1NS(C)(=O)=O]

### Synthesis of compound 9

Synthesized according to synthesis of compound 8, compound 9 (118 mg) was obtained by using compound 7-bromo-heptanoic acid methyl ester instead of compound 6-bromo-hexanoic acid methyl ester. LC-MS (ESI): m/z=593.2 [M+1]⁺; ¹H NMR (400 MHz, DMSO-d₆): δ10.35 (1H, s), 9.51 (1H, s), 8.67 (1H, d, J=1.2 Hz), 8.40 (1H, d, J=2.4 Hz), 7.99 (1H, d, J=2 Hz), 7.20 (1H, dd, J_{I}=2 Hz, J₂=10.4 Hz), 7.07 (1H, dd, J₁=2.4 Hz, J₂=9.6 Hz), 4.13 (2H, t, J=6 Hz), 4.03 (3H, s), 3.93-3.78 (4H, m), 3.78-3.63 (4H, m), 3.11 (3H, s), 1.96 (2H, t, J=7.2 Hz), 1.89-1.73 (2H, m), 1.62-1.43 (4H, m), 1.43-1.27 (2H, m).

### Synthetic route of compound 10

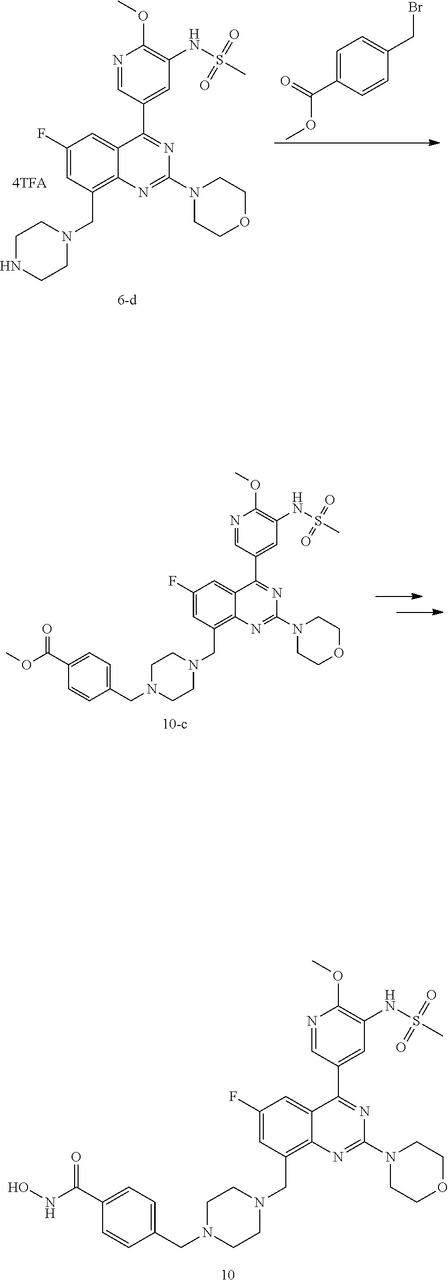

### Synthesis of compound 10-c

Compound 6-d (290 mg, 0.29 mmol) was dissolved in 10 mL DMF, then DIPEA (485 μL, 2.94 mmol) and methyl 4-bromomethylbenzoate (74 mg, 0.32 mmol) were added sequentially to the above solution, and the resulting mixture was stirred at room temperature under nitrogen atmosphere for 16 hours. Water (30 mL) was added, and the mixture was extracted with ethyl acetate (100 mL×2). The organic phase was washed with brine (100 mL×3), dried over anhydrous sodium sulfate, filtered, and concentrate at reduced pressure to obtain the crude product, which was purified by flash chromatography (PE/EA=1:1 to EA) to give compound 10-c (160 mg, 80%). LC-MS (ESI): m/z=680.4 [M+1]⁺.

### Synthesis of compound 10

Synthesized according to synthesis of compound 1, compound 10-c was used instead of compound 1-c to obtain compound 10 (100 mg). LC-MS (ESI): m/z=681.2 [M+1]⁺; ¹H NMR (400 MHz, DMSO-d₆): δ 11.17 (1H, s), 9.53 (1H, s), 9.00 (1H, s), 8.40 (1H, d, J=2.4 Hz), 8.00 (1H, d, J=2.4 Hz), 7.74-7.62 (3H, m), 7.51-7.41 (1H, m), 7.37 (2H, d, J=8.0 Hz), 4.03 (3H, s), 3.99 (2H, s), 3.90-3.79 (4H, m), 3.78-3.65 (4H, m), 3.53 (2H, s), 3.10 (3H, s), 2.64-2.27 (8H, m).

### Synthetic route of compound 11

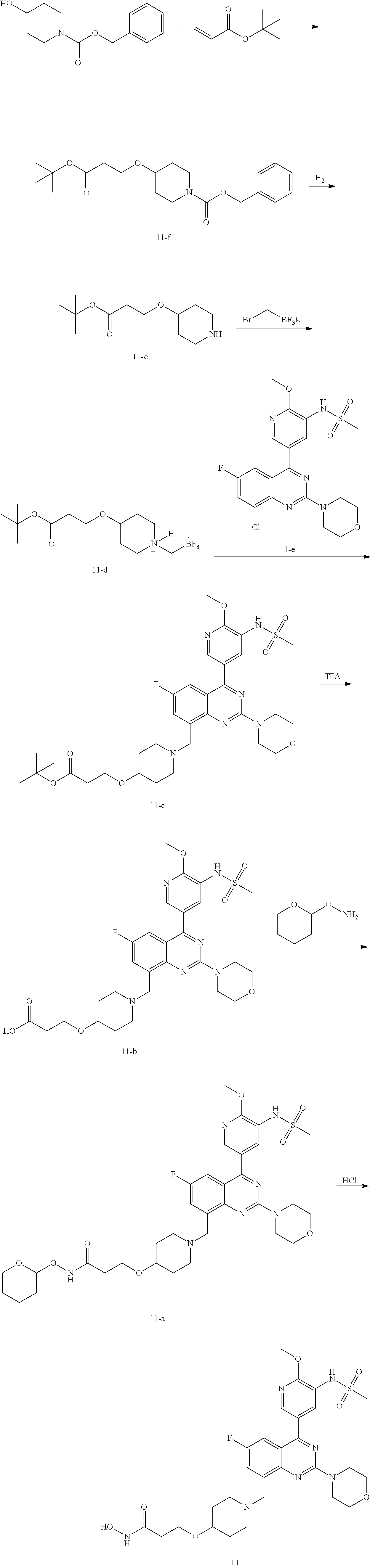

### Synthesis of compound 11-f

To a solution of compound N-Cbz-4-hydroxypiperidine (2.35 g, 10 mmol) in 1,4-dioxane (50 mL.) was added potassium tert-butoxide (60 mg, 0.54 mmol) and tert-butyl acrylate (3.7 mL, 25 mmol) sequentially at room temperature. The mixture was stirred at room temperature under nitrogen atmosphere for 30 min, and then stirred at 110° C. under nitrogen atmosphere for 16 hours. The mixture was cooled to room temperature and concentrated at reduced pressure to obtain the crude product. The crude product was purified by flash chromatography (PE/EA=2:1) to give compound 11-f (1.58 g, 44%). LC-MS (ESI): m/z=386.2 [M+23]⁺.

### Synthesis of compound 11-e

To a solution of compound 11-f (1.58 g, 4.35 mmol) in methanol (100 mL) was added Pd/C (0.5 g) under nitrogen atmosphere at room temperature, and the mixture was stirred at room temperature under hydrogen atmosphere for 16 hours. The mixture was filtered and the filtrate was concentrated at reduced pressure to give compound 11-e (875 mg, 88%). LC-MS (ESI): m/z=230.3 [M+1]⁺.

### Synthesis of compound 11-d

To a solution of compound 11-e (875 mg, 3.82 mmol) in acetonitrile (50 mL) was added potassium iodide (35 mg, 0.21 mmol) and potassium bromomethylfluoroborate (697 mg, 3.47 mmol) sequentially at room temperature. The resulting mixture was stirred at 80° C. under nitrogen atmosphere for 16 hours. The mixture was cooled to room temperature, filtered, and the filter cake was washed with acetonitrile (10 mL). Then the filtrate was concentrated at reduced pressure to give the crude compound 11-d (1.25 g). The crude product was used directly in the next step without purification. LC-MS (ESI): m/z=329.1 [M+18]⁺.

### Synthesis of compound 11-c

Compound 1-e (200 mg, 0.43 mmol) was dissolved in THF (30 mL) at room temperature, then water (3 mL), 11-d (399 mg, 1.28 mmol), palladium acetate (10 mg, 0.043 mmol), x-Phos (41 mg, 0.085 mmol) and cesium carbonate (417 mg, 1.28 mmol) were added sequentially to it. The mixture was stirred at 80° C. under nitrogen atmosphere for 36 hours. To the mixture, water (50 mL) was added, and the mixture was extracted with ethyl acetate (100 mL×2). The organic phase was washed with brine (100 mL×2), dried over anhydrous sodium sulfate, filtered, concentrated at reduced pressure to obtain the crude product. The crude product was purified by flash chromatography (DCM/MeOH=10:1) to give compound 11-c (290 mg, 100%). LC-MS (ESI): m/z =675.2 [M+1]⁺.

### Synthesis of compound 11-b

TFA (2.5 mL) was added into a solution of compound 11-c (100 mg, 0.15 mmol) in DCM (5 mL) in an ice bath. The mixture was brought to room temperature and stirred for 2 hours, concentrated to give crude compound 11-b (92 mg). The crude product used directly without purification in the next step. LC-MS (ESI): m/z=619.2 [M+1]⁺.

### Synthesis of compound 11-a

Compound 11-b (92 mg, 0.15 mmol) was dissolved in DMF (10 mL) at room temperature, followed by serial addition of HATU (84 mg, 0.22 mmol) and DIPEA (244 μL, 1.48 mmol) in an ice bath. The mixture was stirred at 0° C. for 10 min, followed by the addition of O-(tetrahydro-2H-pyran)-2-hydroxylamine (35 mg, 0.30 mmol) and the mixture was brought to room temperature and stirred for 16 hours. The reaction was quenched with water (20 mL), and the mixture was extracted with ethyl acetate (80 mL×2). The organic phase was washed with brine (100 mL×3), dried over anhydrous sodium sulfate, filtered and concentrated at reduced pressure to give the crude product. The crude product was separated and purified by pre-HPLC to give compound 11-a (52 mg, 49%). LC-MS (ESI): m/z=718.2 [M+1]⁺.

### Synthesis of compound 11

A solution of hydrogen chloride in 1,4-dioxane (4 M, 1 mL) was added dropwise slowly into compound 11-a (52 mg, 0.072 mmol) in DCM (20 mL) at 0° C., and the mixture was stirred at 0° C. for 10 min. The reaction was quenched with saturated sodium bicarbonate solution (30 mL), and the mixture was extracted with solvent DCM (100 mL×2). The organic phase was dried over anhydrous sodium sulfate, filtered and concentrated at reduced pressure to give compound 11 (40 mg, 87%). LC-MS (ESI): m/z=634.2 [M+1]⁺; ¹H NMR (400 MHz, DMSO-d₆): δ 10.39 (1H, s), 9.56 (1H, brs), 8.75 (1H, s), 8.40 (1H, d, J=2.0 Hz), 8.00 (1H, d, J=2.0 Hz), 7.75 (1H, brs), 7.48 (1H, brs), 4.13-3.97 (4H, m), 3.91-3.78 (4H, m), 3.79-3.65 (5H, m), 3.60 (2H, t, J=8.0 Hz), 3.58-3.49 (1H, m), 3.10 (3H, s), 3.02-2.70 (2H, m), 2.17 (2H, t, J=6.4 Hz), 1.94-1.72 (2H, m), 1.64-1.32 (4H, m).

### Synthetic route of compound 12

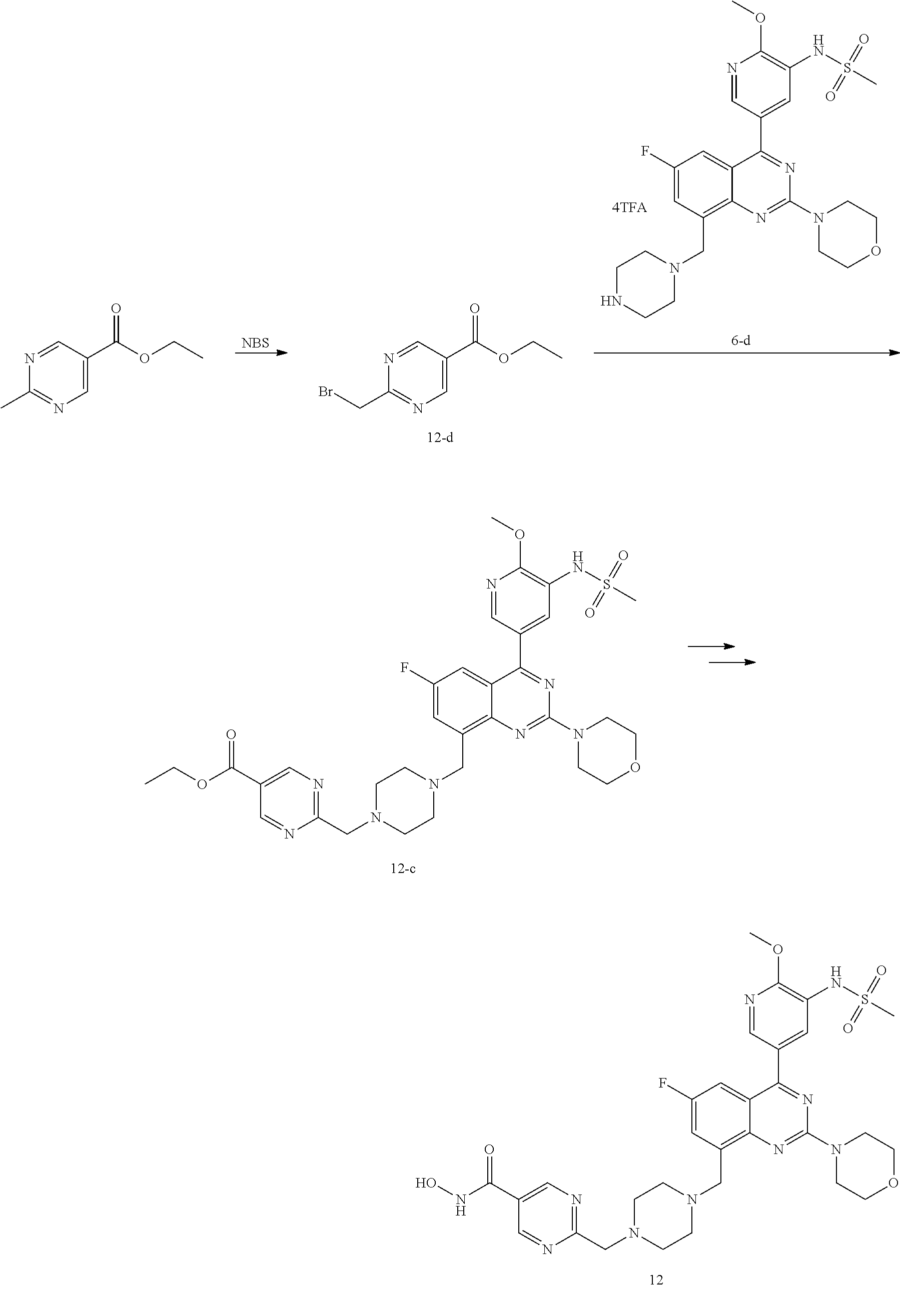

### Synthesis of compound 12-d

Compound ethyl 2-methylpyrimidine-5-carboxylate (1 g, 6.02 mmol) was suspended in carbon tetrachloride(10 mL), and then NBS (1.07 g, 6.02 mmol) and AIBN (49 mg, 0.3 mmol) were added sequentially to the above solution, and the mixture was stirred at 80° C. under nitrogen atmosphere for 16 hours. The mixture was concentrated at reduced pressure, and the residue was suspended in ethyl acetate(100 mL), filtered. The filtrate was washed with saturated sodium bicarbonate (100 mL×2), brine (100 mL). The organic phase was dried over anhydrous sodium sulfate, filtered and concentrated under pressure to obtain the crude product. The crude product was purified by flash chromatography (PE/EA=3:1) to obtain compound 12-d (320 mg, 22%). LC-MS (ESI): m/z=245.1[M+1]⁺.

### Synthesis of compound 12-c

DIPEA (485 μL, 2.94 mmol) and 12-d (79 mg, 0.32 mmol) were added sequentially to a solution of compound 6-d (290 mg, 0.29 mmol) in DMF (10 mL), and then the resulting mixture was stirred at room temperature under nitrogen atmosphere for 16 hours. Water(30 mL) was added and the mixture was extracted with ethyl acetate (100 mL×2). The organic phase was washed with brine (100 mL×3), dried over anhydrous sodium sulfate, filtered and concentrated at reduced pressure to obtain the crude product, which was purified by flash chromatography (DCM/MeOH=10:1) to give compound 12-c (170 mg, 83%). LC-MS (ESI): m/z=696.3 [M+1]⁺.

### Synthesis of compound 12

Synthesized according to synthesis of compound 1, compound 12-c was used instead of compound 1-c to obtain compound 12 (70 mg). LC-MS (ESI): m/z=683.4 [M+1]⁺; ¹H NMR (400 MHz, DMSO-d₆): δ 11.46 (1H, brs), 9.61 (1H, brs), 9.36 (1H, s), 9.03 (2H, s), 8.39 (1H, d, J=2.0 Hz), 7.99 (1H, d, J=2.0 Hz), 7.65 (1H, dd, J=9.2 Hz, J₂=2.0 Hz), 7.43 (1H, dd, J=9.2 Hz, J₂=2.4 Hz), 4.02 (3H, s), 3.95 (2H, s), 3.90-3.79 (4H, m), 3.76 (2H, s), 3.74-3.64 (4H, m), 3.09 (3H, s), 2.65-2.51 (8H, m).

### Synthetic route of compound 13

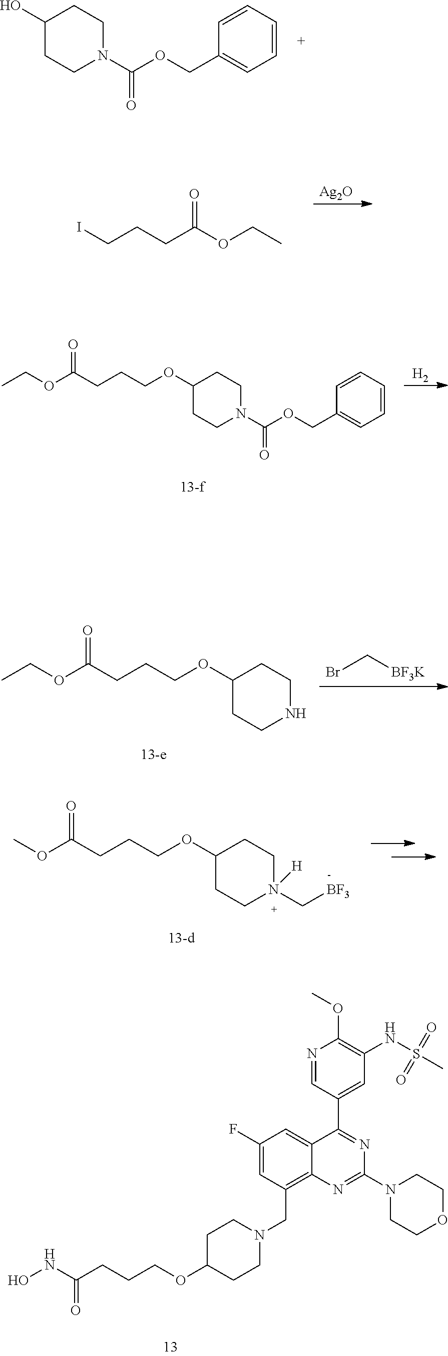

### Synthesis of compound 13-f

Compound N-Cbz-4-hydroxypiperidine (791 mg, 3.36 mmol) mixed with ethyl 4-iodo-butyrate (977 mg, 4.03 mmol), followed by the addition of silver oxide (1.56 g, 6.72 mmol) at room temperature, and the mixture was stirred at 60° C. under nitrogen atmosphere for 5 days. Cooled to room temperature, the mixture was added DCM (100 mL ), filtered and concentrated at reduced pressure to obtain the crude product. The crude product was purified by flash chromatography (PE/EA=2:1) to give compound 13-f (100 mg, 8.5%). LC-MS (ESI): m/z=350.2 [M+1]⁺.

### Synthesis of compound 13-e

Compound 13-f (100 mg, 0.29 mmol) was dissolved in methanol(50 mL), followed by the addition of Pd/C (50 mg) under nitrogen atmosphere at room temperature. The mixture was stirred at room temperature under hydrogen atmosphere for 16 hours. Filtered and concentrated the filatrate at reduced pressure to give the crude compound 13-e (40 mg). LC-MS (ESI): m/z=216.2 [M+1]⁺.

### Synthesis of compound 13-d

Potassium iodide (2 mg, 0.011 mmol) and potassium bromomethylfluoroborate (34 mg, 0.17 mmol) were added sequentially to a solution of compound 13-e (40 mg, 0.19 mmol) in acetonitrile(6 mL) at room temperature and the mixture was stirred at 80° C. under nitrogen atmosphere for 16. The mixture was cooled to room temperature, filtered, and the filter cake was washed with acetonitrile (10 mL), then the filtrate was concentrated at reduced pressure to give the crude compound 13-d (60 mg). The crude product was used directly in the next step without purification.

### Synthesis of compound 13

Synthesized according to synthesis of compound 1, compound 13-d was used instead of compound 1-d to obtain compound 13 (4 mg). LC-MS (ESI): m/z=648.2 [M+1]⁺; ¹H NMR (400 MHz, DMSO-d₆): δ 10.38 (1H, s), 9.56 (1H, s), 8.69 (1H, s), 8.43 (1H, d, J=2.0 Hz), 8.08 (1H, brs), 8.01 (1H, d, J=2.4 Hz), 7.68 (1H, brs), 4.03 (3H, s), 3.96-3.83 (4H, m), 3.79-3.72 (4H, m), 3.72-3.64 (1H, m), 3.59 (2H, s), 3.43-3.39 (2H, m), 3.10 (3H, s), 2.04-1.95 (4H, m), 1.78-1.59 (4H, m), 1.53-1.39 (4H, m).

### Synthetic route of compound 14

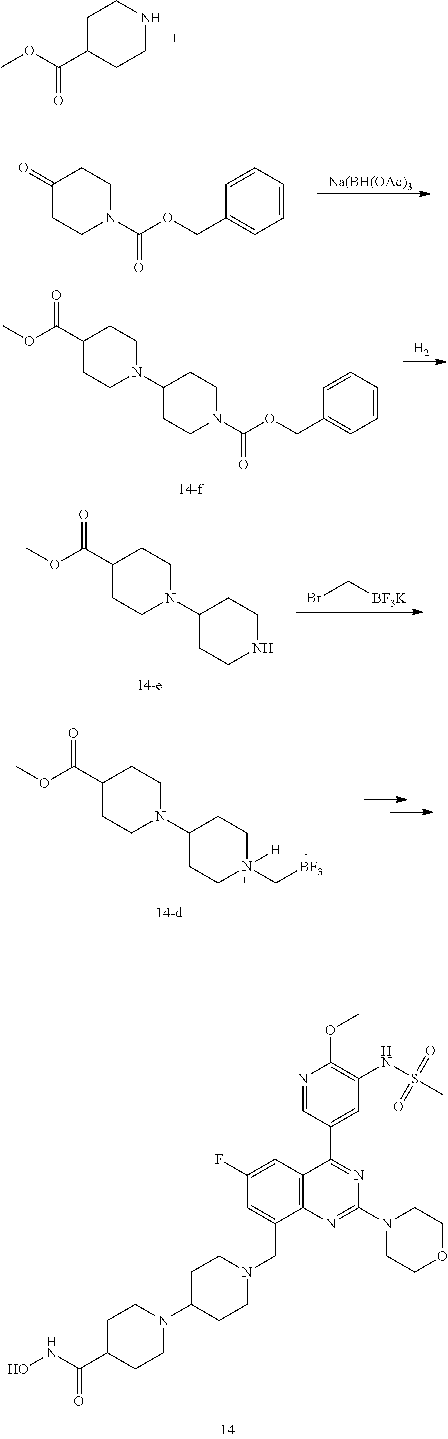

### Synthesis of compound 14-f

Methyl 4-piperidinecarboxylate (1.23 g, 8.57 mmol) and acetic acid (491 μL, 8.57 mmol) were added sequentially to a solution of the compound N-Cbz-4-piperidone (2 g, 8.57 mmol) in DCM (20 mL), and the mixture was stirred at room temperature under nitrogen atmosphere for 30 min, then sodium borohydride acetate (3.63 g, 17.14 mmol) was added to the above mixture, and the mixture was stirred at room temperature under nitrogen atmosphere for 5 days. 100 mL water was added and extracted with DCM (100 mL×2), the organic phase was dried over anhydrous sodium sulfate, filtered and concentrated at reduced pressure, and the crude product was purified by flash chromatography (DCM/MeOH=10:1) to give compound 14-f (600 mg, 19%). LC-MS (ESI): m/z=361.2 [M+1]⁺.

### Synthesis of compound 14-e

Compound 14-f (600 mg, 1.66 mmol) was dissolved in methanol (50 mL) and Pd/C (100 mg) was added under nitrogen atmosphere at room temperature. The mixture was stirred at room temperature under hydrogen atmosphere for 16 hours. Filtered and concentrated at reduced pressure to give the crude compound 14-e (370 mg). LC-MS (ESI): m/z=227.2 [M+1]⁺.

### Synthesis of compound 14-d

Potassium iodide (15 mg, 0.09 mmol) and potassium bromomethylfluoroborate (297 mg, 1.48 mmol) were added sequentially to a mixture of compound 14-e (370 mg, 1.63 mmol) in acetonitrile (20 mL) at room temperature. The mixture was stirred at 80° C. under nitrogen atmosphere for 16 hours. The mixture was cooled to room temperature, filtered, and the filter cake was washed with acetonitrile (10 mL), then the filtrate was concentrated at reduced pressure to give the crude compound 14-d (456 mg). The crude product was used directly in the next step without purification. LC-MS (ESI): m/z=309.1[M+1]⁺.

### Synthesis of compound 14

Synthesized according to synthesis of compound 1, compound 14-d was used instead of compound 1-d to obtain compound 14 (10 mg). LC-MS (ESI): m/z=673.3 [M+1]⁺; ¹H NMR (400 MHz, DMSO-d₆): δ 10.59 (1H, s), 9.57 (1H, s), 8.82 (1H, s), 8.43 (1H, d, J=2.0 Hz), 8.13 (1H, brs), 8.01 (1H, d, J=2.0 Hz), 7.71 (1H, brs), 4.74 (2H, s), 4.04 (3H, s), 3.98-3.81 (4H, m), 3.79-3.68 (4H, m), 3.66-3.43 (4H, m), 3.11 (3H, s), 3.06-2.83 (4H, m), 2.71-2.64 (1H, m), 2.36-2.30 (1H, m), 2.30-2.15 (2H, m), 2.14-1.98 (2H, m), 1.97-1.80 (4H, m).

### Synthetic route of compound 15

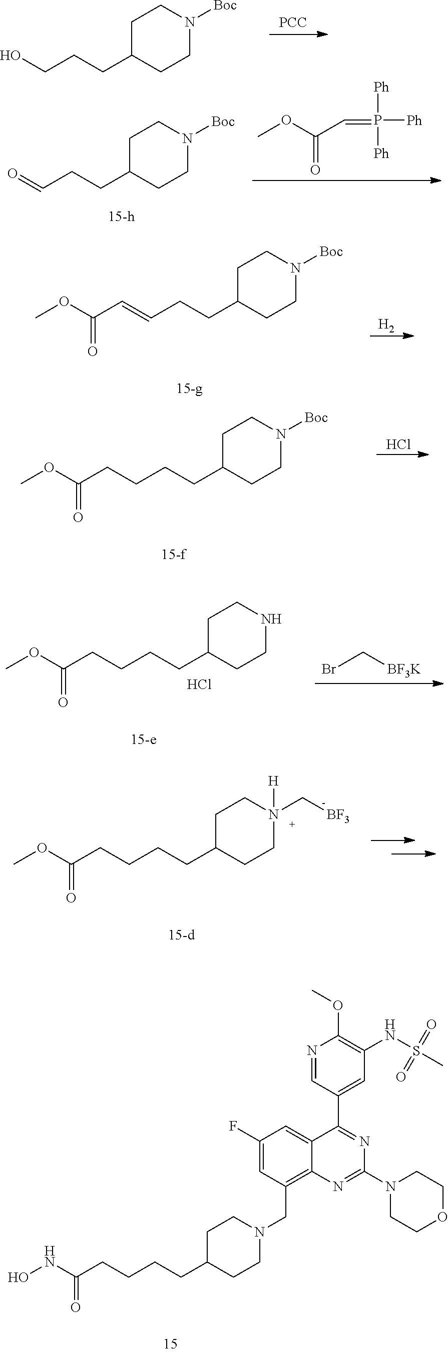

### Synthesis of compound 15-h

To a reaction flask was added 3-(1-Boc-4-piperidinyl)-1-propanol (2.43 g, 10.00 mmol), dichloromethane (100 mL) and PCC (4.30 g, 20.00 mmol), then the mixture was stirred at room temperature overnight for 24 hours. Quenched with saturated sodium bicarbonate (200 mL) and extracted with dichloromethane (200 mL×3). The organic phases were combined, washed with water, brine, dried over anhydrous sodium sulfate, filtered and concentrated to give compound 15-h (1.93 g, 80%). LC-MS (ESI): m/z 186.3 (M-56+H)⁺.

### Synthesis of compound 15-g

To a reaction flask was added 15-h (1.93 g, 8.01 mmol), methoxycarbonylmethyltriphenylphosphine (2.67 g, 8.01 mmol), and toluene (100 mL). The mixture was purged with N₂ and stirred at 120° C. for 12 hours. Concentrated, purified on a silica column (mobile phase: petroleum ether/ethyl acetate 10/1 to 3/1) to give compound 15-g (1.76 g, 74%). LC-MS (ESI): m/z 320.4 (M+Na)*.

### Synthesis of compound 15-f

To a reaction flask was added 15-g (1.76 g, 5.93 mmol), palladium carbon (0.35 g, 10% Pd, 50% wet) and methanol (150 mL). The reaction was stirred at room temperature under hydrogen atmosphere (1000 mL) for 12 hours. The compound 15-f (1.46 g, 83%) was obtained by filtration and concentration. LC-MS (ESI): m/z 200.3 (M-100+H)⁺.

### Synthesis of compound 15-e

To a reaction flask was added 15-f (1.46 g, 4.88 mmol), 1,4-dioxane hydrochloride (50 mL, 200.00 mmol). The mixture was stirred at room temperature under nitrogen atmosphere for 24 hours. The compound 15-e (0.97 g) was obtained by filtration and concentration. LC-MS (ESI): m/z 200.4 (M+H)⁺.

### Synthesis of compound 15-d

15-e (0.76 g, 3.23 mmol), potassium iodide (0.03 g, 0.18 mmol), potassium bromomethylfluoroborate (0.58 g, 2.91 mmol) and potassium carbonate (0.58 g, 4.20 mmol) were suspended in acetonitrile (60 mL). The mixture was stirred at 80° C. under nitrogen atmosphere for 12 hours. Cooled to room temperature, the mixture was filtered, concentrated to obtain the crude product. Later, the crude product was washed with methyl tert-butyl ether and filtered. The organic phase was concentrated to give compound 15-d (0.91 g, 100%). LC-MS (ESI): m/z 299.2 (M+NH₄)

### Synthesis of compound 15

Synthesized according to synthesis of compound 1, compound 15-d was used instead of compound 1-d to obtain compound 15 (30 mg). LC-MS (ESI): m/z 646.3 (M+H)⁺; ¹H NMR (400 MHz, DMSO-d₆): δ 10.35 (1H, s), 9.57 (1H, s), 8.66 (1H, s), 8.43 (1H, d, J=2.0 Hz), 8.11 (1H, s), 8.01 (1H, d, J=1.6 Hz), 7.66 (1H, s), 4.64 (1H, s), 4.04 (3H, s), 3.84-3.95 (4H, m), 3.68-3.76 (4H, m), 3.57 (1H, s), 3.11 (3H, s), 1.90-1.96 (2H, m), 1.70-1.83 (3H, m), 1.36-1.52 (6H, s), 1.10-1.34 (6H, m).

### Synthetic route of compound 16

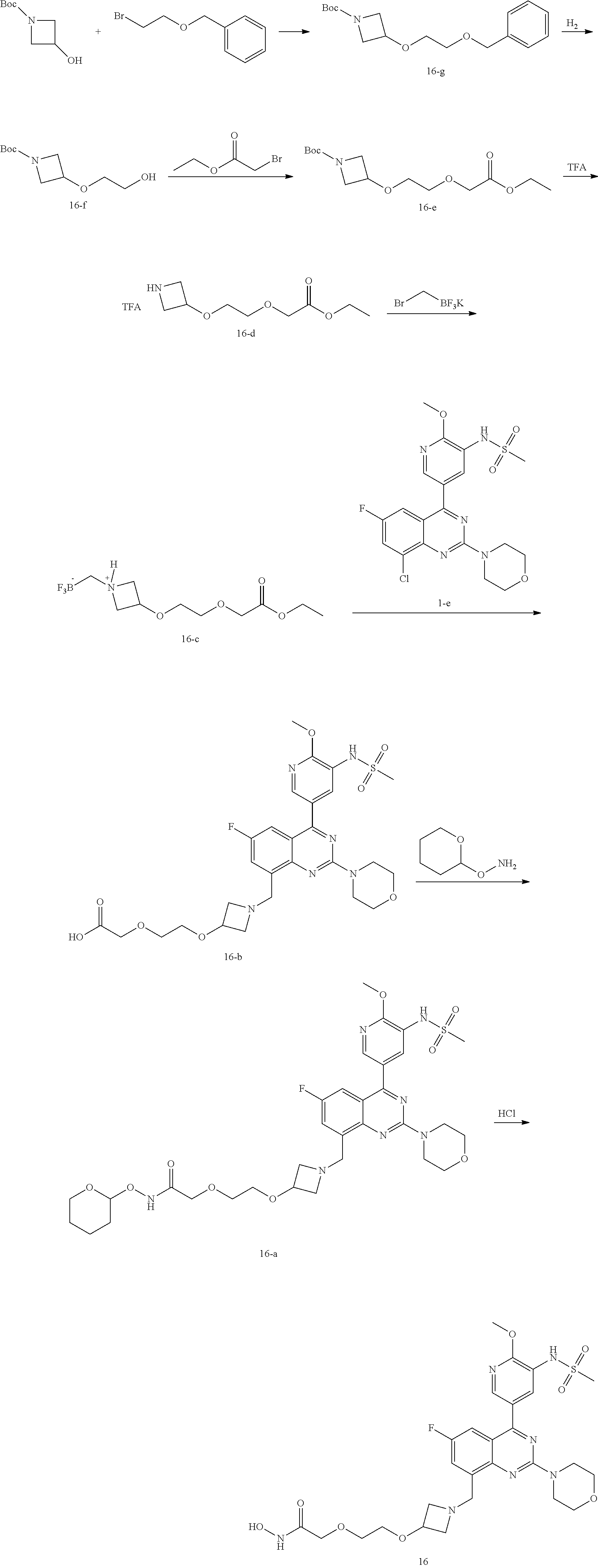

### Synthesis of compound 16-g

NaH (800 mg, 20 mmol, 60%) was added under nitrogen atmosphere to a solution of compound N-Boc-3-hydroxyazetidine (1.73 g, 10 mmol) in DMVF (20 mL.) at 0° C., and then the mixture was warmed to 60° C., benzyl 2-bromoethyl ether (2.37 mL,. 15 mmol) was added dropwise and the resulting mixture was stirred at the same temperature for 16 hours. The mixture was cooled to 0° C., quenched with saturated ammonium chloride solution (20 mL.) and added water (50 mL.), extracted with ethyl acetate (100 mL. x2), and the organic phase was washed with brine (100 mL. x5), dried over anhydrous sodium sulfate, filtered, concentrated at reduced pressure, and the crude product was purified by flash chromatography (PE/EA=1:1) to give compound 16-g (2.58 g, 84%). LC-MS (ESI): m/z=252.1 [M−56+1]⁺.

### Synthesis of compound 16-f

Compound 16-g (2.58 g, 8.39 mmol) was dissolved in methanol (100 mL), followed by addition of Pd/C (10% Pd, 50% wet, 0.5 g) at room temperature under nitrogen atmosphere and the mixture was stirred at room temperature under hydrogen atmosphere for 16 hours. Filtered and concentrated at reduced pressure to give compound 16-f (1.86 g, 100%). LC-MS (ESI): m/z=162.1 [M−56+1]⁺.

### Synthesis of compound 16-e

NaH (177 mg, 4.42 mmol, 60%) was dissolved in THF (10 mL) under nitrogen atmosphere at room temperature, then a solution of compound 16-f (800 mg, 3.68 mmol) in THF (5 mL) was slowly added to the THF solution, and the mixture was stirred at room temperature for 1 hour. After cooled to 0° C., ethyl bromoacetate (490 μL, 4.42 mmol) was added dropwise. After addition, the mixture was stirred at room temperature for 16 hours. The mixture was quenched with saturated ammonium chloride solution (30 ml) at 0° C., water (50 mL) was added, the mixture was extracted with ethyl acetate (100 mL×2), the organic phase was washed with saturated sodium bicarbonate solution (100 mL), dried over anhydrous sodium sulfate, filtered, concentrated at reduced pressure, and the crude product was purified by flash chromatography (PE/EA=1:1). Compound 16-e (518 mg, 46%) was obtained. LC-MS (ESI): m/z=248.1 [M−56+1]⁺.

### Synthesis of compound 16-d

TFA (2 mL) was added to a solution of compound 16-e (518 mg, 1.71 mmol) in DCM (10 mL) at 0° C., and the mixture was raised to room temperature and stirred at room temperature for 2.5 hours. The crude product was dissolved in DCM (20 mL), concentrated at reduced pressure, and the above operation was repeated three times to obtain the crude compound 16-d (542 mg). The crude product was directly used in the next step. LC-MS (ESI): m/z=204.1 [M+1]⁺.

### Synthesis of compound 16-c

Potassium iodide (15 mg, 0.093 mmol), potassium bromomethylfluoroborate (311 mg, 1.55 mmol) and potassium carbonate (321 mg, 2.32 mmol) were added sequentially to a solution of compound 16-d (542 mg, 1.71 mmol) in acetonitrile (20 mL) at room temperature, and the mixture was stirred at 80° C. under nitrogen atmosphere for 16 hours. The mixture was cooled to room temperature, filtered, and the filter cake was washed with acetonitrile (10 mL), then the filtrate was concentrated at reduced pressure to give the crude compound 16-c (1 g, about 44% purity based on theoretical values). The crude product was used directly in the next step without purification. LC-MS (ESI): m/z=303.1 [M+18]⁺.

### Synthesis of compound 16-b

Water (6 mL), 16-c (1 g, 44%, 1.54 mmol), palladium acetate (23 mg, 0.10 mmol), x-Phos (98 mg, 0.21 mmol) and cesium carbonate (1.01 g, 3.1 mmol) were added sequentially to a solution of compound 1-e (482 mg, 1.03 mmol) in THF (60 mL) at room temperature, and the mixture was stirred at 80° C. under nitrogen atmosphere for 36 hours. The mixture was cooled to room temperature and was added water (50 mL), extracted with ethyl acetate (100 mL), the aqueous phase was adjusted to pH=5 with 1M diluted hydrochloric acid, the aqueous phase was concentrated at reduced pressure, the crude product was suspended in a mixture of solvents (DCM/MeOH=10:1, 20 mL), filtered, the filter cake was washed with a mixture of solvents (DCM/MeOH=10:1, 20 mL x 2), and the organic phases were combined. Concentrated at reduced pressure, the crude product was separated and purified by pre-HPLC to give compound 16-b (136 mg, 21%). LC-MS (ESI): m/z=621.2[M+1]⁺.

### Synthesis of compound 16-a

Compound 16-b (50 mg, 0.08 mmol) was dissolved in DMF (3 mL) at room temperature, followed by serial addition of HATU (46 mg, 0.12 mmol) and DIPEA (66 uL, 0.40 mmol) in an ice bath, and the mixture was stirred at 0° C. for 10 min, followed by the addition of O-(tetrahydro-2H-pyran)-2-hydroxylamine (19 mg. 0.16 mmol) and the mixture was brought to room temperature and stirred for 16 hours. The reaction was quenched with water (20 mL), then extracted with ethyl acetate (50 mL×2), the organic phase was washed with brine (100 mL×3), dried over anhydrous sodium sulfate, filtered and concentrated at reduced pressure, and the crude product was purified by flash chromatography (DCM/MeOH=10:1) to give compound 16-a (36 mg, 63%). LC-MS (ESI): m/z=720.2 [M+1]⁺.

### Synthesis of compound 16

A solution of hydrogen chloride in 1,4-dioxane (4 M, 0.25 mL) was added dropwise slowly to a solution of compound 16-a (36 mg, 0.05 mmol) in DCM (5 mL) at 0° C., after addition, the mixture was stirred at 0° C. for 20 min, the reaction was quenched with saturated sodium bicarbonate solution (1 mL), DCM (20 mL) was added, the mixture was dried over anhydrous sodium sulfate, filtered and concentrated at reduced pressure to give compound 16 (33 mg, 100%). LC-MS (ESI): m/z=636.2 [M+1]⁺; ¹H NMR (400 MHz, DMSO-d₆): δ 10.48 (1H, s), 9.52 (1H, brs), 8.87 (1H, s), 8.39 (1H, d, J=2.4 Hz), 8.00 (1H, d, J=1.6 Hz), 7.61-7.53 (1H, m), 7.47-7.37 (1H, m), 4.17 (1H, t, J=5.6 Hz), 4.08 (2H, s), 4.03 (3H, s), 3.91-3.79 (6H, m), 3.77-3.70 (4H, m), 3.70-3.62 (2H, m), 3.59-3.51 (2H, m), 3.51-3.46 (2H, m), 3.10 (3H, s), 3.07-2.96 (2H, m).

### Synthetic route of compound 17

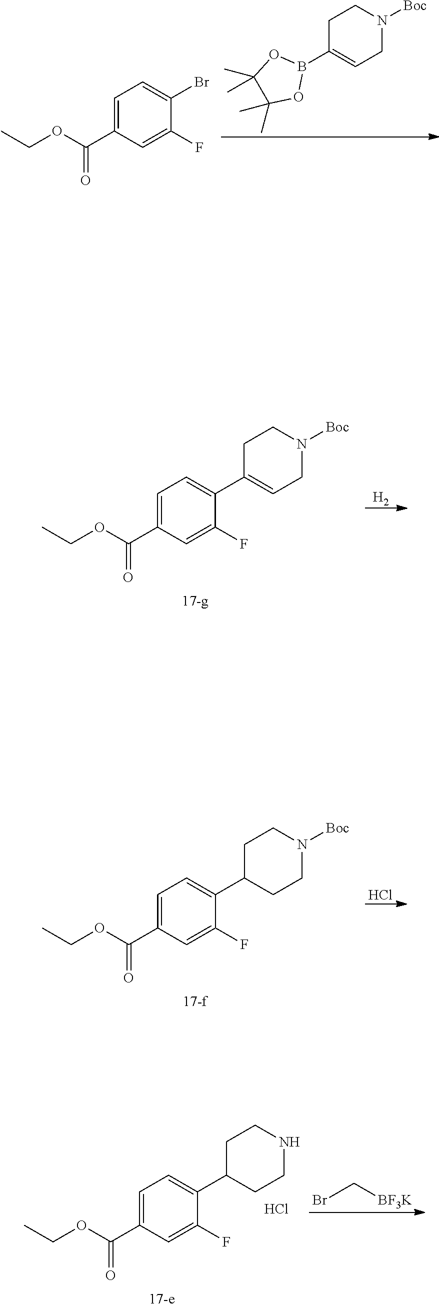

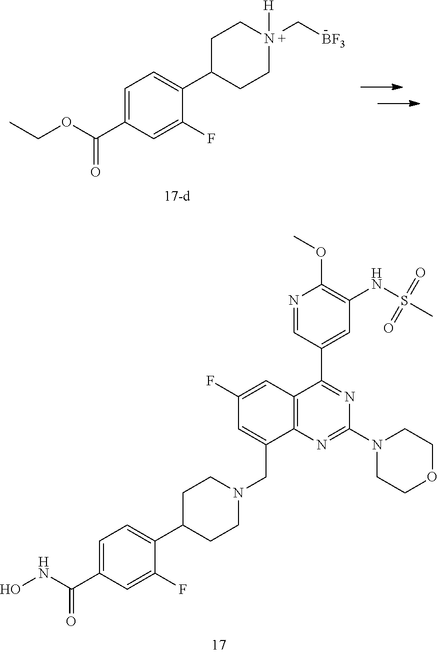

### Synthesis of compound 17-g

N-Boc-1,2,5,6-tetrahydropyridine-4-boronic acid pinacol ester (1.40 g, 4.40 mmol), tetrakis(triphenylphosphine)palladium (462 mg, 0.40 mmol), cesium carbonate (2.60 g, 8.00 mmol) and water (4 mL) were added sequentially to a solution of compound ethyl 3-fluoro-4-bromobenzoate (1 g, 4.00 mmol) in 1,4-dioxane (20 mL) at room temperature and the mixture was stirred at 100° C. under nitrogen atmosphere for 5 hours. The mixture was cooled to room temperature, water was added, and extracted with ethyl acetate. The organic phases were combined, washed with water, brine, dried over anhydrous sodium sulfate, filtered, evaporated and purified on a silica column (mobile phase: DCM/MeOH 10/0 to 10/1) to give compound 17-g (1.1 g, 79%). LC-MS(ESI): m/z=294.2[M-56+H]⁺.

### Synthesis of compound 17-f

Compound 17-g (1.1 g, 3.15 mmol) was dissolved in methanol (50 mL), followed by addition of Pd/C (10% Pd, 50% wet, 1 g) at room temperature and the mixture was stirred at room temperature under hydrogen atmosphere overnight. The mixture was filtered and concentrated to give the crude compound 17-f (980 mg, 88%). LC-MS(ESI): m/z=296.1 [M-56+H]⁺.

### Synthesis of compound 17-e

Compound 17-f (980 mg, 2.79 mmol) was added to a solution of hydrogen chloride in MeOH (4M, 10 mL) and the mixture was stirred at room temperature overnight. The mixture was concentrated to dryness to give the crude compound 17-e (870 mg). The crude product was used directly in the next step. LC-MS (ESI): m/z=252.2 [M+1]⁺.

### Synthesis of compound 17-d

KI (52 mg, 0.30 mmol), potassium bromomethylfluoroborate (632 mg, 3.14 mmol) and K₂CO₃ (652 mg, 4.72 moL) were added sequentially to a solution of Compound 17-e (870 mg, 2.79 mmol) in acetonitrile (10 mL) at room temperature, and the mixture was stirred at 80° C. under nitrogen atmosphere overnight. The mixture was cooled to room temperature, filtered and the filtrate was concentrated to dryness to obtain the crude product. The crude product was dissolved in acetonitrile (5 mL), cooled to 0° C., ethyl ether (50 mL) was added slowly, solid was precipitated, filtered, the solid was collected, and the solid was dried under vacuum for 1 hour to give the crude compound 17-d (900 mg).

### Synthesis of compound 17

Synthesized according to synthesis of Compound 1, Compound 17-d was used instead of compound 1-d to obtain Compound 17 (28 mg). LC-MS (ESI): m/z=684.2 [M+1]⁺; ¹H NMR (400 MHz, DMSO-d₆): δ 11.38 (1H, s), 9.56 (1H, s), 9.13 (1H, s), 8.44 (1H, d, J=2 Hz), 8.27 (1H, d, J=8.0 Hz), 8.02 (1H, d, J=2.0 Hz), 7.71 (1H, d, J=9.2 Hz), 7.61 (1H, d, J=7.2 Hz), 7.54 (1H, d, J=11.2 Hz), 7.34 (1H, t, J=6.8 Hz), 4.78 (2H, d, J=3.2 Hz), 4.03 (3H, s), 3.92 (4H, m), 3.75 (4H, m), 3.67 (1H, s), 3.56-3.48 (2H, m), 3.28-3.25 (2H, m), 3.10 (3H, s), 2.19-2.09 (2H, m), 1.93-1.86 (2H, m).

### Synthetic route of compound 18

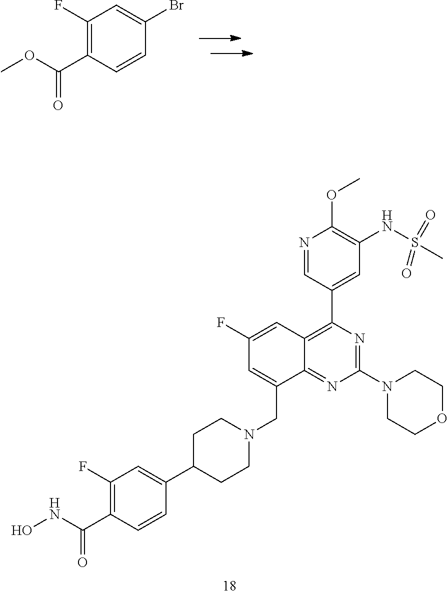 [SMILES: COC(=O)c1ccc(Br)cc1F ; COc1ncc(-c2nc(N3CCOCC3)nc3c(CN4CCC(c5ccc(C(=O)NO)c(F)c5)CC4)cc(F)cc23)cc1NS(C)(=O)=O]

### Synthesis of compound 18

Synthesized according to synthesis of compound 17, compound 18 (81 mg) was obtained by using compound 2-fluoro-4-bromobenzoic acid methyl ester instead of compound 3-fluoro-4-bromobenzoic acid ethyl ester. LC-MS (ESI): m/z=684.2 [M+1]⁺; ¹H NMR (400 MHz, DMSO-d₆): δ 11.16 (1H, s), 10.93 (1H, s), 9.56 (1H, s), 8.43 (1H, d, J=2.0 Hz), 8.33-8.28 (1H, m), 8.02 (1H, d, J=2.0 Hz), 7.69 (1H, m), 7.50 (1H, t, J=7.6 Hz), 7.12 (2H, t, J=9.2 Hz), 4.78 (2H, d, J=3.2 Hz), 4.03 (3H, s), 3.92 (4H, m), 3.75 (4H, m), 3.67 (1H, s), 3.52-3.50 (2H, m), 3.21-3.18 (2H, m), 3.10 (3H, s), 2.22-2.11 (2H, m), 1.96-1.93 (2H, m).

### Synthetic route of compound 19

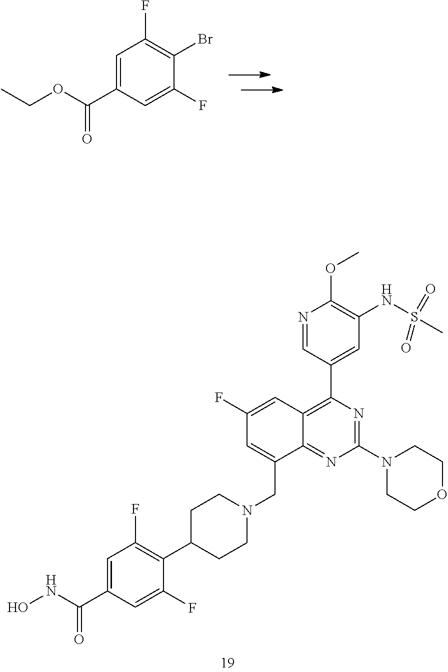 [SMILES: CCOC(=O)c1cc(F)c(Br)c(F)c1 ; COc1ncc(-c2nc(N3CCOCC3)nc3c(CN4CCC(c5c(F)cc(C(=O)NO)cc5F)CC4)cc(F)cc23)cc1NS(C)(=O)=O]

### Synthesis of compound 19

Synthesized according to synthesis of compound 17, compound 19 (2 mg) was obtained using compound 4-bromo-3,5-difluorobenzoic acid ethyl ester instead of compound 3-fluoro-4-bromobenzoic acid ethyl ester. LC-MS (ESI): m/z=702.1 [M+1]⁺.

### Synthetic route of compound 20

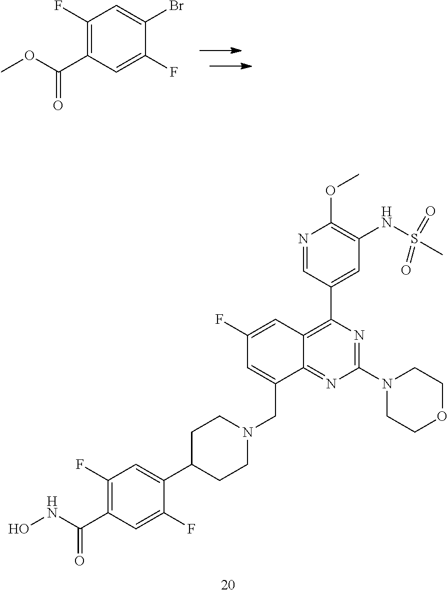 [SMILES: COC(=O)c1cc(F)c(Br)cc1F ; COc1ncc(-c2nc(N3CCOCC3)nc3c(CN4CCC(c5cc(F)c(C(=O)NO)cc5F)CC4)cc(F)cc23)cc1NS(C)(=O)=O]

### Synthesis of compound 20

Synthesized according to synthesis of compound 17, compound 20 (85 mg) was obtained using compound 4-bromo-2,5-difluorobenzoic acid methyl ester instead of compound 3-fluoro-4-bromobenzoic acid ethyl ester. LC-MS (ESI): m/z=702.2 [M+1]⁺.

### Synthetic route of compound 21

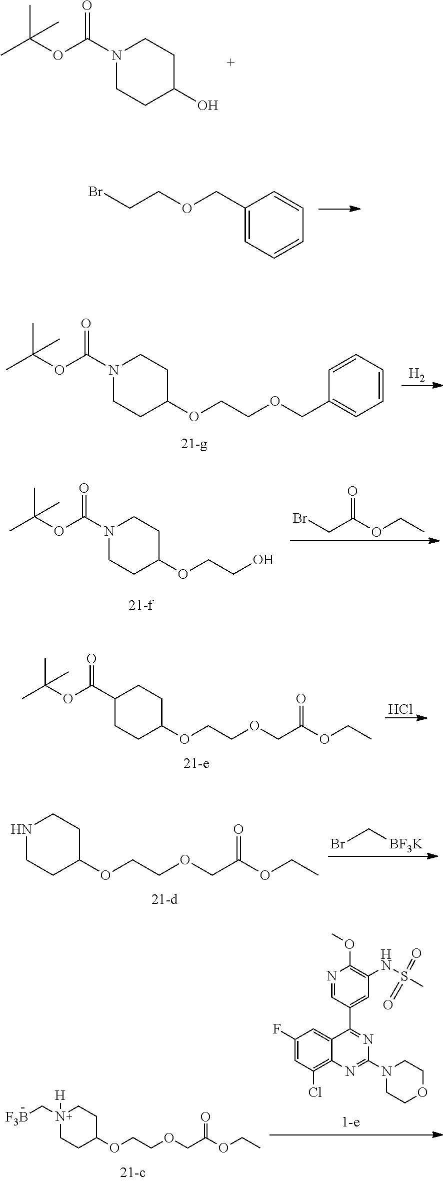

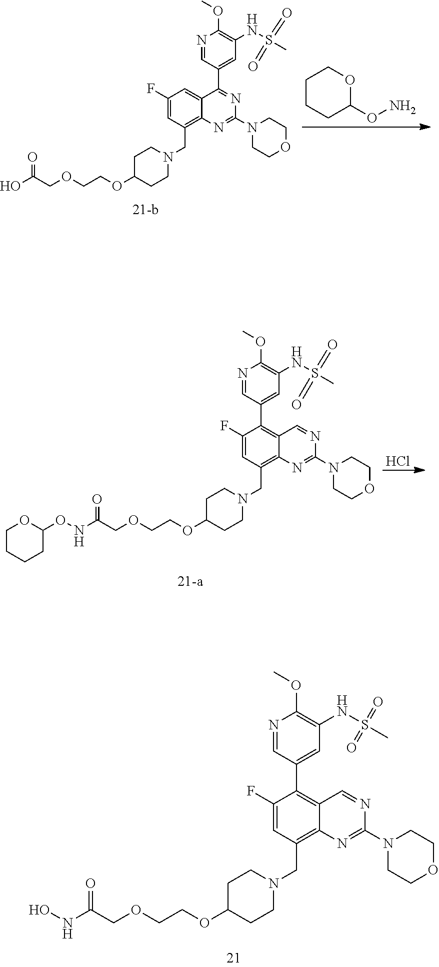 [SMILES: COc1ncc(-c2nc(N3CCOCC3)nc3c(CN4CCC(OCCOCC(=O)O)CC4)cc(F)cc23)cc1NS(C)(=O)=O ; NOC1CCCCO1 ; COc1ncc(-c2c(F)cc(CN3CCC(OCCOCC(=O)NOC4CCCCO4)CC3)c3nc(N4CCOCC4)ncc23)cc1NS(C)(=O)=O ; Cl ; COc1ncc(-c2c(F)cc(CN3CCC(OCCOCC(=O)NO)CC3)c3nc(N4CCOCC4)ncc23)cc1NS(C)(=O)=O]

### Synthesis of compound 21-g

NaH (800 mg, 20 mmol, 60%) was added to a solution of compound N-Boc-4-hydroxypiperidine (2.01 g, 10 mmol) in DMF (20 mL) at 0° C. under nitrogen atmosphere, and the mixture was warmed to 60° C. and stirred at this temperature for 30 min, then benzyl 2-bromoethyl ether (2.37 mL, 15 mmol) was added dropwise and the mixture was stirred under the same condition for 16 hours. The mixture was cooled to 0° C., quenched with saturated ammonium chloride solution (50 mL) and water (50 mL), extracted with ethyl acetate (100 mL×2), the organic phase was washed with brine (100 mL×3), dried over anhydrous sodium sulfate, filtered, concentrated at reduced pressure and the crude product was purified by flash chromatography (PE/EA=2:1) to give compound 21-g (1 g. 30%). LC-MS (ESI): m/z=280.2 [M−56+1]⁺.

### Synthesis of compound 21-f

Pd/C (10% Pd, 50% wet, 0.5 g) was added to a solution of compound 21-g (1 g, 2.98 mmol) in methanol (100 mL) at room temperature under nitrogen atmosphere and the mixture was stirred at room temperature under hydrogen atmosphere for 16 hours. Filtered and concentrated at reduced pressure to give compound 21-f (736 mg, 100%). LC-MS (ESI): m/z=190.2 [M−56+1]⁺.

### Synthesis of compound 21-e

NaH (156 mg, 3.9 mmol, 60%) was dissolved in THF(10 mL) under nitrogen atmosphere at room temperature, followed by slow addition of a solution of compound 21-f (736 mg, 3.0 mmol) in THF (5 mL), and the mixture was stirred at room temperature for 2 hours. After cooled to 0° C., ethyl bromoacetate (432 uL, 3.9 mmol) was added dropwise to the above mixture, and the mixture was brought to room temperature and stirred for 16 hours. The reaction was quenched with saturated ammonium chloride solution (50 ml) at 0° C., and the organic phase was extracted with ethyl acetate (100 mL×2). The organic phase was dried over anhydrous sodium sulfate, filtered and concentrated at reduced pressure, and the crude product was purified by flash chromatography (PE/EA=1:1 to EA) to give compound 21-e (313 mg, 32%). LC-MS (ESI): m/z=276.1 [M−56+1]⁺.

### Synthesis of compound 21-d

A solution of hydrogen chloride in 1,4-dioxane (5 mL) was added to a solution of compound 21-e (313 mg, 0.94 mmol) in DCM (10 mL) at room temperature at 0° C., and the mixture was raised to room temperature and stirred for 16 hours. The mixture was concentrated at reduced pressure to give the crude compound 21-d (252 mg), which was used directly in the next step. LC-MS (ESI): m/z=232.2[M+1]⁺.

### Synthesis of compound 21-c

Potassium iodide (9 mg, 0.054 mmol), potassium bromomethylfluoroborate (180 mg, 0.90 mmol), and potassium carbonate (185 mg, 1.34 mmol) were added sequentially to a solution of compound 21-d (252 mg, 0.94 mmol) in acetonitrile (20 mL) at room temperature, then the mixture was stirred at 80° C. under nitrogen atmosphere for 16 hours. Cooled to room temperature, the mixture was filtered, and the filter cake was washed with acetonitrile (10 mL), then the filtrate was concentrated at reduced pressure, the crude product was dissolved in acetonitrile (5 mL), ethyl ether (50 mL) was added to the mixture, solid was precipitated, filtered, and the filtre cake was washed with ethyl ether (10 mL), and the solid was collected to give compound 21-c (216 mg, 77%). LC-MS (ESI): m/z=331.2 [M+18]⁺.

### Synthesis of compound 21-b

Water (2 mL), 21-c (216 mg, 0.69 mmol), palladium acetate (10 mg, 0.046 mmol), x-Phos (44 mg, 0.092 mmol) and cesium carbonate (450 mg, 1.38 mmol) were added sequentially to a solution of compound 1-e (215 mg, 0.46 mmol) in THF (20 mL) at room temperature, and the mixture was stirred at 80° C. under nitrogen atmosphere for 48 hours. The mixture was cooled to room temperature and water (30 mL) was added, extracted with ethyl acetate (100 mL), the aqueous phase was adjusted to pH=5 with 1M diluted hydrochloric acid, the aqueous phase was concentrated at reduced pressure, the crude product was suspended in a mixture of solvents (DCM/MeOH=10:1, 20 mL), filtered, the filter cake was washed with a mixture of solvents (DCM/MeOH=10:1, 20 mL x2), the organic phases were combined. The organic phase was combined and concentrated at reduced pressure, and the crude product was purified by Pre-HPLC to give compound 21-b (10 mg, 3.3%). LC-MS (ESI): m/z=649.2[M+1]⁺.

### Synthesis of compound 21-a

Compound 21-b (10 mg, 0.015 mmol) was dissolved in DMF (1 mL) at room temperature, HATU (9 mg, 0.023 mmol) and DIPEA (13 μL, 0.077 mmol) were added sequentially to the above solution in an ice bath, and the mixture was stirred at 0° C. for 10 min, followed by the addition of O-(tetrahydro-2H-pyran)-2-hydroxylamine (10 mg, 0.085 mmol) and the mixture was brought to room temperature and stirred for 16 hours. The reaction was quenched with water(10 mL), then extracted with ethyl acetate (20 mL×2), the organic phase was washed with brine (30 mL×3), dried over anhydrous sodium sulfate, filtered and concentrated at reduced pressure, and the crude product was purified by Pre-HPLC to afford compound 21-a (12 mg, 100%). LC-MS (ESI): m/z=748.3 [M+1]⁺.

### Synthesis of compound 21

A solution of hydrogen chloride in 1,4-dioxane (4 M, 0.3 mL) was added dropwise slowly to a solution of compound 21-a (12 mg, 0.016 mmol) in DCM (5 mL) at 0° C., after addition, the mixture was stirred at 0° C. for 20 min, and the reaction was quenched with saturated sodium bicarbonate solution (2 mL), DCM (20 mL) was added, and the mixture was dried over anhydrous sodium sulfate. The mixture was filtered and concentrated at reduced pressure to give compound 21 (6 mg, 54%). LC-MS (ESI): m/z=664.2 [M+1]⁺; ¹H NMR (400 MHz, DMSO-d₆): δ 10.45 (1H, s), 9.53 (1H, brs), 8.88 (1H, s), 8.41 (1H, d, J=1.2 Hz), 8.05 (1H, s), 8.00 (1H, d, J=2.4 Hz), 4.64 (1H, s), 4.02 (3H, s), 3.93-3.79 (4H, m), 3.79-3.65 (4H, m), 3.55 (2H, s), 3.52-3.19 (1OH, m), 3.09 (3H, s), 2.00-1.83 (3H, m), 1.59-1.38 (2H, m).

### Synthetic route of compound 22

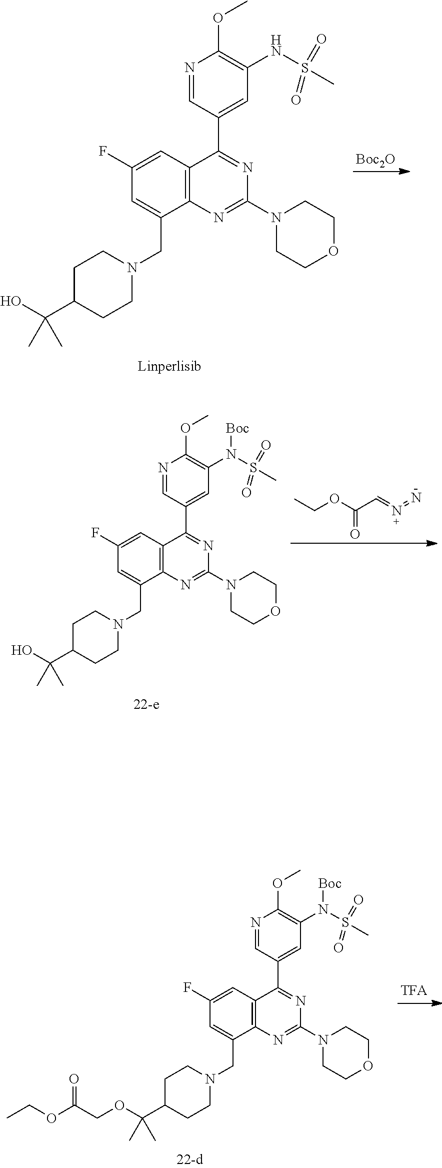

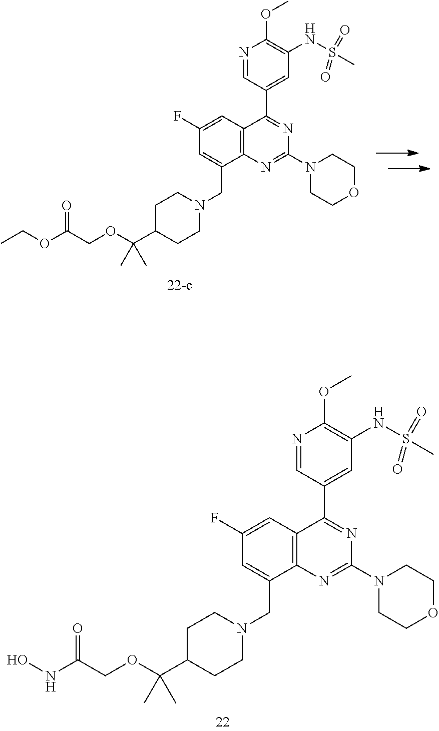 [SMILES: CCOC(=O)COC(C)(C)C1CCN(Cc2cc(F)cc3c(-c4cnc(OC)c(NS(C)(=O)=O)c4)nc(N4CCOCC4)nc23)CC1 ; COc1ncc(-c2nc(N3CCOCC3)nc3c(CN4CCC(C(C)(C)OCC(=O)NO)CC4)cc(F)cc23)cc1NS(C)(=O)=O]

### Synthesis of compound 22-e

DMAP (716 mg, 5.86 mmol) and Boc₂O (1.17 g, 5.35 mmol) were added to a solution of compound Linperlisib (3 g, 5.1 mmol) in DCM (30 mL) under nitrogen atmosphere at room temperature and the mixture was stirred for 16 hours. Concentrated at reduced pressure, the crude product was purified by flash chromatography (DCM/MeOH=10:1) to give compound 22-e (3 g, 86%). LC-MS (ESI): m/z=689.2 [M+1]⁺.

### Synthesis of compound 22-d

Rh₂(OAc)₄ (65 mg, 0.15 mmol) and ethyl diazoacetate (99 mg, 0.87 mmol) were added to a solution of compound 22-e (200 mg, 0.29 mmol) in DCM (20 mL) at room temperature under nitrogen atmosphere, then the mixture was stirred at room temperature under nitrogen atmosphere for 2 days. Supplemented with ethyl diazoacetate(500 μL) and stirred at 30° C. under nitrogen atmosphere for 16 hours. Cooled to room temperature, the mixture was filtered, the filtrate was concentrated at reduced pressure and the crude product was purified by a flash column (DCM/MeOH=10:1) to give the compound 22-d (about 100 mg), which was used directly in the next step. LC-MS (ESI): m/z=775.3 [M+1]⁺.

### Synthesis of compound 22-c

TFA (7 mL) was added to a solution of compound 22-d (200 mg, 0.26 mmol) in DCM (20 mL) at 0° C. under nitrogen atmosphere and the resulting mixture was stirred at 0° C. under nitrogen atmosphere for 5 hours. Concentrated at reduced pressure, the crude product was suspended in saturated sodium bicarbonate solution(50 mL), extracted with DCM (100 mL×2), the organic phase was dried over anhydrous sodium sulfate, filtered, concentrated at reduced pressure, and the crude product was purified by flash chromatography (DCM/MeOH=10:1) to give compound 22-c (115 mg, 66%) as a yellow solid. LC-MS (ESI): m/z=675.3 [M+1]⁺.

### Synthesis of compound 22

Synthesized according to synthesis of compound 1, compound 22-c was used instead of compound 1-c to obtain compound 22 (18 mg). LC-MS (ESI): m/z=662.2 [M+1]⁺; ¹H NMR (400 MHz, DMSO-d₆): δ 10.21 (1H, s), 9.49 (1H, brs), 8.75 (1H, s), 8.41 (1H, d, J=2.0 Hz), 8.00 (1H, d, J=2.0 Hz), 7.72 (1H, brs), 7.47 (1H, brs), 4.03 (3H, s), 3.90-3.78 (4H, m), 3.80-3.61 (6H, m), 3.24-3.20 (1H, m), 3.10 (3H, s), 3.10-2.89 (2H, m), 2.22-1.89 (2H, m), 1.72-1.55 (2H, m), 1.55-1.26 (4H, m), 1.07 (6H, s).

### Synthetic route of compound 23

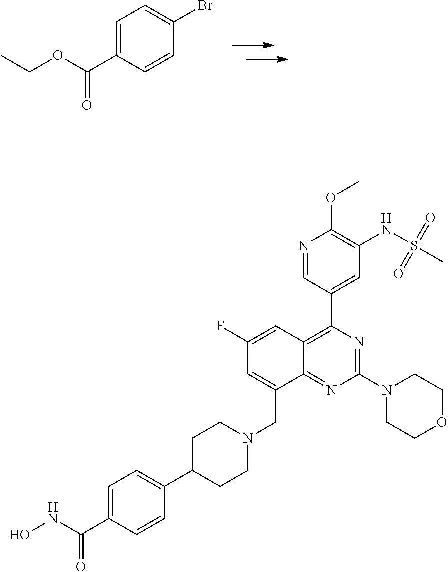 [SMILES: CCOC(=O)c1ccc(Br)cc1 ; COc1ncc(-c2nc(N3CCOCC3)nc3c(CN4CCC(c5ccc(C(=O)NO)cc5)CC4)cc(F)cc23)cc1NS(C)(=O)=O]

### Synthesis of compound 23

Synthesized according to synthesis of compound 17, compound 23 (40 mg) was obtained using compound 4-bromo-ethyl benzoate instead of compound 3-fluoro-4-bromobenzoic acid ethyl ester. LC-MS (ESI): m/z=666.2 [M+1]⁺; ¹H NMR (400 MHz, DMSO-d₆): δ 11.13 (1H, s), 8.98 (1H, s), 8.39 (1H, d, J=1.6 Hz), 7.99 (1H, d, J=2 Hz), 7.75 (1H, d, J=7.6 Hz), 7.67 (2H, d, J=8.0 Hz), 7.45 (1H, d, J=7.6 Hz), 7.33 (2H, d, J=8.0 Hz), 4.14-3.95 (1H, m), 4.01 (3H, s), 3.93-3.78 (4H, m), 3.77-3.65 (4H, m), 3.14-2.97 (2H, m), 3.09 (3H, s), 2.88 (2H, s), 2.66-2.54 (1H, m), 2.40-2.14 (2H, m), 1.86-1.63 (4H, m).

### Synthetic route of compound 24

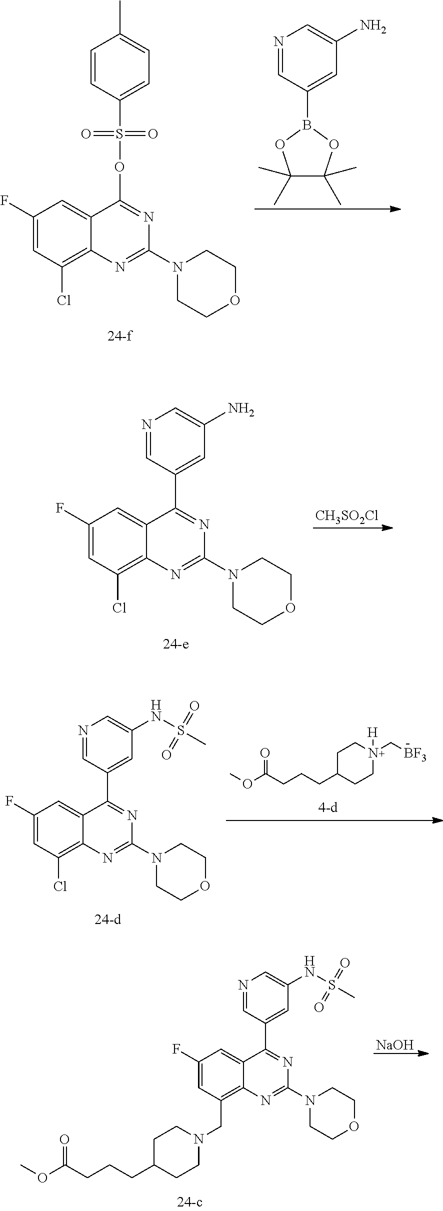

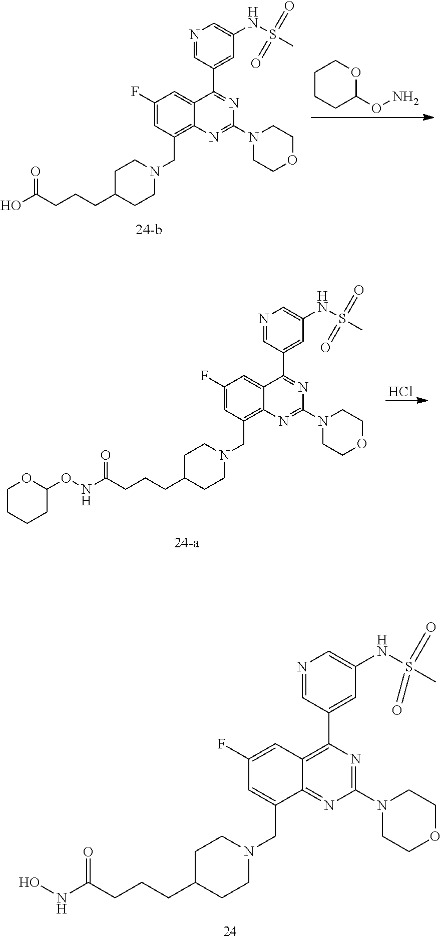 [SMILES: CS(=O)(=O)Nc1cncc(-c2nc(N3CCOCC3)nc3c(CN4CCC(CCCC(=O)O)CC4)cc(F)cc23)c1 ; NOC1CCCCO1 ; CS(=O)(=O)Nc1cncc(-c2nc(N3CCOCC3)nc3c(CN4CCC(CCCC(=O)NOC5CCCCO5)CC4)cc(F)cc23)c1 ; Cl ; CS(=O)(=O)Nc1cncc(-c2nc(N3CCOCC3)nc3c(CN4CCC(CCCC(=O)NO)CC4)cc(F)cc23)c1]

### Synthesis of compound 24-f

The compound 24-f was synthesized according to the patent CN111440142A.

### Synthesis of compound 24-e

To a reaction vial was added compound 24-f (2 g, 4.56 mmol), 5-aminopyridine-3-boronic acid pinacol ester (1.2 g, 5.48 mmol), toluene (30 mL), isopropanol (10 mL), water (10 mL), sodium carbonate (3.8 g, 27.4 mmol) and tetrakis(triphenylphosphine)palladium (264 mg, 0.23 mmol), then the resulting mixture was degassed and purged with nitrogen for three times and stirred at 100° C. under nitrogen atmosphere overnight. After finished, the mixture was spun dry and purified over the column (mobile phase: methanol/dichloromethane 0/100 to 5/95) to give compound 24-e (400 mg, 24%). LC-MS (ESI): m/z=360.0 (M+H)⁺.

### Synthesis of compound 24-d

To a reaction vial was added 24-e (400 mg, 1.11 mmol) and pyridine (5 mL), then methanesulfonyl chloride (191 mg, 1.66 mmol) was added dropwise to the above vial in an ice bath and the mixture was stirred at room temperature overnight. The next day, the reaction was quenched with water, extracted with ethyl acetate (50 mL×2), washed with brine, dried over anhydrous sodium sulfate, concentrated to dryness and purified on a silica column (mobile phase: ethyl acetate/petroleum ether 0/100 to 100/0) to give compound 24-d (350 mg, 72%). LC-MS (ESI): m/z =438.0 (M+H)⁺.

### Synthesis of compound 24-c

To a reaction vial was added 24-d (350 mg, 0.80 mmol), tetrahydrofuran (20 mL), 4-d (641 mg, 2.40 mmol), water (2 mL), X-Phos (76 mg, 0.16 mmol), cesium carbonate (781 mg, 2.40 mmol) and palladium acetate (18 mg, 0.08 mmol). After degassed and purged with nitrogen for three times, the mixture was stirred at 80° C. for 36 hours, concentrated to dryness and purified on a silica column (mobile phase: methanol/dichloromethane 0/100 to 5/95) to give compound 24-c (300 mg, 62%). LC-MS (ESI): m/z=601.2 (M+H)⁺.

### Synthesis of compound 24-b

To a reaction vial was added 24-c (300 mg, 0.50 mmol), tetrahydrofuran (10 mL), methanol (10 mL), water (5 mL) and sodium hydroxide (80 mg, 2 mmol), then the mixture was stirred at room temperature under nitrogen atmosphere overnight. The next day, tetrahydrofuran and methanol were removed, pH was adjusted to 4 with 1M hydrochloric acid, extracted with dichloromethane/methanol=10/1 (15 mL×6). Dried over anhydrous sodium sulfate and concentrated to dryness to give compound 24-b (200 mg, 68%), which was used directly in the next step without purification. LC-MS (ESI): m/z=587.2 (M+H)⁺.

### Synthesis of compound 24-a

To a reaction vial, 24-b (200 mg, 0.34 mmol), DMF (5 mL), O—(tetrahydro-2H-pyran-2-yl)hydroxylamine (60 mg, 0.51 mmol), HATU (260 mg, 0.68 mmol) and DIPEA (132 mg, 1.02 mmol) were added and the resulting mixture was stirred at room temperature under nitrogen atmosphere overnight. The next day, the mixture was purified by Pre-HPLC (ammonium bicarbonate) and lyophilized to give compound 24-a (120 mg, 51%). LC-MS (ESI): m/z=686.2 (M+H)⁺.

### Synthesis of compound 24

A solution of hydrogen chloride in 1,4-dioxane (4M, 1.0 mL) was added dropwise to a solution of 24-a (120 mg, 0.17 mmol) in dichloromethane (20 mL) in an ice bath and the mixture was stirred at the same temperature for 50 min. It was quenched with saturated sodium bicarbonate solution(1.0 mL), then supplemented with solid sodium bicarbonate(1g) and dichloromethane/methanol (10/1, 100 mL), and dried over anhydrous sodium sulfate. The mixture was filtered, the filtrate was concentrated to dryness, and then lyophilized with acetonitrile water to give 24 (70 mg, 66%). LC-MS (ESI): m/z=602.3 (M+H)*; ¹H NMR (400 MHz, DMSO-d₆): δ 10.32-10.65 (2H, m), 8.65-8.69 (3H, m), 8.20-8.32 (1H, m), 7.97-7.98 (1H, m), 7.62-7.64 (1H, m), 4.64-4.77 (2H, m), 3.87-4.02 (4H, m), 3.69-3.79 (4H, m), 3.58 (2H, s), 3.30-3.50 (2H, m), 3.16 (3H, s), 2.91-3.11 (2H, m), 1.88-1.99 (2H, m), 1.73-1.84 (2H, m), 1.39-1.60 (5H, m).

### Synthetic route of compound 25

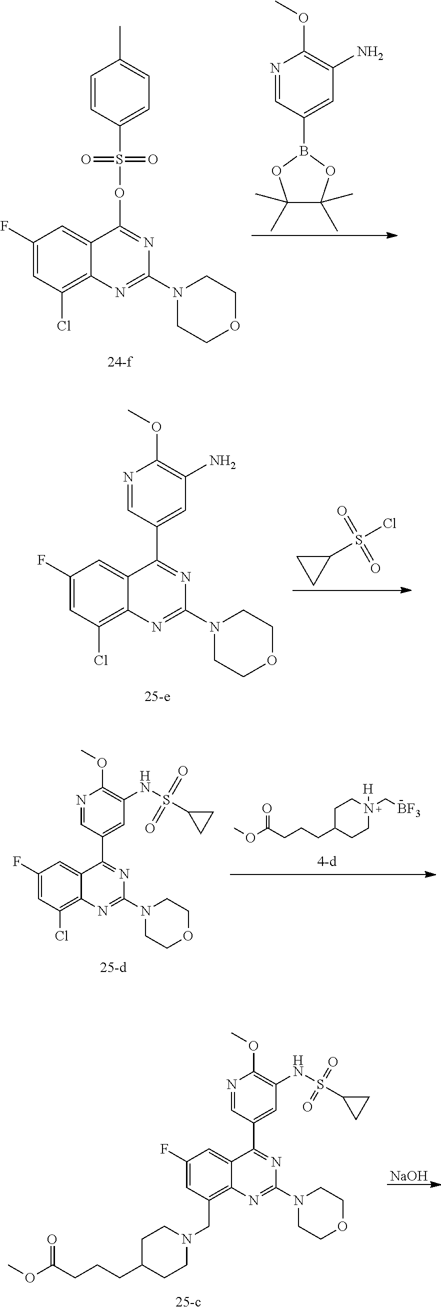

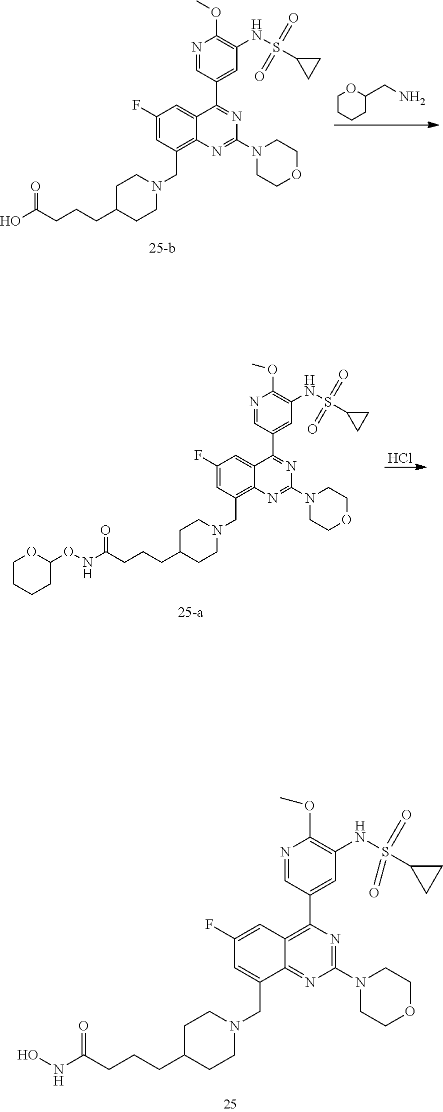 [SMILES: COc1ncc(-c2nc(N3CCOCC3)nc3c(CN4CCC(CCCC(=O)O)CC4)cc(F)cc23)cc1NS(=O)(=O)C1CC1 ; NCC1CCCCO1 ; COc1ncc(-c2nc(N3CCOCC3)nc3c(CN4CCC(CCCC(=O)NOC5CCCCO5)CC4)cc(F)cc23)cc1NS(=O)(=O)C1CC1 ; Cl ; COc1ncc(-c2nc(N3CCOCC3)nc3c(CN4CCC(CCCC(=O)NO)CC4)cc(F)cc23)cc1NS(=O)(=O)C1CC1]

### Synthesis of compound 25-e

Water (10 mL), isopropanol (10 mL), 3-amino-2-methoxypyridine-5-boronic acid pinacol ester (300 mg, 1.2 mmol), tetrakis(triphenylphosphine)palladium (58 mg, 0.05 mmol) and sodium carbonate (636 mg, 6 mmol) were added sequentially to a solution of compound 24-f (438 mg, 1 mmol) in toluene(30 mL ) at room temperature, and the mixture was stirred at 30° C. under nitrogen atmosphere for 16 hours. After concentrated at reduced pressure, water (50 mL) was added to the reaction system, extracted with ethyl acetate (100 mL×2), and the organic phase was washed with brine (100 mL×2), dried over anhydrous sodium sulfate, filtered, and concentrated at reduced pressure to obtain the crude product. The crude product was purified by flash chromatography (PE/EA=1:1) to give compound 25-e (381 mg, 98%). LC-MS (ESI): m/z=390.1[M+1]⁺.

### Synthesis of compound 25-d

Cyclopropylsulfonyl chloride (276 mg, 1.96 mmol) was added dropwise to a solution of compound 25-e (381 mg, 0.98 mmol) in pyridine(6 mL) in an ice bath, and the mixture was raised to 40° C. and stirred for 16 hours. Cooled to room temperature, the mixture was added water (100 mL) and extracted with DCM (100 mL×2), the organic phase was dried over anhydrous sodium sulfate, filtered and concentrated at reduced pressure to obtain the crude product, which was purified by flash chromatography (PE/EA=1:1) to give compound 25-d (400 mg, 83%). LC-MS (ESI): m/z =494.1 [M+1]⁺.

### Synthesis of compound 25-c

Compound 25-d (200 mg, 0.41 mmol) was dissolved in THE (20 mL.) at room temperature, followed by serial addition of water (2 mL), 4-d (325 mg, 1.22 mmol), palladium acetate (10 mg, 0.041 mmol), x-Phos (39 mg, 0.081 mmol) and cesium carbonate (396 mg, 1.22 mmol). After addition the mixture was stirred at 80° C. under nitrogen atmosphere for 48 hours. The reaction system was added water (50 mL), extracted with ethyl acetate (100 mL×2), and the organic phase was washed with brine (100 mL×2), dried over anhydrous sodium sulfate, filtered, and concentrated at reduced pressure to obtain the crude product. The crude product was purified by flash chromatography (PE/EA=1:1, DCM/MeOH=10:1) to give compound 25-c (135 mg, 51%). LC-MS (ESI): m/z=657.2 [M+1]⁺.

### Synthesis of compound 25-b

Compound 25-c (135 mg, 0.21 mmol) was dissolved in methanol (10 mL), followed by serial addition of THF (10 mL), water (5 mL) and sodium hydroxide (82 mg, 2.06 mmol) at room temperature. After addition, the mixture was stirred at room temperature for 12 hours. Concentrated at reduced pressure, the residue was dissolved in water (20 mL), pH was adjusted to 4˜5 with 1M HCl aqueous solution, extracted with mixed solvent (DCM/MeOH=10:1, 100 mL×3), the organic phase was dried over anhydrous sodium sulfate, filtered, and the crude compound 25-b (127 mg, 96%) was concentrated at reduced pressure. LC-MS (ESI): m/z=643.2 [M+1]⁺.

### Synthesis of compound 25-a

Compound 25-b (127 mg, 0.20 mmol) was dissolved in DMF(3 mL )at room temperature, followed by serial addition of HATU (113 mg, 0.30 mmol) and DIPEA (163 uL, 0.99 mmol) in an ice bath, and the mixture was stirred at 0° C. for 10 min, followed by addition of O-(tetrahydro-2H-pyran)-2-hydroxylamine (46 mg, 0.40 mmol), and the mixture was brought to room temperature and stirred for 16 hours. The mixture was purified by pre-HPLC to give compound 25-a (98 mg, 67%). LC-MS (ESI): m/z=742.1 [M+1]⁺.

### Synthesis of compound 25

A solution of hydrogen chloride in 1,4-dioxane (4 M, 0.25 mL) was added dropwise slowly to a solution of compound 25-a (98 mg, 0.13 mmol) in DCM (5 mL) at 0° C., after addition, the mixture was stirred at 0° C. for 20 min, the reaction was quenched with saturated sodium bicarbonate solution (1 mL) and a mixture of solvent (DCM/MeOH=10:1. 50 mL) was added. the system was dried over anhydrous sodium sulfate, filtered and concentrated at reduced pressure to give compound 25 (80 mg, 92%). LC-MS (ESI): m/z=658.3[M+1]⁺; ¹H NMR (400 MHz, DMSO-d₆): δ 10.33 (1H, s), 9.52 (1H, brs), 8.66 (1H, s), 8.39 (1H, d, J=2.4 Hz), 7.99 (1H, d, J=2.0 Hz), 7.67 (1H, d, J=9.6 Hz), 7.42 (1H, dd, J₁=2.0 Hz, J₂=8.8 Hz), 4.01 (3H, s), 3.96 (2H, s), 3.89-3.77 (4H, m), 3.77-3.63 (4H, m), 2.96-2.82 (2H, m), 2.82-2.69 (1H, m), 2.16-2.01 (2H, m), 1.94-1.85 (2H, m), 1.67-1.55 (2H, m), 1.52-1.41 (2H, m), 1.24-1.11 (5H, m), 0.99-0.88 (4H, m).

### Synthetic route of compound 26

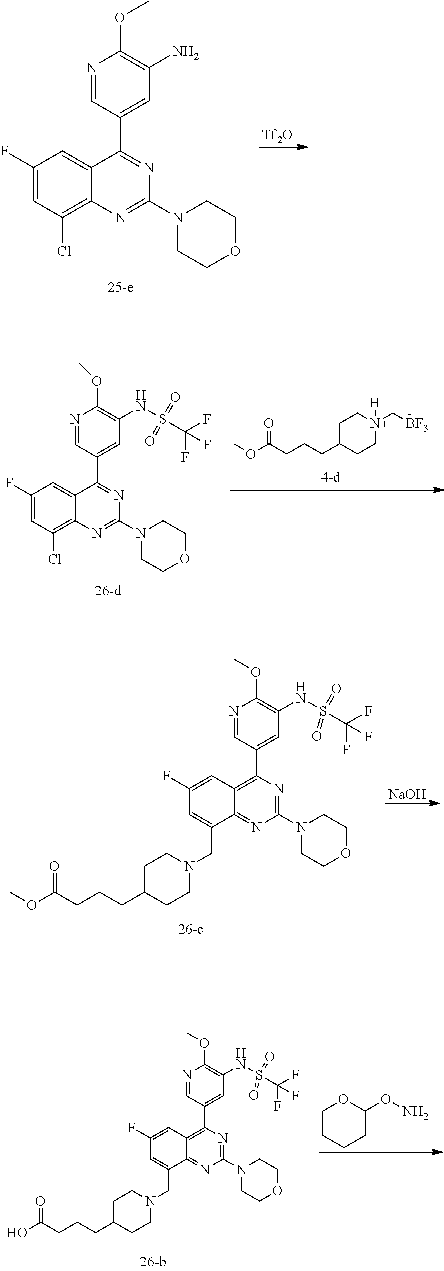

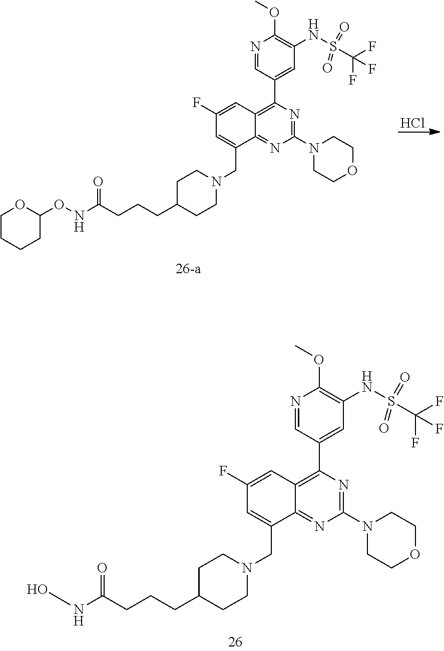 [SMILES: COc1ncc(-c2nc(N3CCOCC3)nc3c(CN4CCC(CCCC(=O)NOC5CCCCO5)CC4)cc(F)cc23)cc1NS(=O)(=O)C(F)(F)F ; Cl ; COc1ncc(-c2nc(N3CCOCC3)nc3c(CN4CCC(CCCC(=O)NO)CC4)cc(F)cc23)cc1NS(=O)(=O)C(F)(F)F]

### Synthesis of compound 26-d

Compound 25-e (200 mg, 0.51 mmol) was dissolved in DCM (10 mL), followed by dropwise addition of trifluoromethanesulfonic anhydride (255 μL, 1.54 mmol) under nitrogen atmosphere at 0° C. The mixture was stirred under nitrogen atmosphere at 0° C. for 30 min. The reaction was quenched with saturated sodium bicarbonate (50 mL) at 0° C., and the mixture was extracted with DCM (100 mL×5). The organic phase was dried over anhydrous sodium sulfate, filtered and concentrated to dryness to give the crude product. The crude product was purified by flash chromatography (dichloromethane: methanol=10:1) to give compound 26-d (260 mg, 97%). LC-MS (ESI): m/z=522.0 [M+1]⁺.

### Synthesis of compound 26-c

Compound 26-d (260 mg, 0.50 mmol) was dissolved in THF (50 mL) at room temperature, followed by serial addition of water (5 mL), 4-d (399 mg, 1.49 mmol), palladium acetate (11 mg, 0.5 mmol), x-Phos (48 mg, 0.10 mmol) and cesium carbonate (487 mg, 1.49 mmol). The mixture was stirred at 80° C. under nitrogen atmosphere for 36 hours. To the reaction system, water(50 mL) was added, extracted with ethyl acetate (100 mL×2), and the organic phase was washed with brine (100 mL×2), dried over anhydrous sodium sulfate, filtered, and concentrated at reduced pressure to obtain the crude product. The crude product was purified by flash column (DCM/MeOH=10:1) to give compound 26-c (290 mg, 85%). LC-MS (ESI): m/z=685.2 [M+1]⁺.

### Synthesis of compound 26-b

Compound 26-c (290 mg, 0.42 mmol) was dissolved in methanol (10 mL), followed by serial addition of THF (10 mL), water (5 mL) and sodium hydroxide (170 mg, 4.24 mmol) at room temperature, and the mixture was stirred at room temperature for 16 hours. Concentrated at reduced pressure, the residue was dissolved in water (20 mL), pH was adjusted to 3˜4 with 1M HCl aqueous solution, extracted with a mixture of DCM/MeOH (10:1) solvent (100 mL×5), the organic phase was dried over anhydrous sodium sulfate, filtered, and concentrated at reduced pressure to obtain the crude compound 26-b (270 mg, 95%). LC-MS (ESI): m/z=671.2 [M+1]⁺.

### Synthesis of compound 26-a

Compound 26-b (100 mg, 0.15 mmol) was dissolved in DMF (3 mL) at room temperature, followed by serial addition of HATU (86 mg, 0.23 mmol) and DIPEA (124 uL, 0.75 mmol) in an ice bath. The mixture was stirred at 0° C. for 10 min, followed by the addition of O-(tetrahydro-2H-pyran)-2-hydroxylamine (35 mg, 0.3 mmol), and the mixture was brought to room temperature and stirred for 16 hours. The mixture was purified by pre-HPLC to give compound 26-a (85 mg, 74%). LC-MS (ESI): m/z=770.2[M+1]⁺.

### Synthesis of compound 26

A solution of hydrogen chloride in methanol (4 M, 1 mL) was slow dropwise added to a solution of compound 26-a (85 mg, 0.11 mmol) in a mixture of DCM/MeOH solvent (10 mL, 10:1) at 0° C., after addition, the resulting mixture was stirred at 0° C. for 20 min and then the reaction was quenched with saturated sodium bicarbonate solution (about 10 mL) at 0° C. (pH=8). The mixture was concentrated at 50° C. at reduced pressure and the residue was suspended in ethanol (50 mL) and concentrated to dryness, and the above operation was repeated 3 times to obtain the dried crude product. The above crude product was suspended in a mixture of DCM/MeOH (100 mL, about 4:1), filtered, and the filtrate was concentrated to dryness at reduced pressure to obtain the crude product. The crude product was suspended in a mixture of DCM/MeOH (50 mL, 8:1), filtered through a microporous membrane, and the filtrate was concentrated at reduced pressure, and the crude product was purified by pre-HPLC (acidic method) to give compound 26 (6.7 mg, 8.9%). LC-MS (ESI): m/z=686.1 [M+1]⁺.

### Synthetic route of compound 27

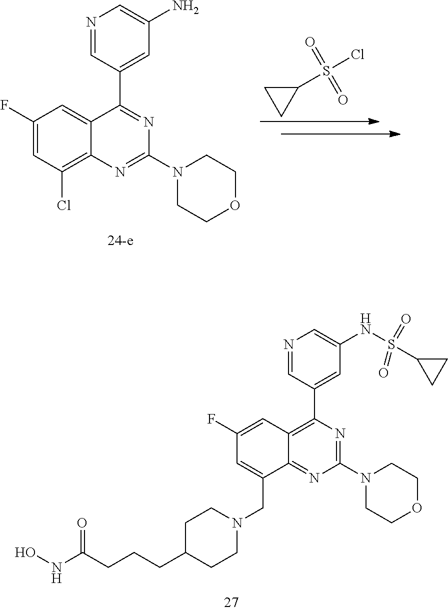 [SMILES: Nc1cncc(-c2nc(N3CCOCC3)nc3c(Cl)cc(F)cc23)c1 ; O=S(=O)(Cl)C1CC1 ; O=C(CCCC1CCN(Cc2cc(F)cc3c(-c4cncc(NS(=O)(=O)C5CC5)c4)nc(N4CCOCC4)nc23)CC1)NO]

### Synthesis of compound 27

Synthesized according to synthesis of compound 24, compound 27 (53 mg) was obtained using compound cyclopropylsulfonyl chloride instead of compound methanesulfonyl chloride. LC-MS (ESI): m/z=628.1 (M+H)*; ¹H NMR (400 MHz, DMSO-d₆): δ 10.34(1H, bs), 8.66 (1H, m), 8.22-8.15 (1H, m), 8.05-7.98 (1H, m), 7.70-7.58 (2H, m), 7.40-7.33 (1H, m), 3.94 (2H, s), 3.90-3.81 (4H, m), 3.77-3.67 (4H, m), 2.90(1H, d, J=10.0 Hz), 2.41-2.30 (1H, m), 2.07(2H, t, J=10.0 Hz), 1.93 (2H, t, J=7.2 Hz), 1.69-1.58 (2H, m), 1.56-1.44 (2H, m), 1.30-1.09 (6H, m), 0.83-0.64 (4H, m).

### Synthetic route of compound 28

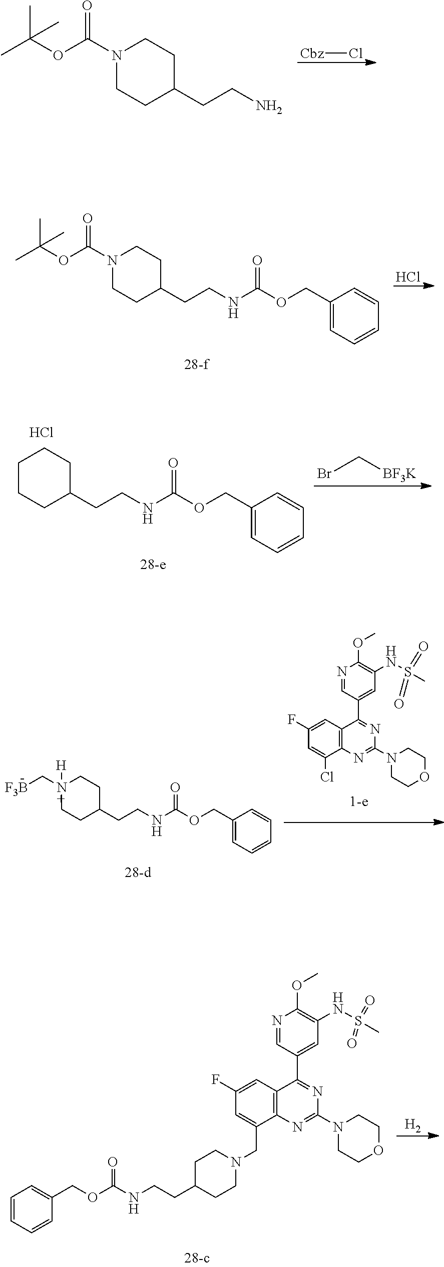

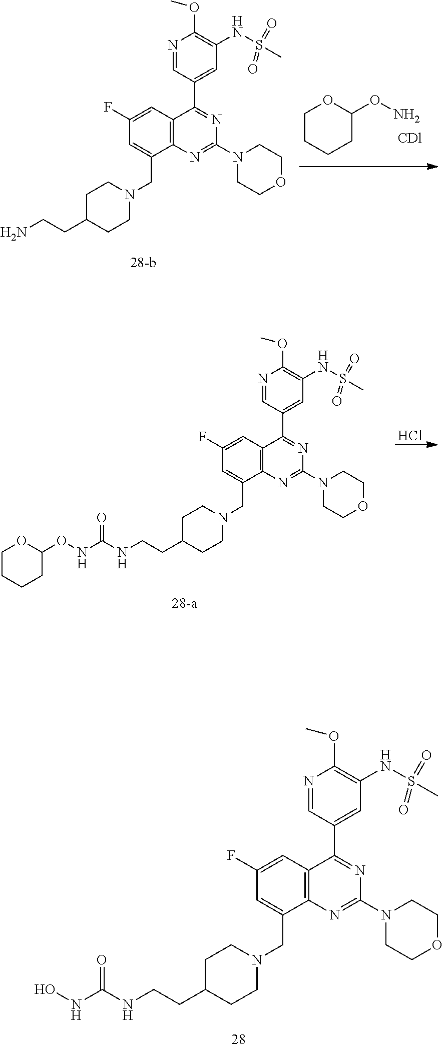

### Synthesis of compound 28-f

Compound N-Boc-4-aminoethyl piperidine (2 g, 8.76 mmol) was dissolved in THF (40 mL) under an ice-water bath, followed by addition of water (5 mL ) and NaHCO₃ (2.2 g, 26.28 mmol), benzyl chloroformate (1.85 mL, 13.14 mmol) was added dropwise to the above mixture and the resulting mixture was brought to room temperature and stirred for 16 hours. The reaction was quenched with water (50 m), extracted with ethyl acetate (100 mL×2), the organic phase was washed with brine (100 mL), dried over anhydrous sodium sulfate, filtered and concentrated at reduced pressure to obtain the crude product. The crude product was purified by flash column (PE/EA=1:1) to obtain the crude compound 28-f (3.81g). LC-MS (ESI): m/z=263.1 [M+1]⁺.

### Synthesis of compound 28-e

Compound 28-f (1.9 g, 80%, 4.19 mmol) was dissolved in DCM (10 mL), followed by addition of a solution of hydrogen chloride in methanol (4 M, 10 mL) in an ice-water bath and the mixture was brought to room temperature and stirred for 2 days. Concentrated at reduced pressure, the crude product was suspended in 1,4-dioxane (50 mL), concentrated at reduced pressure, the crude product was suspended in ethyl acetate (50 mL), the mixture was filtered, the filter cake was washed with ethyl acetate (10 mL), the solid was collected and dried under vacuum for 1 hour to give compound 28-e (1.16 g, 93%). LC-MS (ESI): m/z=263.2 [M+1]⁺.

### Synthesis of compound 28-d

Compound 28-e (1.16 g, 3.88 mmol) was dissolved in acetonitrile(5 mL), followed by serial addition of potassium iodide (37 mg, 0.22 mmol), potassium bromomethylfluoroborate (743 mg, 3.70 mmol), and potassium carbonate (766 mg, 5.55 mmol) at room temperature and the mixture was stirred at 80° C. under nitrogen atmosphere for 16 hours. The mixture was cooled to room temperature, filtered, and the filter cake was washed with acetonitrile (10 mL), then the filtrate was concentrated at reduced pressure to give the crude product, which was dissolved in acetonitrile (5 mL) and ethyl ether (50 mL) was added to precipitate a large amount of solid, filtered, the solid was collected and dried under vacuum to give compound 28-d (1.12 g, 88%). LC-MS (ESI): m/z=362.2 [M+18]⁺.

### Synthesis of compound 28-c

Compound 1-e (763 mg, 1.63 mmol) was dissolved in THF (50 mL) at room temperature, followed by serial addition of water (5 mL), compound 28-d (1.12 g, 3.26 mmol), palladium acetate (37 mg, 0.16 mmol), x-Phos (155 mg, 0.33 mmol), and cesium carbonate (1.59 g, 4.89 mmol), and the mixture was stirred at 80° C. under nitrogen atmosphere for 36 hours. To the reaction system, water (50 mL) was added, extracted with ethyl acetate (100 mL×2), and the organic phase was washed with brine (100 mL×2), dried over anhydrous sodium sulfate, filtered, and concentrated at reduced pressure to obtain the crude product. The crude product was purified by flash chromatography (DCM/MeOH=10:1) to give compound 28-c (1.1 g, 96%). LC-MS (ESI): m/z=708.3 [M+1]⁺.

### Synthesis of compound 28-b

Compound 28-c (1.1 g, 1.55 mmol) was dissolved in methanol (100 ML), followed by addition of palladium carbon (0.5 g, 10% Pd, 50% wet) at room temperature under nitrogen atmosphere, and the mixture was stirred at room temperature under hydrogen atmosphere for 16 hours. The mixture was filtered and washed with mixed solvent (DCM/MeOH(NH₃)=10:1, 500 mL) to give crude compound 28-b (1.0 g), and the crude product was used directly in the next step. LC-MS (ESI): m/z=574.2 [M+1]⁺.

### Synthesis of compound 28-a

Compound 28-b (100 mg, 0.17 mmol) was dissolved in DMF (3 mL), followed by serial addition of CDI (62 mg, 0.38 mmol) and TEA (53 μL, 0.38 mmol) at 0° C. under nitrogen atmosphere. The mixture was stirred at room temperature under nitrogen atmosphere for 30 min, followed by addition of O-(tetrahydro-2H-pyran-2-yl)hydroxylamine (22 mg, 0.19 mmol) in DMF (2 mL), and then stirred at room temperature under nitrogen atmosphere for 16 hours. The mixture was purified by pre-HPLC to give compound 28-a (60 mg, 48%). LC-MS (ESI): m/z=717.3[M+1]⁺.

### Synthesis of compound 28

A solution of hydrogen chloride in 1,4-dioxane (4 M, 0.3 mL) was added dropwise slowly to a mixture of compound 28-a (60 mg, 0.084 mmol) in DCM (5 mL) at 0° C., after addition, the mixture was stirred at 0° C. for 15 min. The reaction was quenched with saturated sodium bicarbonate solution (about 2 mL) at 0° C. (pH=8), then was added the mixed solvent (DCM/MeOH=10:1, 100 mL). The the mixture was dried over anhydrous sodium sulfate, filtered, and the filtrate was concentrated at reduced pressure to give compound 28 (20 mg, 38%). LC-MS (ESI): m/z =633.2[M+1]⁺; ¹H NMR (400 MHz, DMSO-d₆): δ 9.42 (1H, brs), 8.50 (1H, s), 8.38 (1H, d, J=2.0 Hz), 8.20 (1H, s), 7.98 (1H, d, J=2.0 Hz), 7.66 (1H, dd, J₁=9.2 Hz, J₂=2.0 Hz), 7.42 (1H, dd, J₁=8.8 Hz, J₂=2.4 Hz), 6.63 (1H, t, J=5.6 Hz), 4.02 (3H, s), 3.94 (2H, s), 3.89-3.78 (4H, m), 3.78-3.65 (4H, m), 3.09 (3H, s), 3.13-3.00 (2H, m), 2.88 (2H, d, J=10.4 Hz), 2.07 (2H, t, J=10.8 Hz), 1.66 (2H, d, J=11.6 Hz), 1.41-1.31 (2H, m), 1.31-1.09 (3H, m).

### Synthetic route of compound 29

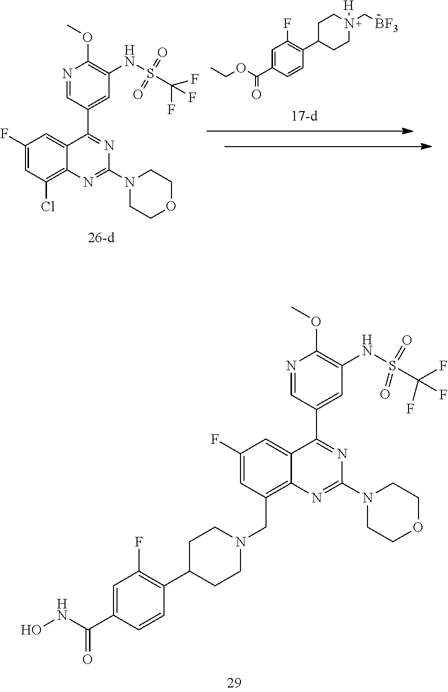

### Synthesis of compound 29

Compound 29 (76 mg) was obtained by replacing compound 4-d with compound 17-d according to synthesis of compound 26. LC-MS (ESI): m/z=738.2 [M+1]⁺; ¹H NMR (400 MHz, DMSO-d₆): δ 7.96 (1H, d, J=1.6 Hz), 7.78 (1H, d, J=1.6 Hz), 7.70 (1H, dd, J₁=2.0 Hz, J₂=9.2 Hz), 7.49 (1H, d, J=7.6 Hz), 7.44 (1H, dd, J=2.4 Hz, J₂=9.2 Hz), 7.36 (1H, d, J=12.0 Hz), 7.24 (1H, t, J=8.0 Hz), 4.11 (1H, brs), 4.00 (2H, s), 3.88-3.79 (7H, m), 3.79-3.65 (4H, m), 3.03 (2H, d, J=10.8 Hz), 2.85-2.71 (1H, m), 2.23 (2H, t, J=11.2 Hz), 2.04-1.90 (1H, m), 1.86-1.64 (4H, m), 1.52-1.30 (1H, m).

### Synthetic route of compound 30

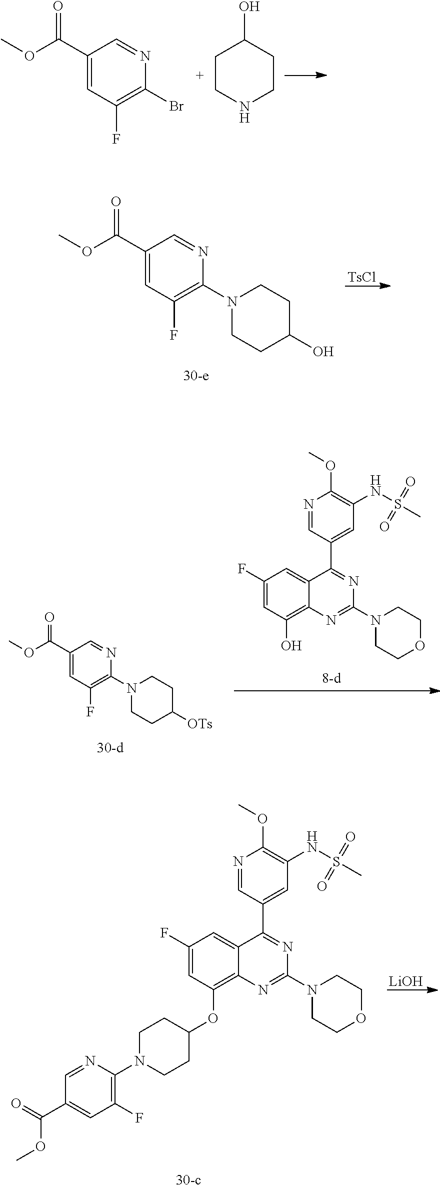

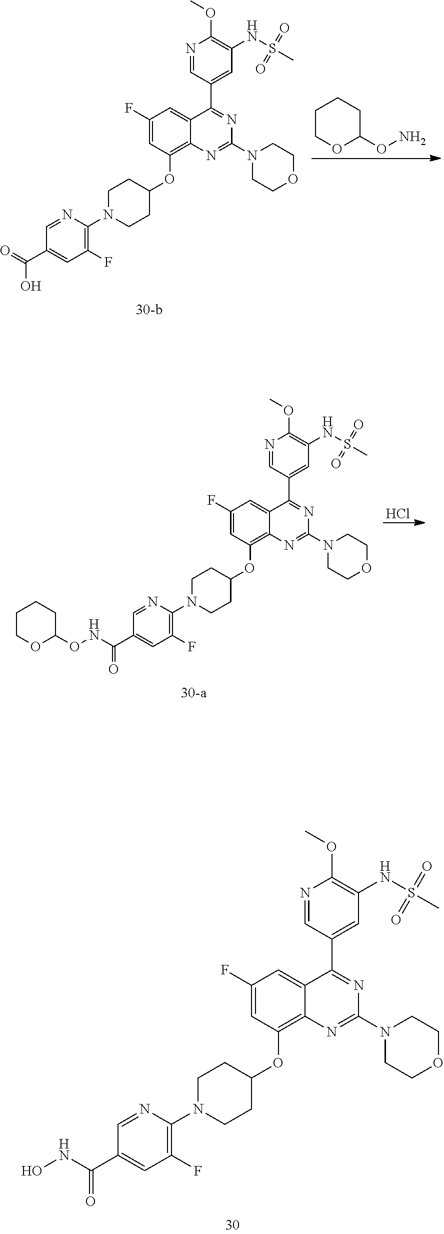

### Synthesis of compound 30-e

Cooled in an ice bath, 6-bromo-5-fluoronicotinic acid methyl ester (234 mg, 1.00 mmol), 4-hydroxypiperidine (101 mg, 1.00 mmol), DMAP (122 mg, 1.00 mmol) and DMF (4 mL) were combined in a reaction vial. The mixture was stirred at 80° C. for 4.5 hours, then ice-water was added to the mixture. The aqueous phase was extracted with ethyl acetate and the organic phases were combined, washed with brine, dried over anhydrous sodium sulfate, filtered, evaporated and purified on a silica column (mobile phase: petroleum ether/ethyl acetate, 100/0 to 100/40) to give compound 30-e (200 mg, 79%). LC-MS (ESI): m/z 255.0 (M+H)⁺.

### Synthesis of compound 30-d

To a reaction flask, 30-e (100 mg, 0.39 mmol), DABCO (88 mg, 0.79 mmol) and dichloromethane (4 mL) were added, followed by addition of TsCl (112 mg, 0.59 mmol) in an ice bath. The mixture was stirred at 0˜25° C. under nitrogen atmosphere for 18 hours. The mixture was evaporated and purified on a silica column (mobile phase: petroleum ether/ethyl acetate, 100/0 to 100/50) to give compound 30-d (70 mg, 44%). 1C-MS (ESI): m/z 409.1 (M+H)⁺.

### Synthesis of compound 30-c

To a reaction vial was added 30-d (70 mg, 0.17 mmol), cesium carbonate (70 mg, 0.21 mmol) and DMF (2.5 mL). The resulting mixture was stirred at 80° C. under nitrogen atmosphere for 2 min. 8-d (64 mg, 0.14 mmol) was added and the mixture was stirred at 80° C. under nitrogen atmosphere for 18 hours. The mixture was cooled to room temperature, diluted with ethyl acetate, washed with water, brine, dried over anhydrous Sodium sulfate, filtered and evaporated to give compound 30-c (150 mg, 100%). LC-MS (ESI): m/z 686.1 (M+H)⁺.

### Synthesis of compound 30-b

To a reaction vial was added 30-c (150 mg, 0.14 mmol), THF (2 mL), methanol (1.5 mL) and water (0.5 mL). The mixture was added with lithium hydroxide (20 mg, 0.83 mmol) at 0° C., and then stirred at 0˜25° C. for 18 hours. The crude product was evaporated to dryness and diluted with ethyl acetate, followed by addition of water, a small amount of citric acid and anhydrous sodium sulfate, the liquid was separated and the aqueous phase was extracted twice with THF, the organic phases were combined and evaporated to dryness to give compound 30-b (120 mg, 100%). LC-MS (ESI): m/z 672.1 (M+H)⁺.

### Synthesis of compound 30-a

To a reaction vial was added 30-b (120 mg, 0.14 mmol), O—(tetrahydro-2H-pyran-2-yl)hydroxylamine (70 mg, 0.61 mmol), DIPEA (70 mg, 0.54 mmol) and DMF (2 mL), followed by addition of HATU (117 mg, 0.33 mmol) in an ice bath. The mixture was stirred at 5˜25° C. for 2 hours. The mixture was diluted with ethyl acetate, washed with water, brine, dried over anhydrous sodium sulfate, filtered, evaporated, purified on a silica column (mobile phase: dichloromethane/methanol 100/0 to 100/5) to give compound 30-a (120 mg, 100%). 1C-MS (ESI): m/z 771.2 (M+H)⁺.

### Synthesis of compound 30

A solution of hydrochloric acid in 1.4-dioxane solution (4M, 0.4 mL) was added to a solution of 30-a (120 mg, 0.14 mmol) in dichloromethane (3 mL) in a reaction vial at 0° C. The mixture was stirred at 0° C. for 1.5 hour. The reaction was quenched with concentrated ammonia (0.2 mL). The mixture was evaporated and purified by pre-HPLC (0.1% aqueous ammonia solution, acetonitrile) to give compound 30 (22 mg, 23%). LC-MS (ESI): m/z 687.0 (M+H)⁺; ¹H NMR (400 MHz, DMSO-d₆): δ 11.16 (1H, s), 9.52 (1H, s), 9.08 (1H, s), 8.41 (1H, t, J=1.7 Hz), 8.39 (1H, d, J=2.3 Hz), 7.99 (1H, d, J=2.3 Hz), 7.76 (1H, dd, J=14.6, 1.9 Hz), 7.44 (1H, dd, J=10.8, 2.6 Hz), 7.16 (1H, dd, J=9.4, 2.6 Hz), 5.04-4.92 (1H, m), 4.03 (3H, s), 3.98-3.87 (2H, m), 3.85-3.81 (4H, m), 3.71-3.67 (4H, m), 3.62-3.50 (2H, m), 3.09 (3H, s), 2.12-2.04 (2H, m), 1.89-1.80 (2H, m).

### Synthetic route of compound 31

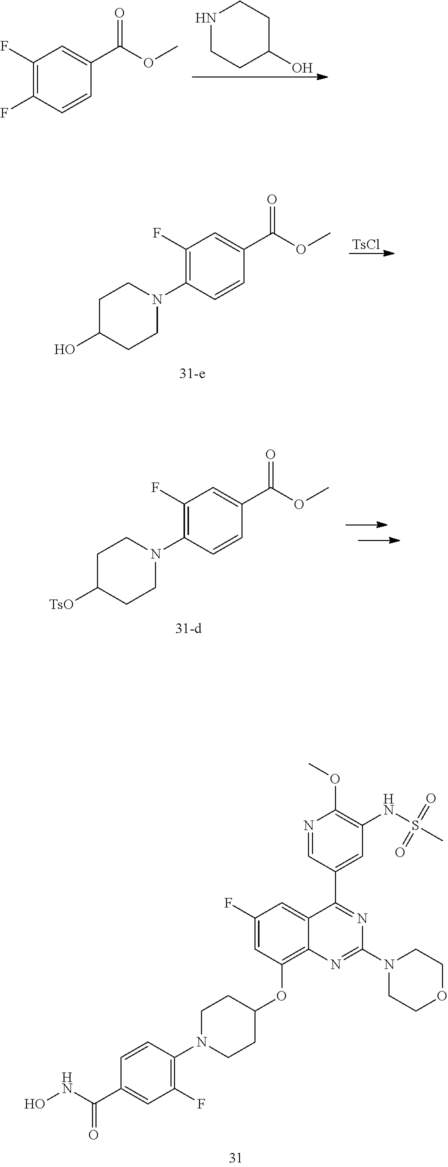

### Synthesis of compound 31-e

Methyl 3,4-difluorobenzoate (200 mg, 1.16 mmol), 4-hydroxypiperidine (117 mg, 1.16 mmol) and DIPEA (0.6 mL) were added in DMSO (5 mL) and the resulting mixture was stirred at 120° C. overnight. The mixture was cooled to room temperature, added saturated ammonium chloride (50 mL), extracted with ethyl acetate (50 mL×3). The organic phase was washed with brine (100 mL×3), dried over anhydrous sodium sulfate, filtered and concentrated at reduced pressure to give compound 31-e (130 mg, 44%).

### Synthesis of compound 31-d

Compound 31-e (113 mg, 0.45 mmol) was added to DCM (5 mL), followed by serial addition of TEA (0.12 mL), DMAP (3 mg, 0.022 mmol) and TsCl (93 mg, 0.49 mmol) at 0° C. and the mixture was stirred at room temperature overnight. The mixture was concentrated at reduced pressure to obtain the crude product. The crude product was purified by flash chromatography (PE/EA=20:1) to give compound 31-d (180 mg, 98%). 1C-MS (ESI):m/z=408.1[M+H]⁺.

### Synthesis of compound 31

Synthesized according to synthesis of compound 30, compound 31-d was used instead of compound 30-d to give compound 31 (13 mg, 72%). LC-MS (ESI):m/z=685.8[M+H]⁺; ¹H NMR (400 MHz, DMSO-d₆): δ 11.14 (1H, s), 9.51 (1H, s), 9.00 (1H, s), 8.39 (1H, d, J=2.0 Hz), 7.99 (1H, d, J=2.0 Hz), 7.66-7.38 (3H, m), 7.21-7.07 (2H, m), 5.03-4.86 (1H, m), 4.02 (3H, s), 3.88-3.78 (4H, m), 3.74-3.61 (4H, m), 3.50-3.39 (2H, m), 3.17-3.04 (5H, m), 2.17-2.04 (2H, m), 1.99-1.84 (2H, m).

### Synthetic route of compound 32

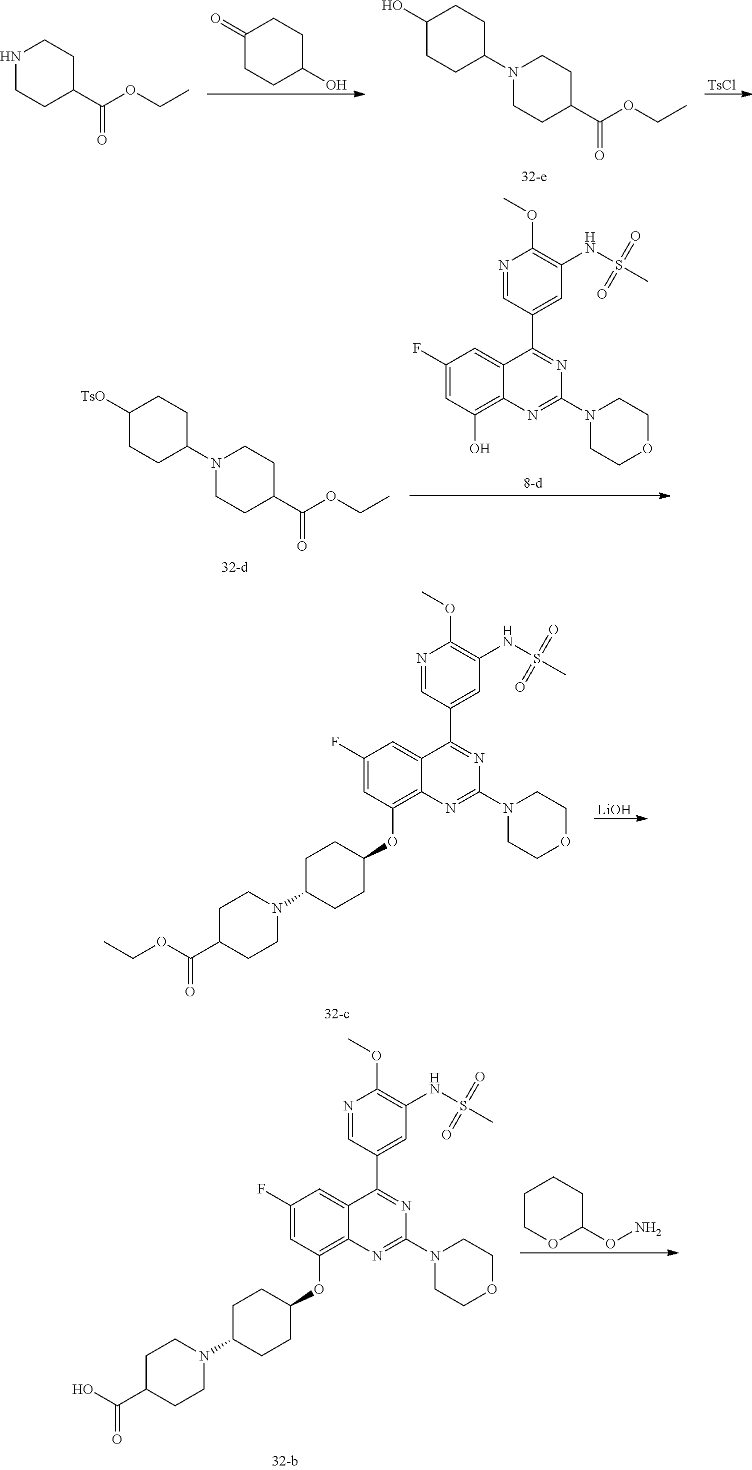

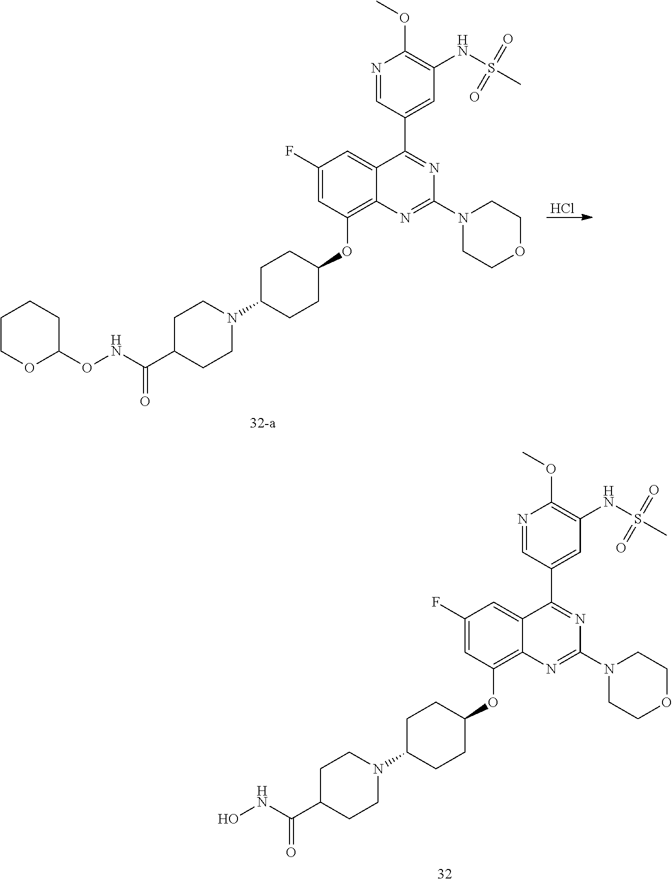 [SMILES: COc1ncc(-c2nc(N3CCOCC3)nc3c(O[C@H]4CC[C@H](N5CCC(C(=O)NOC6CCCCO6)CC5)CC4)cc(F)cc23)cc1NS(C)(=O)=O ; Cl ; COc1ncc(-c2nc(N3CCOCC3)nc3c(O[C@H]4CC[C@H](N5CCC(C(=O)NO)CC5)CC4)cc(F)cc23)cc1NS(C)(=O)=O]

### Synthesis of compound 32-e

Ethyl 4-piperidinecarboxylate (700 mg, 4.45 mmol), 4-hydroxycyclohexanone (1.02 g, 8.94 mmol), acetic acid (100 mg, 1.67 mmol) and dichloromethane (30 mL) were added to a reaction flask while cooled in an ice bath. Sodium triacetoxyborohydride (3.8 g, 17.8 mmol) was added to the above mixture in batches in 2 hours. The mixture was stirred at 0˜25° C. for 18 hours and was added ice-water. The aqueous phase was saturated with sodium bicarbonate and anhydrous sodium sulfate and extracted with dichloromethane. The organic phases were combined, dried over anhydrous Sodium sulfate, filtered and evaporated to give compound 32-e (2.0 g crude, 100%). LC-MS (ESI): m/z 256.1 (M+H)⁺.

### Synthesis of compound 32-d

To a reaction flask, 32-e (2.0 g crude, 4.46 mmol), DABCO (1.5 g, 13.2 mmol) and dichloromethane (20 mL) were added, followed by addition of TsCl (1.65 g, 8.66 mmol) under ice bath condition. The mixture was stirred at 0˜25° C. under nitrogen atmosphere for 18 hours. The mixture was evaporated and purified on a silica column (mobile phase: dichloromethane/methanol, 100/0 to 100/5) to give compound 32-d (900 mg, 48%). LC-MS (ESI): m/z 410.1 (M+H)⁺.

### Synthesis of compound 32-c

To a reaction flask, 32-d (80 mg, 0.20 mmol), cesium carbonate (106 mg, 0.33 mmol) and DMF (2.5 mL) were added. The resulting mixture was stirred at 80° C. under nitrogen atmosphere for 3 min, followed by addition of compound 8-d (74 mg, 0.16 mmol) and the mixture was stirred at 80° C. under nitrogen atmosphere for 18 hours. The mixture was cooled to room temperature, diluted with ethyl acetate, washed with water, brine, dried over anhydrous sodium sulfate, filtered, evaporated and purified on a silica column (mobile phase: dichloromethane/methanol, 100/0 to 100/10) to give compound 32-c (120 mg, 100%). LC-MS (ESI): m/z 687.1 (M+H)⁺.

### Synthesis of compound 32-b

To a reaction vial was added 32-c (120 mg, 0.17 mmol), THF (3 mL), methanol (1.5 mL) and water (0.5 mL). The mixture was added lithium hydroxide (24 mg, 1.0 mmol) at 0° C., and then stirred at 0 to 25° C. for 23 hours. Evaporated to dryness, the crude product was added a small amount of citric acid, extracted with ethyl acetate once and DMF (1.5 mL×3). The organic phases were combined and evaporated to dryness to obtain DMF solution of compound 32-b (4.5 mL). LC-MS (ESI): m/z 659.2 (M+H)⁺.

### Synthesis of compound 32-a

To a reaction vial was added 32-b (4.5 mL DMF solution), O—(tetrahydro-2H-pyran-2-yl)hydroxylamine (80 mg, 0.68 mmol) and DIPEA (100 mg, 0.78 mmol), followed by addition of HATU (160 mg, 0.42 mmol) under ice bath cooling. The mixture was stirred at 5˜25° C. for 2 hours.

The mixture was diluted with ethyl acetate, washed with water, brine, dried over anhydrous sodium sulfate, filtered and evaporated, purified on a silica column (mobile phase: dichloromethane/methanol/5% ammonia 100/0 to 100/10) to give compound 32-a (60 mg, 45%). LC-MS (ESI): m/z 758.0 (M+H)⁺.

### Synthesis of compound 32

To a reaction flask, 32-a (60 mg, 0.052 mmol) and dichloromethane (4 mL) were added, followed by addition of 4M hydrochloric acid/1,4-dioxane solution (0.4 mL) under ice bath cooling. The mixture was stirred at 0° C. for 1.5 hours. The reaction was quenched with concentrated ammonia (0.3 mL). The mixture was evaporated and purified by pre-HPLC (0.1% formic acid aqueous solution, acetonitrile) to give compound 32 (10 mg, 18%). LC-MS (ESI): m/z 674.0 (M+H)⁺; ¹H NMR (400 MHz, DMSO-d₆): δ 10.36 (1H, s), 8.64 (1H, bs), 8.31 (1H, s), 8.27 (1H, d, J=2.0 Hz), 7.92 (1H, d, J=2.4 Hz), 7.29 (1H, dd, J=10.9, 2.7 Hz), 7.11 (1H, dd, J=9.4, 2.6 Hz), 4.57-4.44 (1H, m), 4.00 (3H, s), 3.83 (4H, t, J=4.7 Hz), 3.71 (4H, t, J=4.8 Hz), 3.02 (3H, s), 2.90-2.82 (2H, m), 2.44-2.31 (2H, m), 2.20-2.09 (4H, m), 1.98-1.89 (1H, m), 1.86-1.77 (2H, m), 1.60-1.39 (8H, m).

### Synthetic route of compound 33

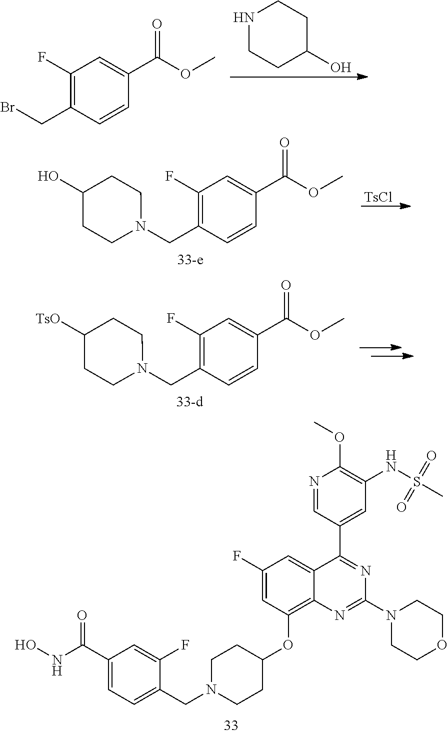

### Synthesis of compound 33-e

Methyl 3-fluoro-4-(bromomethyl)benzoate (733 mg, 2.97 m(mol), 4-hydroxypiperidine (300 mg, 2.97 mmol) and K₂CM₃ (1.23 g, 8.91 mmol) were added in DMF (10 mm) and the mixture was stirred at room temperature overnight. The mixture was added saturated sodium chloride (80 mL), extracted with ethyl acetate (50 m×3), and the organic phase was washed with brine (100 mL×6), dried over anhydrous sodium sulfate, filtered and concentrated at reduced pressure to give compound 33-e (500 mg, 639)).

### Synthesis of compound 33-d

Compound 33-e (200 mg, 0.75 mmol) was added in DCM (5 ML), followed by serial addition of TEA (0.21 mL), DMAP (5 mg, 0.037 mmol) and TsCl (156 mg, 0.82 mmol) at 0° C. and the mixture was stirred at room temperature overnight. The mixture was concentrated at reduced pressure to obtain the crude product. The crude product was purified by flash chromatography (PE/EA=20:1) to give compound 33-d (188 mg, 59%).

### Synthesis of compound 33

Synthesized according to synthesis of compound 30, compound 33-d was used instead of compound 30-d to give compound 33 (5 mg). LC-MS (ESI):m/z=699.8[M+H]⁺.

### Synthetic route of compound 34

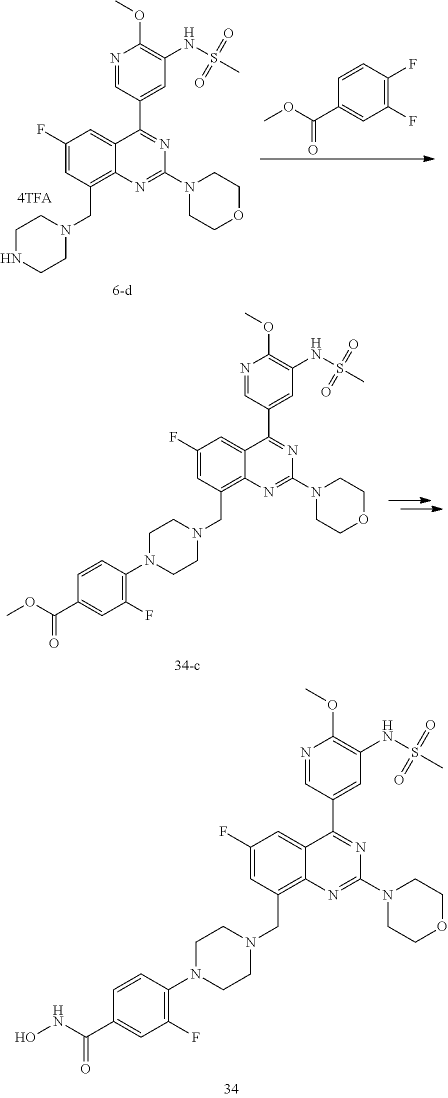

### Synthesis of compound 34-c

Compound 6-d (164 mg, 0.17 mmol) was dissolved in DMF(5 mL), followed by addition of methyl 3,4-difluorobenzoate (162 mg, 0.94 mmol) and potassium carbonate (80 mg, 0.58 mmol) at room temperature, then the mixture was stirred at 100° C. under nitrogen atmosphere for 16 hours. The mixture was cooled to room temperature, added brine (50 mL), extracted with ethyl acetate (50 mL×3), and the organic phase was dried over anhydrous sodium sulfate, filtered and concentrated to obtain the crude compound, which was purified by flash chromatography (DCM/MeOH=10:1) to give the crude compound 34-c (116 mg, 100%). 1C-MS (ESI): m/z=683.8 [M+1]⁺.

### Synthesis of compound 34

Synthesized according to synthesis of compound 1, compound 34-c was used instead of compound 1-c to obtain compound 34 (5 mg). 1C-MS (ESI): m/z=684.9 [M+H]⁺.

### Synthetic route of compound 35

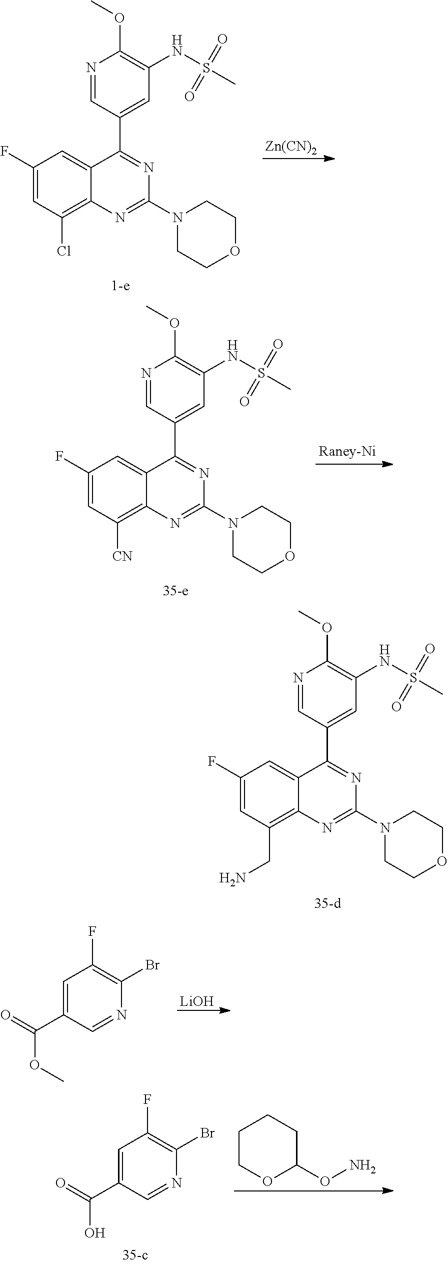

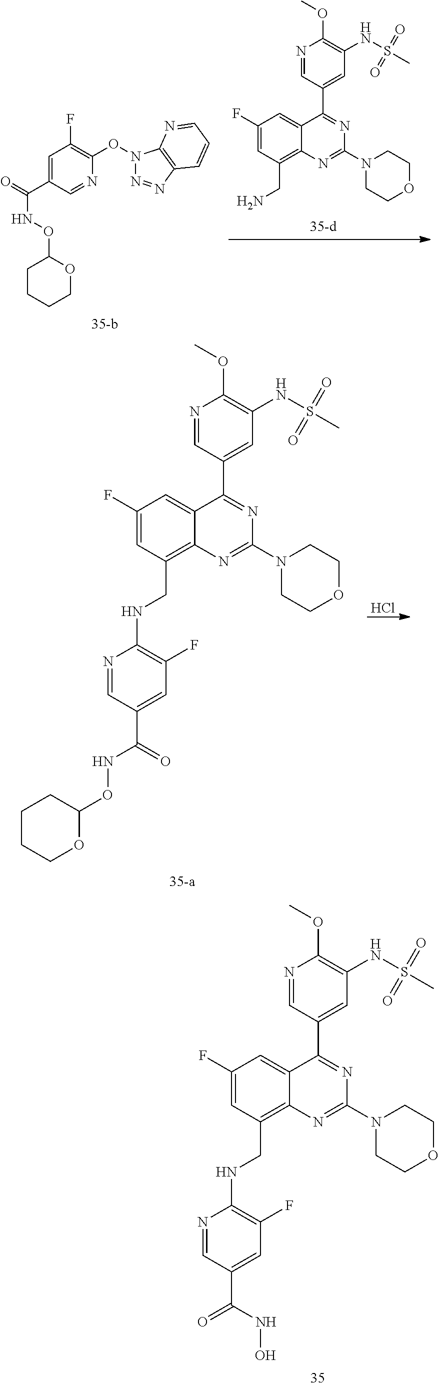

### Synthesis of compound 35-e

Compound 1-e (2.0 g, 4.27 mmol) was dissolved in DMF(30 mL), followed by serial addition of zinc cyanide (999 mg, 8.54 mmol), X-Phos (407 mg, 0.85 mmol), Pd₂(dba)₃ (391 mg, 0.43 mmol), and zinc powder (200 mg, 3.08 mmol). The mixture was stirred at 100° C. under nitrogen atmosphere for 18 hours. The mixture was cooled to room temperature, quenched with water(100 mL), washed with ethyl acetate (100 mL×2), the organic phase was washed with brine (100 mL×3), dried over anhydrous sodium sulfate, filtered and concentrated at reduced pressure to give the crude product, which was purified by flash chromatography (EA/PE=0˜80%) to give compound 35-e (1.2 g, 61%). LC-MS (ESI): m/z=459.1 [M+1]⁺.

### Synthesis of compound 35-d

To a reaction flask was added 35-e (140 mg, 0.31 mmol), Raney nickel (200 mg) and tetrahydrofuran (20 mL). The mixture was stirred at 25° C. under hydrogen atmosphere for 40 hours. Filtered, and the filtrate was evaporated and purified on a silica column (mobile phase: dichloromethane/methanol/ammonia, 100/0/0 to 100/10/5) to give compound 35-d (62 mg, 44%). LC-MS (ESI): m/z 463.1 (M+H)⁺.

### Synthesis of compound 35-c

To a reaction flask, 6-bromo-5-fluoronicotinic acid methyl ester (350 mg, 1.50 mmol), THE (4 mL), methanol (3.2 mL) and water (0.8 mL) were added. The mixture was added lithium hydroxide (72 mg, 3.0 mmol) at 0° C., and then stirred at 0° C. for 2 hours. The reaction was quenched by a small amount of citric acid and was evaporated to dryness, and the crude product was added water and extracted twice with ethyl acetate. The organic phases were combined, washed with brine, dried over anhydrous sodium sulfate, filtered and evaporated to give compound 35-c (320 mg, 97%). LC-MS (ESI): m/z 220.0(M+H)⁺.

### Synthesis of compound 35-b

Cooled in an ice bath, 35-c (320 mg, 1.45 mmol), O—(tetrahydro-2H-pyran-2-yl)hydroxylamine (185 mg, 1.60 mmol), DMF (4 mL), HATU (1.10 g, 2.90 mmol) and DIPEA (561 mg, 4.35 mmol) were added in a reaction flask. The mixture was stirred at 0 ˜ 25° C. for 2 hours. A small amount of citric acid was added, diluted with ethyl acetate, washed with water, brine, dried over anhydrous sodium sulfate, filtered, evaporated and purified on a silica column (mobile phase: petroleum ether/ethyl acetate 100/0 to 100/80) to give compound 35-b (350 mg, 64%). LC-MS (ESI): m/z 375.1 (M+H)⁺.

### Synthesis of compound 35-a

To a reaction flask, compound 35-d (58 mg, 0.13 mmol), compound 35-b (94 mg, 0.25 mmol), DIPEA (65 mg, 0.50 mmol) and DMF (2.5 mL) were added. The mixture was stirred at 80° C. under nitrogen atmosphere for 6 hours. The mixture was cooled to room temperature, diluted with ethyl acetate, washed with water, saturated sodium chloride water, dried over anhydrous sodium sulfate, filtered, evaporated and purified by pre-HPLC (mobile phase: 0.1% formic acid/water, acetonitrile) to give compound 35-a (30 mg, 34%). LC-MS (ESI): m/z 701.1 (M+H)⁺.

### Synthesis of compound 35

To a reaction flask, compound 35-a (30 mg, 0.043 mmol) and dichloromethane (3 mL) were added, followed by addition of 4 M hydrochloric acid/1,4-dioxane solution (0.3 mL) in an ice bath. The mixture was stirred at 0° C. for 40 min. The reaction was quenched with concentrated ammonia (0.2 mL). The mixture was evaporated to dryness, purified by pre-HPLC (0.1% aqueous ammonia solution, acetonitrile) to give compound 35 (8 mg, 30%). LC-MS (ESI): m/z 617.0 (M+H) ⁺; ¹H NMR (400 MHz, DMSO-d₆): δ 11.02 (1H, s), 9.51 (1H, bs), 8.98 (1H, s), 8.42 (1H, d, J=2.2 Hz), 8.21 (1H, d, J=1.5 Hz), 8.01 (1H, d, J=2.2 Hz), 7.78 (1H, t, J=6.5 Hz), 7.68 (1H, dd, J=12.2, 1.8 Hz), 7.51-7.38 (2H, m), 5.06 (2H, d, J=5.9 Hz), 4.03 (3H, s), 3.93-3.82 (4H, m), 3.71 (4H, t, J=4.7 Hz), 3.10 (3H, s).

### Synthetic route of compound 36

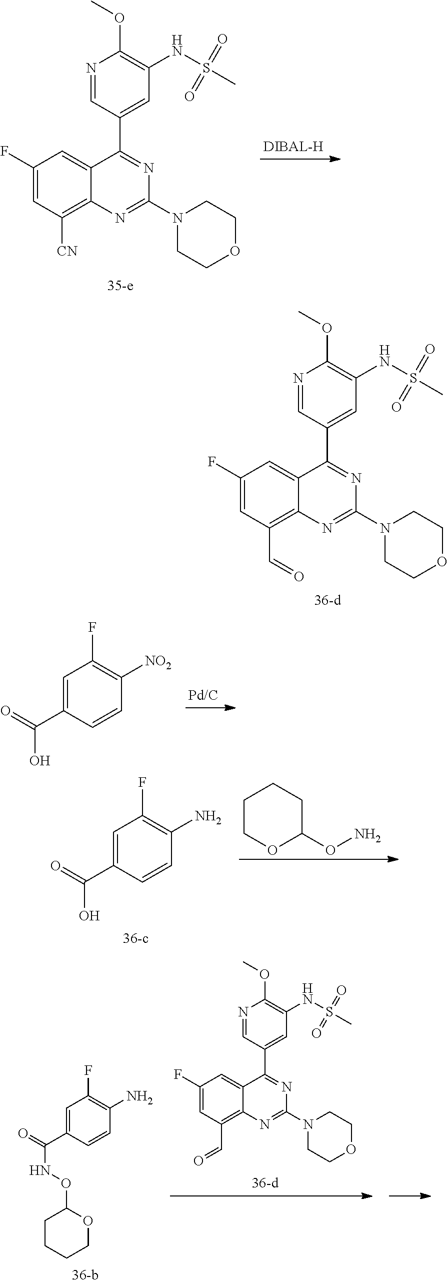

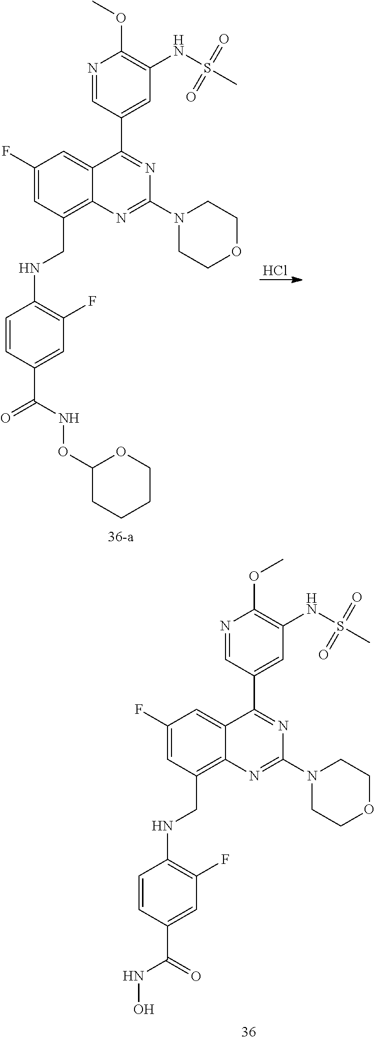 [SMILES: COc1ncc(-c2nc(N3CCOCC3)nc3c(CNc4ccc(C(=O)NOC5CCCCO5)cc4F)cc(F)cc23)cc1NS(C)(=O)=O ; Cl ; COc1ncc(-c2nc(N3CCOCC3)nc3c(CNc4ccc(C(=O)NO)cc4F)cc(F)cc23)cc1NS(C)(=O)=O]

### Synthesis of compound 36-d

Compound 35-e (160 mg, 0.35 mmol) was dissolved in DCM(15 mL), cooled at −78° C., DIBAL-H (1.05 mL, 1.05 mmol, 1 M toluene solution) was added to the above solution. The mixture was brought to room temperature and stirred for 2.5 hours, then cooled to 0° C., quenched with 50% sodium potassium tartrate solution (30 mL), added ethyl acetate(100 mL) and stirred for 1.5 hours at room temperature. The organic phase was separated, and the aqueous phase was extracted with ethyl acetate(100 mL). The organic phases were combined and washed with brine (100 mL×2), dried over anhydrous sodium sulfate, filtered and concentrated at reduced pressure to give the crude product, which was purified by flash column (EA/PE=0100o %) to give compound 36-d (41 mg, 25%). LC-MS (ESI): m/z=462.1 [M+1]⁺.

### Synthesis of compound 36-c

To a reaction flask was added 3-fluoro-4-nitrobenzoic acid (1.0 g, 5.4 mmol), palladium carbon (200 mg), methanol (5 mL) and tetrahydrofuran (5 mL). The mixture was stirred at 25° C. under hydrogen atmosphere for 18 hours. Filtered and evaporated to obtain compound 36-c (750 mg, 89%). LC-MS (ESI): m/z 156.0 (M+H)⁺.

### Synthesis of compound 36-b

Cooled in an ice bath, 36-c(200 mg, 1.29 mmol), O—(tetrahydro-2H-pyran-2-yl)hydroxylamine (453 mg, 3.87 mmol), DMF (4 mL), DIEA (500 mg, 3.87 mmol) and HATU (640 mg, 1.68 mmol) were added to a reaction flask. The mixture was stirred at 0° C. for 1 hour then diluted with ethyl acetate, washed with water, brine, dried over anhydrous sodium sulfate, filtered, evaporated and purified on a silica column (mobile phase: petroleum ether/ethyl acetate 100/0 to 100/80) to obtain the crude product, dissolved in ethyl acetate, washed with aqueous citric acid (3%) three times and evaporated to obtain compound 36-b (350 mg, 100%). LC-MS (ESI): m/z 255.1 (M+H)⁺.

### Synthesis of compound 36-a

36-d (60 mg, 0.13 mmol), 36-b (66 mg, 0.26 mmol) and methanol (3 mL) were mixed in a reaction flask. The mixture was stirred at room temperature for 2 hours, followed by addition of 2-methylpyridine borane complex (21 mg, 0.195 mmol) and the mixture was stirred at 25° C. under nitrogen atmosphere for 18 hours. Acetone (0.2 mL) was added and stirred at room temperature for 10 min, evaporated and purified on a reversed phase column (mobile phase: 0.1% FA/water, acetonitrile) to give compound 36-a (40 mg, 44%). LC-MS (ESI): m/z 700.2 (M+H)⁺.

### Synthesis of compound 36

To a reaction flask, 36-a (40 mg, 0.057 mmol) and dichloromethane (4 mL) were added, followed by 4M hydrochloric acid/1,4-dioxane solution (0.4 mL) was added in an ice bath. The mixture was stirred at 0° C. for 40 min. The reaction was quenched with concentrated ammonia (0.3 mL). The mixture was evaporated to dryness and purified by pre-HPLC (0.1% aqueous ammonia solution, acetonitrile) to give compound 36 (5 mg, 14%). LC-MS (ESI): m/z 616.0 (M+H)⁺; ¹H NMR (400 MHz, DMSO-d₆): δ 10.89 (1H, s), 9.50 (1H, bs), 8.83 (1H, s), 8.42 (1H, d, J=2.0 Hz), 8.02 (1H, d, J=2.3 Hz), 7.53 (1H, dd, J=9.4, 2.9 Hz), 7.50-7.42 (2H, m), 7.36 (1H, dd, J=8.5, 2.0 Hz), 6.77 (1H, s), 6.63 (1H, t, J=8.7 Hz), 4.85 (2H, d, J=6.2 Hz), 4.03 (3H, s), 3.90 (4H, t, J=4.9 Hz), 3.74 (4H, t, J=4.9 Hz), 3.10 (3H, s).

### Synthetic route of compound 37

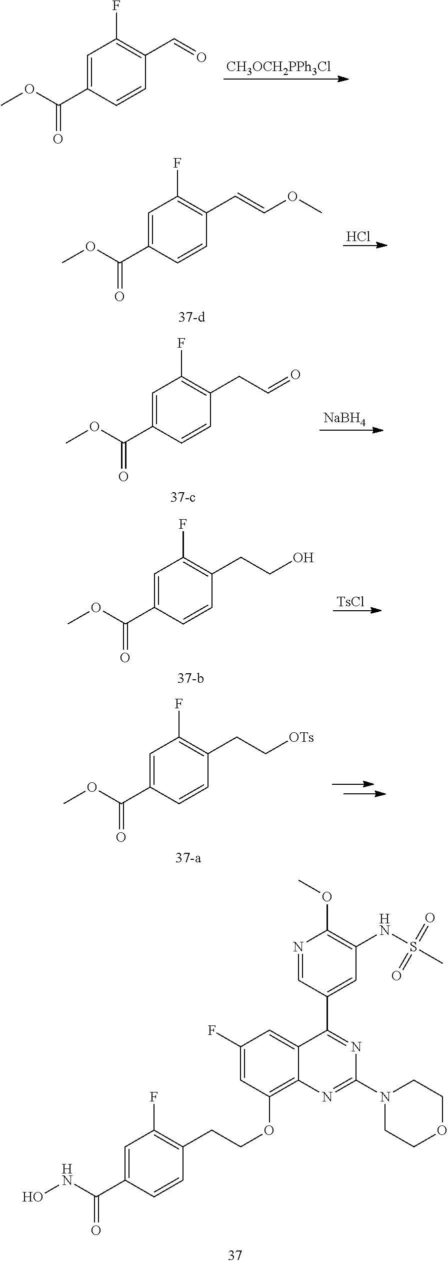

### Synthesis of compound 37-d

Cooled in an ice bath, methoxymethyltriphenylphosphonium chloride (989 mg, 2.88 mmol) and tetrahydrofuran (10 mL) were mixed in a reaction flask, then added potassium tert-butoxide (308 mg, 2.75 mmol). The mixture was stirred at 0° C. for 5 min, and then was added methyl 3-fluoro-4-formylbenzoate (250 mg, 1.37 mmol). The mixture was stirred at 0° C. for 4 hours, was removed solvent and the residue was washed with an ethyl acetate/petroleum ether and the washings were evaporated and purified on a silica column (mobile phase: petroleum ether/ethyl acetate, 100/0 to 100/15) to give compound 37-d (200 mg, 69%).

### Synthesis of compound 37-c

To a reaction flask was added 37-d (200 mg, 0.95 mmol), THF (5 mL) and aqueous hydrochloric acid (6M, 0.5 mL). The mixture was stirred at 60° C. for 4 hours. The solvent was spun off and the residue was diluted with ethyl acetate, washed with water, brine, dried over anhydrous sodium sulfate, filtered, evaporated and purified on a silica column (mobile phase: petroleum ether/ethyl acetate, 100/0 to 100/30) to give compound 37-c (160 mg, 85%).

### Synthesis of compound 37-b

Cooled in an ice bath, 37-c (160 mg, 0.82 mmol) and methanol (5 mL) were added to a reaction flask. sodium borohydride (61 mg, 1.62 mmol) was added in batches in 10 min. The mixture was stirred at 0° C. for 2 hours. The mixture was added acetone (0.5 mL) and continued stirring at 0° C. for 0.5 hour. The mixture was evaporated and purified on a silica column (mobile phase: petroleum ether/ethyl acetate, 100/0 to 100/60) to give compound 37-b (140 mg, 87%).

### Synthesis of compound 37-a

37-b (140 mg, 0.71 mmol), DABCO (246 mg, 2.12 mmol) and dichloromethane (5 mL) were added in a reaction flask, followed by addition of TsCl (200 mg, 1.06 mmol) under ice bath cooling. The mixture was stirred at 0° C. under nitrogen atmosphere for 1 hour. The mixture was washed with water, brine, dried over anhydrous sodium sulfate, filtered, evaporated and purified on a silica column (mobile phase: petroleum ether/ethyl acetate, 100/0 to 100/20) to give compound 37-a (225 mg, 90%).

### Synthesis of compound 37

Synthesized according to synthesis of compound 8, compound 37-a was used in place of compound 6-bromo-hexanoic acid methyl ester to give compound 37 (11.3 mg, 28%). LC-MS (ESI): m/z 631.0 (M+H)*; ¹H NMR (400 MHz, DMSO-d₆): δ 11.30 (1H, s), 9.49 (1H, bs), 9.14 (1H, s), 8.32 (1H, s), 7.94 (1H, d, J=2.2 Hz), 7.67 (1H, t, J=7.8 Hz), 7.59-7.50 (2H, m), 7.25 (1H, dd, J=10.9, 2.6 Hz), 7.08 (1H, dd, J=9.5, 2.5 Hz), 4.38 (2H, t, J=6.2 Hz), 4.01 (3H, s), 3.87-3.81 (4H, m), 3.72 (4H, t, J=4.7 Hz), 3.23 (2H, t, J=6.2 Hz), 3.05 (3H, s).

### Synthetic route of compound 38

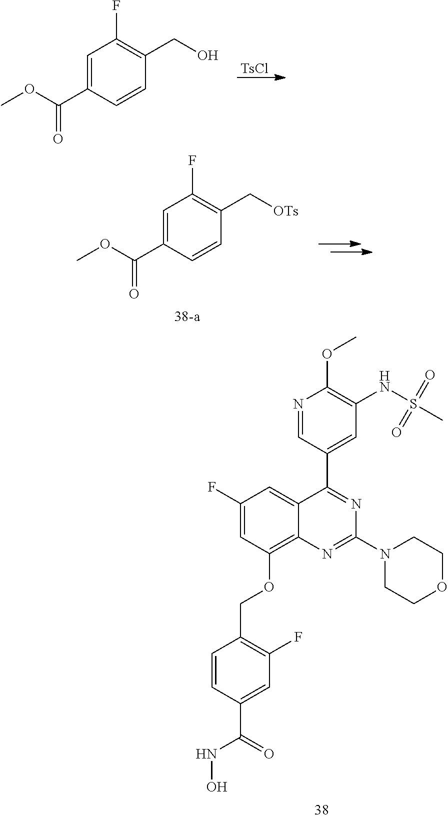

### Synthesis of compound 38-a

The compound methyl 3-fluoro-4-hydroxymethylbenzoate (300 mg, 1.63 mmol) was added into DCM (5 mL), followed by addition of TEA (0.45 mL), DMAP (10 mg, 0.082 mmol), TsCl (341 mg, 1.79 mmol) at 0° C. and the mixture was stirred at room temperature overnight. The mixture was concentrated at reduced pressure to obtain the crude product. The crude product was purified by flash chromatography (PE/EA=20:1) to give compound 38-a (150 mg, 27%).

### Synthesis of compound 38

Synthesized according to synthesis of compound 8, compound 38-a was used instead of compound 6-bromo-hexanoic acid methyl ester to obtain compound 38 (2 mg). LC-MS (ESI):m/z=617.0[M+H]⁺.

### Synthesis of compound 39

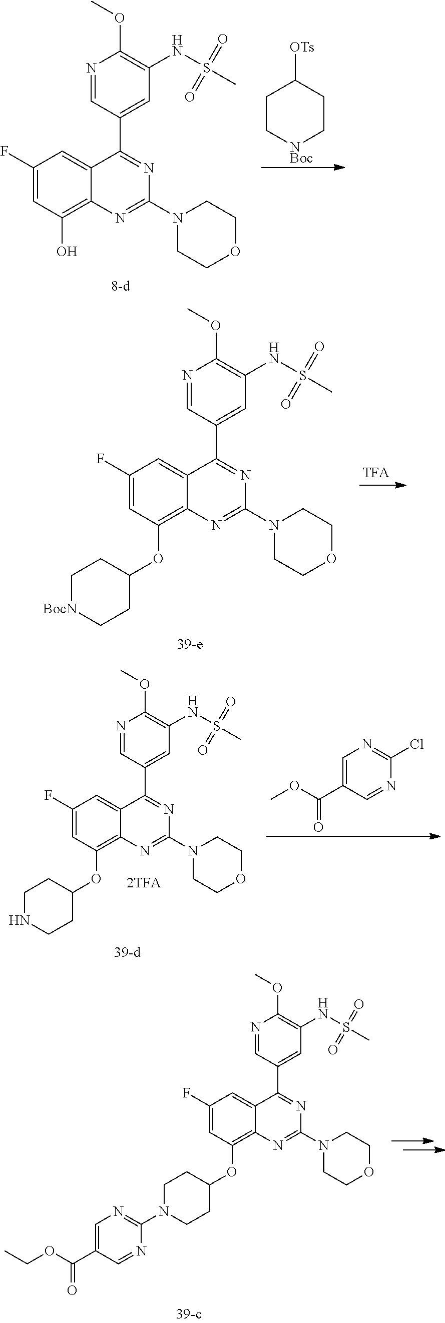

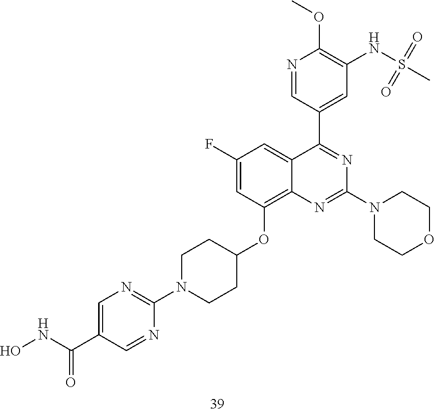 [SMILES: COc1ncc(-c2nc(N3CCOCC3)nc3c(OC4CCN(c5ncc(C(=O)NO)cn5)CC4)cc(F)cc23)cc1NS(C)(=O)=O]

### Synthesis of compound 39-e

1-tert-butoxycarbonyl-4-(p-tosyloxy)piperidine (500 mg, 1.41 mmol), cesium carbonate (1.02 g, 3.14 mmol) and DMF (8 mL) were placed in a reaction flask. The mixture was stirred at 80° C. under nitrogen atmosphere for 5 min, followed by addition of compound 8-d (500 mg, 1.11 mmol) and the mixture was stirred at 80° C. under nitrogen atmosphere for 4 hours. The mixture was cooled to room temperature, diluted with ethyl acetate, washed with water, brine, dried over anhydrous sodium sulfate, filtered, evaporated and purified on a silica column (mobile phases: petroleum ether/ethyl acetate, dichloromethane/methanol, 100/0 to 100/100, 100/4) to give compound 39-e (550 mg, 78%). LC-MS (ESI): m/z 633.2(M+H)⁺.

### Synthesis of compound 39-d

Cooled in an ice bath, 39-e (550 mg, 0.87 mmol), dichloromethane (15 mL) and trifluoroacetic acid (1 mL) were added to a reaction flask. The mixture was stirred at 0° C. for 3 hours. The mixture was evaporated and the residue was washed with methyl tert-butyl ether and dried under vacuum to give compound 39-d (600 mg, 95%). LC-MS (ESI): m/z 533.2 (M+H)⁺.

### Synthesis of compound 39-c

Cooled in an ice bath, 39-d (120 mg, 0.17 mmol), ethyl 2-chloropyrimidine-5-carboxylate (44 mg, 0.24 mmol), potassium carbonate (109 mg, 0.79 mmol) and acetonitrile (5 mL) were placed in a reaction flask. The mixture was stirred at 70° C. for 6 hours and evaporated to dryness, and the residue was added ice-water. The aqueous phase was extracted with ethyl acetate. The organic phases were combined, washed with brine, dried over anhydrous sodium sulfate, filtered and evaporated to give compound 39-c (150 mg, 100%). LC-MS (ESI): m/z 683.2 (M+H)⁺.

### Synthesis of compound 39

Synthesized according to synthesis of compound 30, compound 39-c was used instead of compound 30-c to obtain compound 39 (24 mg). LC-MS (ESI): m/z 670.0 (M+H)⁺; ¹H NMR (400 MHz, DMSO-d₆): δ 11.10 (1H, s), 9.53 (1H, s), 9.04 (1H, s), 8.69 (2H, s), 8.39 (1H, d, J=2.1 Hz), 7.99 (1H, d, J=2.3 Hz), 7.45 (1H, dd, J=10.8, 2.5 Hz), 7.16 (1H, dd, J=9.5, 2.6 Hz), 5.01 (1H, s), 4.21-4.08 (2H, m), 4.03 (3H, s), 3.93-3.77 (6H, m), 3.72-3.67 (4H, m), 3.10 (3H, s), 2.10-1.94 (2H, m), 1.88-1.73 (2H, m).

### Synthetic route of compound 40

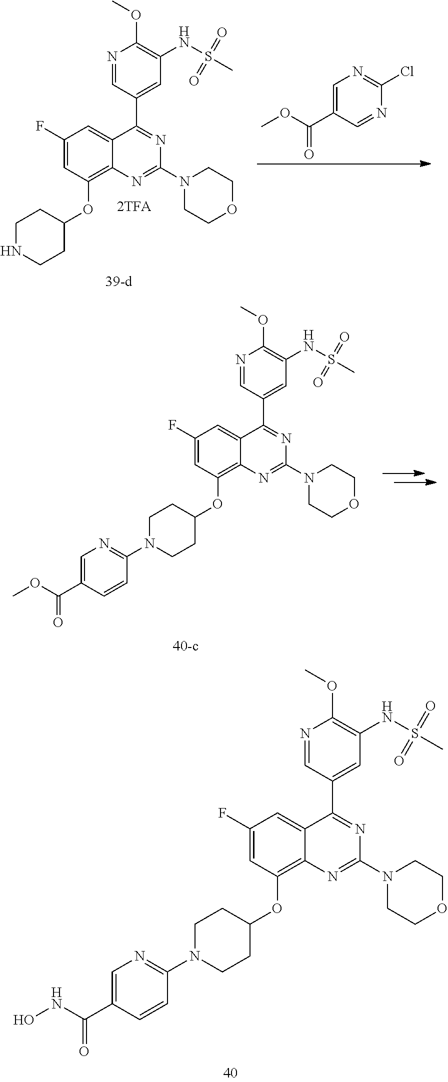

### Synthesis of compound 40-c

Under ice bath cooling, 39-d (120 mg, 0.17 mmol), methyl 6-chloronicotinate (44 mg, 0.26 mmol), potassium carbonate (109 mg, 0.79 mmol) and acetonitrile (2 mL) were added to a reaction flask. The mixture was stirred at 80° C. for 22 hours, and then DMF (2 mL) were added to it. The mixture was stirred at 80° C. for 20 hours. The mixture was evaporated to dryness and then ice-water was added to the residue. The aqueous phase was extracted with ethyl acetate. The organic phases were combined, washed with brine, dried over anhydrous sodium sulfate, filtered and evaporated to give compound 40-c (150 mg, 100%). LC-MS (ESI): m/z 668.3 (M+H)⁺.

### Synthesis of compound 40

Synthesized according to synthesis of compound 30, compound 40-c was used instead of compound 30-c to obtain compound 40 (28 mg). LC-MS (ESI): m/z 669.0 (M+H)⁺; ¹H NMR (400 MHz, DMSO-d₆): δ 11.00 (1H, s), 9.51 (1H, bs), 8.90 (1H, s), 8.52 (1H, d, J=2.4 Hz), 8.39 (1H, d, J=2.2 Hz), 7.99 (1H, d, J=2.2 Hz), 7.87 (1H, dd, J=9.0, 2.5 Hz), 7.44 (1H, dd, J=10.8, 2.7 Hz), 7.15 (1H, dd, J=9.5, 2.7 Hz), 6.92 (1H, d, J=9.0 Hz), 4.98 (1H, dt, J=7.5, 3.8 Hz), 4.03 (3H, s), 3.98 (2H, td, J=8.2, 7.3, 3.6 Hz), 3.82 (4H, t, J=4.7 Hz), 3.67 (4H, t, J=4.8 Hz), 3.62 (2H, td, J=7.6, 6.9, 3.6 Hz), 3.10 (3H, s), 2.07-1.93 (2H, m), 1.86-1.72 (2H, m).

### Synthetic route of compound 41

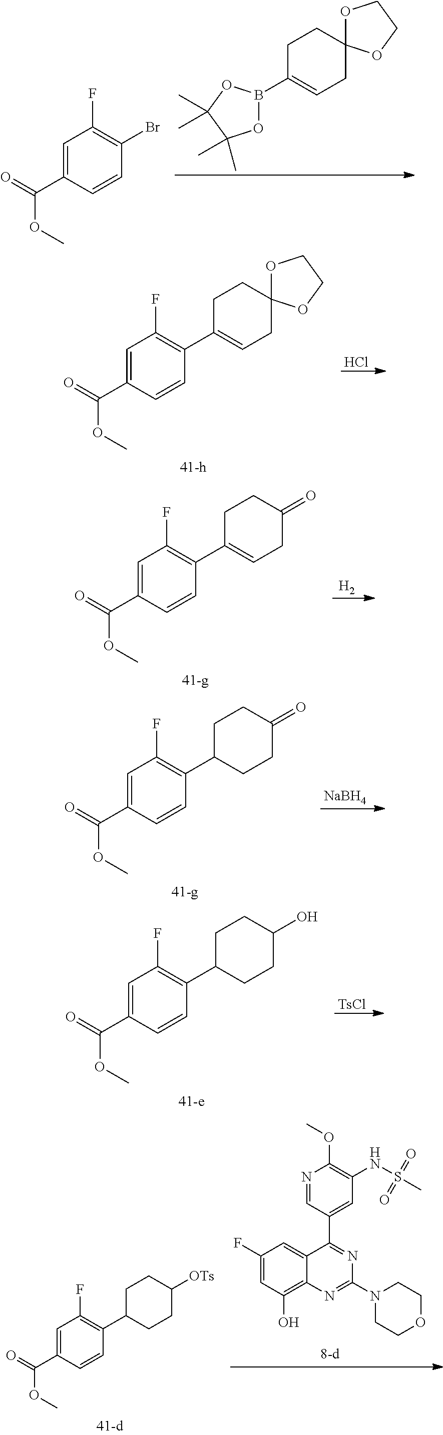

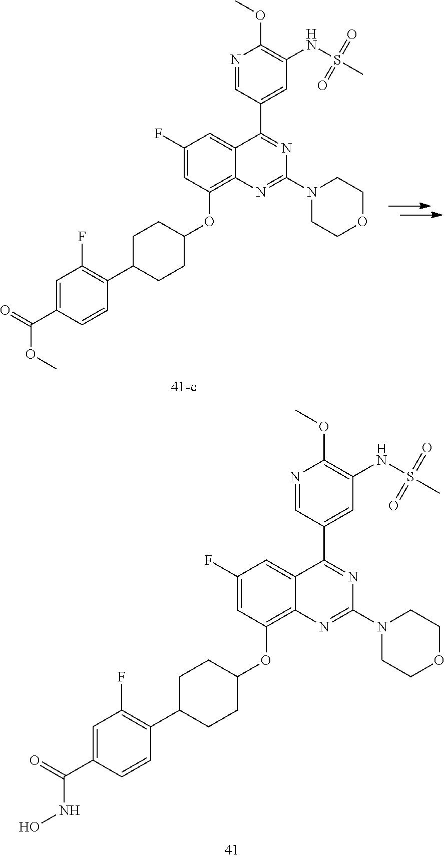 [SMILES: COC(=O)c1ccc(C2CCC(Oc3cc(F)cc4c(-c5cnc(OC)c(NS(C)(=O)=O)c5)nc(N5CCOCC5)nc34)CC2)c(F)c1 ; COc1ncc(-c2nc(N3CCOCC3)nc3c(OC4CCC(c5ccc(C(=O)NO)cc5F)CC4)cc(F)cc23)cc1NS(C)(=O)=O]

### Synthesis of compound 41-h

Compound methyl 4-bromo-3-fluorobenzoate (3.00 g, 12.87 mmol), 1,4-dioxa-spiro[4,5]dec-7-ene-8-boronic acid pinacol ester (3.77 g, 14.16 mmol), Pd(PPh₃)₄(1.49 g, 1.29 mmol) and Cs₂CO₃ (8.39 g, 25.74 mmol) were added to a mixed solvent of 1,4-dioxane (60 mL) and water (12 mL), and the resulting mixture was stirred at 100° C. under nitrogen atmosphere overnight. Cooled to room temperature, the mixture was added brine (50 mL), extracted with ethyl acetate (50 mL×3), the organic phase was washed with brine (100 mL×3), dried over anhydrous sodium sulfate, filtered, and concentrated at reduced pressure to obtain the crude product. The crude product was purified by flash chromatography (PE/EA=10:1) to give compound 41-h (3.74 g, 99%).

### Synthesis of compound 41-g

A solution of HCl in 1,4-Dioxane (4 M, 15 mL) was added to a solution of compound 41-h (1.50 g, 5.13 mmol) in ethyl acetate (30 mL) a 0° C., the resulting mixture was stirred at room temperature for 1 hour, recooled to 0° C., the mixture was added water (50 mL), extracted with ethyl acetate (50 mL×3), and the organic phase was washed with brine (100 mL×3), dried over anhydrous sodium sulfate, filtered and concentrated at reduced pressure to give compound 41-g (1.4 g).

### Synthesis of compound 41-f

Compound 41-g (1.40 g) was added to methanol (30 mL), followed by addition of Pd/C (140 mg) and the mixture was stirred at room temperature under hydrogen atmosphere overnight. The Pd/C in the mixture was filtered off and the filtrate was concentrated at reduced pressure to afford compound 41-f (1.10 g). LC-MS (ESI): m/z=251.1[M+H]⁺.

### Synthesis of compound 41-e

NaBH₄ (160 mg, 4.24 mmol) was added to a solution of compound 41-f (530 mg, 2.12 mmol) in THF (15 mL) in three batches at 0° C. The mixture was stirred at room temperature for 2 hours, recooled to 0° C., quenched by adding saturated ammonium chloride to the mixture, extracted with ethyl acetate (50 mL×3), The organic phase was washed with brine (100 mL×3), dried over anhydrous sodium sulfate, filtered and concentrated at reduced pressure to obtain the product. Compound 41-e (530 mg, 99%) was obtained.

### Synthesis of compound 41-d

Compound 41-e (530 mg, 2.10 mmol) was added to DCM (10 mL), followed by serial addition of TEA (0.584 mL), DMAP (13 mg, 0.11 mmol) and TsCl (440 mg, 2.31 mmol) at 0° C. and the resulting mixture was stirred at room temperature overnight. The mixture was concentrated at reduced pressure to obtain the crude product. The crude product was purified by flash chromatography (DCM/MeOH=20:1, 10:1) to give compound 41-d (354 mg, 41%).

### Synthesis of compound 41-c

Compound 41-d (150 mg, 0.37 mmol) and Cs₂CO₃ (98 mg, 0.30 mmol) were suspended in DMF (5 mL) and the mixture was stirred at 80° C. under nitrogen atmosphere for 15 min, then a solution of 8-d (166 mg, 0.37 mmol) in DMF (1 mL) was added into the above mixture, and the resulting mixture was stirred at 80° C. under nitrogen atmosphere overnight. The mixture was cooled to room temperature, was added saturated sodium chloride (50 mL), extracted with ethyl acetate (50 mL×3), the organic phase was washed with brine (100 mL×3), dried over anhydrous sodium sulfate, filtered and concentrated at reduced pressure to obtain the crude product, which was purified by flash chromatography (DCM/MeOH=20:1) to obtain compound 41-c (144 mg, 57%).

### Synthesis of compound 41-b

Compound 41-c (144 mg, 0.21 mmol) and LiOH—H₂O (26 mg, 0.62 mmol) were suspended in a mixed solvents of methanol (2 mL), 1,4-dioxane (2 mL) and water (0.5 mL) and the resulting mixture was stirred at room temperature overnight. The mixture was adjusted to pH=7 with HCl (1 M) solution, extracted with ethyl acetate (50 mL×3), and the organic phase was washed with brine (100 mL×3), dried over anhydrous sodium sulfate, filtered and concentrated at reduced pressure to give compound 41-b (117 mg, 83%).

### Synthesis of compound 41-a

Compound 41-b (117 mg, 0.17 mmol), O—(tetrahydro-2H-pyran)-2-hydroxylamine (40 mg, 0.34 mmol), HATU (99 mg, 0.26 mmol) and DIPEA (0.091 mL) were suspended in DMF (5 mL) at room temperature and the mixture was stirred at room temperature overnight. The mixture was added brine (50 mL), extracted with ethyl acetate (50 mL×3), and the organic phase was washed with brine (100 mL×3), dried over anhydrous sodium sulfate, filtered, and concentrated at reduced pressure to obtain the crude product, which was purified by preparative HPLC (acid HCOOH) to give compound 41-a (28 mg, 22%).

### Synthesis of compound 41

A solution of hydrogen chloride in 1,4-dioxane (4 M, 0.5 mL) was added dropwise slowly to a solution of compound 41-a (28 mg, 0.037 mmol) in DCM (5 mL) at 0° C., after addition, the mixture was stirred at 0° C. for 30 min, and the mixture was quenched with saturated sodium bicarbonate solution at 0° C. and adjusted pH=8, extracted with DCM (50 mL×3) and the aqueous phase was washed with brine (100 mL×3), dried over anhydrous sodium sulfate, filtered and concentrated at reduced pressure to give compound 41 (18 mg, 72%). LC-MS (ESI): m/z=684.9[M+H]⁺; ¹H NMR (400 MHz, DMSO-d₆): δ 11.34 (1H, s), 9.52 (1H, s), 9.13 (1H, s), 8.41(1H, d, J=2.0 Hz), 8.00 (1H, d, J=2.4 Hz), 7.63 (1H, dd, J=8.0 Hz, J=1.2 Hz), 7.54(1H, dd, J=11.2 Hz, J=1.6 Hz), 7.47-7.34 (2H, m), 7.12 (1H, d, J=9.2 Hz, J=2.4 Hz), 5.11-4.98 (1H, m), 4.03 (3H, s), 3.89 (4H, t, J=4.8 Hz), 3.72 (4H, t, J=4.4 Hz), 3.10 (3H, s), 3.01 (1H, t, J=12.4 Hz), 2.20-1.96 (4H, m), 1.86-1.53 (4H, m).

### Synthetic route of compound 42

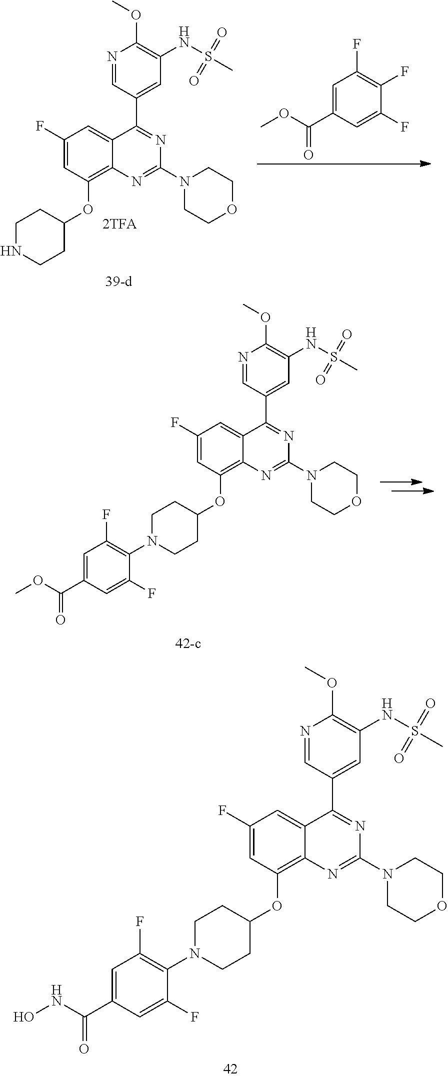

### Synthesis of compound 42-c

A microwave tube was charged with 39-d (150 mg, 0.21 mmol), methyl 3,4,5-trifluorobenzoate (126 mg, 0.66 mmol), potassium carbonate (142 mg 1.00 mmol) and DMF (1.5 mL). Purged with nitrogen for 1 min, the resulting mixture was heated at 80° C. for 2.5 hours. The mixture was cooled to room temperature, added ice-water, extracted with ethyl acetate, washed with citric acid in water, washed with saturated salt in water, dried over anhydrous sodium sulfate, filtered and evaporated to obtain 42-c (180 mg, crude) directly for the next step. 1C-MS (ESI): m/z 703.2 (M+H)⁺.

### Synthesis of compound 42

Synthesized according to synthesis of compound 30, compound 42-c was used instead of compound 30-c to give 42 (35 mg). LC-MS (ESI): m/z 704.3 (M+H)*; ¹H NMR (400 MHz, DMSO-d₆): δ11.26 (1H, bs), 9.49 (1H, bs), 9.16 (1H, s), 8.39 (1H, d, J=2.2 Hz), 7.99 (1H, d, J=2.2 Hz), 7.50-7.38 (3H, m), 7.15 (1H, dd, J=9.5, 2.7 Hz), 4.97-4.91 (1H, m), 4.03 (3H, s), 3.87 (4H, t, J=4.7 Hz), 3.72 (4H, t, J=4.7 Hz), 3.58-3.47 (2H, m), 3.20-3.10 (2H, m), 3.10 (3H, s), 2.12-2.01 (2H, m), 1.95-1.84 (2H, m).

### Synthesis of compound 43

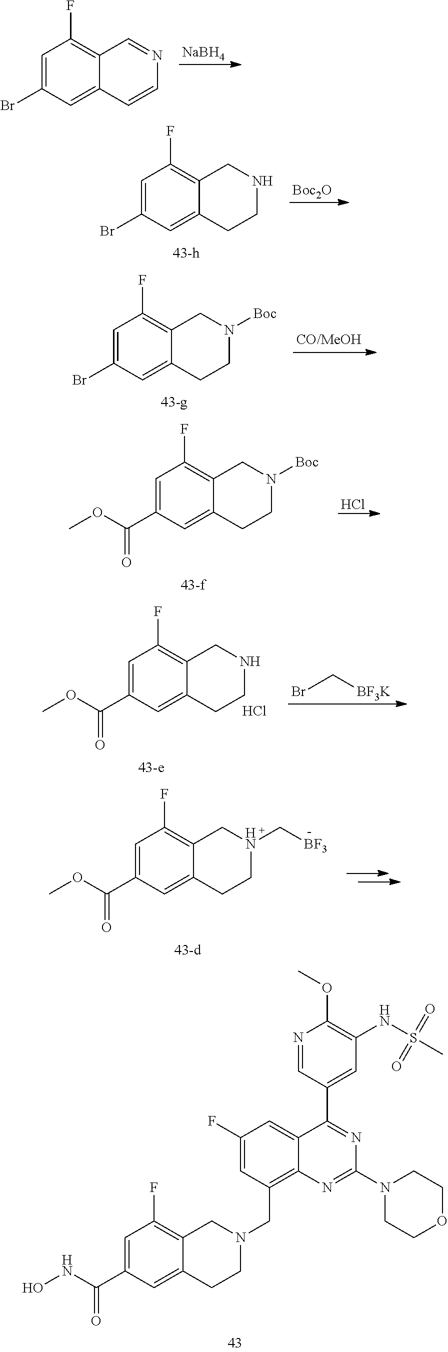

### Synthesis of compound 43-h

6-Bromo-8-fluoroisoquinoline (900 mg, 3.98 mmol) was added acetic acid (10 mL) and the resulting mixture was cooled to 0° C., NaBH₄ (527 mg, 13.93 mol) was added to it in three batches, stirred at room temperature overnight, and recooled to 0° C. The mixture was quenched with saturated sodium bicarbonate, adjusted pH=7-8 after quenching, and extracted with ethyl acetate (50 mL×3), the organic phase was washed with brine (100 mL×3), dried over anhydrous sodium sulfate, filtered and concentrated at reduced pressure to obtain the product compound 43-h (760 mg, 83%).

### Synthesis of compound 43-g

Compound 43-h (760 mg, 3.30 mmol), Boc₂O (2.16 g, 9.90 mmol), TEA (1.38 mL, 9.90 mmol) were suspended in DCM (20 mL) and the resulting mixture was stirred at room temperature overnight. The mixture was added 2 g silica gel, and concentrated at reduced pressure, and the remain residue was purified by flash chromatography (PE/EA=20:1) to obtain compound 43-g (913 mg, 84%).

### Synthesis of compound 43-f

43-g (813 mg, 2.46 mmol), Pd(dppf)Cl₂ (183 mg, 0.25 mmol) and triethylamine (1.03 mL, 7.39 mmol) were suspended in MeOH (10 mL), the resulting mixture was stirred at 100° C. under CO atmosphere overnight, cooled to room temperature. The mixture was added 2 g silica gel and t concentrated at reduced pressure. The residue was purified by flash column (PE/EA=20:1) to obtain compound 43-f (430 mg, 57%).

### Synthesis of compound 43-e

Compound 43-f (430 mg, 1.39 mmol) was added to a solution of hydrogen chloride in 1,4-dioxane (4 M, 8 mL) and stirred at room temperature for 1 hour, and the mixture was concentrated at reduced pressure to give compound 43-e (319 mg, 94%).

### Synthesis of compound 43-d

Compound 43-e (319 mg, 1.30 mmol) in acetonitrile (20 mL) was added serially potassium iodide (13 mg, 0.078 mmol), potassium bromomethylfluoroborate (783 mg, 3.90 mmol), and potassium carbonate (539 mg, 3.90 mmol) at room temperature and the resulting mixture was stirred at 80° C. under nitrogen atmosphere overnight. The mixture was cooled to room temperature, filtered and concentrated at reduced pressure to give compound 43-d (120 mg, 32%).

### Synthesis of compound 43

Synthesized according to synthesis of compound 1, compound 43-d was used instead of compound 1-d to give compound 43 (8 mg). LC-MS (ESI): m/z=656.3[M+H]⁺.

### Synthetic route of compound 44

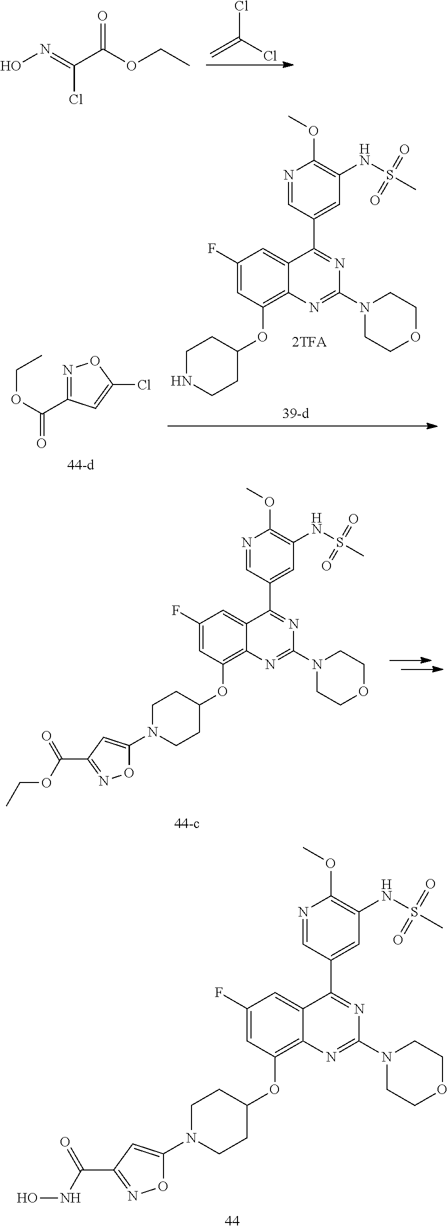

### Synthesis of compound 44-d

The reaction flask was charged with ethyl chlorooximeacetate (5.5 g, 36.29 mmol) and 1,1-dichloroethylene (50 mL), and a solution of triethylamine(13.5 mL, 95.18 mmol) in 1,1-dichloroethylene (50 mL) was added dropwise at 10° C. under nitrogen atmosphere in 2 hours. The water bath was withdrawn in 1 hour. The resulting mixture was stirred at room temperature for 30 minutes. Poured into a mixture of dichloromethane and water, partied. The dichloromethane phase was washed the with 1M hydrochloric acid, saturated sodium chloride, dried over anhydrous sodium sulfate, filtered, and the solvent was evaporated to dryness, and the residue was purified on a silica gel column (mobile phase: petroleum ether/ethyl acetate=100/0 to 80/20) to give 44-d (1.00 g, 16%).

### Synthesis of compound 44-c

A microwave tube charged with 39-d (150 mg, 0.21 mmol), 44-d (150 mg, 0.85 mmol), potassium carbonate (145 mg, 1.02 mmol) and DMF (1.5 mL) was purged with nitrogen for 1 min. The resulting mixture was heated at 80° C. for 3.5 hours. The mixture was cooled to room temperature, added into ice-water, extracted with ethyl acetate, washed serially with citric acid in water and saturated salt in water, dried over anhydrous sodium sulfate, filtered, evaporated and purified on a silica column (mobile phase: dichloromethane/methanol, 100/0 to 96/4) to give 44-c (67 mg, 47%) for direct use in the next step. LC-MS (ESI): m/z 672.4 (M+H)⁺.

### Synthesis of compound 44

Synthesized according to synthesis of compound 30, compound 44-c was used in place of compound 30-c to give 44 (12 mg). LC-MS (ESI): m/z 659.3 (M+H)⁺; ¹H NMR (DMSO-d₆, 400 MHz): δ 11.29 (1H, s), 9.52 (1H, s), 9.26 (1H, s), 8.39 (1H, d, J=2.2 Hz), 7.99 (1H, d, J=2.3 Hz), 7.47 (1H, dd, J=10.7, 2.6 Hz), 7.17 (1H, dd, J=9.4, 2.6 Hz), 5.63 (1H, s), 5.00-4.93 (1H, m), 4.03 (3H, s), 3.88-3.75 (4H, m), 3.71-3.61 (6H, m), 3.44-3.33 (2H, m), 3.10 (3H, s), 2.11-1.95 (2H, m), 1.92-1.79 (2H, m).

### Synthetic route of compound 45

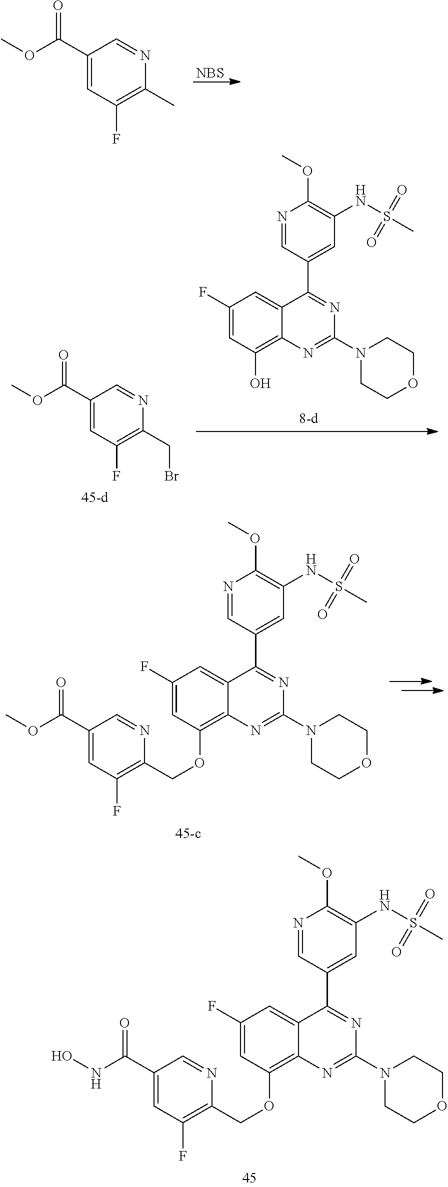

### Synthesis of compound 45-d

Compound 5-fluoro-6-methylnicotinic acid methyl ester (140 mg, 0.83 mmol), NBS (148 mg, 0.83 mmol) and AIBN (14 mg, 0.083 mmol) in CCl₄ (5 mL) was stirred at reflux under nitrogen atmosphere overnight. The mixture was reduced to room temperature and concentrated at reduced pressure to obtain the crude product. The crude product was purified by flash chromatography (PE/EA=10:1) to give compound 45-d (128 mg, 62%). LC-MS (ESI): m/z=249.7 [M+H]⁺.

### Synthesis of compound 45-c

Compound 45-d (128 mg, 0.52 mmol), 8-d (126 mg, 0.28 mmol), K₂CO₃ (79 mg, 0.57 mmol) were added DMF (5 mL) and the resulting mixture was stirred at room temperature overnight. The mixture was added saturated sodium chloride (80 mL), extracted with ethyl acetate (50 mL×3), and the organic phase was washed with brine (100 mL×6), dried over anhydrous sodium sulfate, filtered, and the filtrate was concentrated at reduced pressure to give compound 45-c (170 mg, 98%).

### Synthesis of compound 45

Synthesized according to synthesis of compound 30, compound 45 (1 mg) was obtained using compound 39-c instead of compound 30-c. LC-MS (ESI): m/z=618.0 [M+H]⁺.

Effec Example 1 IC₅₀ evaluation assay for PI3Kα, PI3Kδ, PI3Kβ and PI3Kγ enzyme activity inhibition

The experiment was performed using the ADP-Glo Kinase Assay kit, following the instructions. The following buffer solutions were prepared: 50 mM HEPES, pH 7.5, 3 mM MgCl₂, 1 mM EGTA, 100 mM NaCl, 0.03% CHAPS, 2 mM DTT. The test compound samples were dissolved in DMSO and added to the screening system at a certain starting concentration such as 10 μM, 3-fold dilution, while setting up both DMSO control and no kinase control. The optimal concentrations of PI3Kα, PI3Kδ, PI3Kβ and PI3Kγ enzymes, substrate (PIP2) and ATP were prepared in buffer. The enzyme reaction system included: buffer, ATP 25 μM, kinase substrate (PIP2, 50 μg/mL), kinases [PI3Kα (0.15 μg/mL), PI3Kδ (1.2 μg/mL), PI3Kβ (0.3 μg/mL) and PI3Kγ (2.5 μg/mL)], etc. The reaction system was reacted at room temperature for 1 hour. The reaction was terminated by adding a termination reagent (ADP-Glo reagent, 5 μL), and the ADP content in the system was detected using a detection reagent (Kinase Detection Reagent, 10 μL). Signal data were collected using the Envision instrument. Calculate the percentage of (%) inhibition according to the following formula: % inhibition=(DMSO control signal value−sample signal value)/(DMSO control signal value−no-kinase control signal value). The IC₅₀ value was obtained by fitting the curve using the formula Y=Bottom+(Top-Bottom)/(1+(IC₅₀/X)\hat{ }HillSlope).

Effect Example 2 HDAC1, HDAC2, HDAC3, HDAC6 enzyme activity inhibition IC₅₀ evaluation experiment

Buffer solution (50 mM Tris 7.5, 0.01% Tween-20, 50 mM NaCl) was prepared according to the kit instructions, and the enzyme solution and substrate solution (acetylated peptide with the addition of trypsin) were prepared using the buffer solution. The test compound samples were dissolved with DMSO and added to the screening system at a certain starting concentration such as 10 μM, 3-fold dilution, while setting up a DMSO control and no-enzyme control. The enzyme reaction system includes: buffer, enzyme [final concentration: HDAC1=10 nM, HDAC2=4 nM, HDAC3=7 nM, HDAC6=5 nM], substrate (acetylated peptide) [final concentration: Peptide(HDAC1)=8 μM, Peptide(HDAC2)=10 μM, Peptide(HDAC3)=5 μM, Peptide(HDAC6)=11 μM], trypsin [final concentration: Trypsin(HDAC1,2,3)=0.05 μM, Trypsin(HDAC6)=0.1 μM], etc. The enzymes and compounds were reacted at room temperature for 15 min. After adding the substrate and Trypsin, centrifugation was performed for 1 min, and the fluorescence signal data (excitation wavelength 355 nM/emission wavelength 460 nm) was read continuously for 60 min using Envision, and the linear reaction segment was selected to obtain the slope and thus calculate the inhibition rate at that concentration. The IC₅₀ value was obtained by fitting the curve using the formula Y=Bottom+(Top-Bottom)/(1+(IC₅₀/X)\hat{ }HillSlope).

Effect Example 3 HDAC8, HDAC10 enzyme activity inhibition IC₅₀ evaluation experiment

Prepare buffer solution (HDAC8: 50 mM Tris 8.0, 2.7 mM KCl, 137 mM NaCl, 1 mM MgCl₂, 1 mg/mL BSA; HDAC10: 50 mM Tris 7.5, 0.01% Tween-20, 50 mM NaCl, 0.05 mg/mL BSA) according to the kit instructions, prepared Enzyme solution, substrate solution (acetylated peptide) by using buffer. The test compound samples were dissolved in DMSO and was added to the screening system at a certain starting concentration such as 10 μM, 3-fold dilution, while setting up the DMSO control and the no-enzyme control. The enzyme reaction system includes: buffer, enzyme [final concentration: HDAC8=10 nM, HDAC10=10 nM], substrate (acetylated peptide) [final concentration: Peptide(HDAC8)=20 μM, Peptide(HDAC10)=4 μM], etc. The enzymes and compounds were reacted at room temperature for 15 minutes. After adding the substrate peptide the reaction system was reacted at room temperature for 240 min. Trypsin solution was added [final concentration: Trypsin=50p M] and incubation continued for 120 min. The fluorescence signal data (excitation wavelength 355 nM/emission wavelength 460 nm) was read with Envision instrument. Calculate the inhibition rate at this concentration based on the relative fluorescence units (RFU). The IC₅₀ value was obtained by fitting a curve using the formula Y=Bottom+(Top-Bottom)/(1+(IC₅₀/X)\hat{ }HillSlope). The activity results of the representative compounds are shown in Table 1 below. where “IC₅₀>10 M” is indicated by “*” and “10 μM>IC₅₀>1 μM” is denoted by “**”, “1 μM>IC₅₀>100nV” is denoted by “***”, “100 nM>IC₅₀>10 nMV” with “****”, “IC₅₀≤10 nM” is denoted by “*****”.

TABLE 1
Activity data of representative compounds of the present disclosure

| Compound | | | | | | | | | | |
|---|---|---|---|---|---|---|---|---|---|---|
| No. | PI3K-α | PI3K-β | PI3K-γ | PI3K-δ | HDAC6 | HDAC1 | HDAC2 | HDAC3 | HDAC8 | HDAC10 |
| 1 | ** | — | — | **** | ** | — | — | — | — | — |
| 2 | — | — | — | **** | ** | — | — | — | — | — |
| 3 | — | — | — | ***** | *** | — | — | — | — | — |
| 4 | ** | *** | * | ***** | **** | *** | *** | *** | — | ** |
| 5 | — | — | — | ***** | *** | — | — | — | — | — |
| 6 | — | — | — | ***** | *** | — | — | — | — | — |
| 7 | *** | *** | ** | ***** | **** | **** | **** | **** | — | *** |
| 8 | *** | — | — | **** | ***** | — | — | — | — | — |
| 9 | ** | — | — | **** | ***** | ***** | ***** | **** | — | *** |
| 10 | *** | — | — | ***** | **** | — | — | — | — | — |
| 11 | ** | — | — | ***** | *** | — | — | — | — | — |
| 12 | *** | — | — | ***** | *** | — | — | — | — | — |
| 13 | ** | — | — | ***** | *** | — | — | — | — | — |
| 14 | ** | — | — | **** | ** | — | — | — | — | — |
| 15 | *** | — | — | ***** | *** | — | — | — | — | — |
| 16 | *** | — | — | **** | ** | — | — | — | — | — |
| 17 | ** | — | — | ***** | **** | — | — | — | — | — |
| 18 | ** | — | — | ***** | *** | — | — | — | — | — |
| 19 | ** | — | — | ***** | *** | — | — | — | — | — |
| 20 | ** | — | — | ***** | *** | — | — | — | — | — |
| 21 | ** | — | — | ***** | * | — | — | — | — | — |
| 22 | *** | — | — | ***** | ** | — | — | — | — | — |
| 23 | *** | — | — | ***** | **** | — | — | — | — | — |
| 24 | ** | — | — | **** | **** | — | — | — | — | — |
| 25 | ** | — | — | **** | **** | — | — | — | — | — |
| 26 | *** | — | — | ***** | **** | — | — | — | — | — |
| 27 | ** | — | — | **** | — | — | — | — | — | — |
| 28 | *** | — | — | ***** | * | — | — | — | — | — |
| 29 | *** | — | — | ***** | — | — | — | — | — | — |
| 30 | *** | ** | ** | **** | **** | *** | *** | *** | *** | *** |
| 31 | — | — | — | **** | **** | — | — | — | — | — |
| 32 | — | — | — | ***** | ** | — | — | — | — | — |
| 33 | — | — | — | **** | **** | — | — | — | — | — |
| 34 | — | — | — | **** | **** | — | — | — | — | — |
| 35 | — | — | — | ***** | **** | ***** | — | — | — | — |
| 36 | — | — | — | **** | **** | *** | — | — | — | — |
| 37 | — | — | — | *** | — | — | — | — | — | — |
| 38 | — | — | — | **** | — | — | — | — | — | — |
| 39 | — | — | — | ***** | *** | ***** | **** | **** | *** | **** |
| 40 | — | — | — | ***** | **** | **** | *** | *** | ** | — |
| 41 | — | — | — | *** | *** | ** | — | — | — | — |
| 42 | — | — | — | **** | *** | ** | — | — | — | — |
| 43 | — | — | — | **** | *** | ** | — | — | — | — |
| 44 | — | — | — | **** | **** | *** | *** | — | — | — |
| 45 | — | — | — | ** | **** | *** | ** | — | — | — |
| YY-20394 | *** | *** | * | ***** | * | — | — | — | — | — |

Although the specific embodiments of the present disclosure have been described above, it will be understood by those of skill in the art that these are merely illustrative, and that various alterations or modifications can be made to these embodiments without departing from the principle and essence of the present disclosure. Therefore, the scope of protection of the present disclosure is defined by the appended claims.

1. A morpholinylquinazoline compound represented by formula I, a pharmaceutically acceptable salt thereof, a solvate thereof, a solvate of the pharmaceutically acceptable salt thereof, a polycrystalline form thereof or a precursor thereof, 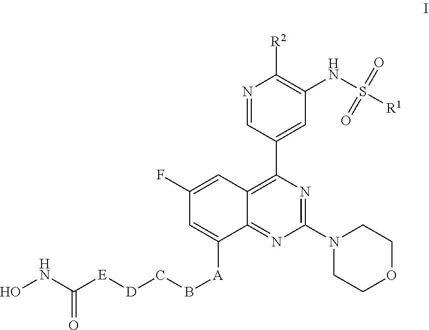
wherein;
R¹ is C₁₋₄ alkyl, C₁₋₄ alkyl substituted with one or more halogen, or C₃₋₈ cycloalkyl;
R² is hydrogen or —OC₁₋₄ alkyl;
A, C and E are independently a bond, or —(U₁)ₙ₁—(R^{L-1})ₙ₂—(U₂)ₙ₃—(R^{L-2})ₙ₄—(U₃)ₙ₅—(R^{L-3})ₙ₆—;
U₁, U₂ and U₃ are independently —O—, —S—, —NH— or —NR³—, R³ is C₁₋₆ alkyl;
R^{L-1}, R^{L-2} and R^{L-3} are independently C₁₋₆ alkylene, C₂₋₆ alkenyl or C₂₋₆ alkynyl;
n1, n3 and n5 are independently 0 or 1;
n2, n4 and n6 are independently 0, 1, 2, 3 or 4;
B and D are independently a bond, C₃₋₁₀ cycloalkyl, C₃₋₁₀ cycloalkyl substituted with one or more R¹⁻¹, “4-10 membered heterocycloalkyl containing 1-3 heteroatoms selected from O and N”, “4-10 membered heterocycloalkyl containing 1-3 heteroatoms selected from O and N” substituted with one or more R¹⁻², C₆₋₂₀ aryl, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, “8-12 membered benzoheterocycloalkenyl containing 1-3 heteroatoms selected from O, S and N”, “8-12 membered benzoheterocycloalkenyl containing 1-3 heteroatoms selected from O, S and N” substituted with one or more R¹⁻⁵-5, 5-7 membered cycloalkenyl, or 5-7 membered cycloalkenyl substituted with one or more R¹⁻⁶; when multiple substituents are present, the substituents are the same or different;
R¹⁻¹, R¹⁻², R¹⁻³, R¹⁻⁴, R¹⁻⁵ and R¹⁻⁶ are independently halogen or C₁₋₆ alkyl.

2. The morpholinylquinazoline compound represented by formula I as defined in claim 1, a pharmaceutically acceptable salt thereof, a solvate thereof, a solvate of the pharmaceutically acceptable salt thereof, a polycrystalline form thereof or a precursor thereof, wherein,
R¹ is C₁₋₄ alkyl, C₁₋₄ alkyl substituted with one or more halogen, or C₃₋₈ cycloalkyl;
R² is hydrogen or —OC₁₋₄ alkyl;
A is —O—, C₁₋₆ alkylene, —OC₁₋₆ alkylene, or C₁₋₆ alkylene-NH—;
B is a bond, C₃₋₁₀ cycloalkyl, “4-10 membered heterocycloalkyl containing 1-3 heteroatoms selected from O and N”, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or “8-12 membered benzoheterocycloalkenyl containing 1-3 heteroatoms selected from O, S and N” substituted with one or more R¹⁻⁵;
C is a bond, C₁₋₆ alkylene, C₂₋₆ alkenyl, —OC₁₋₆ alkylene, —OC₁₋₆ alkylene-OC₁₋₆ alkylene, C₁₋₆ alkylene-OC₁₋₆ alkylene or C₁₋₆ alkylene-NH—;
D is a bond, C₆₋₂₀ aryl, C₆₋₂₀ aryl substituted with one or more R¹⁻³, C₃₋₁₀ cycloalkyl, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or “4-10 membered heterocycloalkyl containing 1-3 heteroatoms selected from O and N”;
E is a bond.

3. The morpholinylquinazoline compound represented by formula I as defined in claim 1, a pharmaceutically acceptable salt thereof, a solvate thereof, a solvate of the pharmaceutically acceptable salt thereof, a polycrystalline form thereof or a precursor thereof, wherein,
R¹ is C₁₋₄ alkyl, C₁₋₄ alkyl substituted with one or more halogen, or C₃₋₈ cycloalkyl;
R² is hydrogen or —OC₁₋₄ alkyl;
A is —O—, C₁₋₆ alkylene, —OC₁₋₆ alkylene, or C₁₋₆ alkylene-NH—;
B is a bond, C₃₋₁₀ cycloalkyl, “4-10 membered heterocycloalkyl containing 1-3 heteroatoms selected from O and N”, C₆₋₂₀ aryl substituted with one or more R¹⁻³, or “5-12 membered heteroaryl containing 1-4 heteroatoms selected from O, S and N” substituted with one or more R¹⁻⁴;
C is a bond or C₁₋₆ alkylene;
D is a bond, C₆₋₂₀ aryl, C₆₋₂₀ aryl substituted with one or more R¹⁻³, “5-12 membered heteroaryl containing 1-4 heteroatoms selected from O, S and N”, or “5-12 membered heteroaryl containing 1-4 heteroatoms selected from O, S and N” substituted with one or more R¹⁻⁴;
E is a bond.

4. The morpholinylquinazoline compound represented by formula I as defined in claim 1, a pharmaceutically acceptable salt thereof, a solvate thereof, a solvate of the pharmaceutically acceptable salt thereof, a polycrystalline form thereof or a precursor thereof, wherein,
A is —O—, C₁₋₆ alkylene, —OC₁₋₆ alkylene or C₁₋₆ alkylene-NH—;
and/or, B is a bond, C₃₋₁₀ cycloalkyl, “4-10 membered heterocycloalkyl containing 1-3 heteroatoms selected from O and N”, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or “8-12 membered benzoheterocycloalkenyl containing 1-3 heteroatoms selected from 0, S and N” substituted with one or more R¹⁻⁵;
and/or, C is a bond, C₁₋₆ alkylene, C₂₋₆ alkenyl, —OC₁₋₆ alkylene, —OC₁₋₆ alkylene-OC₁₋₆ alkylene, C₁₋₆ alkylene-OC₁₋₆ alkylene or C₁₋₆ alkylene-NH—;
and/or, D is a bond, C₆₋₂₀ aryl, C₆₋₂₀ aryl substituted with one or more R¹⁻³, “5-12 membered heteroaryl containing 1-4 heteroatoms selected from O, S and N”, “5-12 membered heteroaryl containing 1-4 heteroatoms selected from O, S and N” substituted with one or more R¹⁻⁴, or “4-10 membered heterocycloalkyl containing 1-3 heteroatoms selected from O and N”;
E is a bond.

5. The morpholinylquinazoline compound represented by formula I as defined in claim 4, a pharmaceutically acceptable salt thereof, a solvate thereof, a solvate of the pharmaceutically acceptable salt thereof, a polycrystalline form thereof or a precursor thereof, wherein,
when R¹ is C₁₋₄ alkyl or C₁₋₄ alkyl substituted with one or more halogens, the C₁₋₄ alkyl and the C₁₋₄ alkyl in the C₁₋₄ alkyl substituted with one or more halogens are methyl, ethyl, n-propyl, isopropyl, n-butyl, sec-butyl, isobutyl or tert-butyl;
and/or, when R¹ is C₃₋₈ cycloalkyl, the C₃₋₈ cycloalkyl is cyclohexyl, cyclopentyl, cyclobutyl or cyclopropyl;
and/or, when R² is —OC₁₋₄ alkyl, the C₁₋₄ alkyl in the —OC₁₋₄ alkyl is -methyl, -ethyl, n-propyl, isopropyl, n-butyl, sec-butyl, isobutyl or tert-butyl;
and/or, when R¹⁻¹, R¹⁻², R¹⁻³, R¹⁻⁴, R¹⁻⁵ and R¹⁻⁶ are independently halogen, the halogen is fluorine, chlorine, bromine or iodine;
and/or, when R³, R¹⁻¹, R¹⁻², R¹⁻³, R¹⁻⁴, R¹⁻⁵ and R¹⁻⁶ are independently C₁₋₆ alkyl, the C₁₋₆ alkyl is C₁₋₄ alkyl, preferably methyl, ethyl, n-propyl, isopropyl, n-butyl, sec-butyl, isobutyl or tert-butyl;
and/or, when A is C₁₋₆ alkylene, —OC₁₋₆ alkylene or C₁₋₆ alkylene-NH—, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—;
and/or, when B is C₃₋₁₀ cycloalkyl, the C₃₋₁₀ cycloalkyl is cyclohexyl, cyclopentyl, cyclobutyl or cyclopropyl;
and/or, when B is “4- to 10-membered heterocycloalkyl containing 1-3 heteroatoms selected from O, S and N”, the “4- to 10-membered heterocycloalkyl containing 1-3 heteroatoms selected from O, S and N” is “3- to 6-membered heterocycloalkyl containing 1-2 heteroatoms selected from N”;
and/or, when B is C₆₋₂₀ aryl substituted with one or more R¹⁻³, the C₆₋₂₀ aryl is phenyl;
and/or, when B is “5- to 12-membered heteroaryl containing 1-4 heteroatoms selected from O, S and N” substituted with one or more R¹⁻⁴, the “5- to 12-membered heteroaryl containing 1-4 heteroatoms selected from O, S and N” is “5- to 6-membered heteroaryl containing 1-2 heteroatoms selected from N”;
and/or, when B is “5- to 12-membered heteroaryl containing 1-4 heteroatoms selected from O, S and N” substituted with one or more R¹⁻⁴, the B is 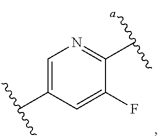 [SMILES: *c1cnc(*)c(F)c1]
preferably, the position a is connected with A;
and/or, when B is “8- to 12-membered benzoheterocycloalkenyl containing 1-3 heteroatoms selected from O, S and N” substituted with one or more R¹⁻⁵, the B is “9- to 10-membered benzoheterocycloalkenyl containing 1 heteroatoms of N”;
and/or, when B is “8- to 12-membered benzocyclcloalkenyl containing 1-3 heteroatoms selected from O, S and N” substituted with one or more R¹⁻⁵, the B is 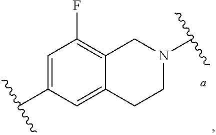 [SMILES: *c1cc(F)c2c(c1)CCN(*)C2]
preferably, the position a is connected with A;
and/or, when C is C₁₋₆ alkylene, —OC₁₋₆ alkylene-OC₁₋₆ alkylene, C₁₋₆ alkylene-OC₁₋₆ alkylene or C₁₋₆ alkylene-NH—, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—;
and/or, when C is —OC₁₋₆ alkylene or —OC₁₋₆ alkylene-OC₁₋₆ alkylene, the —O end is connected with B;
and/or, when C is C₁₋₆ alkylene-NH—, the C₁₋₆ alkylene end is connected with B; and/or, when C is C₂₋₆ alkenyl, the C is vinyl, propenyl or allyl, preferably, when C is vinyl, the vinyl is 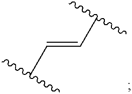 [SMILES: */C=C/*]
and/or, when D is C₆₋₂₀ aryl or C₆₋₂₀ aryl substituted with one or more R¹⁻³, the C₆₋₂₀ aryl is phenyl or naphthyl; and/or, when D is C₆₋₂₀ aryl substituted with one or more R¹⁻³, the D is 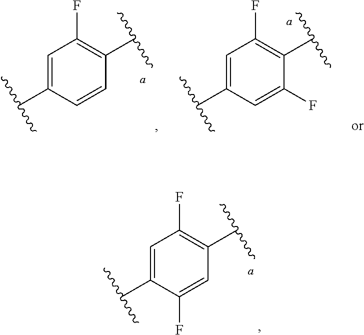 [SMILES: *c1ccc(*)c(F)c1 ; *c1cc(F)c(*)c(F)c1 ; *c1cc(F)c(*)cc1F]
preferably, the position a is connected with A; and/or, when D is “5- to 12-membered heteroaryl containing 1-4 heteroatoms selected from O, S and N” or “5- to 12-membered heteroaryl containing 1-4 heteroatoms selected from O, S and N” substituted with one or more R¹⁻⁴, the “5- to 12-membered heteroaryl containing 1-4 heteroatoms selected from O, S and N” is “5- to 6-membered heteroaryl containing 1-2 heteroatoms selected from O and N”;
and/or, when D is “5- to 12-membered heteroaryl containing 1-4 heteroatoms selected from O, S and N” substituted with one or more R¹⁻⁴, the D is 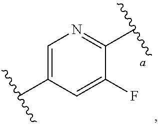 [SMILES: *c1cnc(*)c(F)c1]
preferably, the position a is connected with C;
and/or, when D is “4- to 10-membered heterocycloalkyl containing 1-3 heteroatoms selected from O, S and N”, the D is “5- to 6-membered heterocycloalkyl containing 1-2 heteroatoms selected from O and N”.

6. The morpholinylquinazoline compound represented by formula I as defined in claim 1, a pharmaceutically acceptable salt thereof, a solvate thereof, a solvate of the pharmaceutically acceptable salt thereof, a polycrystalline form thereof or a precursor thereof, wherein,
A is —O—, —CH₂—, 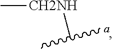
—OCH₂CH₂—^{a} 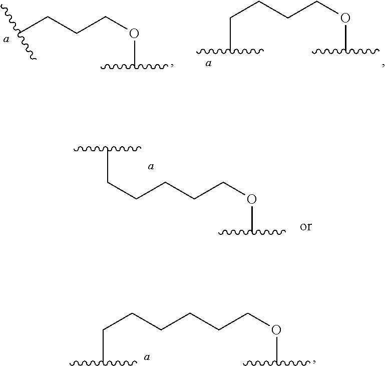 [SMILES: *CCCO* ; *CCCCO* ; *CCCCCO* ; *CCCCCCO*]
preferably, the position a is connected with B; and/or, B is a bond, 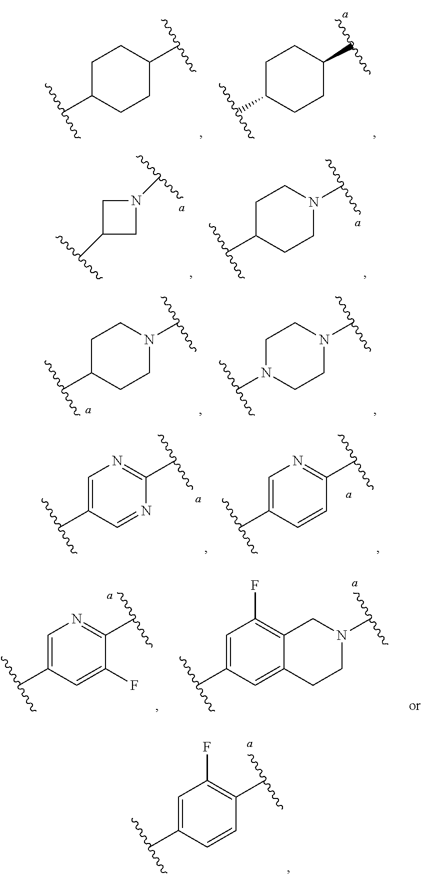
preferably, the position a is connected with A; and/or, C is —CH₂—, ^{a}OCH₂CH₂—, 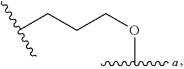 [SMILES: *CCCO*]
—CH₂CH₂—, —CH₂CH₂CH₂—, — 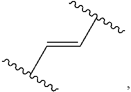 [SMILES: */C=C/*]
—CH₂CH₂CH₂CH₂—,^{a}—OCH₂CH₂OCH₂—or ^{a}—C(CH₃)₂OCH₂—, preferably, the position a is connected with B; and/or, D is a bond, 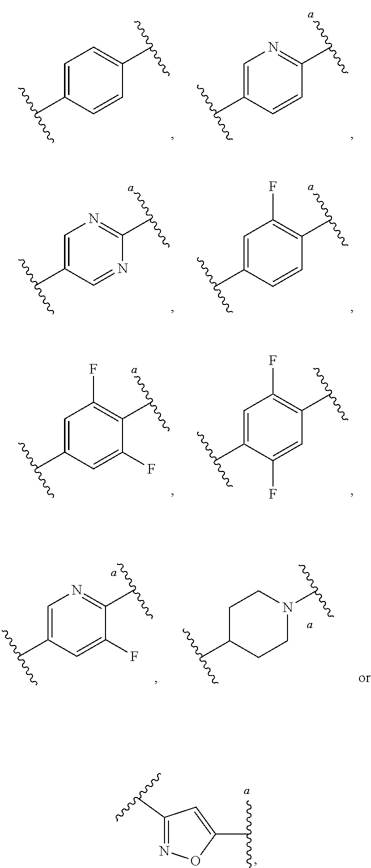
preferably, the position a is connected with C.

7. The morpholinylquinazoline compound represented by formula I as defined in claim 6, a pharmaceutically acceptable salt thereof, a solvate thereof, a solvate of the pharmaceutically acceptable salt thereof, a polycrystalline form thereof or a precursor thereof, wherein, -D-C-B-A- is

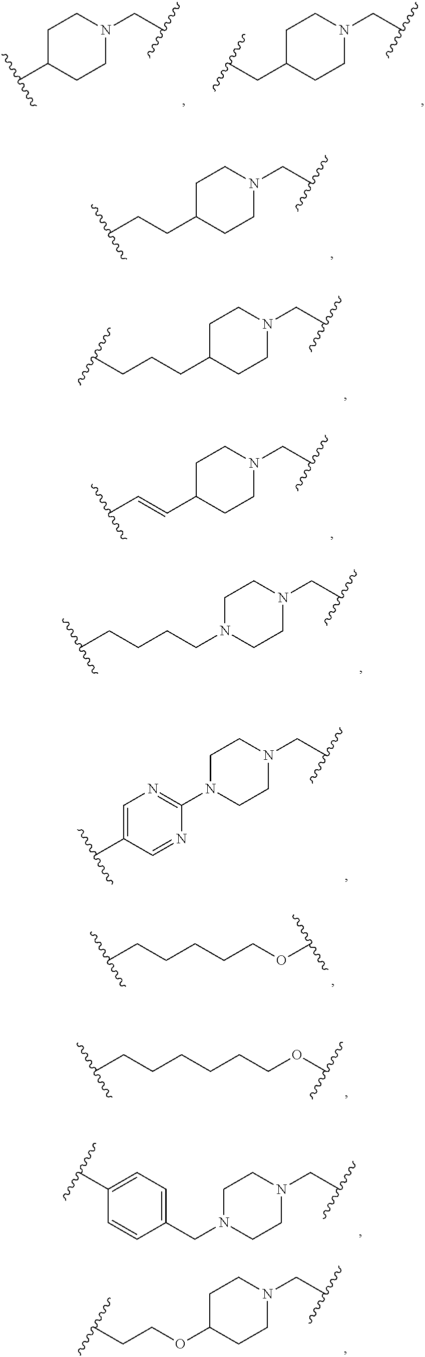

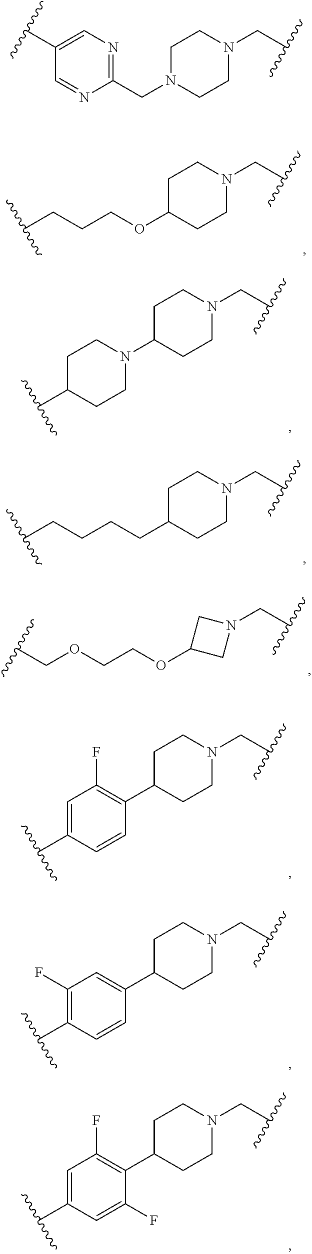

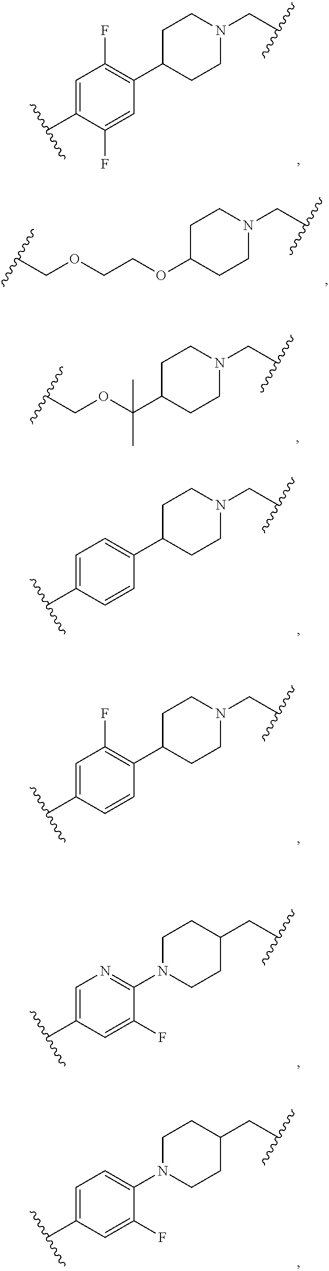

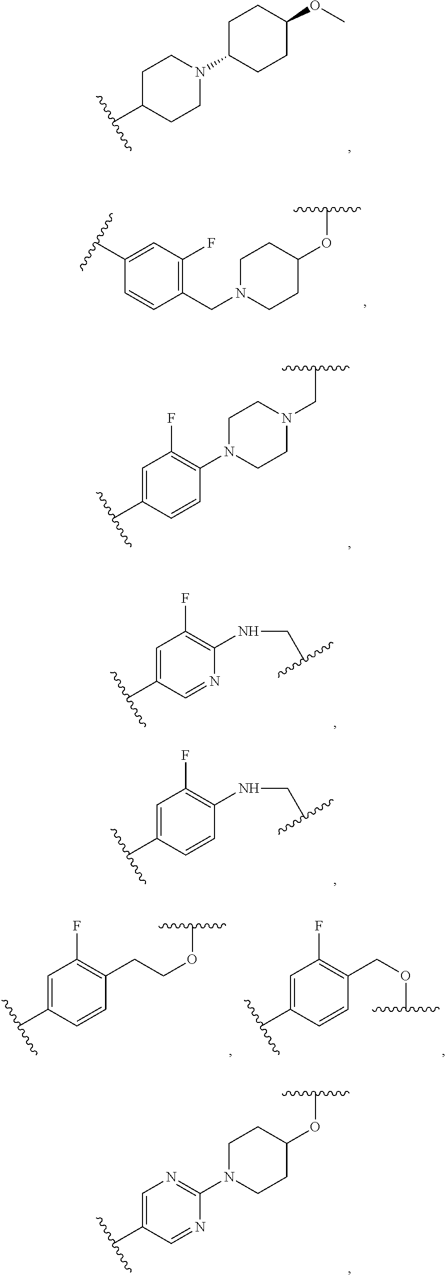

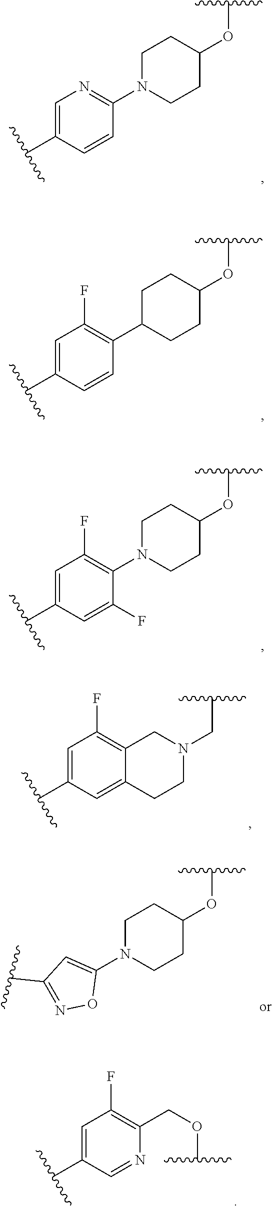 [SMILES: *OC1CCN(c2ccc(*)cn2)CC1 ; *OC1CCC(c2ccc(*)cc2F)CC1 ; *OC1CCN(c2c(F)cc(*)cc2F)CC1 ; *CN1CCc2cc(*)cc(F)c2C1 ; *OC1CCN(c2cc(*)no2)CC1 ; *OCc1ncc(*)cc1F]

8. The morpholinylquinazoline compound represented by formula I as defined in claim 7, a pharmaceutically acceptable salt thereof, a solvate thereof, a solvate of the pharmaceutically acceptable salt thereof, a polycrystalline form thereof or a precursor thereof, wherein, -D-C-B-A- is 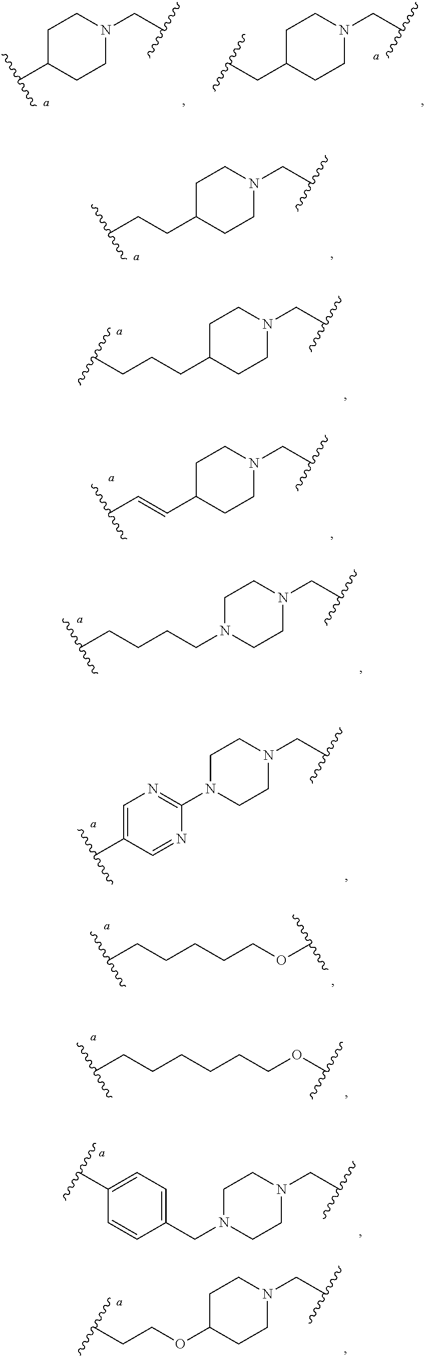 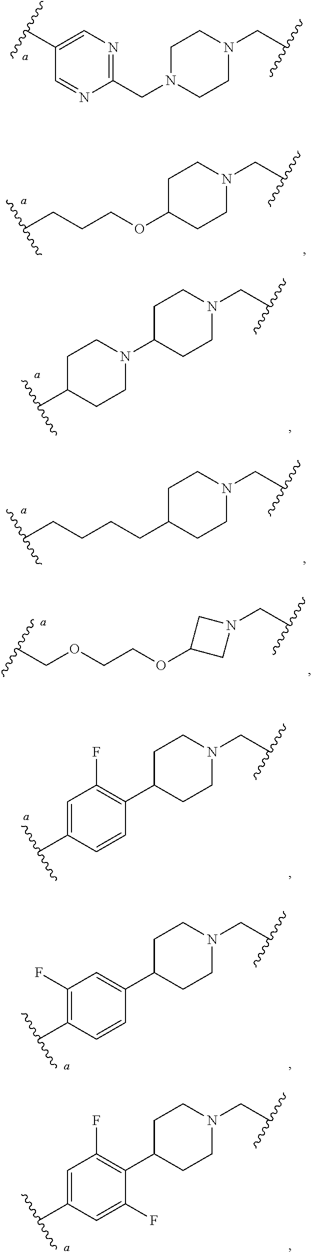 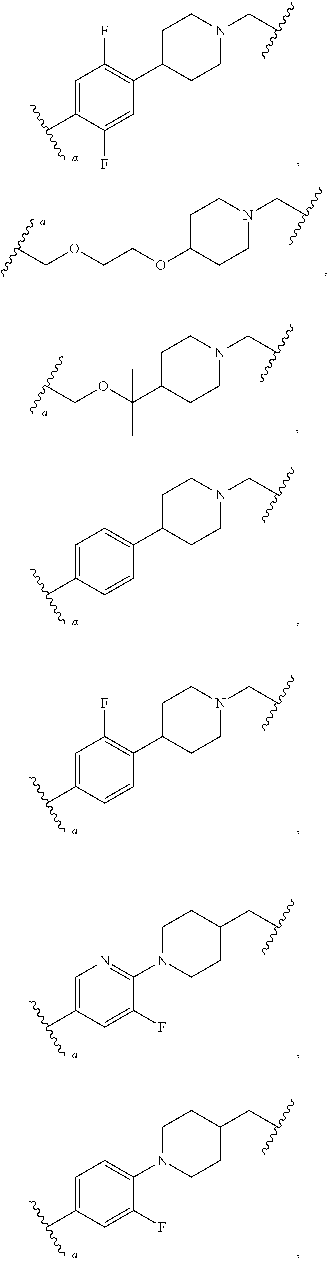 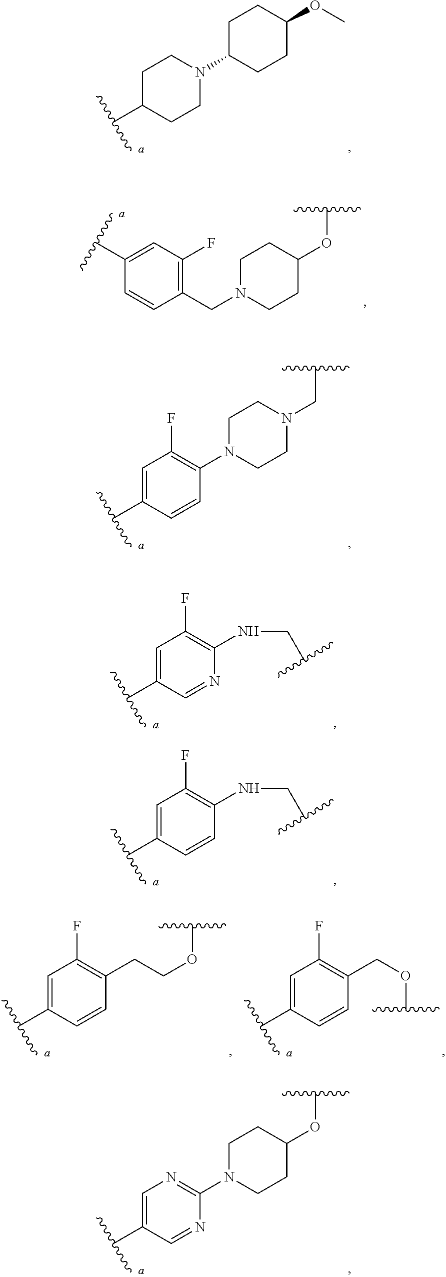 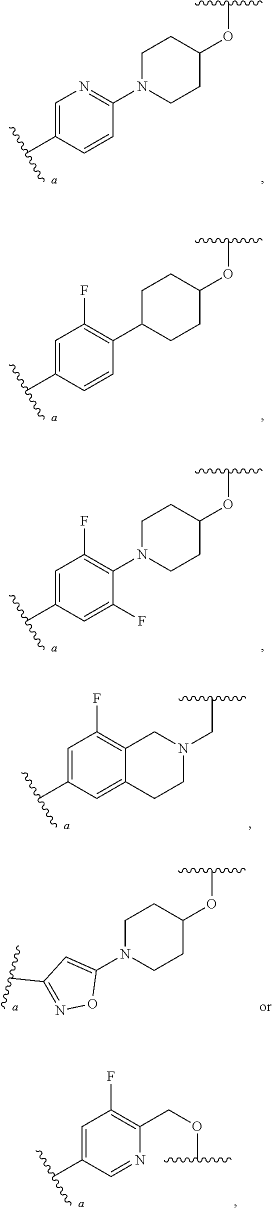 [SMILES: *OC1CCN(c2ccc(*)cn2)CC1 ; *OC1CCC(c2ccc(*)cc2F)CC1 ; *OC1CCN(c2c(F)cc(*)cc2F)CC1 ; *CN1CCc2cc(*)cc(F)c2C1 ; *OC1CCN(c2cc(*)no2)CC1 ; *OCc1ncc(*)cc1F]
the position a is connected with E.

9. The morpholinylquinazoline compound represented by formula I as defined in claim 1, a pharmaceutically acceptable salt thereof, a solvate thereof, a solvate of the pharmaceutically acceptable salt thereof, a polycrystalline form thereof or a precursor thereof, wherein the morpholinylquinazoline compound represented by formula I has any one of the following structures:

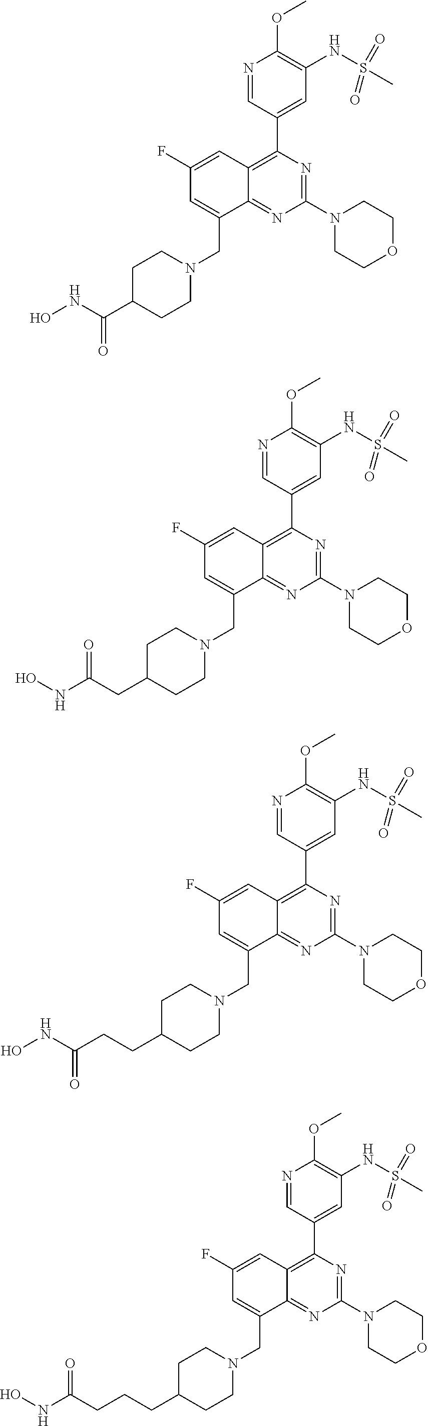 [SMILES: COc1ncc(-c2nc(N3CCOCC3)nc3c(CN4CCC(C(=O)NO)CC4)cc(F)cc23)cc1NS(C)(=O)=O ; COc1ncc(-c2nc(N3CCOCC3)nc3c(CN4CCC(CC(=O)NO)CC4)cc(F)cc23)cc1NS(C)(=O)=O ; COc1ncc(-c2nc(N3CCOCC3)nc3c(CN4CCC(CCC(=O)NO)CC4)cc(F)cc23)cc1NS(C)(=O)=O ; COc1ncc(-c2nc(N3CCOCC3)nc3c(CN4CCC(CCCC(=O)NO)CC4)cc(F)cc23)cc1NS(C)(=O)=O]

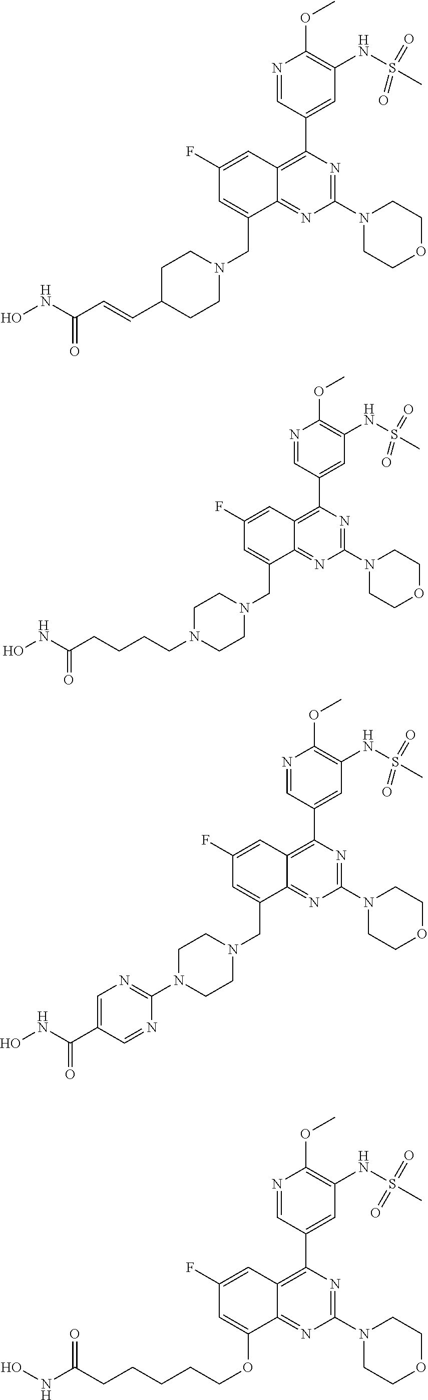 [SMILES: COc1ncc(-c2nc(N3CCOCC3)nc3c(CN4CCC(/C=C/C(=O)NO)CC4)cc(F)cc23)cc1NS(C)(=O)=O ; COc1ncc(-c2nc(N3CCOCC3)nc3c(CN4CCN(CCCCC(=O)NO)CC4)cc(F)cc23)cc1NS(C)(=O)=O ; COc1ncc(-c2nc(N3CCOCC3)nc3c(CN4CCN(c5ncc(C(=O)NO)cn5)CC4)cc(F)cc23)cc1NS(C)(=O)=O ; COc1ncc(-c2nc(N3CCOCC3)nc3c(OCCCCCC(=O)NO)cc(F)cc23)cc1NS(C)(=O)=O]

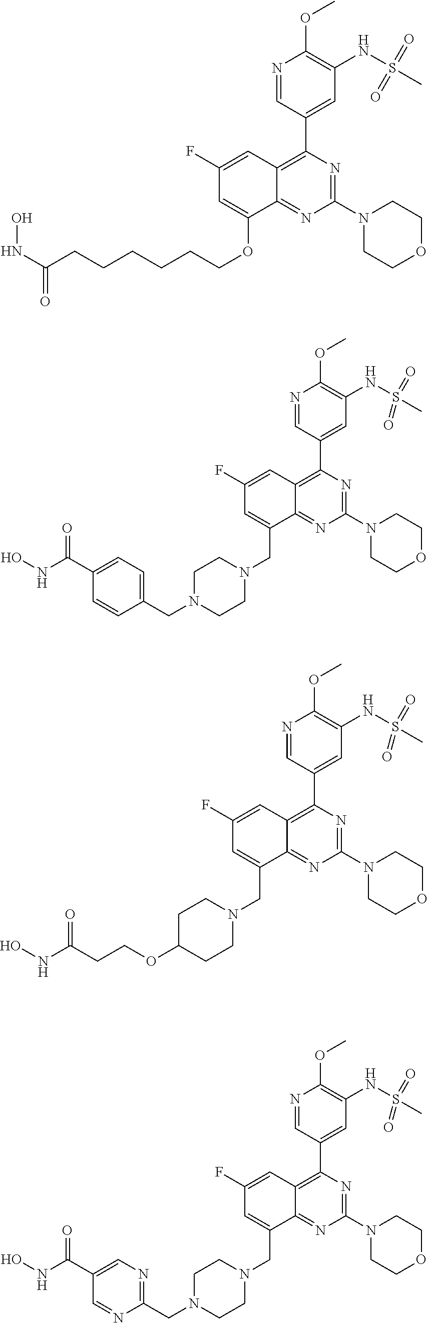 [SMILES: COc1ncc(-c2nc(N3CCOCC3)nc3c(OCCCCCCC(=O)NO)cc(F)cc23)cc1NS(C)(=O)=O ; COc1ncc(-c2nc(N3CCOCC3)nc3c(CN4CCN(Cc5ccc(C(=O)NO)cc5)CC4)cc(F)cc23)cc1NS(C)(=O)=O ; COc1ncc(-c2nc(N3CCOCC3)nc3c(CN4CCC(OCCC(=O)NO)CC4)cc(F)cc23)cc1NS(C)(=O)=O ; COc1ncc(-c2nc(N3CCOCC3)nc3c(CN4CCN(Cc5ncc(C(=O)NO)cn5)CC4)cc(F)cc23)cc1NS(C)(=O)=O]

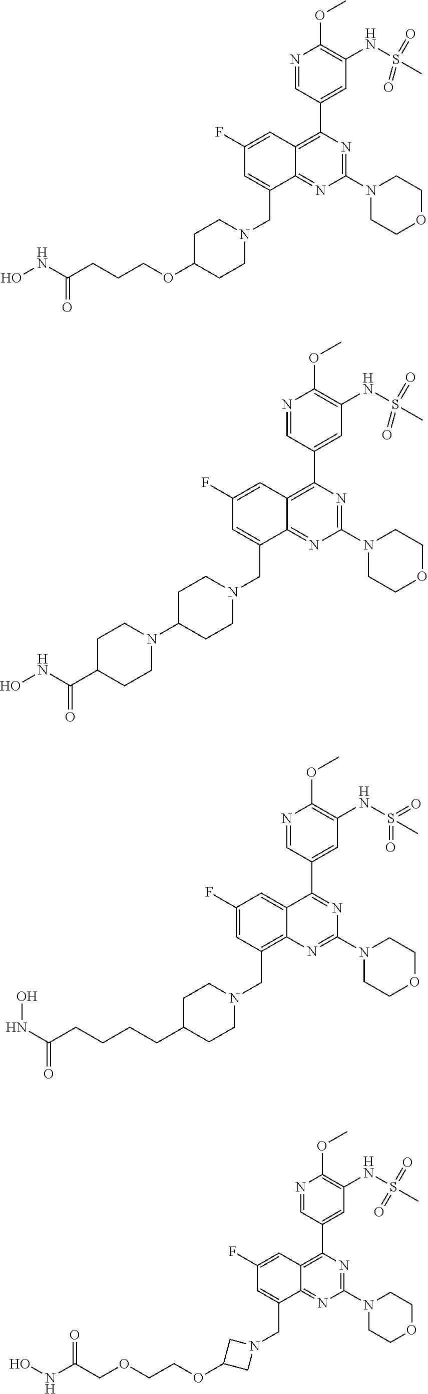 [SMILES: COc1ncc(-c2nc(N3CCOCC3)nc3c(CN4CCC(OCCCC(=O)NO)CC4)cc(F)cc23)cc1NS(C)(=O)=O ; COc1ncc(-c2nc(N3CCOCC3)nc3c(CN4CCC(N5CCC(C(=O)NO)CC5)CC4)cc(F)cc23)cc1NS(C)(=O)=O ; COc1ncc(-c2nc(N3CCOCC3)nc3c(CN4CCC(CCCCC(=O)NO)CC4)cc(F)cc23)cc1NS(C)(=O)=O ; COc1ncc(-c2nc(N3CCOCC3)nc3c(CN4CC(OCCOCC(=O)NO)C4)cc(F)cc23)cc1NS(C)(=O)=O]

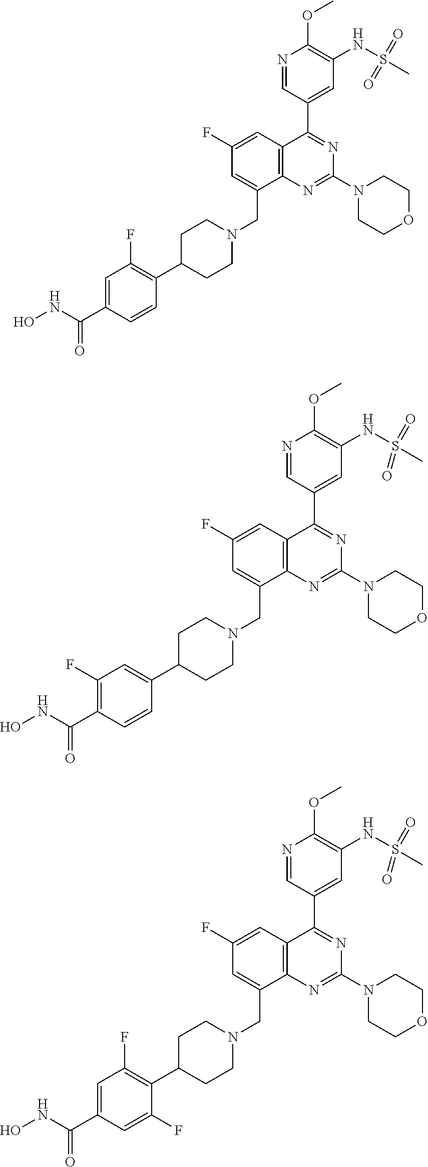 [SMILES: COc1ncc(-c2nc(N3CCOCC3)nc3c(CN4CCC(c5ccc(C(=O)NO)cc5F)CC4)cc(F)cc23)cc1NS(C)(=O)=O ; COc1ncc(-c2nc(N3CCOCC3)nc3c(CN4CCC(c5ccc(C(=O)NO)c(F)c5)CC4)cc(F)cc23)cc1NS(C)(=O)=O ; COc1ncc(-c2nc(N3CCOCC3)nc3c(CN4CCC(c5c(F)cc(C(=O)NO)cc5F)CC4)cc(F)cc23)cc1NS(C)(=O)=O]

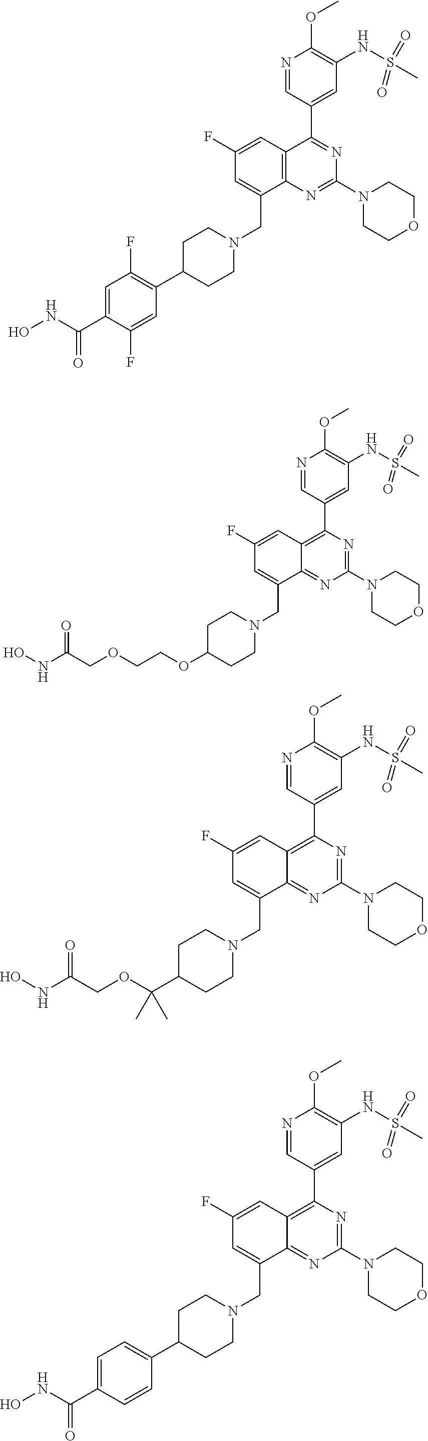 [SMILES: COc1ncc(-c2nc(N3CCOCC3)nc3c(CN4CCC(c5cc(F)c(C(=O)NO)cc5F)CC4)cc(F)cc23)cc1NS(C)(=O)=O ; COc1ncc(-c2nc(N3CCOCC3)nc3c(CN4CCC(OCCOCC(=O)NO)CC4)cc(F)cc23)cc1NS(C)(=O)=O ; COc1ncc(-c2nc(N3CCOCC3)nc3c(CN4CCC(C(C)(C)OCC(=O)NO)CC4)cc(F)cc23)cc1NS(C)(=O)=O ; COc1ncc(-c2nc(N3CCOCC3)nc3c(CN4CCC(c5ccc(C(=O)NO)cc5)CC4)cc(F)cc23)cc1NS(C)(=O)=O]

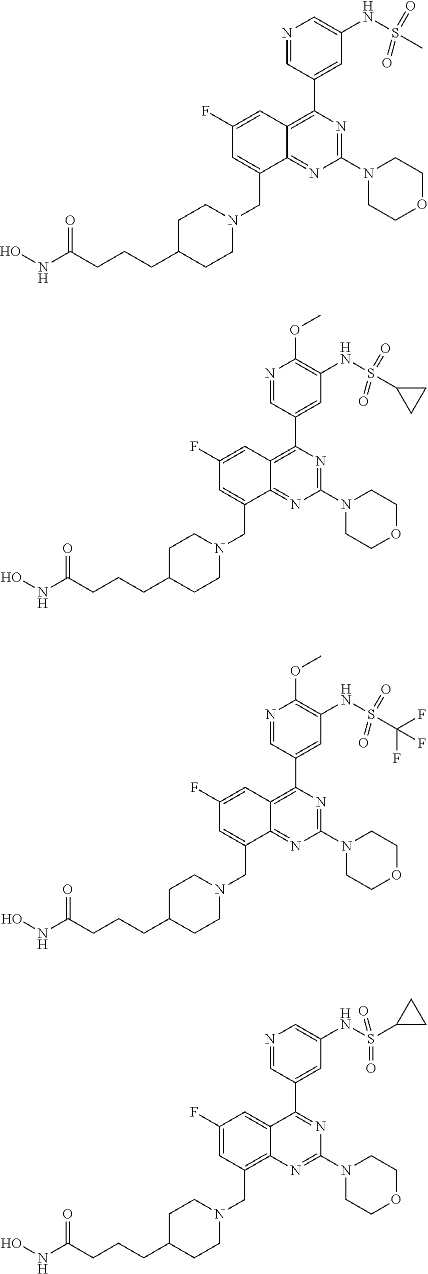 [SMILES: CS(=O)(=O)Nc1cncc(-c2nc(N3CCOCC3)nc3c(CN4CCC(CCCC(=O)NO)CC4)cc(F)cc23)c1 ; COc1ncc(-c2nc(N3CCOCC3)nc3c(CN4CCC(CCCC(=O)NO)CC4)cc(F)cc23)cc1NS(=O)(=O)C1CC1 ; COc1ncc(-c2nc(N3CCOCC3)nc3c(CN4CCC(CCCC(=O)NO)CC4)cc(F)cc23)cc1NS(=O)(=O)C(F)(F)F ; O=C(CCCC1CCN(Cc2cc(F)cc3c(-c4cncc(NS(=O)(=O)C5CC5)c4)nc(N4CCOCC4)nc23)CC1)NO]

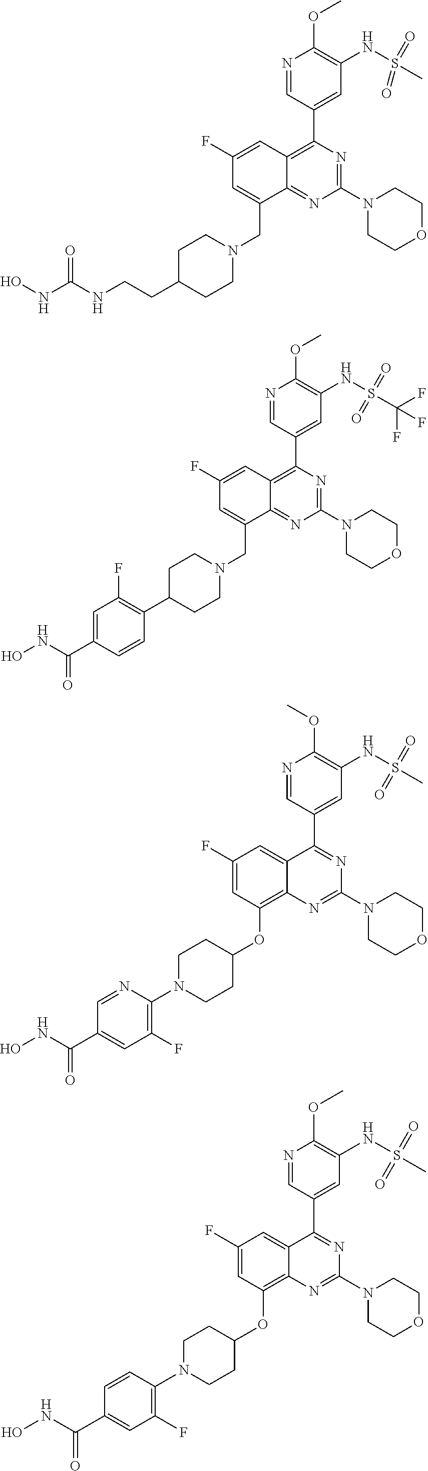 [SMILES: COc1ncc(-c2nc(N3CCOCC3)nc3c(CN4CCC(CCNC(=O)NO)CC4)cc(F)cc23)cc1NS(C)(=O)=O ; COc1ncc(-c2nc(N3CCOCC3)nc3c(CN4CCC(c5ccc(C(=O)NO)cc5F)CC4)cc(F)cc23)cc1NS(=O)(=O)C(F)(F)F ; COc1ncc(-c2nc(N3CCOCC3)nc3c(OC4CCN(c5ncc(C(=O)NO)cc5F)CC4)cc(F)cc23)cc1NS(C)(=O)=O ; COc1ncc(-c2nc(N3CCOCC3)nc3c(OC4CCN(c5ccc(C(=O)NO)cc5F)CC4)cc(F)cc23)cc1NS(C)(=O)=O]

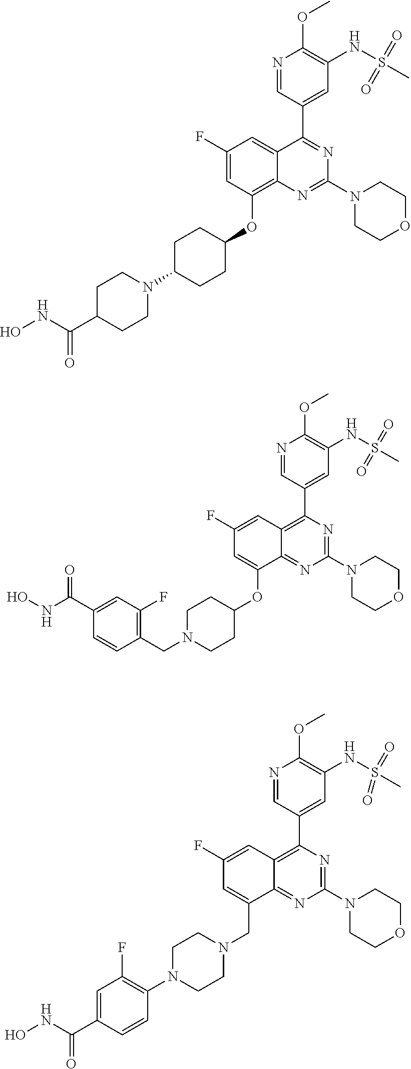 [SMILES: COc1ncc(-c2nc(N3CCOCC3)nc3c(O[C@H]4CC[C@H](N5CCC(C(=O)NO)CC5)CC4)cc(F)cc23)cc1NS(C)(=O)=O ; COc1ncc(-c2nc(N3CCOCC3)nc3c(OC4CCN(Cc5ccc(C(=O)NO)cc5F)CC4)cc(F)cc23)cc1NS(C)(=O)=O ; COc1ncc(-c2nc(N3CCOCC3)nc3c(CN4CCN(c5ccc(C(=O)NO)cc5F)CC4)cc(F)cc23)cc1NS(C)(=O)=O]

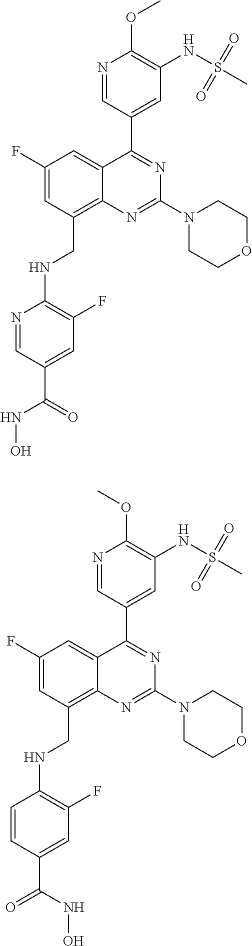 [SMILES: COc1ncc(-c2nc(N3CCOCC3)nc3c(CNc4ncc(C(=O)NO)cc4F)cc(F)cc23)cc1NS(C)(=O)=O ; COc1ncc(-c2nc(N3CCOCC3)nc3c(CNc4ccc(C(=O)NO)cc4F)cc(F)cc23)cc1NS(C)(=O)=O]

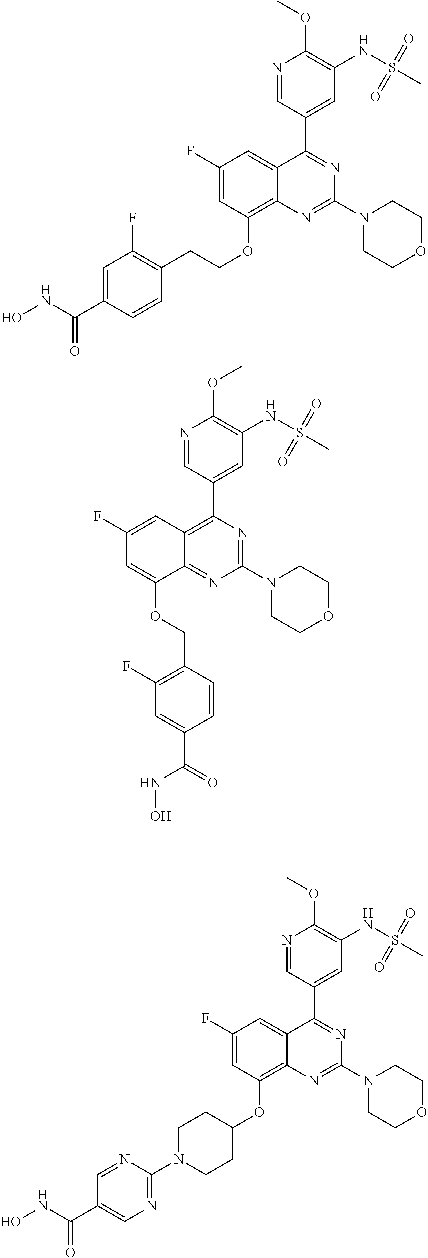 [SMILES: COc1ncc(-c2nc(N3CCOCC3)nc3c(OCCc4ccc(C(=O)NO)cc4F)cc(F)cc23)cc1NS(C)(=O)=O ; COc1ncc(-c2nc(N3CCOCC3)nc3c(OCc4ccc(C(=O)NO)cc4F)cc(F)cc23)cc1NS(C)(=O)=O ; COc1ncc(-c2nc(N3CCOCC3)nc3c(OC4CCN(c5ncc(C(=O)NO)cn5)CC4)cc(F)cc23)cc1NS(C)(=O)=O]

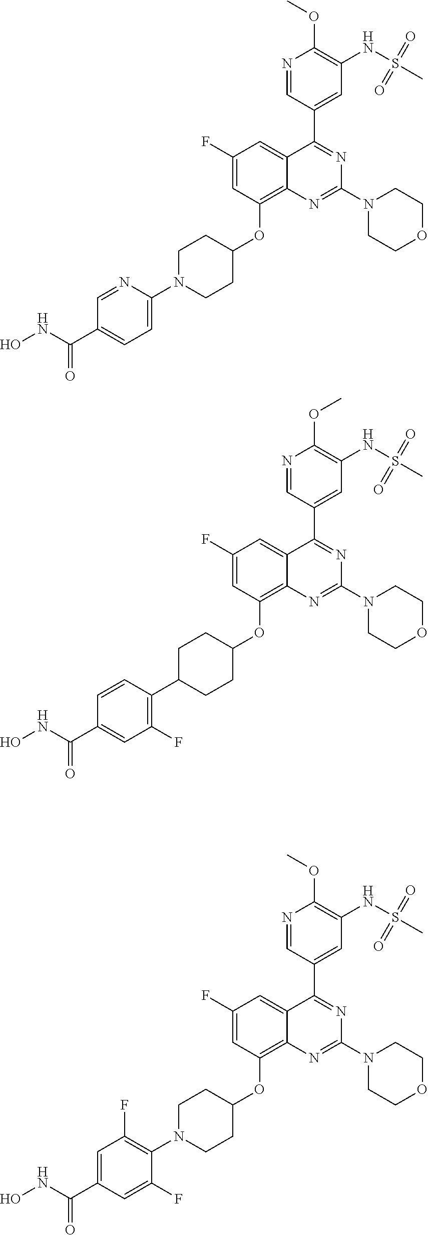 [SMILES: COc1ncc(-c2nc(N3CCOCC3)nc3c(OC4CCN(c5ccc(C(=O)NO)cn5)CC4)cc(F)cc23)cc1NS(C)(=O)=O ; COc1ncc(-c2nc(N3CCOCC3)nc3c(OC4CCC(c5ccc(C(=O)NO)cc5F)CC4)cc(F)cc23)cc1NS(C)(=O)=O ; COc1ncc(-c2nc(N3CCOCC3)nc3c(OC4CCN(c5c(F)cc(C(=O)NO)cc5F)CC4)cc(F)cc23)cc1NS(C)(=O)=O]

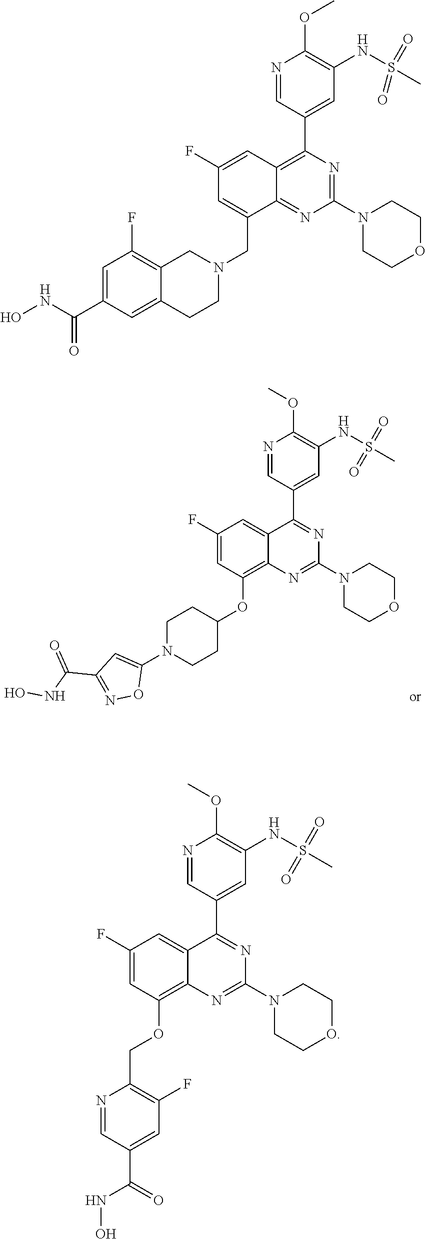 [SMILES: COc1ncc(-c2nc(N3CCOCC3)nc3c(CN4CCc5cc(C(=O)NO)cc(F)c5C4)cc(F)cc23)cc1NS(C)(=O)=O ; COc1ncc(-c2nc(N3CCOCC3)nc3c(OC4CCN(c5cc(C(=O)NO)no5)CC4)cc(F)cc23)cc1NS(C)(=O)=O ; COc1ncc(-c2nc(N3CCOCC3)nc3c(OCc4ncc(C(=O)NO)cc4F)cc(F)cc23)cc1NS(C)(=O)=O]

10. A compound,

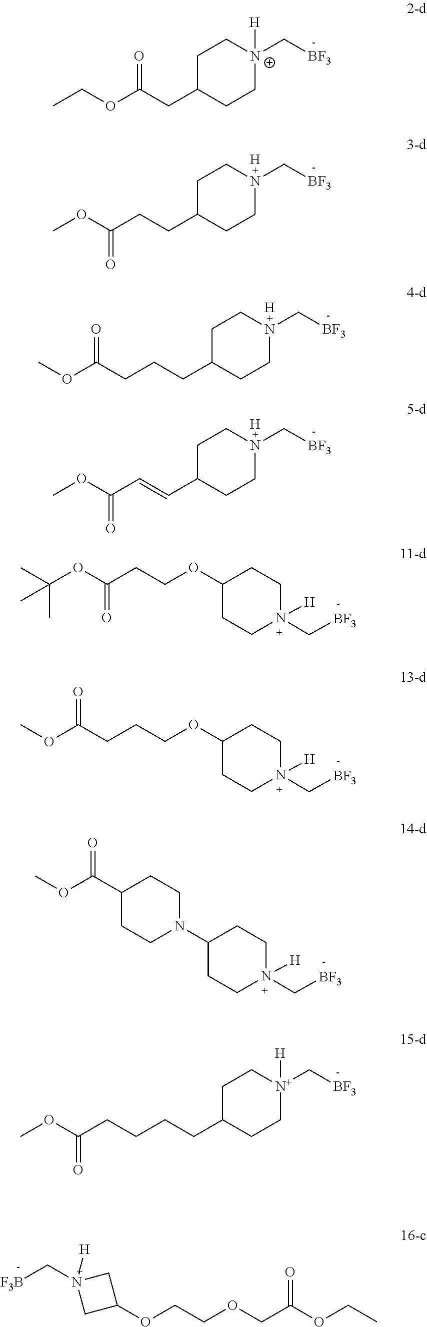

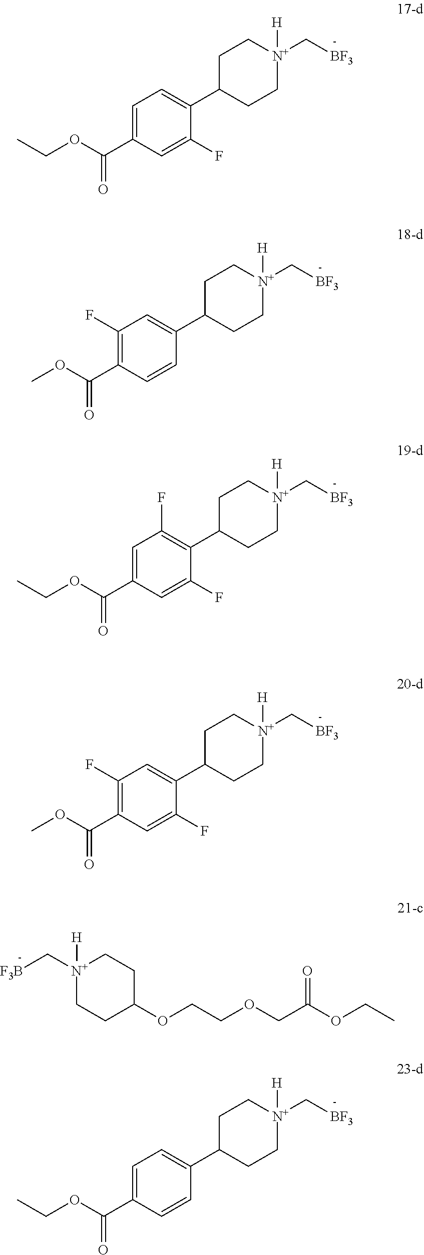

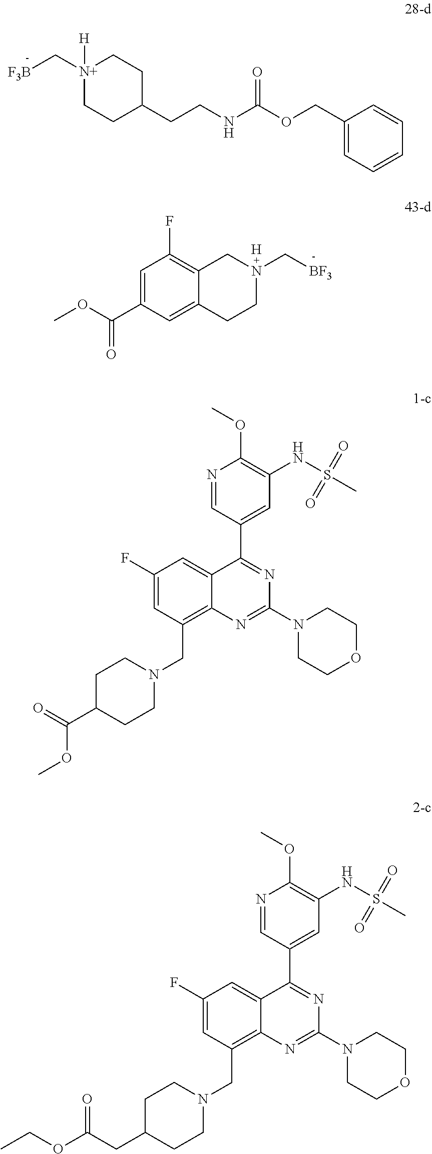

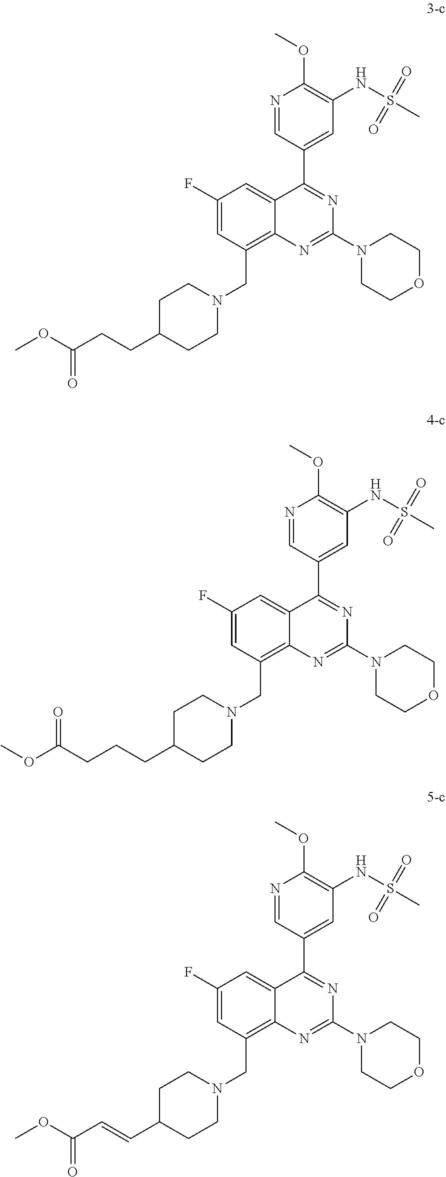 [SMILES: COC(=O)CCC1CCN(Cc2cc(F)cc3c(-c4cnc(OC)c(NS(C)(=O)=O)c4)nc(N4CCOCC4)nc23)CC1 ; COC(=O)CCCC1CCN(Cc2cc(F)cc3c(-c4cnc(OC)c(NS(C)(=O)=O)c4)nc(N4CCOCC4)nc23)CC1 ; COC(=O)/C=C/C1CCN(Cc2cc(F)cc3c(-c4cnc(OC)c(NS(C)(=O)=O)c4)nc(N4CCOCC4)nc23)CC1]

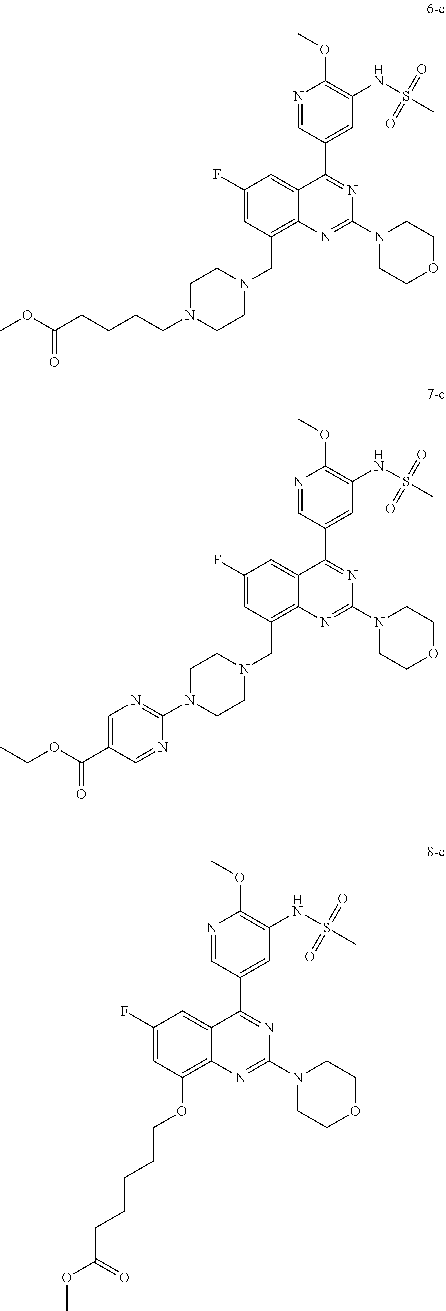 [SMILES: COC(=O)CCCCN1CCN(Cc2cc(F)cc3c(-c4cnc(OC)c(NS(C)(=O)=O)c4)nc(N4CCOCC4)nc23)CC1 ; CCOC(=O)c1cnc(N2CCN(Cc3cc(F)cc4c(-c5cnc(OC)c(NS(C)(=O)=O)c5)nc(N5CCOCC5)nc34)CC2)nc1 ; COC(=O)CCCCCOc1cc(F)cc2c(-c3cnc(OC)c(NS(C)(=O)=O)c3)nc(N3CCOCC3)nc12]

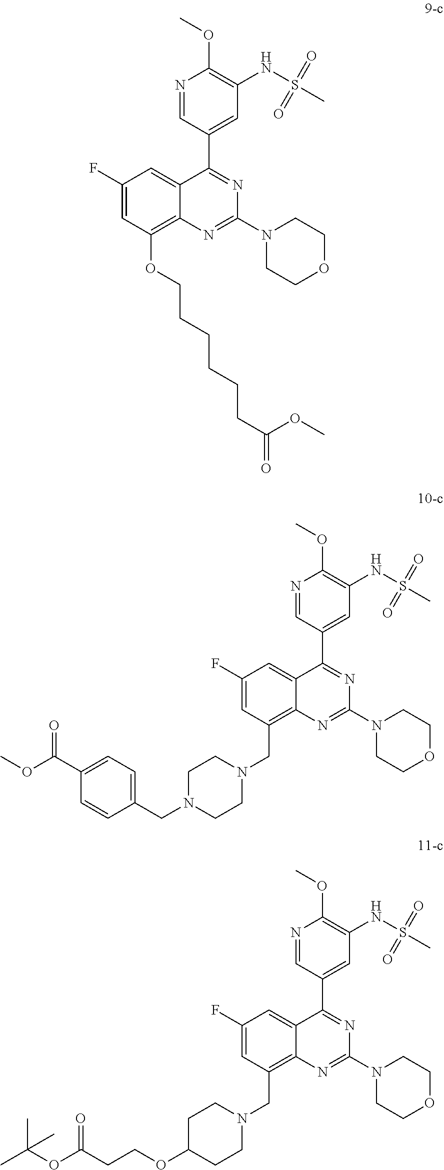 [SMILES: COC(=O)CCCCCCOc1cc(F)cc2c(-c3cnc(OC)c(NS(C)(=O)=O)c3)nc(N3CCOCC3)nc12 ; COC(=O)c1ccc(CN2CCN(Cc3cc(F)cc4c(-c5cnc(OC)c(NS(C)(=O)=O)c5)nc(N5CCOCC5)nc34)CC2)cc1 ; COc1ncc(-c2nc(N3CCOCC3)nc3c(CN4CCC(OCCC(=O)OC(C)(C)C)CC4)cc(F)cc23)cc1NS(C)(=O)=O]

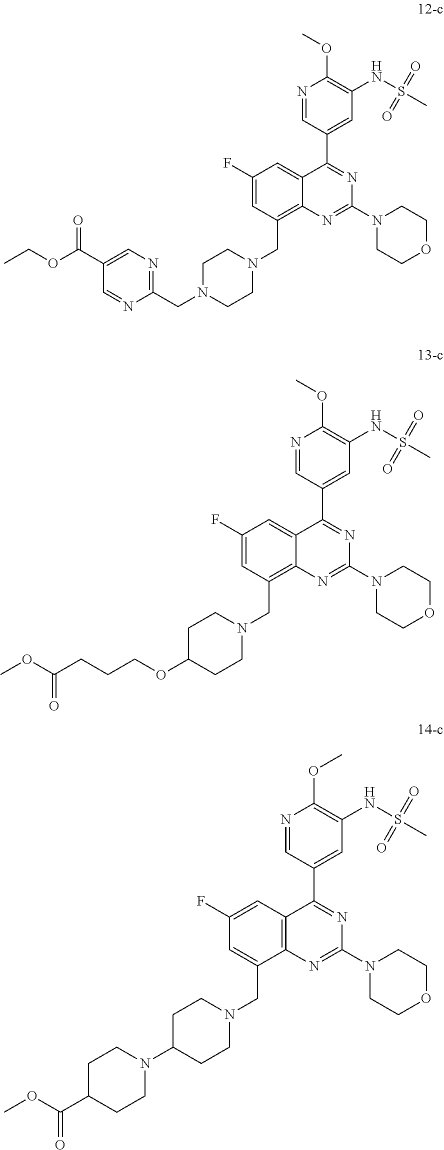 [SMILES: CCOC(=O)c1cnc(CN2CCN(Cc3cc(F)cc4c(-c5cnc(OC)c(NS(C)(=O)=O)c5)nc(N5CCOCC5)nc34)CC2)nc1 ; COC(=O)CCCOC1CCN(Cc2cc(F)cc3c(-c4cnc(OC)c(NS(C)(=O)=O)c4)nc(N4CCOCC4)nc23)CC1 ; COC(=O)C1CCN(C2CCN(Cc3cc(F)cc4c(-c5cnc(OC)c(NS(C)(=O)=O)c5)nc(N5CCOCC5)nc34)CC2)CC1]

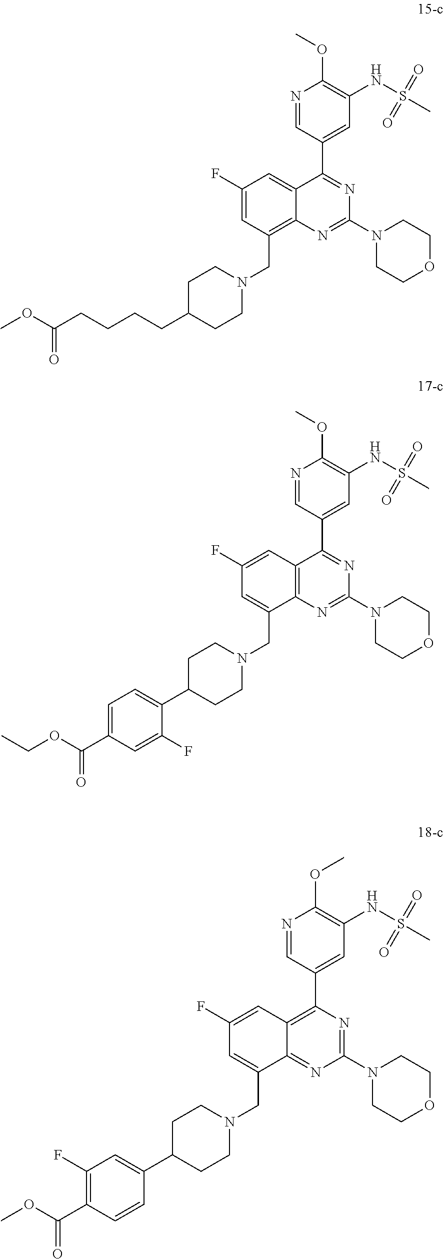 [SMILES: COC(=O)CCCCC1CCN(Cc2cc(F)cc3c(-c4cnc(OC)c(NS(C)(=O)=O)c4)nc(N4CCOCC4)nc23)CC1 ; CCOC(=O)c1ccc(C2CCN(Cc3cc(F)cc4c(-c5cnc(OC)c(NS(C)(=O)=O)c5)nc(N5CCOCC5)nc34)CC2)c(F)c1 ; COC(=O)c1ccc(C2CCN(Cc3cc(F)cc4c(-c5cnc(OC)c(NS(C)(=O)=O)c5)nc(N5CCOCC5)nc34)CC2)cc1F]

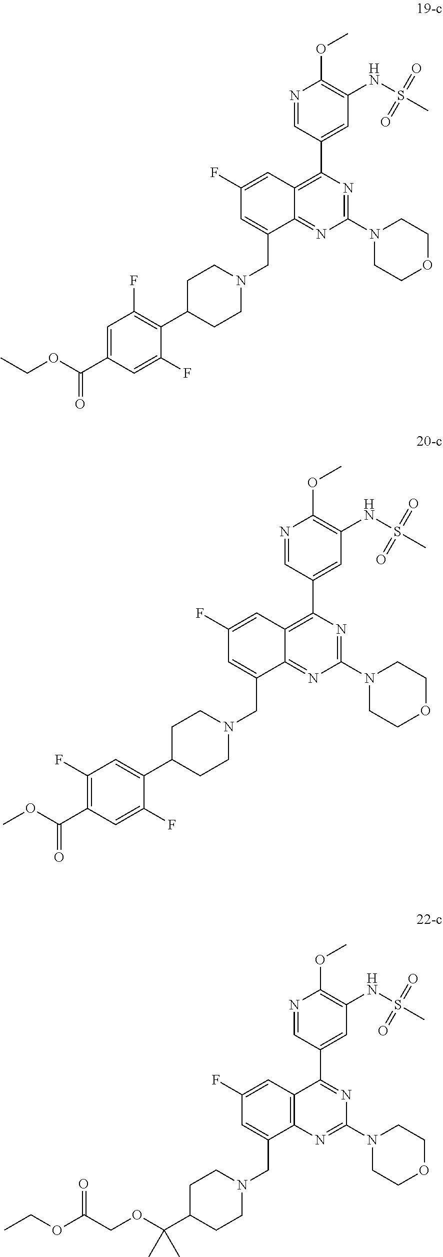 [SMILES: CCOC(=O)c1cc(F)c(C2CCN(Cc3cc(F)cc4c(-c5cnc(OC)c(NS(C)(=O)=O)c5)nc(N5CCOCC5)nc34)CC2)c(F)c1 ; COC(=O)c1cc(F)c(C2CCN(Cc3cc(F)cc4c(-c5cnc(OC)c(NS(C)(=O)=O)c5)nc(N5CCOCC5)nc34)CC2)cc1F ; CCOC(=O)COC(C)(C)C1CCN(Cc2cc(F)cc3c(-c4cnc(OC)c(NS(C)(=O)=O)c4)nc(N4CCOCC4)nc23)CC1]

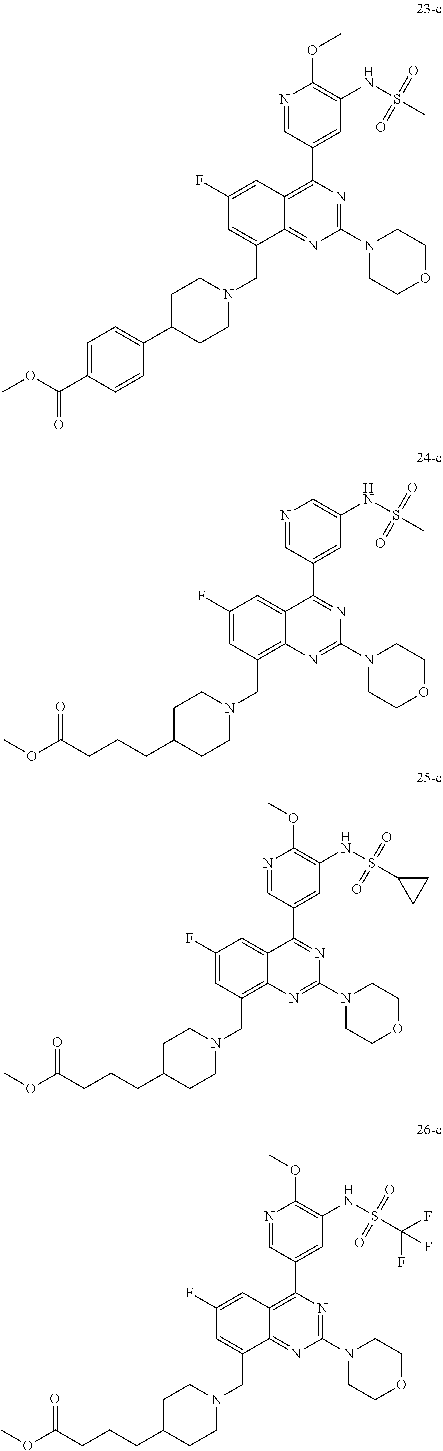 [SMILES: COC(=O)c1ccc(C2CCN(Cc3cc(F)cc4c(-c5cnc(OC)c(NS(C)(=O)=O)c5)nc(N5CCOCC5)nc34)CC2)cc1 ; COC(=O)CCCC1CCN(Cc2cc(F)cc3c(-c4cncc(NS(C)(=O)=O)c4)nc(N4CCOCC4)nc23)CC1 ; COC(=O)CCCC1CCN(Cc2cc(F)cc3c(-c4cnc(OC)c(NS(=O)(=O)C5CC5)c4)nc(N4CCOCC4)nc23)CC1 ; COC(=O)CCCC1CCN(Cc2cc(F)cc3c(-c4cnc(OC)c(NS(=O)(=O)C(F)(F)F)c4)nc(N4CCOCC4)nc23)CC1]

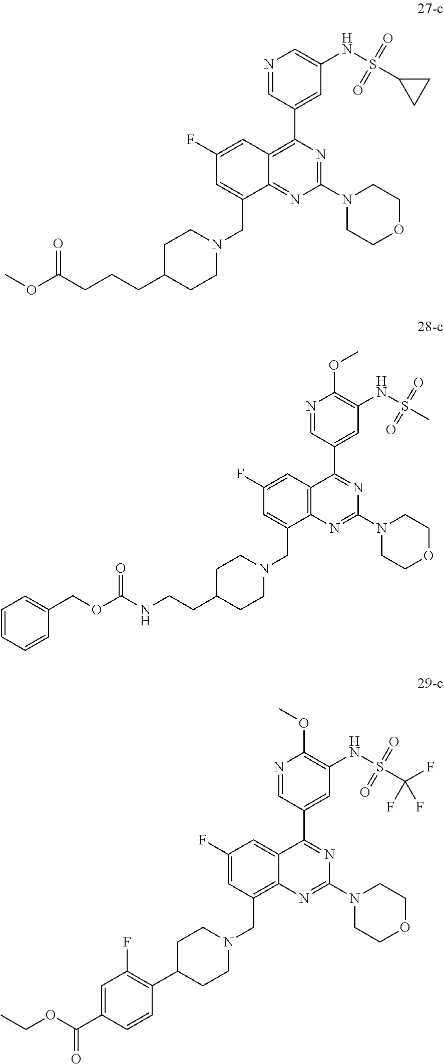

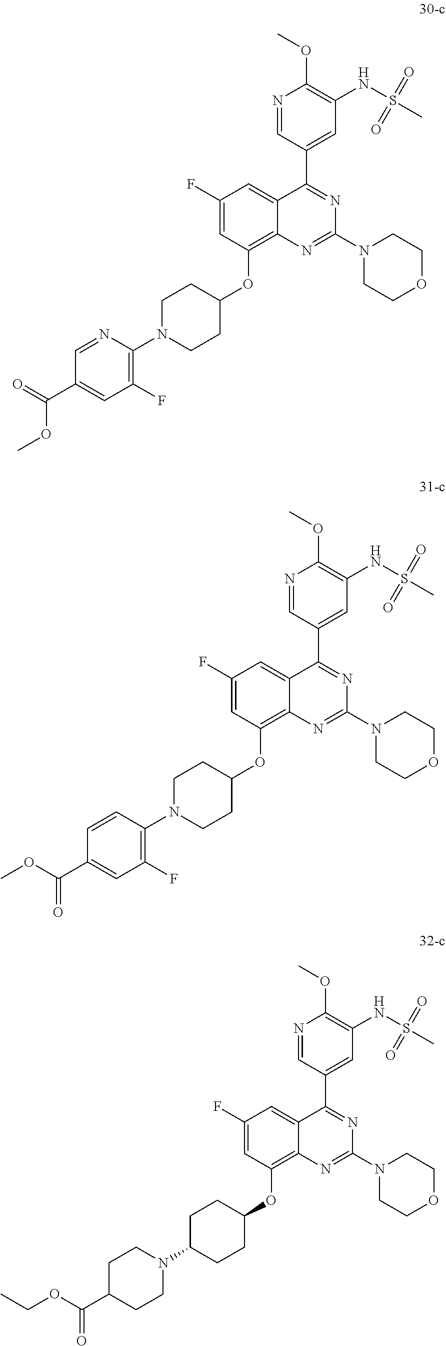

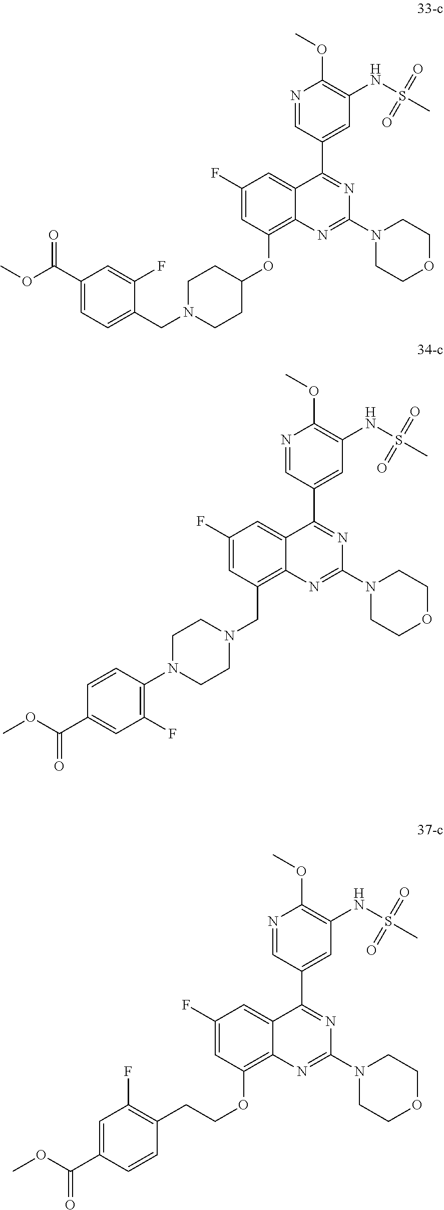

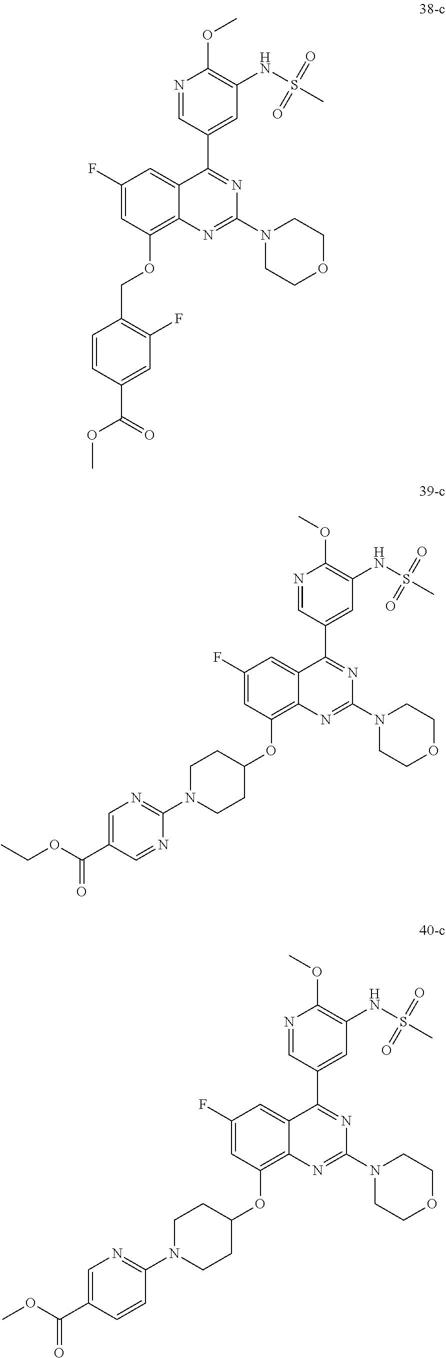

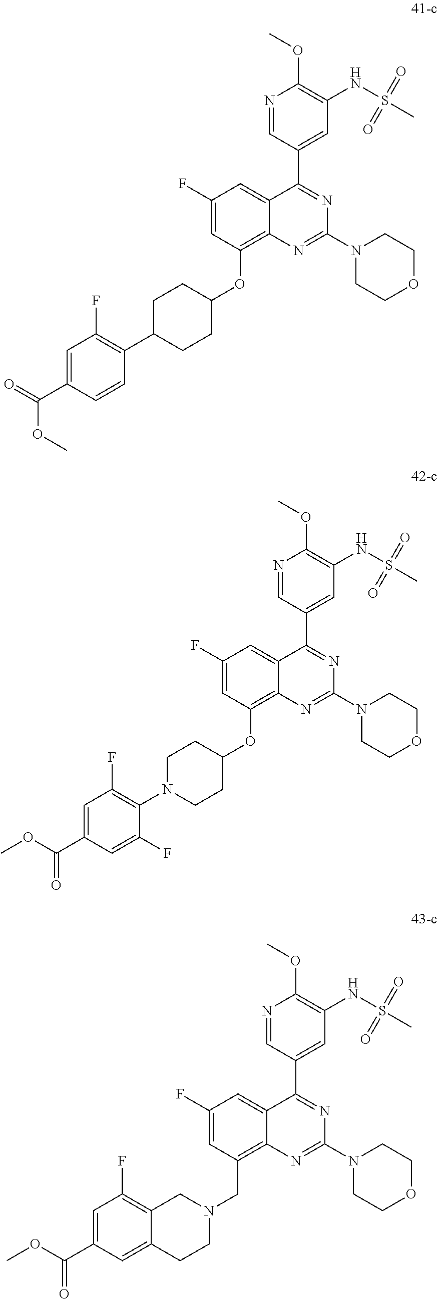

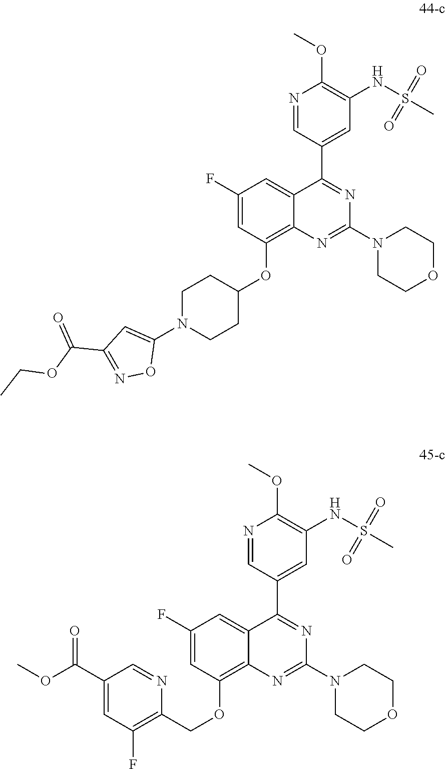

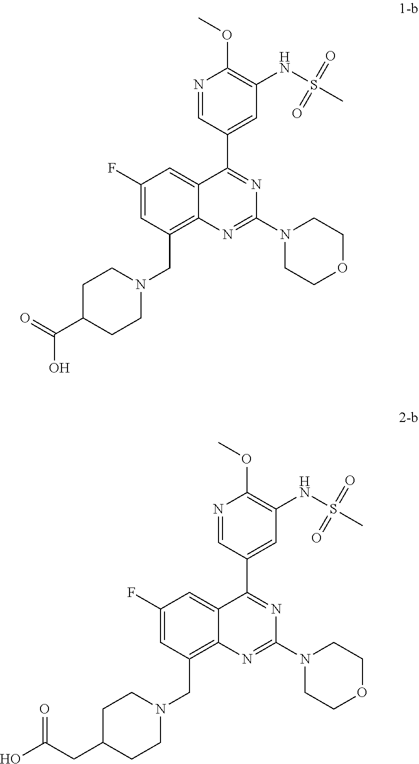 [SMILES: COc1ncc(-c2nc(N3CCOCC3)nc3c(CN4CCC(C(=O)O)CC4)cc(F)cc23)cc1NS(C)(=O)=O ; COc1ncc(-c2nc(N3CCOCC3)nc3c(CN4CCC(CC(=O)O)CC4)cc(F)cc23)cc1NS(C)(=O)=O]

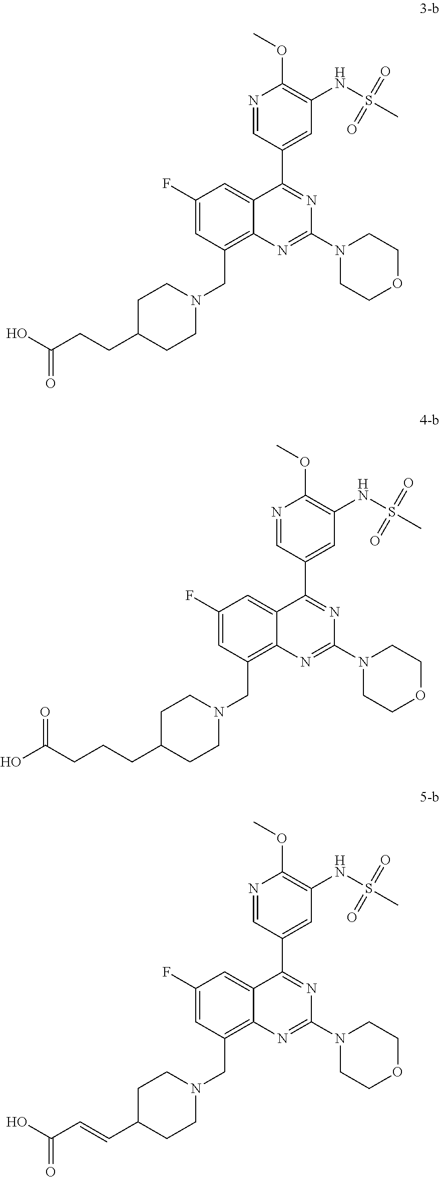 [SMILES: COc1ncc(-c2nc(N3CCOCC3)nc3c(CN4CCC(CCC(=O)O)CC4)cc(F)cc23)cc1NS(C)(=O)=O ; COc1ncc(-c2nc(N3CCOCC3)nc3c(CN4CCC(CCCC(=O)O)CC4)cc(F)cc23)cc1NS(C)(=O)=O ; COc1ncc(-c2nc(N3CCOCC3)nc3c(CN4CCC(/C=C/C(=O)O)CC4)cc(F)cc23)cc1NS(C)(=O)=O]

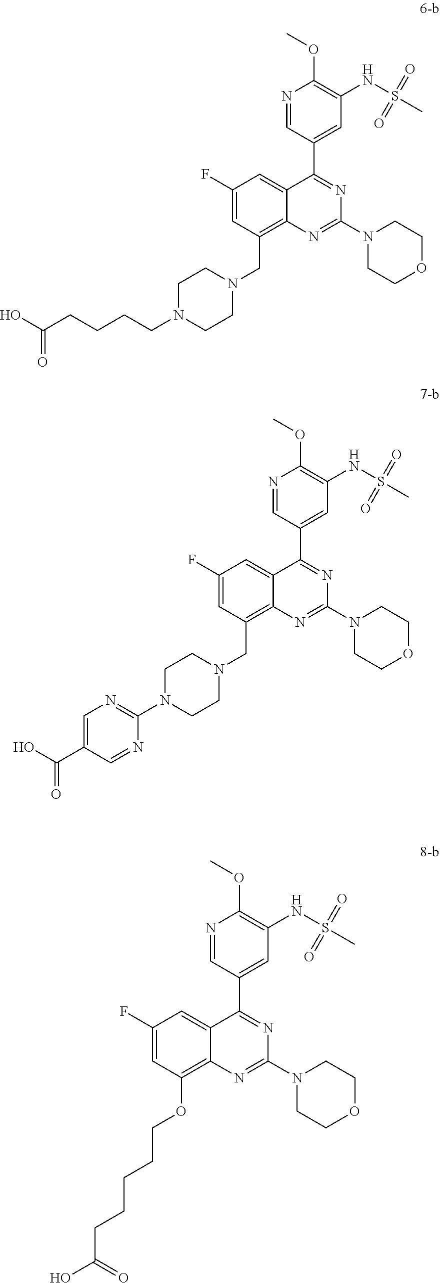 [SMILES: COc1ncc(-c2nc(N3CCOCC3)nc3c(CN4CCN(CCCCC(=O)O)CC4)cc(F)cc23)cc1NS(C)(=O)=O ; COc1ncc(-c2nc(N3CCOCC3)nc3c(CN4CCN(c5ncc(C(=O)O)cn5)CC4)cc(F)cc23)cc1NS(C)(=O)=O ; COc1ncc(-c2nc(N3CCOCC3)nc3c(OCCCCCC(=O)O)cc(F)cc23)cc1NS(C)(=O)=O]

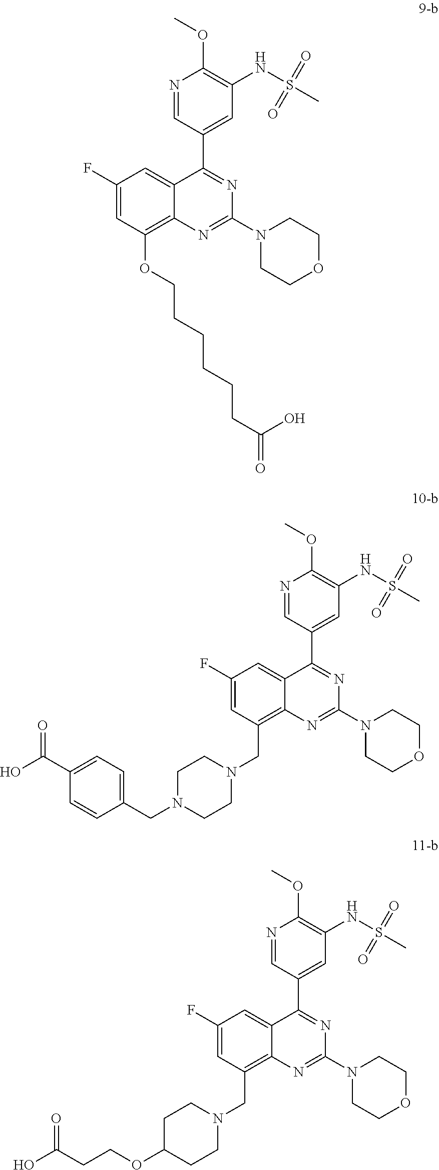 [SMILES: COc1ncc(-c2nc(N3CCOCC3)nc3c(OCCCCCCC(=O)O)cc(F)cc23)cc1NS(C)(=O)=O ; COc1ncc(-c2nc(N3CCOCC3)nc3c(CN4CCN(Cc5ccc(C(=O)O)cc5)CC4)cc(F)cc23)cc1NS(C)(=O)=O ; COc1ncc(-c2nc(N3CCOCC3)nc3c(CN4CCC(OCCC(=O)O)CC4)cc(F)cc23)cc1NS(C)(=O)=O]

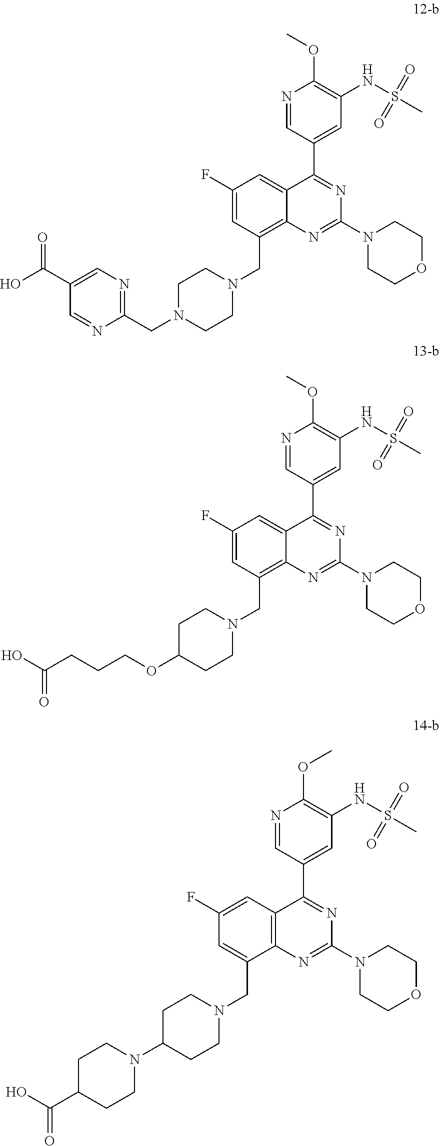 [SMILES: COc1ncc(-c2nc(N3CCOCC3)nc3c(CN4CCN(Cc5ncc(C(=O)O)cn5)CC4)cc(F)cc23)cc1NS(C)(=O)=O ; COc1ncc(-c2nc(N3CCOCC3)nc3c(CN4CCC(OCCCC(=O)O)CC4)cc(F)cc23)cc1NS(C)(=O)=O ; COc1ncc(-c2nc(N3CCOCC3)nc3c(CN4CCC(N5CCC(C(=O)O)CC5)CC4)cc(F)cc23)cc1NS(C)(=O)=O]

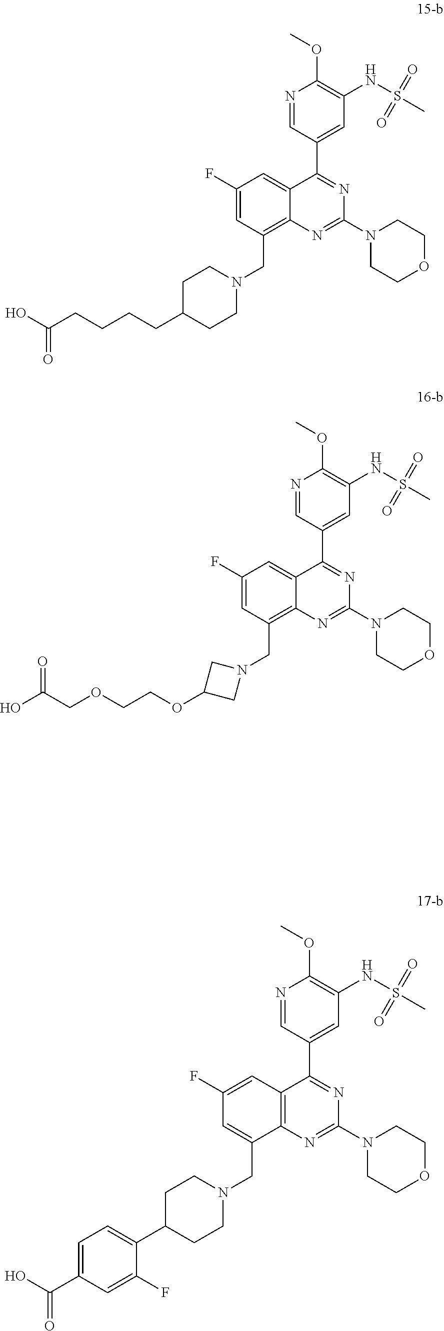 [SMILES: COc1ncc(-c2nc(N3CCOCC3)nc3c(CN4CCC(CCCCC(=O)O)CC4)cc(F)cc23)cc1NS(C)(=O)=O ; COc1ncc(-c2nc(N3CCOCC3)nc3c(CN4CC(OCCOCC(=O)O)C4)cc(F)cc23)cc1NS(C)(=O)=O ; COc1ncc(-c2nc(N3CCOCC3)nc3c(CN4CCC(c5ccc(C(=O)O)cc5F)CC4)cc(F)cc23)cc1NS(C)(=O)=O]

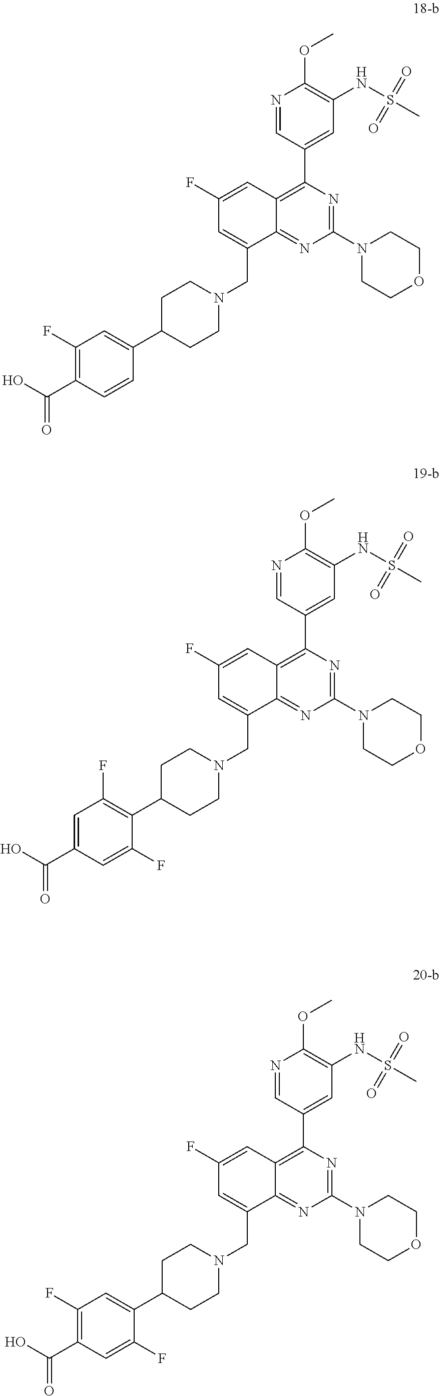 [SMILES: COc1ncc(-c2nc(N3CCOCC3)nc3c(CN4CCC(c5ccc(C(=O)O)c(F)c5)CC4)cc(F)cc23)cc1NS(C)(=O)=O ; COc1ncc(-c2nc(N3CCOCC3)nc3c(CN4CCC(c5c(F)cc(C(=O)O)cc5F)CC4)cc(F)cc23)cc1NS(C)(=O)=O ; COc1ncc(-c2nc(N3CCOCC3)nc3c(CN4CCC(c5cc(F)c(C(=O)O)cc5F)CC4)cc(F)cc23)cc1NS(C)(=O)=O]

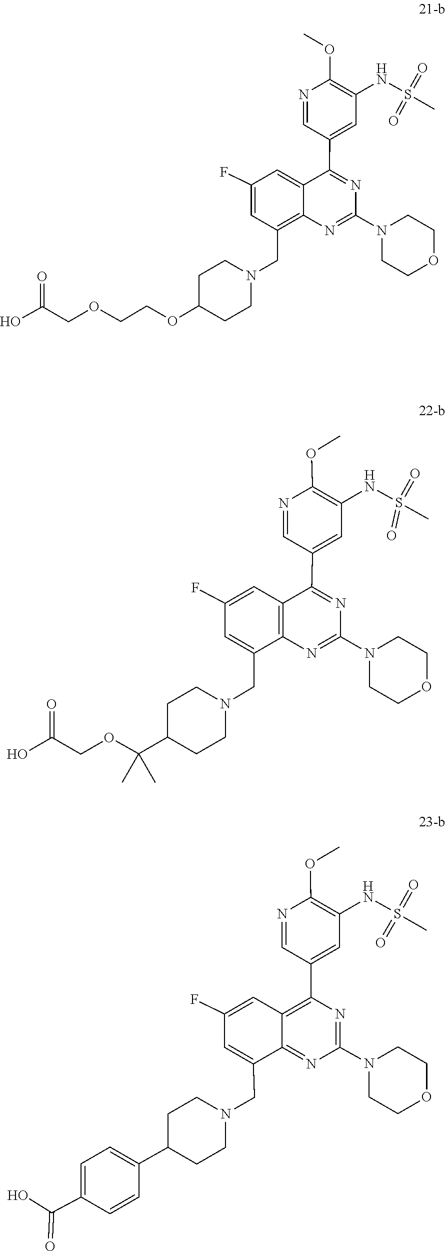 [SMILES: COc1ncc(-c2nc(N3CCOCC3)nc3c(CN4CCC(OCCOCC(=O)O)CC4)cc(F)cc23)cc1NS(C)(=O)=O ; COc1ncc(-c2nc(N3CCOCC3)nc3c(CN4CCC(C(C)(C)OCC(=O)O)CC4)cc(F)cc23)cc1NS(C)(=O)=O ; COc1ncc(-c2nc(N3CCOCC3)nc3c(CN4CCC(c5ccc(C(=O)O)cc5)CC4)cc(F)cc23)cc1NS(C)(=O)=O]

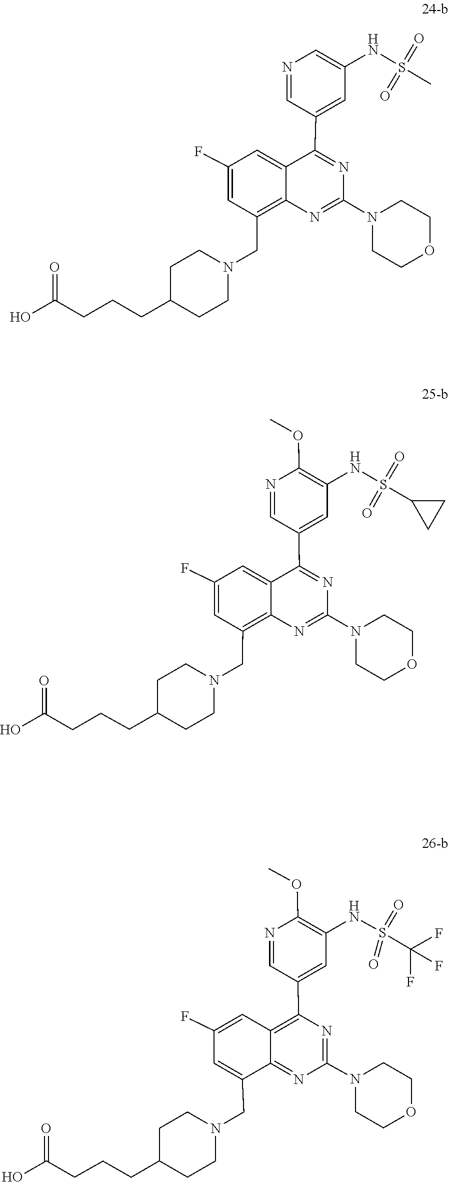 [SMILES: CS(=O)(=O)Nc1cncc(-c2nc(N3CCOCC3)nc3c(CN4CCC(CCCC(=O)O)CC4)cc(F)cc23)c1 ; COc1ncc(-c2nc(N3CCOCC3)nc3c(CN4CCC(CCCC(=O)O)CC4)cc(F)cc23)cc1NS(=O)(=O)C1CC1 ; COc1ncc(-c2nc(N3CCOCC3)nc3c(CN4CCC(CCCC(=O)O)CC4)cc(F)cc23)cc1NS(=O)(=O)C(F)(F)F]

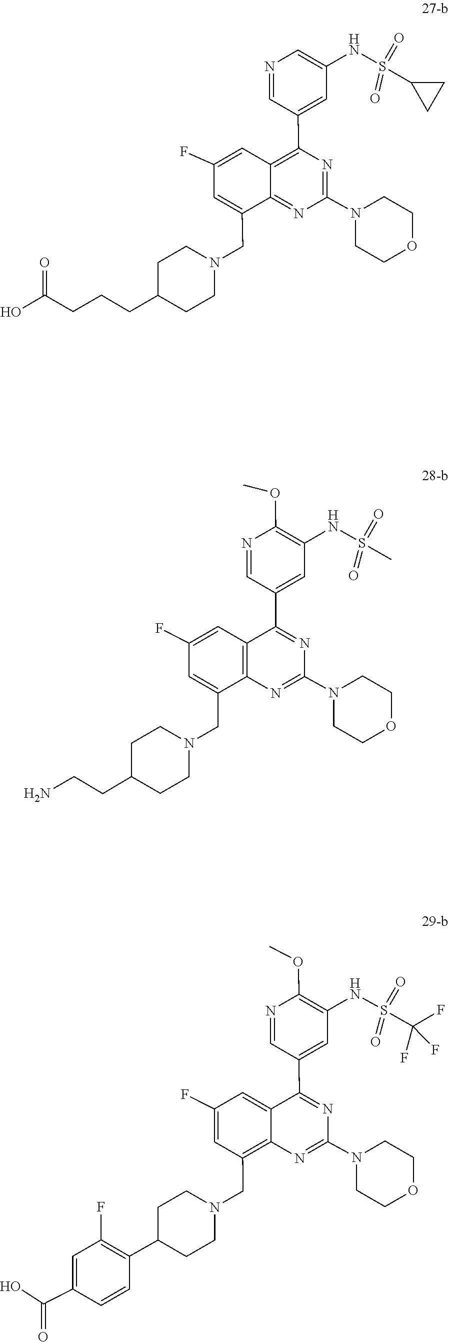 [SMILES: O=C(O)CCCC1CCN(Cc2cc(F)cc3c(-c4cncc(NS(=O)(=O)C5CC5)c4)nc(N4CCOCC4)nc23)CC1 ; COc1ncc(-c2nc(N3CCOCC3)nc3c(CN4CCC(CCN)CC4)cc(F)cc23)cc1NS(C)(=O)=O ; COc1ncc(-c2nc(N3CCOCC3)nc3c(CN4CCC(c5ccc(C(=O)O)cc5F)CC4)cc(F)cc23)cc1NS(=O)(=O)C(F)(F)F]

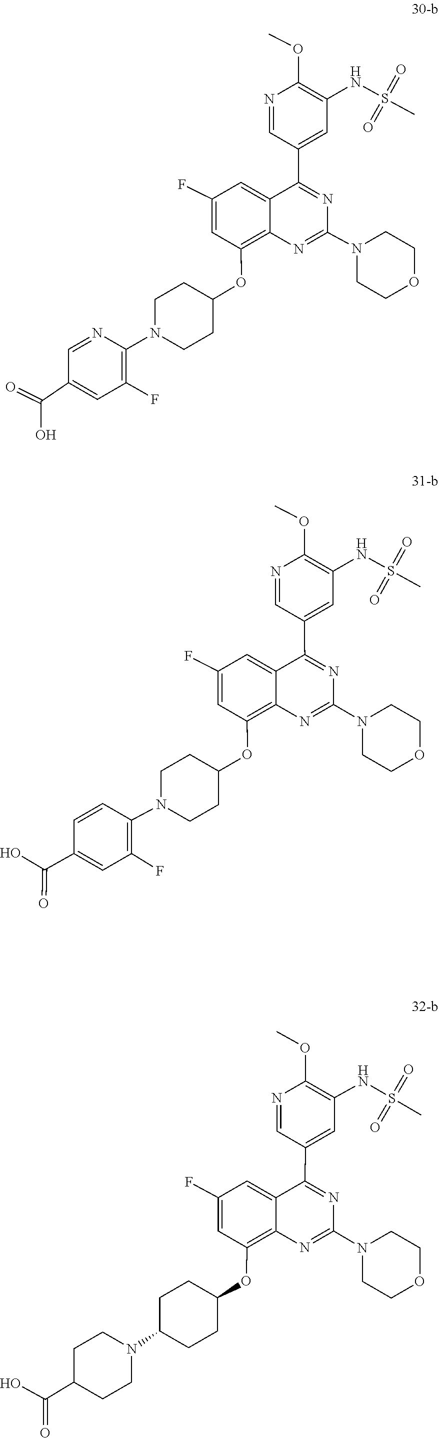 [SMILES: COc1ncc(-c2nc(N3CCOCC3)nc3c(OC4CCN(c5ncc(C(=O)O)cc5F)CC4)cc(F)cc23)cc1NS(C)(=O)=O ; COc1ncc(-c2nc(N3CCOCC3)nc3c(OC4CCN(c5ccc(C(=O)O)cc5F)CC4)cc(F)cc23)cc1NS(C)(=O)=O ; COc1ncc(-c2nc(N3CCOCC3)nc3c(O[C@H]4CC[C@H](N5CCC(C(=O)O)CC5)CC4)cc(F)cc23)cc1NS(C)(=O)=O]

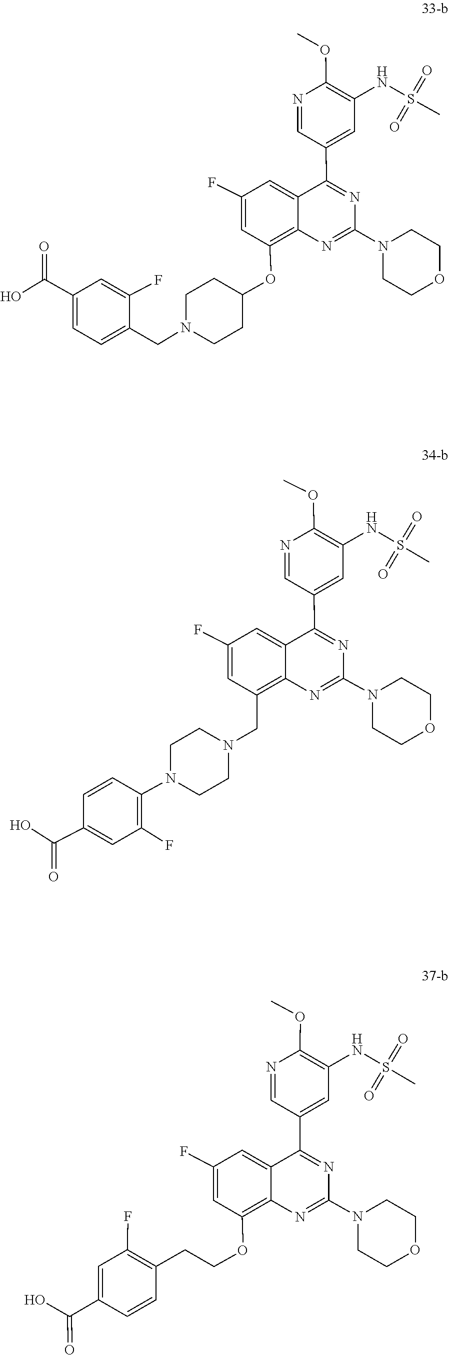 [SMILES: COc1ncc(-c2nc(N3CCOCC3)nc3c(OC4CCN(Cc5ccc(C(=O)O)cc5F)CC4)cc(F)cc23)cc1NS(C)(=O)=O ; COc1ncc(-c2nc(N3CCOCC3)nc3c(CN4CCN(c5ccc(C(=O)O)cc5F)CC4)cc(F)cc23)cc1NS(C)(=O)=O ; COc1ncc(-c2nc(N3CCOCC3)nc3c(OCCc4ccc(C(=O)O)cc4F)cc(F)cc23)cc1NS(C)(=O)=O]

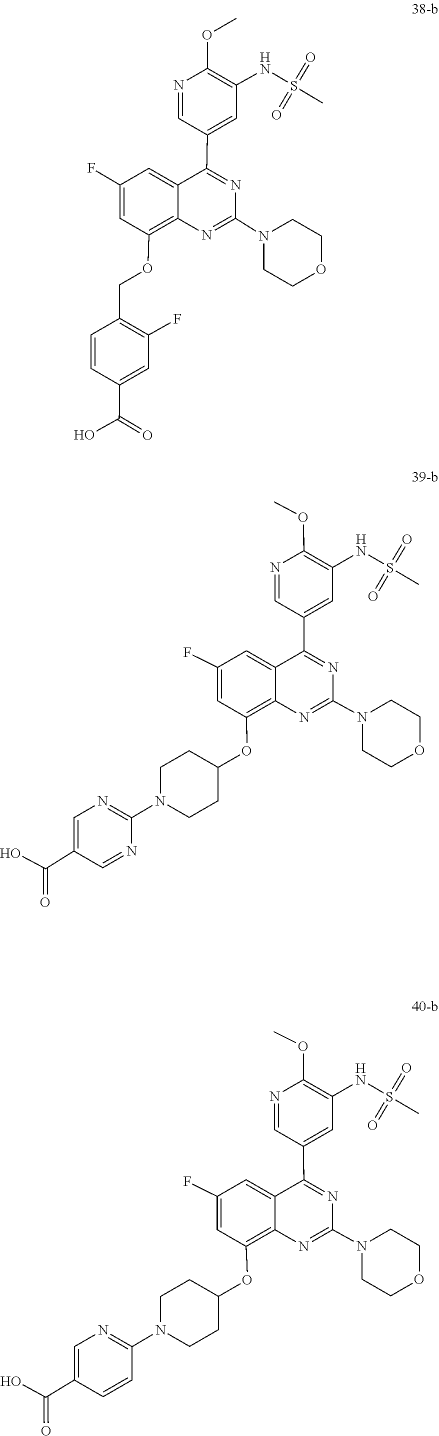 [SMILES: COc1ncc(-c2nc(N3CCOCC3)nc3c(OCc4ccc(C(=O)O)cc4F)cc(F)cc23)cc1NS(C)(=O)=O ; COc1ncc(-c2nc(N3CCOCC3)nc3c(OC4CCN(c5ncc(C(=O)O)cn5)CC4)cc(F)cc23)cc1NS(C)(=O)=O ; COc1ncc(-c2nc(N3CCOCC3)nc3c(OC4CCN(c5ccc(C(=O)O)cn5)CC4)cc(F)cc23)cc1NS(C)(=O)=O]

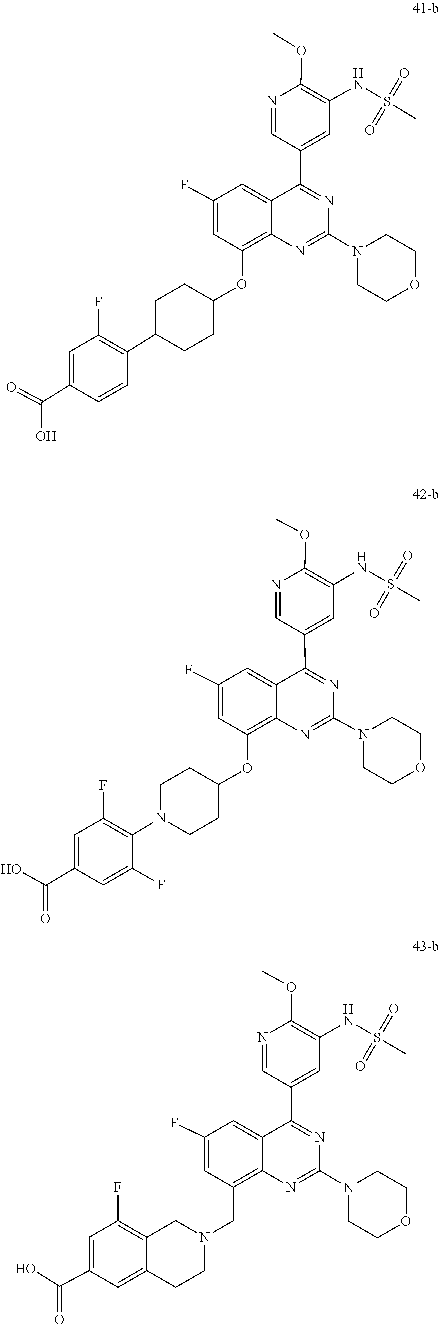 [SMILES: COc1ncc(-c2nc(N3CCOCC3)nc3c(OC4CCC(c5ccc(C(=O)O)cc5F)CC4)cc(F)cc23)cc1NS(C)(=O)=O ; COc1ncc(-c2nc(N3CCOCC3)nc3c(OC4CCN(c5c(F)cc(C(=O)O)cc5F)CC4)cc(F)cc23)cc1NS(C)(=O)=O ; COc1ncc(-c2nc(N3CCOCC3)nc3c(CN4CCc5cc(C(=O)O)cc(F)c5C4)cc(F)cc23)cc1NS(C)(=O)=O]

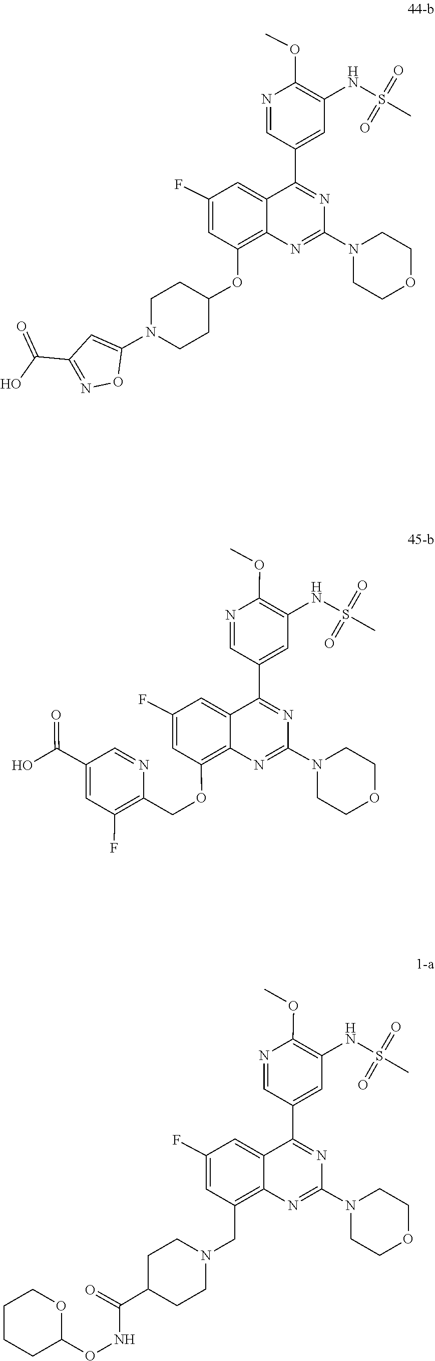 [SMILES: COc1ncc(-c2nc(N3CCOCC3)nc3c(OC4CCN(c5cc(C(=O)O)no5)CC4)cc(F)cc23)cc1NS(C)(=O)=O ; COc1ncc(-c2nc(N3CCOCC3)nc3c(OCc4ncc(C(=O)O)cc4F)cc(F)cc23)cc1NS(C)(=O)=O ; COc1ncc(-c2nc(N3CCOCC3)nc3c(CN4CCC(C(=O)NOC5CCCCO5)CC4)cc(F)cc23)cc1NS(C)(=O)=O]

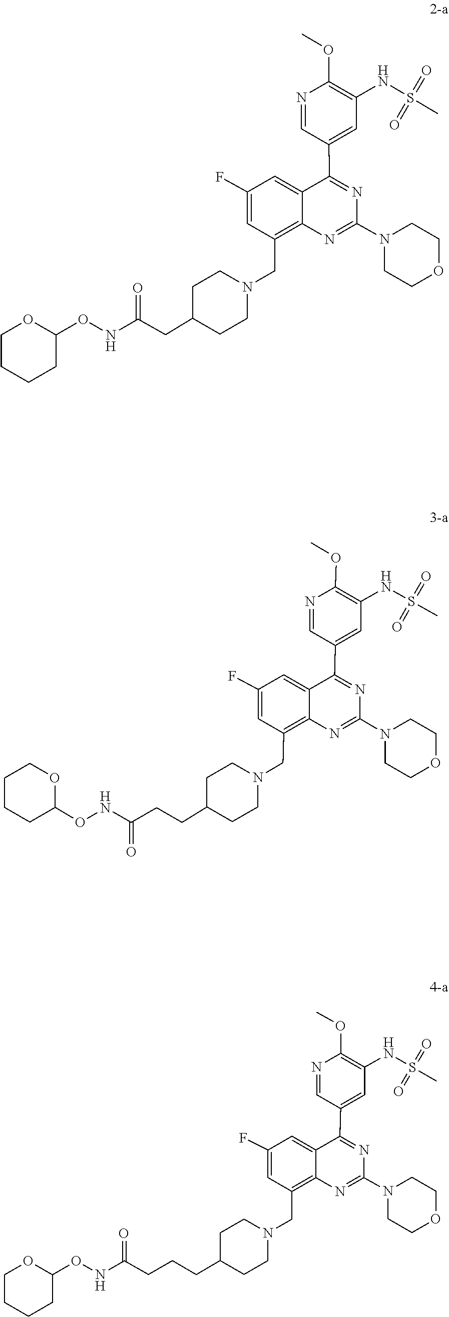 [SMILES: COc1ncc(-c2nc(N3CCOCC3)nc3c(CN4CCC(CC(=O)NOC5CCCCO5)CC4)cc(F)cc23)cc1NS(C)(=O)=O ; COc1ncc(-c2nc(N3CCOCC3)nc3c(CN4CCC(CCC(=O)NOC5CCCCO5)CC4)cc(F)cc23)cc1NS(C)(=O)=O ; COc1ncc(-c2nc(N3CCOCC3)nc3c(CN4CCC(CCCC(=O)NOC5CCCCO5)CC4)cc(F)cc23)cc1NS(C)(=O)=O]

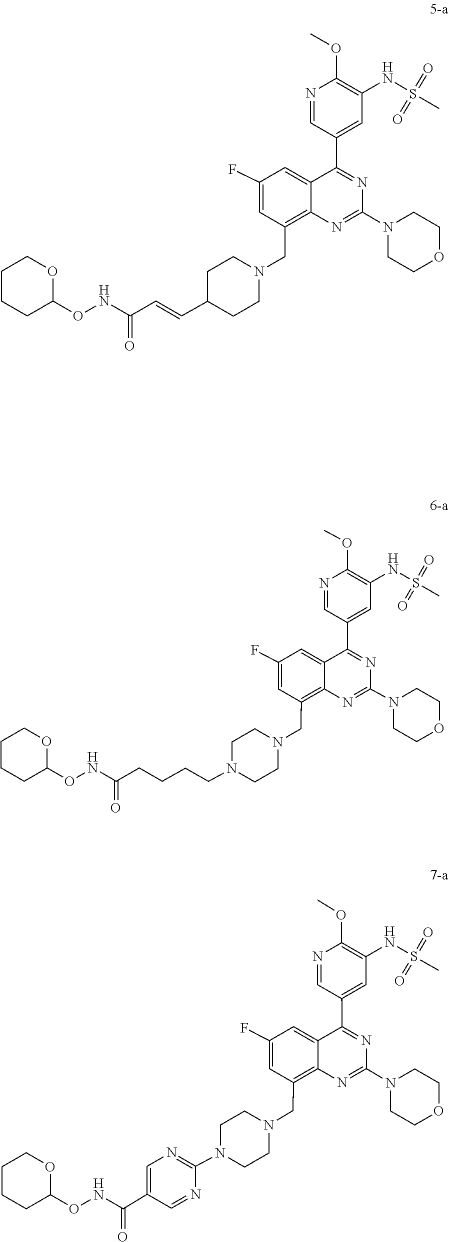 [SMILES: COc1ncc(-c2nc(N3CCOCC3)nc3c(CN4CCC(/C=C/C(=O)NOC5CCCCO5)CC4)cc(F)cc23)cc1NS(C)(=O)=O ; COc1ncc(-c2nc(N3CCOCC3)nc3c(CN4CCN(CCCCC(=O)NOC5CCCCO5)CC4)cc(F)cc23)cc1NS(C)(=O)=O ; COc1ncc(-c2nc(N3CCOCC3)nc3c(CN4CCN(c5ncc(C(=O)NOC6CCCCO6)cn5)CC4)cc(F)cc23)cc1NS(C)(=O)=O]

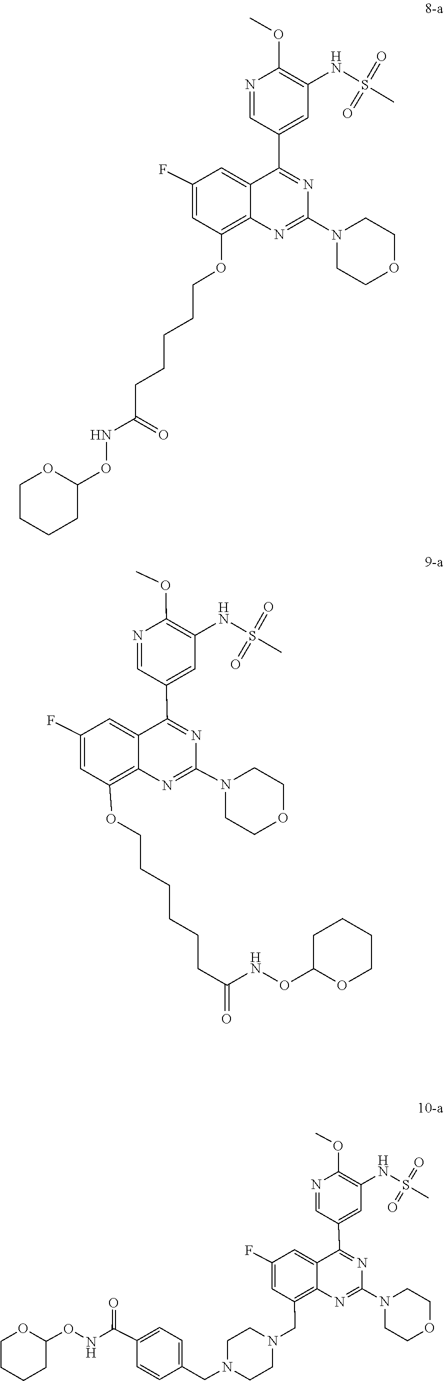 [SMILES: COc1ncc(-c2nc(N3CCOCC3)nc3c(OCCCCCC(=O)NOC4CCCCO4)cc(F)cc23)cc1NS(C)(=O)=O ; COc1ncc(-c2nc(N3CCOCC3)nc3c(OCCCCCCC(=O)NOC4CCCCO4)cc(F)cc23)cc1NS(C)(=O)=O ; COc1ncc(-c2nc(N3CCOCC3)nc3c(CN4CCN(Cc5ccc(C(=O)NOC6CCCCO6)cc5)CC4)cc(F)cc23)cc1NS(C)(=O)=O]

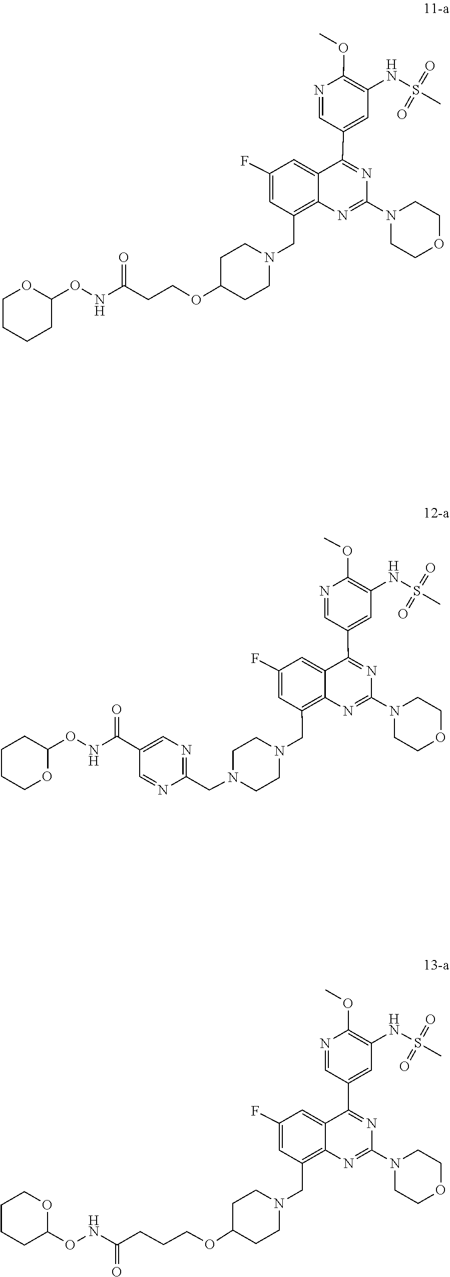 [SMILES: COc1ncc(-c2nc(N3CCOCC3)nc3c(CN4CCC(OCCC(=O)NOC5CCCCO5)CC4)cc(F)cc23)cc1NS(C)(=O)=O ; COc1ncc(-c2nc(N3CCOCC3)nc3c(CN4CCN(Cc5ncc(C(=O)NOC6CCCCO6)cn5)CC4)cc(F)cc23)cc1NS(C)(=O)=O ; COc1ncc(-c2nc(N3CCOCC3)nc3c(CN4CCC(OCCCC(=O)NOC5CCCCO5)CC4)cc(F)cc23)cc1NS(C)(=O)=O]

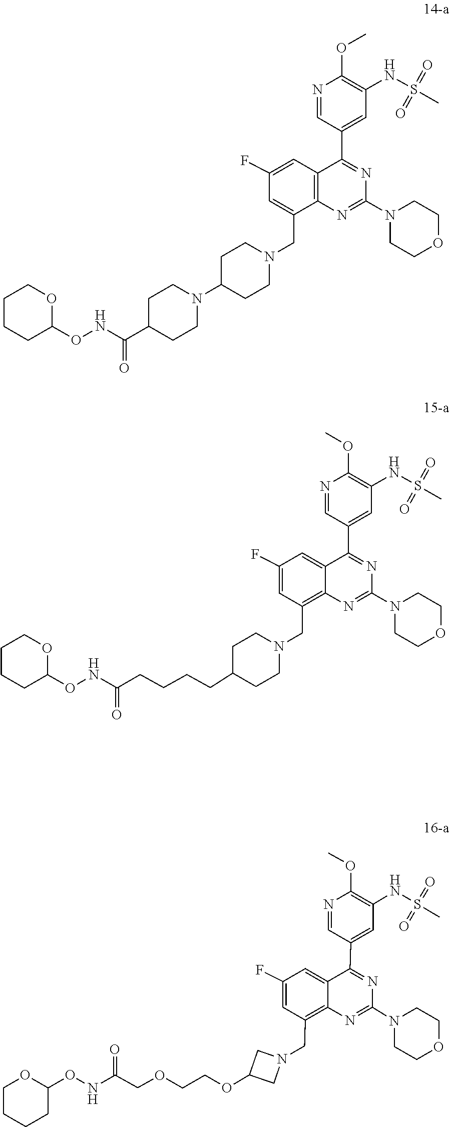 [SMILES: COc1ncc(-c2nc(N3CCOCC3)nc3c(CN4CCC(N5CCC(C(=O)NOC6CCCCO6)CC5)CC4)cc(F)cc23)cc1NS(C)(=O)=O ; COc1ncc(-c2nc(N3CCOCC3)nc3c(CN4CCC(CCCCC(=O)NOC5CCCCO5)CC4)cc(F)cc23)cc1NS(C)(=O)=O ; COc1ncc(-c2nc(N3CCOCC3)nc3c(CN4CC(OCCOCC(=O)NOC5CCCCO5)C4)cc(F)cc23)cc1NS(C)(=O)=O]

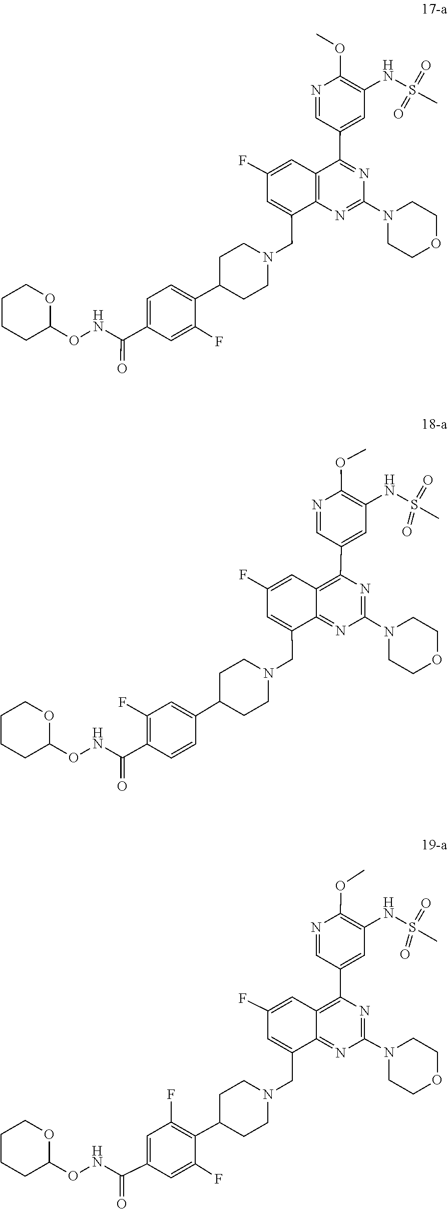 [SMILES: COc1ncc(-c2nc(N3CCOCC3)nc3c(CN4CCC(c5ccc(C(=O)NOC6CCCCO6)cc5F)CC4)cc(F)cc23)cc1NS(C)(=O)=O ; COc1ncc(-c2nc(N3CCOCC3)nc3c(CN4CCC(c5ccc(C(=O)NOC6CCCCO6)c(F)c5)CC4)cc(F)cc23)cc1NS(C)(=O)=O ; COc1ncc(-c2nc(N3CCOCC3)nc3c(CN4CCC(c5c(F)cc(C(=O)NOC6CCCCO6)cc5F)CC4)cc(F)cc23)cc1NS(C)(=O)=O]

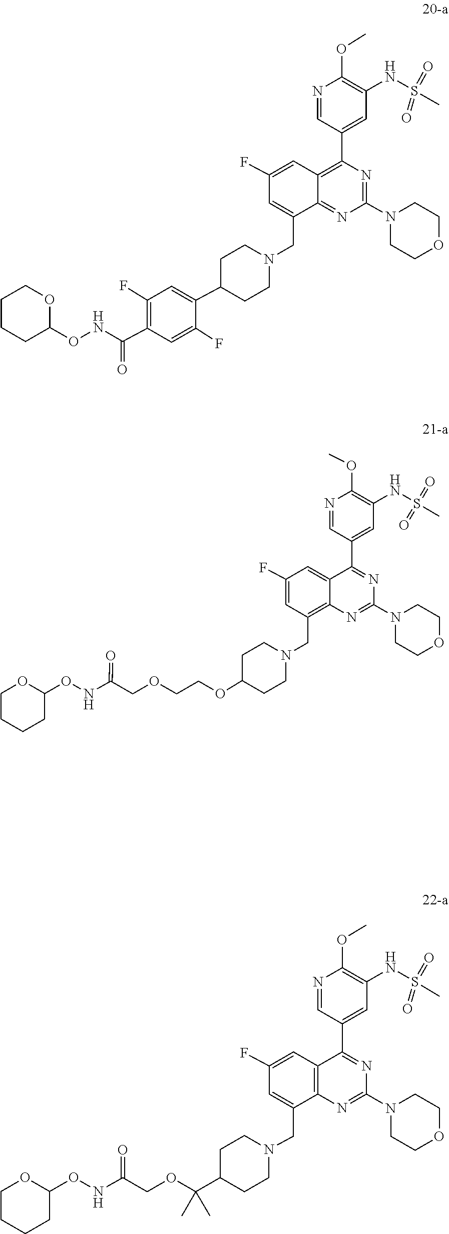 [SMILES: COc1ncc(-c2nc(N3CCOCC3)nc3c(CN4CCC(c5cc(F)c(C(=O)NOC6CCCCO6)cc5F)CC4)cc(F)cc23)cc1NS(C)(=O)=O ; COc1ncc(-c2nc(N3CCOCC3)nc3c(CN4CCC(OCCOCC(=O)NOC5CCCCO5)CC4)cc(F)cc23)cc1NS(C)(=O)=O ; COc1ncc(-c2nc(N3CCOCC3)nc3c(CN4CCC(C(C)(C)OCC(=O)NOC5CCCCO5)CC4)cc(F)cc23)cc1NS(C)(=O)=O]

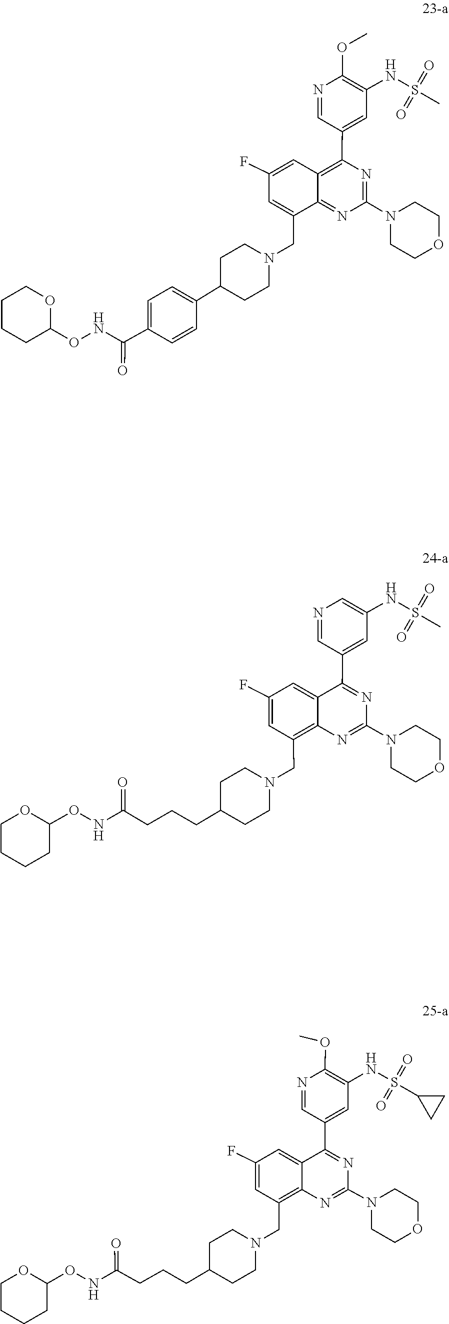 [SMILES: COc1ncc(-c2nc(N3CCOCC3)nc3c(CN4CCC(c5ccc(C(=O)NOC6CCCCO6)cc5)CC4)cc(F)cc23)cc1NS(C)(=O)=O ; CS(=O)(=O)Nc1cncc(-c2nc(N3CCOCC3)nc3c(CN4CCC(CCCC(=O)NOC5CCCCO5)CC4)cc(F)cc23)c1 ; COc1ncc(-c2nc(N3CCOCC3)nc3c(CN4CCC(CCCC(=O)NOC5CCCCO5)CC4)cc(F)cc23)cc1NS(=O)(=O)C1CC1]

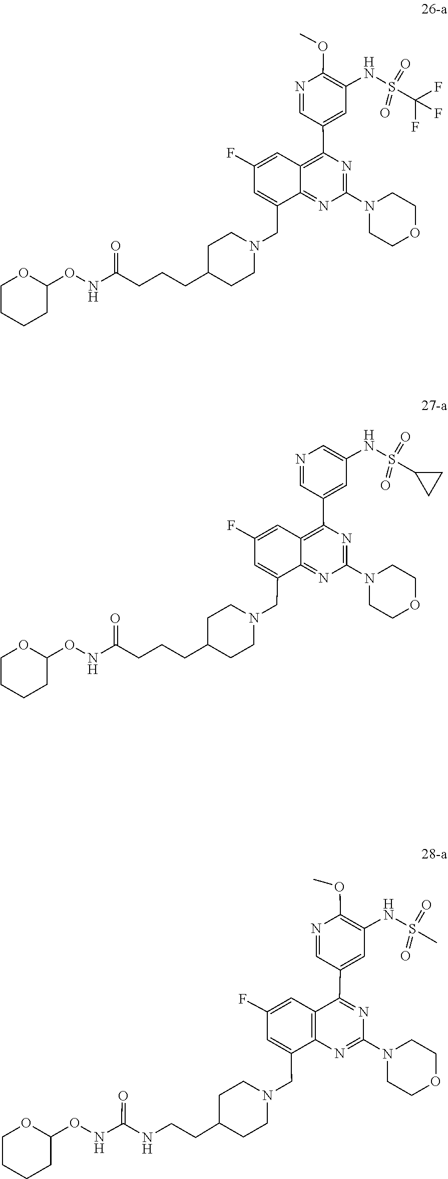 [SMILES: COc1ncc(-c2nc(N3CCOCC3)nc3c(CN4CCC(CCCC(=O)NOC5CCCCO5)CC4)cc(F)cc23)cc1NS(=O)(=O)C(F)(F)F ; O=C(CCCC1CCN(Cc2cc(F)cc3c(-c4cncc(NS(=O)(=O)C5CC5)c4)nc(N4CCOCC4)nc23)CC1)NOC1CCCCO1 ; COc1ncc(-c2nc(N3CCOCC3)nc3c(CN4CCC(CCNC(=O)NOC5CCCCO5)CC4)cc(F)cc23)cc1NS(C)(=O)=O]

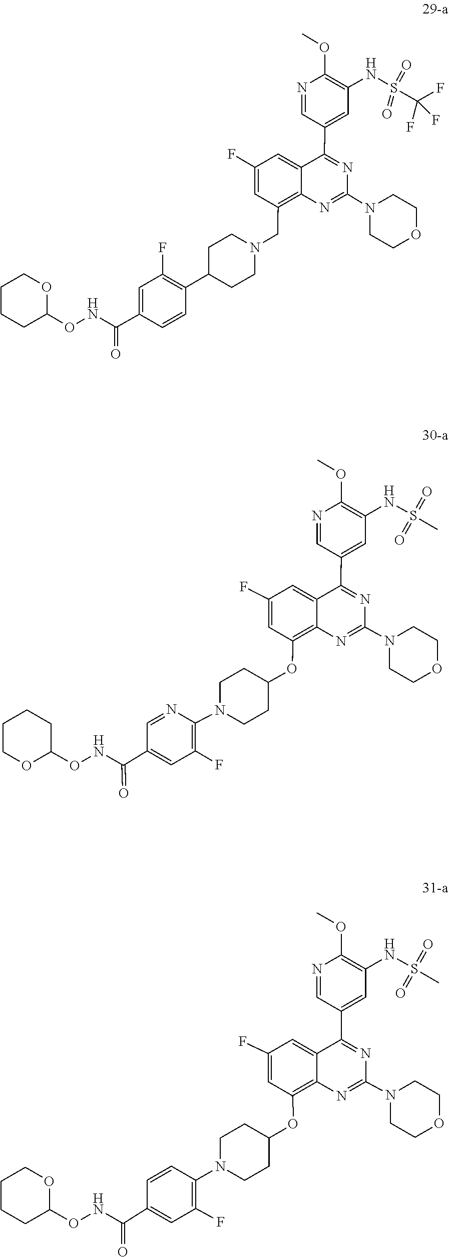 [SMILES: COc1ncc(-c2nc(N3CCOCC3)nc3c(CN4CCC(c5ccc(C(=O)NOC6CCCCO6)cc5F)CC4)cc(F)cc23)cc1NS(=O)(=O)C(F)(F)F ; COc1ncc(-c2nc(N3CCOCC3)nc3c(OC4CCN(c5ncc(C(=O)NOC6CCCCO6)cc5F)CC4)cc(F)cc23)cc1NS(C)(=O)=O ; COc1ncc(-c2nc(N3CCOCC3)nc3c(OC4CCN(c5ccc(C(=O)NOC6CCCCO6)cc5F)CC4)cc(F)cc23)cc1NS(C)(=O)=O]

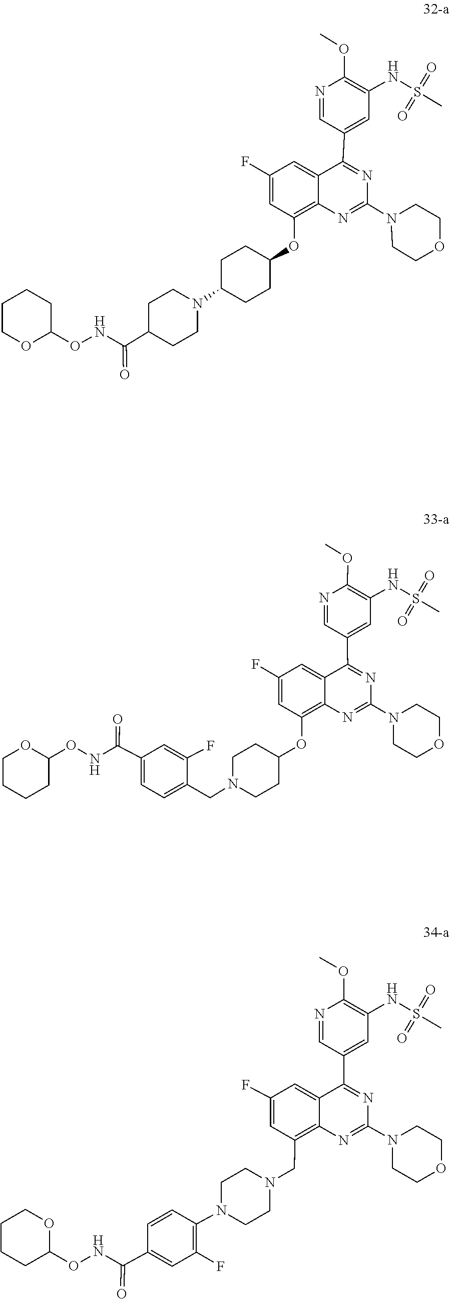 [SMILES: COc1ncc(-c2nc(N3CCOCC3)nc3c(O[C@H]4CC[C@H](N5CCC(C(=O)NOC6CCCCO6)CC5)CC4)cc(F)cc23)cc1NS(C)(=O)=O ; COc1ncc(-c2nc(N3CCOCC3)nc3c(OC4CCN(Cc5ccc(C(=O)NOC6CCCCO6)cc5F)CC4)cc(F)cc23)cc1NS(C)(=O)=O ; COc1ncc(-c2nc(N3CCOCC3)nc3c(CN4CCN(c5ccc(C(=O)NOC6CCCCO6)cc5F)CC4)cc(F)cc23)cc1NS(C)(=O)=O]

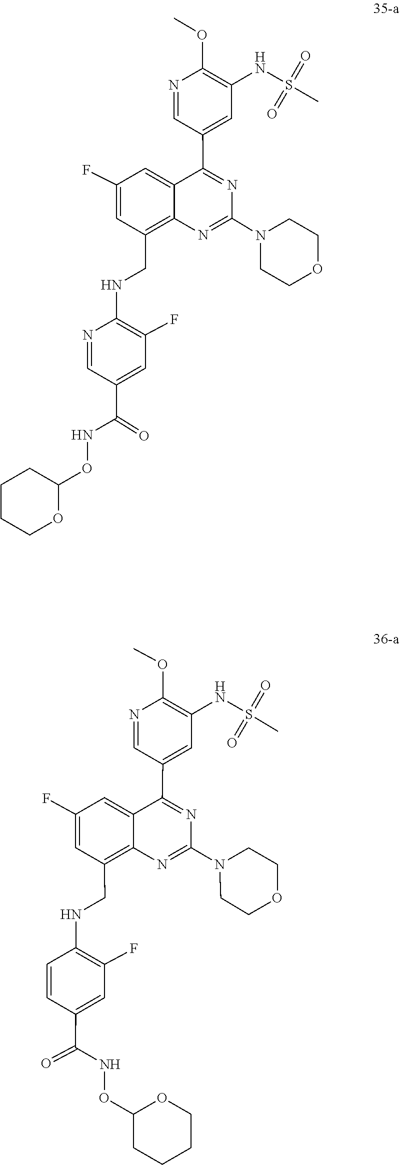 [SMILES: COc1ncc(-c2nc(N3CCOCC3)nc3c(CNc4ncc(C(=O)NOC5CCCCO5)cc4F)cc(F)cc23)cc1NS(C)(=O)=O ; COc1ncc(-c2nc(N3CCOCC3)nc3c(CNc4ccc(C(=O)NOC5CCCCO5)cc4F)cc(F)cc23)cc1NS(C)(=O)=O]

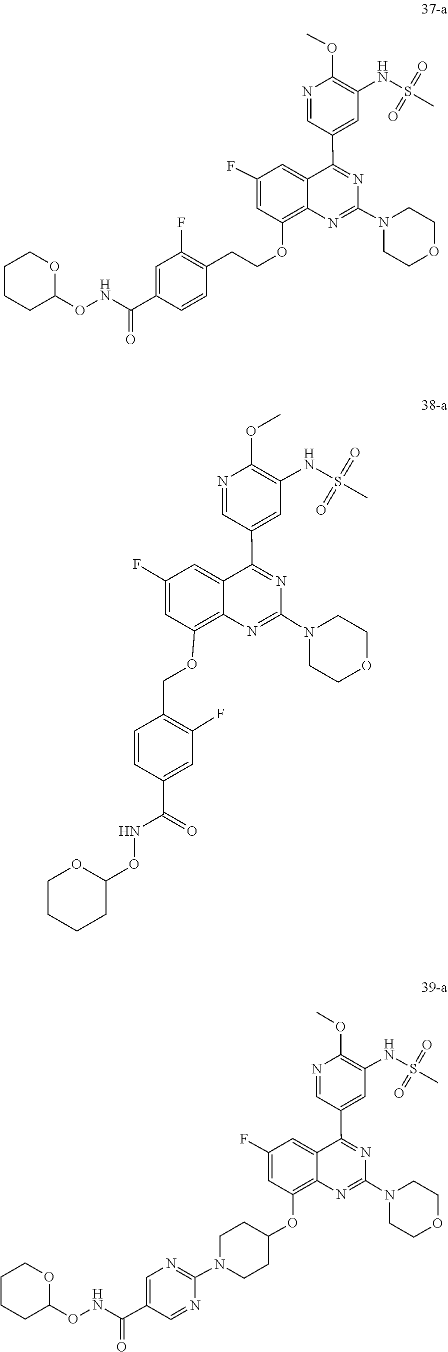 [SMILES: COc1ncc(-c2nc(N3CCOCC3)nc3c(OCCc4ccc(C(=O)NOC5CCCCO5)cc4F)cc(F)cc23)cc1NS(C)(=O)=O ; COc1ncc(-c2nc(N3CCOCC3)nc3c(OCc4ccc(C(=O)NOC5CCCCO5)cc4F)cc(F)cc23)cc1NS(C)(=O)=O ; COc1ncc(-c2nc(N3CCOCC3)nc3c(OC4CCN(c5ncc(C(=O)NOC6CCCCO6)cn5)CC4)cc(F)cc23)cc1NS(C)(=O)=O]

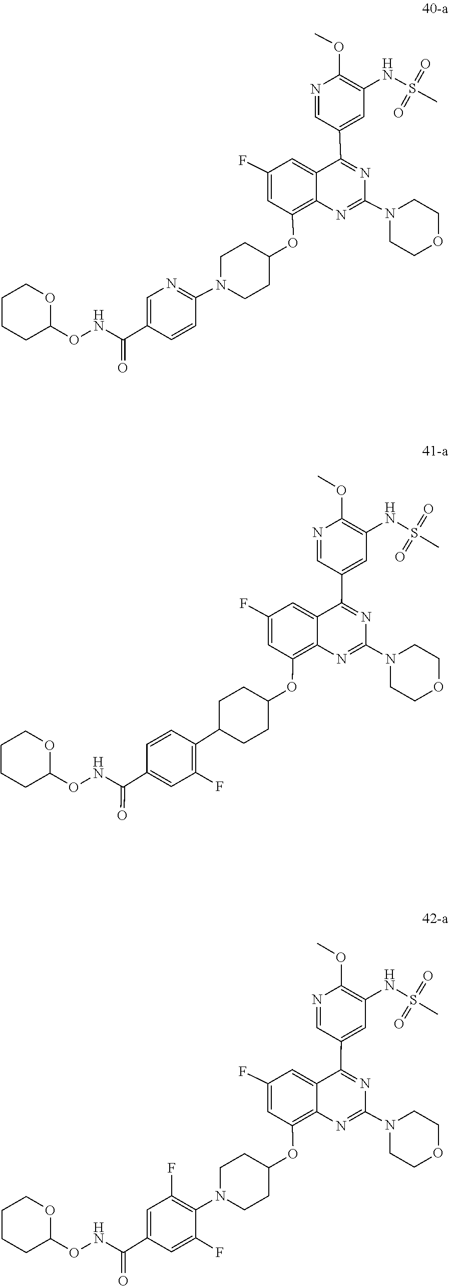 [SMILES: COc1ncc(-c2nc(N3CCOCC3)nc3c(OC4CCN(c5ccc(C(=O)NOC6CCCCO6)cn5)CC4)cc(F)cc23)cc1NS(C)(=O)=O ; COc1ncc(-c2nc(N3CCOCC3)nc3c(OC4CCC(c5ccc(C(=O)NOC6CCCCO6)cc5F)CC4)cc(F)cc23)cc1NS(C)(=O)=O ; COc1ncc(-c2nc(N3CCOCC3)nc3c(OC4CCN(c5c(F)cc(C(=O)NOC6CCCCO6)cc5F)CC4)cc(F)cc23)cc1NS(C)(=O)=O]

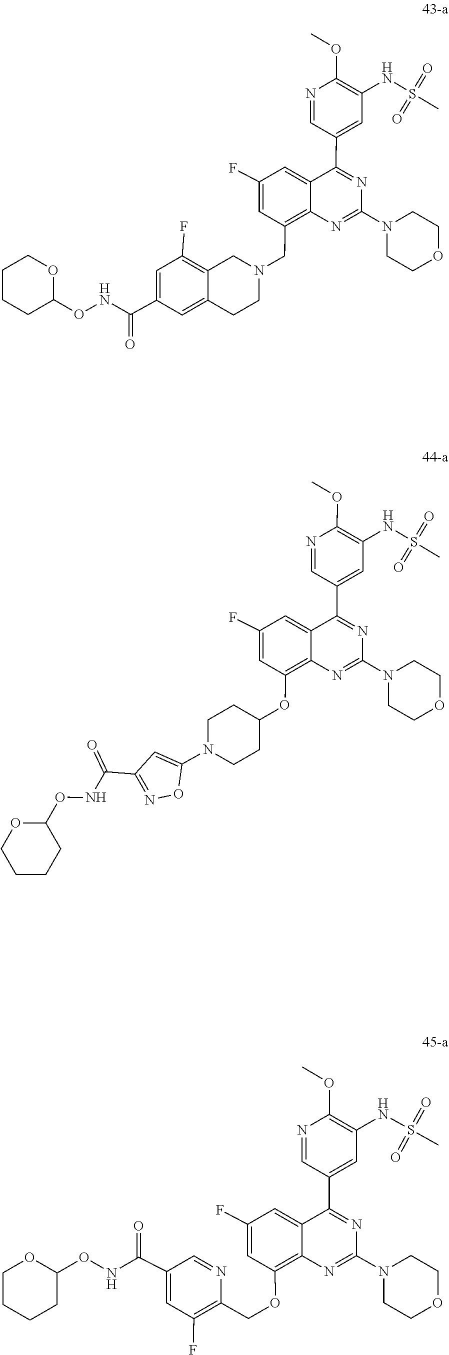 [SMILES: COc1ncc(-c2nc(N3CCOCC3)nc3c(CN4CCc5cc(C(=O)NOC6CCCCO6)cc(F)c5C4)cc(F)cc23)cc1NS(C)(=O)=O ; COc1ncc(-c2nc(N3CCOCC3)nc3c(OC4CCN(c5cc(C(=O)NOC6CCCCO6)no5)CC4)cc(F)cc23)cc1NS(C)(=O)=O ; COc1ncc(-c2nc(N3CCOCC3)nc3c(OCc4ncc(C(=O)NOC5CCCCO5)cc4F)cc(F)cc23)cc1NS(C)(=O)=O]

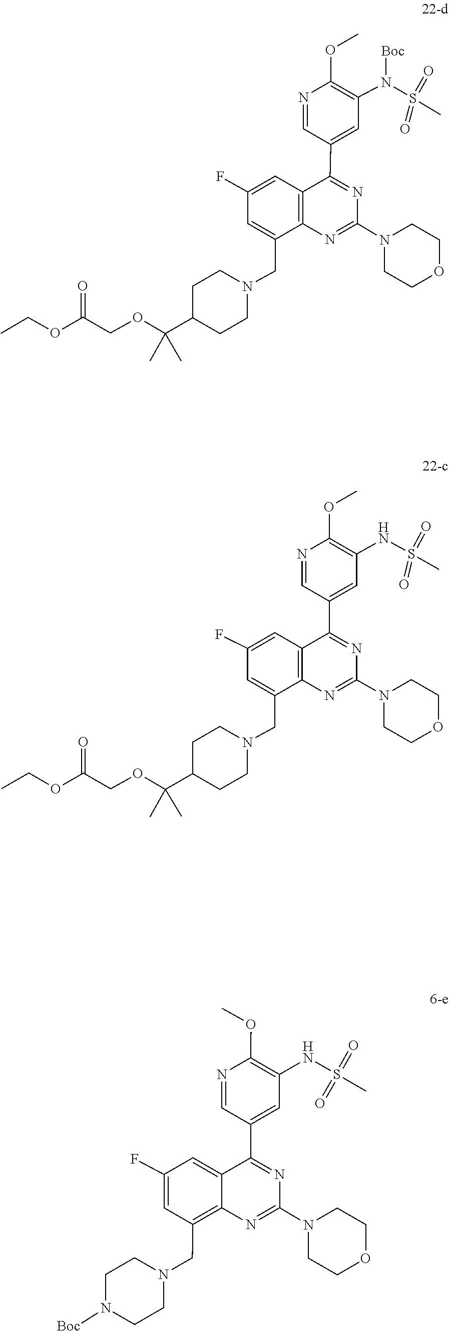

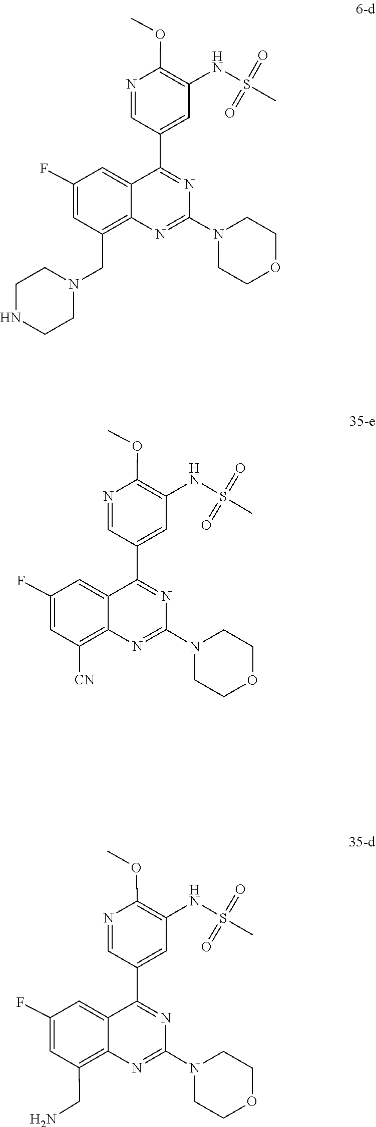 [SMILES: COc1ncc(-c2nc(N3CCOCC3)nc3c(CN4CCNCC4)cc(F)cc23)cc1NS(C)(=O)=O ; COc1ncc(-c2nc(N3CCOCC3)nc3c(C#N)cc(F)cc23)cc1NS(C)(=O)=O ; COc1ncc(-c2nc(N3CCOCC3)nc3c(CN)cc(F)cc23)cc1NS(C)(=O)=O]

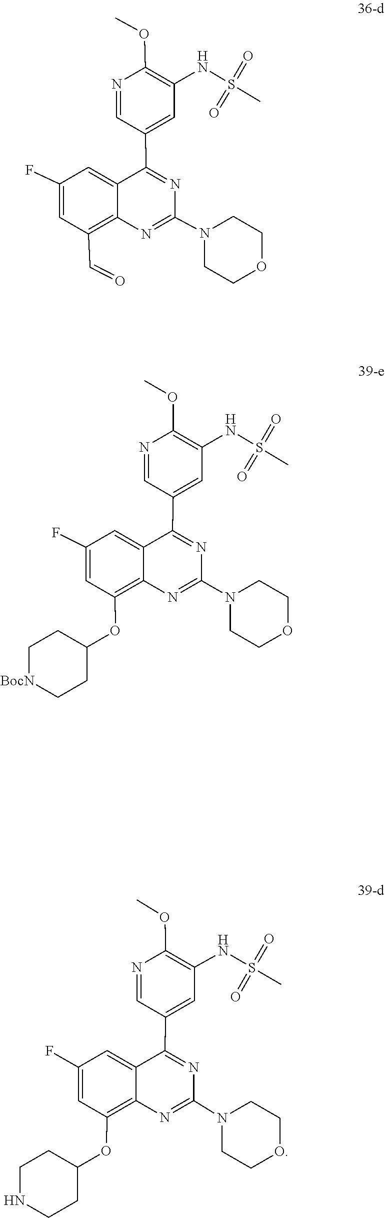 [SMILES: COc1ncc(-c2nc(N3CCOCC3)nc3c(C=O)cc(F)cc23)cc1NS(C)(=O)=O ; COc1ncc(-c2nc(N3CCOCC3)nc3c(OC4CCN(C(=O)OC(C)(C)C)CC4)cc(F)cc23)cc1NS(C)(=O)=O ; COc1ncc(-c2nc(N3CCOCC3)nc3c(OC4CCNCC4)cc(F)cc23)cc1NS(C)(=O)=O]

11. A pharmaceutical composition comprising a substance A and a pharmaceutically acceptable excipient, wherein the substance A is a therapeutically effective amount of the morpholinylquinazoline compound of formula I, the pharmaceutically acceptable salt thereof, the solvate thereof, the solvate of the pharmaceutically acceptable salt thereof, the polycrystalline form thereof or the precursor thereof according to claim 1.

12. A method for preventing or treating disease involving abnormal cell proliferation, differentiation or survival, comprising: administering an effective amount of a substance A to a subject, wherein the substance A is the morpholinylquinazoline compound of formula I, the pharmaceutically acceptable salt thereof, the solvate thereof, the solvate of the pharmaceutically acceptable salt thereof, the polycrystalline form thereof or the precursor thereof according to claim 1; the disease is cancer; The cancer is preferably caused by the proliferation of malignant neoplastic cells, such as tumors, flab, scarcoma, leukemia or lymphoma.

13. A method for reducing the number of lymphocytes circulating in a subject, comprising: administering an effective amount of a substance A, wherein the substance A is the morpholinylquinazoline compound of formula I, the pharmaceutically acceptable salt thereof, the solvate thereof, the solvate of the pharmaceutically acceptable salt thereof, the polycrystalline form thereof or the precursor thereof according to claim 1: The subject preferably is subject with hematologic underlying conditions, subject with autoimmune diseases, and subject requiring modulation of the immune response.
